(19) United States
(12) Patent Application Publication
(10) Pub. No.: US 2005/0042674 A9
(43) Pub. Date: Feb. 24, 2005

(54) Common ligand mimics: thiazolidinediones and rhodanines

(76) Inventors: Lin Yu, San Diego, CA (US); Qing Dong, San Diego, CA (US); Fabrice Pierre, La Jolla, CA (US); Edcon Chang, San Diego, CA (US); Hengyuan Lang, San Diego, CA (US); Yong Qin, Poway, CA (US); Yunfeng Fang, San Diego, CA (US); Mark Hansen, San Diego, CA (US); Maurizio Pellecchia, San Diego, CA (US)

Correspondence Address: CAMPBELL & FLORES LLP, 4370 LA JOLLA VILLAGE DRIVE, 7TH FLOOR, SAN DIEGO, CA, 92122, US

(21) Appl. No.: 10/081,989

(22) Filed: Feb. 21, 2002

(51) Int. Cl. G01N 33/53; C07D 413/02; C07K 5/06; G01N 33/543

(52) U.S. Cl. 435/7.1; 530/331; 548/183; 436/518

(57) ABSTRACT

The present invention provides common ligand mimics that act as common ligands for a receptor family. The present invention also provides bi-ligands containing these common ligand mimics. Bi-ligands of the invention provide enhanced affinity and/or selectivity of ligand binding to a receptor or receptor family through the synergistic action of the common ligand mimic and specificity ligand which compose the bi-ligand. The present invention also provides combinatorial libraries containing the common ligand mimics and bi-ligands of the invention. Further, the present invention provides methods for manufacturing the common ligand mimics and bi-ligands of the invention and methods for assaying the combinatorial libraries of the invention.

### BACKGROUND OF THE INVENTION

1. Field of the Invention

The present invention relates generally to receptor/ligand interactions and to combinatorial libraries of ligand compounds. The present invention also relates to the manufacture of thiazolidinediones and rhodanines and combinatorial libraries containing such compounds.

2. Background Information

Two general approaches have traditionally been used for drug discovery: screening for lead compounds and structure-based drug design. Both of these approaches are laborious and time-consuming and often produce compounds that lack the desired affinity or specificity.

Screening for lead compounds involves generating a pool of candidate compounds, often using combinatorial chemistry approaches in which compounds are synthesized by combining chemical groups to generate a large number of diverse candidate compounds that bind to the target or that inhibit binding to the target. The candidate compounds are screened with a drug target of interest to identify lead compounds that bind to the target or inhibit binding to the target. However, the screening process to identify a lead compound can be laborious and time consuming.

Structure-based drug design is an alternative approach to identifying drug candidates. Structure-based drug design uses three-dimensional structural data of the drug target as a template to model compounds that bind to the drug target and alter its activity. The compounds identified as potential drug candidates using structural modeling are used as lead compounds for the development of drug candidates that exhibit a desired activity toward the drug target.

Identifying compounds using structure-based drug design can be advantageous when compared to the screening approach in that modifications to the compound can often be predicted by modeling studies. However, obtaining structures of relevant drug targets and of drug targets complexed with test compounds is extremely time-consuming and laborious, often taking years to accomplish. The long time period required to obtain structural information useful for developing drug candidates is particularly limiting with regard to the growing number of newly discovered genes, which are potential drug targets, identified in genomics studies.

Despite the time-consuming and laborious nature of these approaches to drug discovery, both screening for lead compounds and structure-based drug design have led to the identification of a number of useful drugs, such as receptor agonists and antagonists. However, many of the drugs identified by these approaches have unwanted toxicity or side effects. Therefore, there is a need in the art for drugs that have high specificity and reduced toxicity. For example, in addition to binding to the drug target in a pathogenic organism or cancer cell, in some cases the drug also binds to an analogous protein in the patient being treated with the drug, which can result in toxic or unwanted side effects. Therefore, drugs that have high affinity and specificity for a target are particularly useful because administration of a more specific drug at lower dosages will minimize toxicity and side effects.

In addition to drug toxicity and side effects, a number of drugs that were previously highly effective for treating certain diseases have become less effective during prolonged clinical use due to the development of resistance. Drug resistance has become increasingly problematic, particularly with regard to administration of antibiotics. A number of pathogenic organisms have become resistant to several drugs due to prolonged clinical use and, in some cases, have become almost totally resistant to currently available drugs. Furthermore, certain types of cancer develop resistance to cancer therapeutic agents. Therefore, drugs that are refractile to the development of resistance would be particularly desirable for treatment of a variety of diseases.

One approach to developing such drugs is to find compounds that bind to a target protein such as a receptor or enzyme. When such a target protein has two adjacent binding sites, it is especially useful to find “bi-ligand” drugs that can bind at both sites simultaneously. However, the rapid identification of bi-ligand drugs having the optimum combination of affinity and specificity has been difficult. Bi-ligand drug candidates have been identified using rational drug design, but previous methods are time-consuming and require a precise knowledge of structural features of the receptor. Recent advances in nuclear magnetic spectroscopy (NMR) have allowed the determination of the three-dimensional interactions between a ligand and a receptor in a few instances. However, these efforts have been limited by the size of the receptor and can take years to map and analyze the complete structure of the complexes of receptor and ligand.

Thus, there exists a need for compounds that bind to multiple members of a receptor family. There is also a need for receptor bi-ligands containing such compounds coupled to ligands having a high specificity for the receptor.

There is a further need in the art for methods of preparing such compounds and bi-ligands. There is also a need in the art for methods of preparing combinatorial libraries of the bi-ligands and methods of screening these libraries to find bi-ligands that interact with a drug target with improved affinity and/or specificity. The present invention satisfies these needs and provides related advantages as well.

### SUMMARY OF THE INVENTION

The present invention provides compounds that function as mimics to a natural common ligand for a receptor family. These compounds interact with a conserved binding site on multiple receptors within the receptor family.

In one aspect, the present invention provides compounds that are common ligand mimics for NAD. NAD is a natural common ligand for many oxidoreductases. Thus, compounds of the invention that are common ligand mimics for NAD interact selectively with conserved sites on oxidoreductases.

In one embodiment, the present invention provides compounds of Formula I, 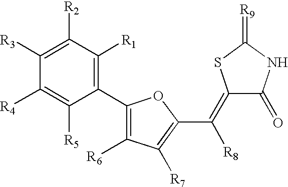
wherein R₁ to R₈ each independently are H, alkyl, alkenyl, alkynyl, aryl, heterocycle, COOH, COOAlkyl, CONR₁₀R₁₁, C(O)R₁₂, OH, OAlkyl, OAc, SH, SR₁₂, SO₃H, S(O)R₁₂, SO₂NR₁₀R₁₁, S(O)₂R₁₂, NH₂, NHR₁₂, NR₁₀R₁₁, NHCOR₁₂, NR₁₀COR₁₂, N₃, NO₂, PH₃, PH₂R₁₂, H₂PO₄, H₂PO₃, H₂PO₂, HPO₄R₁₂, PO₂R₁₁R₁₂, CN, or X. R₉ is an oxygen, sulfur, or nitrogen atom, where the nitrogen atom can be substituted, e.g. NR₁₂; and R₁₀, R₁₁, and R₁₂ each independently are hydrogen, alkyl, alkenyl, alkynyl, aryl, or heterocycle, or R₁₀ and R₁₁ together with the nitrogen to which they are attached can be joined to form a heterocyclic ring.

In another embodiment, the invention provides thiazolidinedione compounds of Formula II, 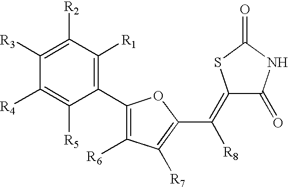
wherein R₁ to R₈ each independently are H, alkyl, alkenyl, alkynyl, aryl, heterocycle, COOH, COOAlkyl, CONR₁₀R₁₁, C(O)R₁₂, OH, OAlkyl, OAc, SH, SR₁₂, SO₃H, S(O)R₁₂, SO₂NR₁₀R₁₁, S(O)₂R₁₂, NH₂, NHR₁₂, NR₁₀R₁₁, NHCOR₁₂, NR₁₀COR₁₂, N₃, NO₂, PH₃, PH₂R₁₂, H₂PO₄, H₂PO₃, H₂PO₂, HPO₄R₁₂, PO₂R₁₁R₁₂, CN, or X, R₁₀, R₁₁, and R₁₂ each independently are hydrogen, alkyl, alkenyl, alkynyl, aryl, or heterocycle, or R₁₀ and R₁₁ together with the nitrogen to which they are attached can be joined to form a heterocyclic ring.

In still another embodiment, the invention provides rhodanine compounds of Formula III, 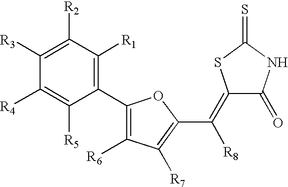
wherein R₁ to R₈ each independently are H, alkyl, alkenyl, alkynyl, aryl, heterocycle, COOH, COOAlkyl, CONR₁₀R₁₁, OH, OAlkyl, OAc, SH, SR₁₂, SO₃H, S(O)R₁₂, SO₂NR₁₀R₁₁, S(O)₂R₁₂, NH₂, NHR₁₂, NR₁₀R₁₁, NHCOR₁₂, NR₁₀COR₁₂, N₃, NO₂, PH₃, PH₂R₁₂, H₂PO₄, H₂PO₃, H₂PO₂, HPO₄R₁₂, PO₂R₁₁R₁₂, CN, or X. R₁₀, R₁₁, and R₁₂ each independently are hydrogen, alkyl, alkenyl, alkynyl, aryl, or heterocycle, or R₁₀ and R₁₁ together with the nitrogen to which they are attached can be joined to form a heterocyclic ring.

In a second aspect, the present invention provides methods for preparing compounds of Formula I. These methods generally comprise two steps. In the first step of each method, a furaldehyde intermediate is formed. In the second step, the furaldehyde intermediate is reacted either with 2,4-thiazolidinedione to form a compound of Formula II or with rhodanine to form a compound of Formula III.

In a third aspect, the present invention provides bi-ligands containing a common ligand mimic and a specificity ligand which interact with distinct sites on a receptor. In one embodiment, the present invention provides bi-ligands that are the reaction products of compounds of Formula I with specificity ligands. In another embodiment, the invention provides bi-ligands containing the reaction products of compounds of Formula II with specificity ligands. In yet another embodiment, the invention provides bi-ligands that are reaction products of compounds of Formula III and specificity ligands. In yet another aspect, the invention provides methods for preparing bi-ligands that are reaction products of the common ligand mimics of general Formulas I, II, and III and a pyridine dicarboxylate specificity ligand.

The present invention further provides combinatorial libraries containing one or more common ligand variants of the compounds of the invention. In one embodiment, the combinatorial libraries of the invention contain one or more common ligand variants of the compounds of Formula I. In other embodiments, the combinatorial libraries of the invention contain one or more common ligand variants of the compounds of Formula II or Formula III.

The present invention also provides combinatorial libraries comprised of one or more bi-ligands that are reaction products of common ligand mimics and specificity ligands. In one embodiment, such combinatorial libraries contain one or more bi-ligands that are the reaction product of compounds of Formula I and specificity ligands. In another embodiment, such combinatorial libraries contain one or more bi-ligands that are the reaction product of compounds of Formula II and specificity ligands. In still another embodiment, such combinatorial libraries contain one or more bi-ligands that are the reaction product of compounds of Formula III and specificity ligands.

The present invention also provides methods for producing and screening combinatorial libraries of bi-ligands for binding to a receptor and families of such receptors.

### BRIEF DESCRIPTION OF THE DRAWINGS

FIG. 1 shows Scheme 1 for the synthesis of thiazolidinedione compounds of Formula II where R₁ to R₈ each independently are H, alkyl, alkenyl, alkynyl, aryl, heterocycle, COOH, COOAlkyl, CONR₁₀R₁₁, C(O)R₁₂, OH, OAlkyl, OAc, SH, SR₁₂, SO₃H, S(O)R₁₂ SO₂NR₁₀R₁₁, S(O)₂R₁₂, NH₂, NHR₁₂, NR₁₀R₁₁, NHCOR₁₂, NR₁₀COR₁₂, N₃, NO₂, PH₃, PH₂R₁₂, H₂PO₄, H₂PO₃, H₂PO₂, HPO₄R₁₂, PO₂R₁₁R₁₂, CN, or X. R₁₀, R₁₁, and R₁₂ each independently are hydrogen, alkyl, alkenyl, alkynyl, aryl, or heterocycle, or R₁₀ and R₁₁ together with the nitrogen to which they are attached can be joined to form a heterocyclic ring. The reaction steps are as follows: (a) an aminobenzoic acid and 2-furaldehyde are reacted in the presence of HNO₂ and CuCl₂/CuCl to form a furaldehyde intermediate; (b) the furaldehyde intermediate is reacted with 2,4-thiazolidinedione, while heating, to form the corresponding thiazolidinedione.

FIG. 2 shows Scheme 1 for the synthesis of rhodanine compounds of Formula III where R₁ to R₈ each independently are H, alkyl, alkenyl, alkynyl, aryl, heterocycle, COOH, COOAlkyl, CONR₁₀R₁₁, C(O)R₁₂, OH, OAlkyl, OAc, SH, SR₁₂, SO₃H, S(O)R₁₂, SO₂NR₁₀R₁₁, S(O)₂R₁₂, NH₂, NHR₁₂, NR₁₀R₁₁, NHCOR₁₂, NR₁₀COR₁₂, N₃, NO₂, PH₃, PH₂R₁₂, H₂PO₄, H₂PO₃, H₂PO₂, HPO₄R₁₂, PO₂R₁₁R₁₂, CN, or X. R₁₀ R₁₁, and R₁₂ each independently are hydrogen, alkyl, alkenyl, alkynyl, aryl, or heterocycle, or R₁₀ and R₁₁ together with the nitrogen to which they are attached can be joined to form a heterocyclic ring. The reaction steps are as follows: (a) an aminobenzoic acid and 2-furaldehyde are reacted in the presence of HNO₂ and CuCl₂/CuCl to form a furaldehyde intermediate; (b) the furaldehyde intermediate is reacted with rhodanine, while heating, to form the corresponding rhodanine compound.

FIG. 3 shows Scheme 2 for the synthesis of thiazolidinedione compounds of Formula II where R₁ to R₈ each independently are H, alkyl, alkenyl, alkynyl, aryl, heterocycle, COOH, COOAlkyl, CONR₁₀R₁₁, C(O)R₁₂, OH, OAlkyl, OAc, SH, SR₁₂, SO₃H, S(O)R₁₂, SO₂NR₁₀R₁₁, S(O)₂R₁₂, NH₂, NHR₁₂, NR₁₀R₁₁, NHCOR₁₂, NR₁₀COR₁₂, N₃, NO₂, PH₃, PH₂R₁₂, H₂PO₄, H₂PO₃, H₂PO₂, HPO₄R₁₂, PO₂R₁₁R₁₂, CN, or X. R₁₀, R₁₁, and R₁₂ each independently are hydrogen, alkyl, alkenyl, alkynyl, aryl, or heterocycle, or R₁₀ and R₁₁, together with the nitrogen to which they are attached can be joined to form a heterocyclic ring. The reaction steps are as follows: (a) a halobenzoate and 5-trimethylstannanyl-furan-2-carbaldehyde are reacted in the presence of Pd(PPh₃)₄ to form a furaldehyde intermediate; (b) the furaldehyde intermediate is reacted with 2,4-thiazolidinedione while heating, to form the corresponding thiazolidinedione.

FIG. 4 shows Scheme 2 for the synthesis of rhodanine compounds of Formula III where R₁ to R₈ each independently are H, alkyl, alkenyl, alkynyl, aryl, heterocycle, COOH, COOAlkyl, CONR₁₀R₁₁, C(O)R₁₂, OH, OAlkyl, OAc, SH, SR₁₂, SO₃H, S(O)R₁₂, SO₂NR₁₀R₁₁, S(O)₂R₁₂, NH₂, NHR₁₂, NR₁₀R₁₁, NHCOR₁₂, NR₁₀COR₁₂, N₃, NO₂, PH₃, PH₂R₁₂, H₂PO₄, H₂PO₃, H₂PO₂, HPO₄R₁₂, PO₂R₁₁R₁₂, CN, or X. R₁₀ R₁₁, and R₁₂ each independently are hydrogen, alkyl, alkenyl, alkynyl, aryl, or heterocycle, or R₁₀ and R₁₁ together with the nitrogen to which they are attached can be joined to form a heterocyclic ring. The reaction steps are as follows: (a) a halobenzoate and 5-trimethylstannanyl-furan-2-carbaldehyde are reacted in the presence of Pd(PPh₃)₄ to form a furaldehyde intermediate; (b) the furaldehyde intermediate is reacted with rhodanine, while heating, to form the corresponding rhodanine compound.

FIG. 5 shows Scheme 3 for the synthesis of thiazolidinedione compounds of Formula II where R₁ to R₈ each independently are H, alkyl, alkenyl, alkynyl, aryl, heterocycle, COOH, COOAlkyl, CONR₁₀R₁₁, C(O)R₁₂, OH, OAlkyl, OAc, SH, SR₁₂, SO₃H, S(O)R₁₂, SO₂NR₁₀R₁₁, S(O)₂R₁₂, NH₂, NHR₁₂, NR₁₀R₁₁, NHCOR₁₂, NR₁₀COR₁₂, N₃, NO₂, PH₃, PH₂R₁₂, H₂PO₄, H₂PO₃, H₂PO₂, HPO₄R₁₂, PO₂R₁₁R₁₂, CN, or X. R₁₀, R₁₁, and R₁₂ each independently are hydrogen, alkyl, alkenyl, alkynyl, aryl, or heterocycle, or R₁₀ and R₁₁, together with the nitrogen to which they are attached can be joined to form a heterocyclic ring. The reaction steps are as follows: (a) a halofuraldehyde and phenylboronic acid are reacted in the presence of Pd(PPh₃)₄ to form a furaldehyde intermediate; (b) the furaldehyde intermediate is reacted with 2,4-thiazolidinedione, while heating, to form the corresponding thiazolidinedione.

FIG. 6 shows Scheme 3 for the synthesis of rhodanine compounds of Formula III where R₁ to R₈ each independently are H, alkyl, alkenyl, alkynyl, aryl, heterocycle, COOH, COOAlkyl, CONR₁₀R₁₁, C(O)R₁₂, OH, OAlkyl, OAc, SH, SR₁₂, SO₃H, S(O)R₁₂, SO₂NR₁₀R₁₁, S(O)₂R₁₂, NH₂, NHR₁₂, NR₁₀R₁₁, NHCOR₁₂, NR₁₀COR₁₂, N₃, NO₂, PH₃, PH₂R₁₂, H₂PO₄, H₂PO₃, H₂PO₂, HPO₄R₁₂, PO₂R₁₁R₁₂, CN, or X. R₁₀, R₁₁, and R₁₂ each independently are hydrogen, alkyl, alkenyl, alkynyl, aryl, or heterocycle, or R₁₀ and R₁₁ together with the nitrogen to which they are attached can be joined to form a heterocyclic ring. The reaction steps are as follows: (a) a halofuraldehyde and phenylboronic acid are reacted in the presence of Pd(PPh₃)₄ to form a furaldehyde intermediate; (b) the furaldehyde intermediate is reacted with rhodanine, while heating, to form the corresponding rhodanine compound.

FIG. 7 shows Scheme 4 for modification of substituents attached to the common ligand mimics of the invention.

FIG. 8 shows Scheme 5 for modification of substituents attached to the common ligand mimics of the invention.

FIG. 9 shows Scheme 6 for modification of substituents attached to the common ligand mimics of the invention.

FIG. 10 shows Scheme 7 for the preparation of common ligand mimics of the present invention containing linker molecules.

FIG. 11 shows Scheme 8 for the preparation of common ligand mimics of the present invention containing linker molecules.

FIGS. 12a-c show various reaction schemes by which combinatorial libraries of the present invention can be made. FIG. 12a shows the reaction scheme for reaction of common ligand mimics of the present invention having a carboxylic acid group with an amine in the presence of hydroxybenzotriazole (HOBt). FIG. 12b shows the reaction of common ligand mimics of the invention having an amine terminal amide substituent with a carboxylic acid in the presence of HOBt. FIG. 12c shows the reaction scheme for reaction of common ligand mimics of the invention having an amine terminal amide substituent with an isocyanate or thioisocyanate.

FIG. 13 shows a reaction scheme by which combinatorial libraries of the present invention can be made employing amines. The reaction steps are as follows: (a) reacting a halopyridine with a thiol in the presence of DBU under microwave irradiation to form a thiopyridine; (b) reacting the thiopyridine with LiOH to free the acid group; (c) adding diverse elements to the resulting acid through formation of an amide bond, catalyzed by HOBt resin; (d) treating the amide with TFA in DCE to remove the Boc-protecting group; and (e) reacting the pyridine derivative with a common ligand mimic of the invention to yield bi-ligand libraries of the invention.

FIG. 14 shows a reaction scheme by which combinatorial libraries of the present invention can be made employing alkyl halides. The reaction steps are as follows: (a) mixing 4-mercaptobenzoic acid and an alkylhalide in CH₃CN; (b) adding Et₃N resin to the mixture; (c) reacting the product of step (b) with HOBt resin; and (d) adding a common ligand mimic of the present invention.

FIG. 15 shows Scheme 9 for the synthesis of bi-ligands containing thiazolidinedione common ligand mimics and pyridine dicarboxylate specificity ligands.

FIG. 16 shows the results of an oxidoreductase enzymatic panel study of selected thiazolidinedione compounds of the invention.

FIG. 17 shows the results of an enzymatic panel study of selected thiazolidinedione compounds of the invention.

FIG. 18 shows the results of an oxidoreductase assay of selected bi-ligands of the invention.

FIGS. 19a-c show the names and corresponding structures for exemplified thiazolidinedione and rhodanine common ligand mimics of the invention.

FIG. 20 shows examples of bi-ligands of the invention.

### DETAILED DESCRIPTION OF THE INVENTION

The present invention is directed to bi-ligands and the development of combinatorial libraries associated with these bi-ligands. The invention can be used advantageously to develop bi-ligands that bind to two distinct sites on a receptor, a common site and a specificity site. Tailoring of the two portions of the bi-ligand provides optimal binding characteristics. These optimal binding characteristics provide increased diversity within a library, while simultaneously focusing the library on a particular receptor family or a particular member of a receptor family. The two portions of the bi-ligand, a common ligand mimic and a specificity ligand act synergistically to provide higher affinity and/or specificity than either ligand alone.

The technology of the present invention can be applied across receptor families or can be used to screen for specific members of a family. For example, the present invention can be used to screen libraries for common ligand mimics that bind to any oxidoreductase. Alternatively, the present invention can be used to screen for a particular oxidoreductase that will bind a particular specificity ligand.

The present invention provides common ligand mimics that bind selectively to a conserved site on a receptor. The compounds advantageously can be used to develop combinatorial libraries of bi-ligands more efficiently than conventional methods. The present invention takes advantage of NMR spectroscopy to identify the interactions between the common ligand mimic and the receptor, which allows for improved tailoring of the ligand to the receptor.

The present invention also provides bi-ligands containing these common ligand mimics. The bi-ligands of the invention contain a common ligand mimic coupled to a specificity ligand. These bi-ligands provide the ability to tailor the affinity and/or specificity of the ligands to the binding sites on the receptor.

The present invention further provides combinatorial libraries containing bi-ligands of the invention as well as formation of such libraries from the common ligand mimics of the invention. These libraries provide an enhanced number of bi-ligands that bind multiple members of a receptor family than is provided with standard combinatorial techniques due to specific positioning of the specificity ligand on the common ligand mimic. Optimal positioning of the specificity ligand can be determined through NMR studies of the receptor and the common ligand mimic to be employed.

The present invention also provides methods for the preparation of two categories of common ligand mimics useful in the present invention and methods for the preparation of bi-ligands containing these common ligand mimics. In general, such methods involve formation of a furaldehyde intermediate followed by reaction of the intermediate with 2,4-thiazolidinedione or rhodanine. The present invention also provides methods for modification of the common ligand mimics to form additional common ligand mimics having different bi-ligand directing/binding substituents to yield enhanced specificity and potency. The common ligand mimics can be used to create bi-ligands having improved affinity, improved specificity, or both. These and other aspects of the invention are described below.

The present invention provides common ligand mimics. As used herein, the term “ligand” refers to a molecule that can selectively bind to a receptor. The term “selectively” means that the binding interaction is detectable over non-specific interactions as measured by a quantifiable assay. A ligand can be essentially any type of molecule such as an amino acid, peptide, polypeptide, nucleic acid, carbohydrate, lipid, or small organic compound. The term ligand refers both to a molecule capable of binding to a receptor and to a portion of such a molecule, if that portion of a molecule is capable of binding to a receptor. For example, a bi-ligand, which contains a common ligand and specificity ligand, is considered a ligand, as would the common ligand and specificity ligand portions since they can bind to a conserved site and specificity site, respectively. As used herein, the term “ligand” excludes a single atom, for example, a metal atom. Derivatives, analogues, and mimetic compounds also are included within the definition of this term. These derivatives, analogues and mimetic compounds include those containing metals or other inorganic molecules, so long as the metal or inorganic molecule is covalently attached to the ligand in such a manner that the dissociation constant of the metal from the ligand is less than 10⁻¹⁴ M. A ligand can be multi-partite, comprising multiple ligands capable of binding to different sites on one or more receptors, such as a bi-ligand. The ligand components of a multi-partite ligand can be joined together directly, for example, through functional groups on the individual ligand components or can be joined together indirectly, for example, through an expansion linker.

As used herein, the term “common ligand” refers to a ligand that binds to a conserved site on receptors in a receptor family. A “natural common ligand” refers to a ligand that is found in nature and binds to a common site on receptors in a receptor family. As used herein, a “common ligand mimic (CLM)” refers to a common ligand that has structural and/or functional similarities to a natural common ligand but is not naturally occurring. Thus, a common ligand mimic can be a modified natural common ligand, for example, an analogue or derivative of a natural common ligand. A common ligand mimic also can be a synthetic compound or a portion of a synthetic compound that is structurally similar to a natural common ligand.

As used herein, a “common ligand variant” refers to a derivative of a common ligand. A common ligand variant has structural and/or functional similarities to a parent common ligand. A common ligand variant differs from another variant, including the parent common ligand, by at least one atom. For example, as with NAD and NADH, the reduced and oxidized forms differ by an atom and are therefore considered to be variants of each other. A common ligand variant includes reactive forms of a common ligand mimic, such as an anion or cation of the common ligand mimic. As used herein, the term “reactive form” refers to a form of a compound that can react with another compound to form a chemical bond, such as an ionic or covalent bond. For example, where the common ligand mimic is an acid of the form ROOH or an ester of the form ROOR′, the common ligand variant can be ROO⁻.

As used herein, the term “conserved site” on a receptor refers to a site that has structural and/or functional characteristics common to members of a receptor family. A conserved site contains amino acid residues sufficient for activity and/or function of the receptor that are accessible to binding of a natural common ligand. For example, the amino acid residues sufficient for activity and/or function of a receptor that is an enzyme can be amino acid residues in a substrate binding site of the enzyme. Also, the conserved site in an enzyme that binds a cofactor or coenzyme can be amino acid residues that bind the cofactor or coenzyme.

As used herein, the term “receptor” refers to a polypeptide that is capable of selectively binding a ligand. The function or activity of a receptor can be enzymatic activity or ligand binding. Receptors can include, for example, enzymes such as kinases, dehydrogenases, oxidoreductases, GTPases, carboxyl transferases, acyl transferases, decarboxylases, transaminases, racemases, methyl transferases, formyl transferases, and α-ketodecarboxylases.

Furthermore, the receptor can be a functional fragment or modified form of the entire polypeptide so long as the receptor exhibits selective binding to a ligand. A functional fragment of a receptor is a fragment exhibiting binding to a common ligand and a specificity ligand. As used herein, the term “enzyme” refers to a molecule that carries out a catalytic reaction by converting a substrate to a product.

Enzymes can be classified based on Enzyme Commission (EC) nomenclature recommended by the Nomenclature Committee of the International Union of Biochemistry and Molecular Biology (IUBMB)(see, for example, www.expasy.ch/sprot/enzyme.html)(which is incorporated herein by reference). For example, oxidoreductases are classified as oxidoreductases acting on the CH—OH group of donors with NAD⁺ or NADP⁺ as an acceptor (EC 1.1.1); oxidoreductases acting on the aldehyde or oxo group of donors with NAD⁺ or NADP⁺ as an acceptor (EC 1.2.1); oxidoreductases acting on the CH—CH group of donors with NAD⁺ or NADP⁺ as an acceptor (EC 1.3.1); oxidoreductases acting on the CH—NH₂ group of donors with NAD⁺ or NADP⁺ as an acceptor (EC 1.4.1); oxidoreductases acting on the CH—NH group of donors with NAD⁺ or NADP⁺ as an acceptor (EC 1.5.1); oxidoreductases acting on NADH or NADPH (EC 1.6); and oxidoreductases acting on NADH or NADPH with NAD⁺ or NADP⁺ as an acceptor (EC 1.6.1).

Additional oxidoreductases include oxidoreductases acting on a sulfur group of donors with NAD⁺ or NADP⁺ as an acceptor (EC 1.8.1); oxidoreductases acting on diphenols and related substances as donors with NAD⁺ or NADP⁺ as an acceptor (EC 1.10.1); oxidoreductases acting on hydrogen as donor with NAD⁺ or NADP⁺ as an acceptor (EC 1.12.1); oxidoreductases acting on paired donors with incorporation of molecular oxygen with NADH or NADPH as one donor and incorporation of two atoms (EC 1.14.12) and with NADH or NADPH as one donor and incorporation of one atom (EC 1.14.13); oxidoreductases oxidizing metal ions with NAD⁺ or NADP⁺ as an acceptor (EC 1.16.1); oxidoreductases acting on —CH₂ groups with NAD⁺ or NADP⁺ as an acceptor (EC 1.17.1); and oxidoreductases acting on reduced ferredoxin as donor, with NAD⁺ or NADP⁺ as an acceptor (EC 1.18.1).

Enzymes can also bind coenzymes or cofactors such as nicotinamide adenine dinucleotide (NAD) and nicotinamide adenine dinucleotide phosphate (NADP), thiamine pyrophosphate, flavin adenine dinucleotide (FAD) and flavin mononucleotide (FMN), pyridoxal phosphate, coenzyme A, and tetrahydrofolate or other cofactors or substrates such as ATP, GTP and S-adenosyl methionine (SAM). In addition, enzymes that bind newly identified cofactors or enzymes can also be receptors.

As used herein, the term “receptor family” refers to a group of two or more receptors that share a common, recognizable amino acid motif. A motif in a related family of receptors occurs because certain amino acid residues, or residues having similar chemical characteristics, are required for the structure, function and/or activity of the receptor and are, therefore, conserved between members of the receptor family. Methods of identifying related members of a receptor family are well known to those skilled in the art and include sequence alignment algorithms and identification of conserved patterns or motifs in a group of polypeptides, which are described in more detail below. Members of a receptor family also can be identified by determination of binding to a common ligand.

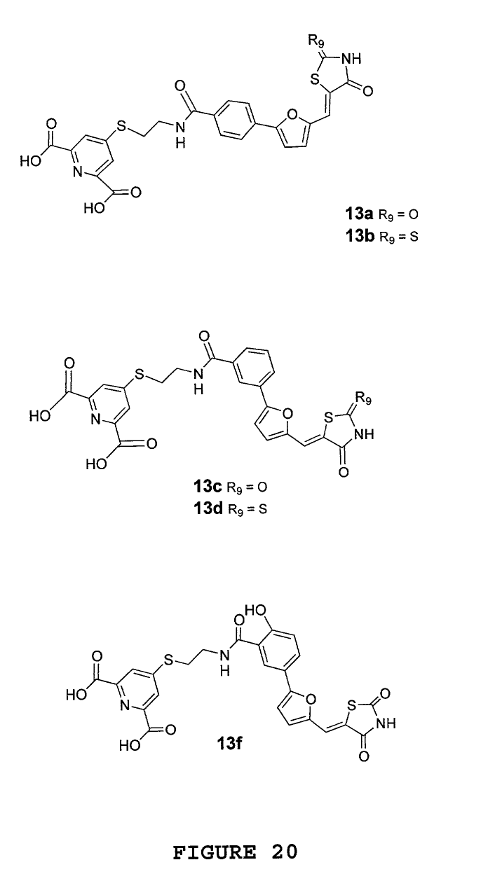

In another aspect, the present invention provides bi-ligands that contain a common ligand mimic as described above and a specificity ligand. As used herein, the term “bi-ligand” refers to a ligand comprising two ligands that bind to independent sites on a receptor. One of the ligands of a bi-ligand is a specificity ligand capable of binding to a site that is specific for a given member of a receptor family when joined to a common ligand. The second ligand of a bi-ligand is a common ligand mimic that binds to a conserved site in a receptor family. The common ligand mimic and specificity ligand are bonded together. Bonding of the two ligands can be direct or indirect, such as through a linking molecule or group. A depiction of exemplary bi-ligands is shown in FIG. 20.

As used herein the term “specificity” refers to the ability of a ligand to differentially bind to one receptor over another receptor in the same receptor family. The differential binding of a particular ligand to a receptor is measurably higher than the binding of the ligand to at least one other receptor in the same receptor family. A ligand having specificity for a receptor refers to a ligand exhibiting specific binding that is at least two-fold higher for one receptor over another receptor in the same receptor family.

As used herein, the term “specificity ligand” refers to a ligand that binds to a specificity site on a receptor. A specificity ligand can bind to a specificity site as an isolated molecule or can bind to a specificity site when attached to a common ligand, as in a bi-ligand. When a specificity ligand is part of a bi-ligand, the specificity ligand can bind to a specificity site that is proximal to a conserved site on a receptor.

As used herein, the term “specificity site” refers to a site on a receptor that provides the binding site for a ligand exhibiting specificity for a receptor. A specificity site on a receptor imparts molecular properties that distinguish the receptor from other receptors in the same receptor family. For example, if the receptor is an enzyme, the specificity site can be a substrate binding site that distinguishes two members of a receptor family which exhibit substrate specificity. A substrate specificity site can be exploited as a potential binding site for the identification of a ligand that has specificity for one receptor over another member of the same receptor family. A specificity site is distinct from the common ligand binding site in that the natural common ligand does not bind to the specificity site.

As used herein, the term “linker” refers to a chemical group that can be attached to either the common ligand or the specificity ligand of a bi-ligand. The linker provides the functional groups through which the common ligand mimic and specificity ligand are indirectly bound to one another. The linker can be a simple functional group, such as COOH, NH₂, OH, or the like. Alternatively, the linker can be a complex chemical group containing one or more unsaturation, one or more substituent, and/or one or more heterocyclic atom. Nonlimiting examples of complex linkers are depicted in Tables 6 to 12.

The present invention provides common ligand mimics that are common mimics of NAD and combinatorial libraries containing these common ligand mimics. For example, in one embodiment, compounds of the invention are ligands for conserved sites on dehydrogenases and reductases. Examples of such receptors include, but are not limited to, HMG CoA reductase (HMGCoAR), inosine-5′-monophosphate dehydrogenase (IMPDH), 1-deoxy-D-xylulose-5-phosphate reductase (DOXPR), dihydrodipicolinate reductase (DHPR), dihydrofolate reductase (DHFR), 3-isopropylmalate (IPMDH), glyceraldehyde-3-phosphate dehydrogenase (GAPDH), aldose reductase (AR), alcohol dehydrogenase (ADH), and lactate dehydrogenase (LDH), and enoyl ACP reductase.

The present invention also provides compounds and combinatorial libraries of compounds of the formula: 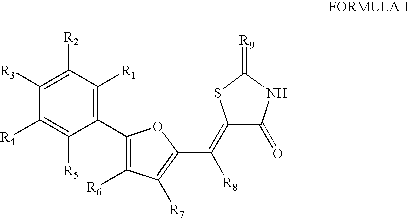
wherein R₁ to R₈ each independently are H, alkyl, alkenyl, alkynyl, aryl, heterocycle, COOH, COOAlkyl, CONR₁₀R₁₁, C(O)R₁₂, OH, OAlkyl, OAc, SH, SR₁₂, SO₃H, S(O)R₁₂, SO₂NR₁₀R₁₁, S(O)₂R₁₂, NH₂, NHR₁₂, NR₁₀R₁₁ NHCOR₁₂, NR₁₀COR₁₂, N₃, NO₂, PH₃, PH₂R₁₂, H₂PO₄, H₂PO₃, H₂PO₂, HPO₄R₁₂, PO₂R₁₁R₁₂, CN, or X. R₉ is an oxygen, sulfur, or nitrogen atom, where the nitrogen atom can be substituted, e.g. NR₁₂. R₁₀, R₁₁, and R₁₂ each independently are hydrogen, alkyl, alkenyl, alkynyl, aryl, or heterocycle, or R₁₀ and R₁₁ together with the nitrogen to which they are attached can be joined to form a heterocyclic ring.

As used herein, “alkyl” means a carbon chain having from one to twenty carbon atoms. The alkyl group of the present invention can be straight chain or branched. It can be unsubstituted or can be substituted. When substituted, the alkyl group can have up to ten substituent groups, such as COOH, COOAlkyl, CONR₁₀R₁₁, C(O)R₁₂, OH, OAlkyl, OAc, SH, SR₁₂, SO₃H, S(O)R₁₂, SO₂NR₁₀R₁₁, S(O) 2R₁₂, NH₂, NHR₁₂, NR₁₀R₁₁, NHCOR₁₂, NR₁₀COR₁₂, N₃, NO₂, PH₃, PH₂R₁₂, H₂PO₄, H₂PO₃, H₂PO₂, HPO₄R₁₂, PO₂R₁₁R₁₂, CN, or X, ═O, CR₁₀R₁₁, aryl, heterocycle and the like. In such instances, R₁₀, R₁₁, and R₁₂ each independently can be, for example, hydrogen, alkyl, alkenyl, alkynyl, aryl, or heterocycle, or R₁₀ and R₁₁ together with the carbon or nitrogen atom to which they are attached can be joined to form a ring.

Additionally, the alkyl group present in the compounds of the invention, whether substituted or unsubstituted, can have one or more of its carbon atoms replaced by a heterocyclic atom, such as an oxygen, nitrogen, or sulfur atom. For example, alkyl as used herein includes groups such as (OCH₂CH₂)ₙ or (OCH₂CH₂ CH₂)ₙ, where n has a value such that there are twenty or less carbon atoms in the alkyl group. Similar compounds having alkyl groups containing a nitrogen or sulfur atom are also encompassed by the present invention.

As used herein “alkenyl” means an unsaturated alkyl groups as defined above, where the unsaturation is in the form of a double bond. The alkenyl groups of the present invention can have one or more unsaturations. Nonlimiting examples of such groups include CH═CH₂, CH₂CH₂CH═CHCH₂CH₃, and CH₂CH═CHCH₃. As used herein “alkynyl” means an unsaturated alkyl group as defined above, where the unsaturation is in the form of a triple bond. Alkynyl groups of the present invention can include one or more unsaturations. Nonlimiting examples of such groups include C≡CH, CH₂CH₂C≡CCH₂CH₃, and CH₂C≡CCH₃.

The compounds of the present invention can include compounds in which R₁ to R₈ each independently are complex substituents containing one or more unsaturation, one or more substituent, and/or one or more heterocyclic atom. These complex substituents are also referred to herein as “linkers” or “expansion linkers.” Nonlimiting examples of complex substituents that can be used in the present invention are presented in Tables 6 to 12.

As used herein, “aromatic group” refers to a group that has a planar ring with 4n+2 pi-electrons, where in is a positive integer. The term “aryl” as used herein denotes a nonheterocyclic aromatic compound or group. For example, a benzene ring or naphthalene ring.

As used herein, “heterocyclic group” or “heterocycle” refers to an aromatic compound or group containing one or more heterocyclic atom. Nonlimiting examples of heterocyclic atoms that can be present in the heterocyclic groups of the invention include nitrogen, oxygen and sulfur. In general, heterocycles of the present invention will have from five to seven atoms and can be substituted or unsubstituted. When substituted, substituents include, for example, those groups provided for R₁ to R₈. Nonlimiting examples of heterocyclic groups of the invention include pyroles, pyrazoles, imidazoles, pyridines, pyrimidines, pyridazines, pyrazines, triazines, furans, oxazoles, thiazoles, thiophenes, diazoles, triazoles, tetrazoles, oxadiazoles, thiodiazoles, and fused heterocyclic rings, for example, indoles, benzofurans, benzothoiphenes, benzoimidazoles, benzodiazoles, benzotriazoles, benzotetrazoles, and quinolines.

As used herein, the variable “X” indicates a halogen atom. Halogens suitable for use in the present invention include chlorine, fluorine, iodine, and bromine, with bromine being particularly useful. As used herein, “Ac” denotes an acyl group. Suitable acyl groups can have, for example, an alkyl, alkenyl, alkynyl, aromatic, or heterocyclic group as defined above attached to the carbonyl group.

The phenyl ring in Formula I can be substituted with one or multiple substituents. Variation in the substitution on the phenyl ring provides compounds that allow for addition of a specificity ligand to directed sites on the phenyl ring. Direction of the specificity ligand improves the ease and efficiency of manufacture of combinatorial libraries containing bi-ligands having the common ligand mimic bound to a specificity ligand.

In one embodiment of the invention, only one of R₁ to R₅ is a substituent other than hydrogen. In such instances, R₁ to R₅ independently can be, alkyl, alkenyl, alkynyl, aryl, heterocycle, COOH, COOAlkyl, CONR₁₀R₁₁, C(O)R₁₂, OH, OAlkyl, OAc, SH, SR₁₂, SO₃H, S(O)R₁₂, SO₂NR₁₀R₁₁, S(O)₂R₁₂, NH₂, NHR₁₂, NR₁₀R₁₁, NHCOR₁₂, NR₁₀COR₁₂, N₃, NO₂, PH₃, PH₂R₁₂, H₂PO₄, H₂PO₃, H₂PO₂, HPO₄R₁₂, PO₂R₁₁R₁₂, CN, or X, where R₁₀, R₁₁, and R₁₂ are as defined in Formula I. For example, R₁ to R₅ independently can be an amide, a hydroxy group, a thiol group, or an acid group, such as a carboxylic acid. Additionally, R₁ to R₅ independently can be any of the complex substituents provided in Tables 6 to 12. When compounds of the invention contain an active hydroxy group, they also can be present in the form of an ether or ester, for example, an alkyl ether or alkyl ester. Thus, the invention encompasses compounds in which R₁ to R₅ can be an OAlkyl group or a COOAlkyl group. Non-limiting examples of OAlkyl groups include OMe (OCH₃), OEt (OCH₂CH₃), OPr (OCH₂CH₂CH₃), and the like. Non-limiting examples of COOAlkyl groups include COOMe, COOEt, COOPr, COOBu, COO-tBu, and the like.

In another embodiment, two or more of R₁ to R₅ are substituents other than hydrogen. In such instances, the substituent groups can be the same or different. For example, the phenyl ring of the compounds can be substituted with two OAlkyl groups, such as two OMe groups or one OMe group and one OPr group. Alternatively, the phenyl ring of the compounds can be substituted with an OH group and either a COOH or COOAlkyl group. Any combination of the above listed substituents for R₁ to R₅, including complex substituents such as those in Tables 6 to 12, is contemplated by the present invention. Similarly, where the compounds of the invention contain three or more substituents any combination of R₁ to R₅ is encompassed by the invention.

Similarly, the furan ring in Formula I can be substituted with one or two substituents. In one embodiment of the invention, only one of R₆ or R₇ is a substituent other than hydrogen. In such instances, R₆ or R₇ can be alkyl, alkenyl, alkynyl, aryl, heterocycle, COOH, COOAlkyl, CONR₁₀R₁₁, C(O)R₁₂, OH, OAlkyl, OAc, SH, SR₁₂, SO₃H, S(O)R₁₂, SO₂NR₁₀R₁₁, S(O)₂R₁₂, NH₂, NHR₁₂, NR₁₀R₁₁, NHCOR₁₂, NR₁₀COR₁₂, N₃, NO₂, PH₃, PH₂R₁₂, H₂PO₄, H₂PO₃, H₂PO₂, HPO₄R₁₂, PO₂R₁₁R₁₂, CN, or X, where R₁₀, R₁₁, and R₁₂ are as defined in Formula I. When R₆ or R₇ contains an active hydroxy group, it also can be present in the form of an ether or ester, for example, an alkyl ether or alkyl ester. Thus, the invention encompasses compounds in which R₆ and R₇ can be an OAlkyl group or a COOAlkyl group.

In another embodiment, both of R₆ and R₇ are substituents other than hydrogen. In such instances, the substituent groups can be the same or different. Any combination of the above listed substituents for R₆ to R₇, including complex substituents such as those in Tables 6 to 12, is contemplated by the present invention.

Likewise, the substituent R₈ attached to the carbon atom between the furan and thiazolidinedone rings can be either hydrogen or a substituent other than hydrogen. Where R₈ is a substituent other than hydrogen, it can be alkyl, alkenyl, alkynyl, aryl, heterocycle, COOH, COOAlkyl, CONR₁₀R₁₁, C(O)R₁₂, OH, OAlkyl, OAc, SH, SR₁₂, SO₃H, S(O)R₁₂, SO₂NR₁₀R₁₁, S(O)₂R₁₂, NH₂, NHR₁₂, NR₁₀R₁₁, NHCOR₁₂, NR₁₀COR₁₂, N₃, NO₂, PH₃, PH₂R₁₂, H₂PO₄, H₂PO₃, H₂PO₂, HPO₄R₁₂, PO₂R₁₁R₁₂, CN, or X, where R₁₀, R₁₁, and R₁₂ are as defined in Formula I. When R₈ contains an active hydroxy group, it also can be present in the form of an ether or ester, for example, an alkyl ether or alkyl ester. Thus, the invention encompasses compounds in which R₈ can be an OAlkyl group or a COOAlkyl group. The present invention further encompasses compounds in which R₈ is a complex substituent such as those provided in Tables 6 to 12.

In one aspect, the invention provides compounds in which R₁ to R₈ are not all hydrogen. In other words, the invention includes compounds in which at least one of R₁ to R₈ is a substituent other than hydrogen.

Compounds having complex substituents are encompassed by the invention. The following formulas are representative of such compounds. In each of the formula, any combination of the variables listed can exist. Nonlimiting examples of thiazolidinedione compounds corresponding to formulas Ia to Ik and IIa to IIk are provided in Tables 6 to 12. However, it is understood that the invention also encompasses corresponding rhodanine compounds in accordance with formulas Ia to Ik and IIIa to IIIk. The compounds represented in Tables 6 to 12 are only examples of compounds of the invention and are not intended to be all-inclusive. One having ordinary skill in the art would readily recognize other compounds within the scope of formula I which are also part of the invention.

In one embodiment, the invention provides compounds and combinatorial libraries of compounds having formula Ia 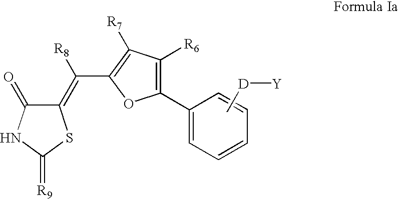
wherein R₉ is O, S, or NR₁₂. R₆, R₇, and R₈ each independently are as defined above. D is alkylene, alkenylene, alkynylene, aryl, or heterocycle; Y is OH, NHR₁₂, SR₁₂, COOH, SO₂OH, X, CN, C(O)R₁₂, N₃, CONH₂, C≡CH, or CH═CH₂; and R₉ is S, O, or NR₁₂. R₁₂ is hydrogen, alkyl, alkenyl, alkynyl, aryl, or heterocycle.

As used herein, the terms “alkylene,” “alkenylene,” and “alkynylene” refer to alkyl, alkenyl, and alkynyl groups as defined above in which one additional atom has been removed such that the group is divalent. Nonlimiting examples of such groups include —CH₂CH₂CH₂—, —CH₂CH—CHCH₂—, and —CH₂C≡CCH₂—.

In a second embodiment, the invention provides compounds and combinatorial libraries of compounds having formula Ib 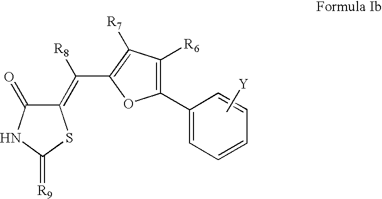
wherein R₉ is O, S, or NR₁₂, and Y is OH, NHR₁₂, SR₁₂, COOH, SO₂OH, X, CN, C(O)R₁₂, N₃, CONH₂, CONHR₁₂, C≡CH, or CH═CH₂. R₁₂ is hydrogen, alkyl, alkenyl, alkynyl, aryl, or heterocycle. R₆, R₇, and R₈ each independently are as defined above.

In another embodiment, the invention provides compounds and combinatorial libraries of compounds having formula Ic 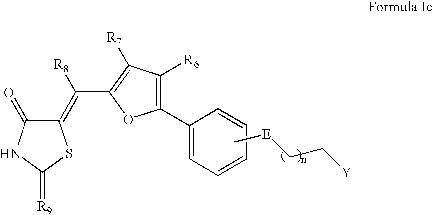
wherein R₉ is O, S, or NR₁₂. R₆, R₇, and R₈ each independently are as defined above. E is O, S, NR₁₂, CR₁₁C₁₂, CONR₁₂, SO₂NR₁₂, NR₁₀CONR₁₂, NR₁₁CNHNR₁₂, NR₁₂COO, C≡C, or CH═CH. Y is OH, NHR₁₂, SH, COOH, SO₂OH, X, CN, C(O)R₁₂, N₃, CONH₂, CONHR₁₂, C≡CH, or CH═CH₂; and n is an integer between 0 and 5, inclusive. R₃₁ and R₁₂ each independently are hydrogen, alkyl, alkenyl, alkynyl, aryl, or heterocycle.

In yet another embodiment, the invention provides compounds and combinatorial libraries of compounds having formula Id 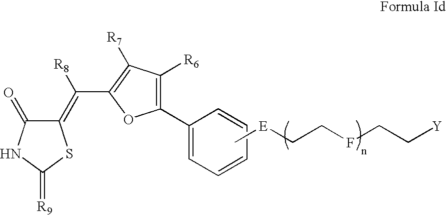
wherein R₉ is O, S, or NR₁₂. R₆, R₇, and R₈ each independently are as defined above. E and F each independently are O, S, NR₁₂, CR₁₁C₁₂, CONR₁₂, SO₂NR₁₂, NR₁₁CONR₁₂, NR₁₁CNHNR₁₂, NR₁₂COO, C≡C, or CH═CH. Y is OH, NHR₁₂, SH, COOH, SO₂OH, X, CN, C(O)R₁₂, N₃, CONH₂, CONHR₁₂, C≡CH, or CH═CH₂; and n is an integer between 0 and 5, inclusive. R₁₁ and R₁₂ each independently are hydrogen, alkyl, alkenyl, alkynyl, aryl, or heterocycle.

In a further embodiment, the invention provides compounds and combinatorial libraries of compounds having formula Ie 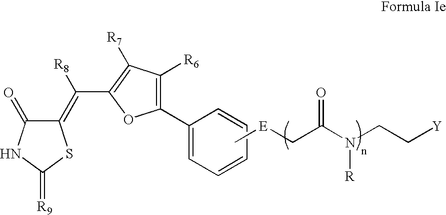
wherein R₉ is O, S, or NR₁₂. R₆, R₇, and R₈ each independently are as defined above. E is O, S, NR₁₂, CR₁₁C₁₂, CONR₁₂, SO₂NR₁₂, NR₁₁CONR₁₂, NR₁₁CNHNR₁₂, NR₁₂COO, C≡C, or CH═CH. Y is OH, NHR₁₂, SH, COOH, SO₂OH, X, CN, C(O)R₁₂, N₃, CONH₂, CONHR₁₂, C≡CH, or CH═CH₂; and n is an integer between 0 and 5, inclusive. R, R₁₁, R₁₂, and R₁₃ each independently are hydrogen, alkyl, alkenyl, alkynyl, aryl, or heterocycle.

In another embodiment, the invention provides compounds and combinatorial libraries of compounds having formula If 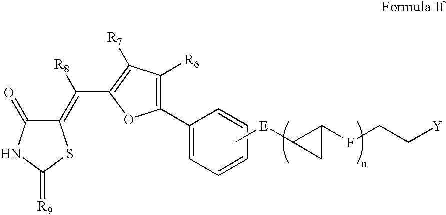
wherein R₉ is O, S, or NR₁₂. R₆, R₇, and R₈ each independently are as defined above. E and F each independently are O, S, NR₁₂, CR₁₁C₁₂, CONR₁₂, SO₂NR₁₂, NR₁₁CONR₁₂, NR₁₁CNHNR₁₂, NR₁₂COO, C≡C, or CH═CH. Y is OH, NHR₁₂, SH, COOH, SO₂OH, X, CN, C(O)R₁₂, N₃, CONH₂, CONHR₁₂, C≡CH, or CH═CH₂; and n is an integer between 0 and 5, inclusive. R₁₁ and R₁₂ each independently are hydrogen, alkyl, alkenyl, alkynyl, aryl, or heterocycle.

In yet another embodiment, the invention provides compounds and combinatorial libraries of compounds having formula Ig 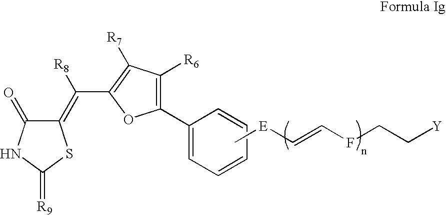
wherein R₉ is O, S, or NR₁₂. R₆, R₇, and R₈ each independently are as defined above. E is O, S, NR₁₂, CR₁₁C₁₂, CONR₁₂, SO₂NR₁₂, NR₁₁CONR₁₂, NR₁₁CNHNR₁₂, NR₁₂COO, C≡C, or CH═CH. Each F independently is O, S, NR₁₂, CR₁₁R₁₂, CONR₁₂, NR₁₁CONR₁₂, NR₁₁CNHNR₁₂, NR₁₂COO, C═C, or CH═CH. Y is OH, NHR₁₂, SH, COOH, SO₂OH, X, CN, C(O)R₁₂, N₃, CONH₂, CONHR₁₂, C≡CH, or CH═CH₂; and n is an integer between 0 and 5, inclusive. R₁₁ and R₁₂ each independently are hydrogen, alkyl, alkenyl, alkynyl, aryl, or heterocycle.

In a further embodiment, the invention provides compounds and combinatorial libraries of compounds having formula Ih 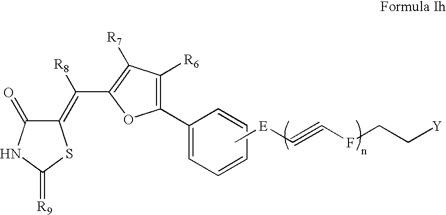
wherein R₉ is O, S, or NR₁₂. R₆, R₇, and R₈ each independently are as defined above. E is O, S, NR₁₂, CR₁₁C₁₂, CONR₁₂, SO₂NR₁₂, NR₁₁CONR₁₂, NR₁₁CNHNR₁₂, NR₁₂COO, C≡C, or CH═CH. Each F independently is O, S, NR₁₂, CR₁₁R₁₂, CONR₁₂, NR₁₁CONR₁₂, NR₁₁CNHNR₁₂, NR₁₂COO, C═C, or CH═CH. Y is OH, NHR₁₂, SH, COOH, SO₂OH, X, CN, C(O)R₁₂, N₃, CONH₂, CONHR₁₂, C═CH, or CH═CH₂; and n is an integer between 0 and 5, inclusive. R₁₁ and R₁₂ each independently are hydrogen, alkyl, alkenyl, alkynyl, aryl, or heterocycle.

In another embodiment, the invention provides compounds and combinatorial libraries of compounds having formula Ii 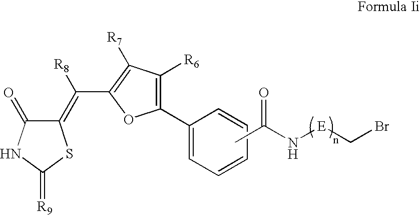
wherein E is CH₂, CH₂CH₂OCH, or CH₂CH₂SCH and n is an integer between 1 and 10, inclusive. In certain embodiments of the invention, when n is greater than 4, E is CH₂CH₂OCH or CH₂CH₂SCH. R₉ is O, S, or NR₁₂. R₆, R₇, and R₈ each independently are as defined above.

In another embodiment, the invention provides compounds and combinatorial libraries of compounds having formula Ij 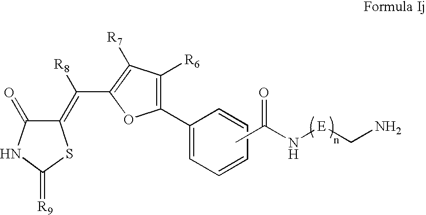
wherein E is CH₂, CH₂CH₂OCH, or CH₂CH₂SCH and n is an integer between 1 and 10, inclusive. In certain embodiments of the invention, when n is greater than 4, E is CH₂CH₂OCH or CH₂CH₂SCH. R₉ is O, S, or NR₁₂. R₆, R₇, and R₈ each independently are as defined above.

In another embodiment, the invention provides compounds and combinatorial libraries of compounds having formula Ik 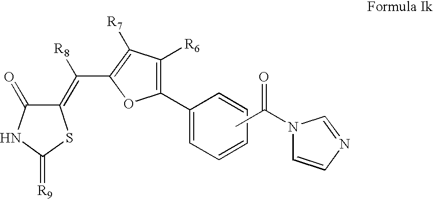
wherein R₆, R₇, and R₈ each independently are as defined above.

In one aspect, the invention provides compounds and combinatorial libraries of compounds having the formula 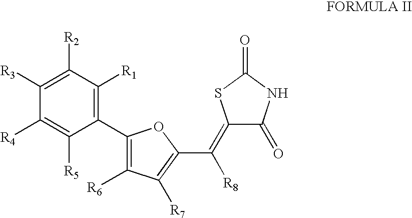
wherein R₁ to R₈ each independently are H, alkyl, alkenyl, alkynyl, aryl, heterocycle, COOH, COOAlkyl, CONR₁₀R₁₁, C(O)R₁₂, OH, OAlkyl, OAc, SH, SR₁₂, SO₃H, S(O)R₁₂, SO₂NR₁₀R₁₁, S(O)₂R₁₂, NR₁₂, NHR₁₂, NR₁₀R₁₁, NHCOR₁₂, NR₁₀COR₁₂, N₃, NO₂, PH₃, PH₂R₁₂, H₂PO₄, H₂PO₃, H₂PO₂, HPO₄R₁₂, PO₂R₁₁R₁₂, CN, or X. R₁₀, R₁₁, and R₁₂ each independently are hydrogen, alkyl, alkenyl, alkynyl, aryl, or heterocycle, or R₁₀ and R₁₁ together with the nitrogen to which they are attached can be joined to form a heterocyclic ring. Such compounds include all manner of combinations for R₁ to R₈ as discussed above with regard to compounds of Formula I. Exemplified compounds of this formula include, but are not limited to, 4-[5-(2,4-dioxo-thiazolidin-5-ylidenemethyl)-furan-2-yl]benzoic acid; 3-[5-(2,4-dioxo-thiazolidin-5-ylidenemethyl)-furan-2-yl]benzoic acid; 5-[5-(4-hydroxy-phenyl)-furan-2-ylmethylene]-thiazolidine-2,4-dione; 5-[5-(2,4-dioxo-thiazolidin-5-ylidenemethyl)-furan-2-yl]-2-hydroxy-benzoic acid methyl ester; 5-[5-(2,4-dioxo-thiazolidin-5-ylidenemethyl)-furan-2-yl]-2-hydroxy-benzoic acid; N-{3-[5-(2,4-dioxo-thiazolidin-5-ylidenemethyl)-furan-2-yl]phenyl}acetamide; and 5-[5-(3,4-dimethoxy-phenyl)-furan-2-ylmethylene]-thiazolidine-2,4-dione.

In one embodiment, the invention provides compounds and combinatorial libraries of compounds having formula IIa 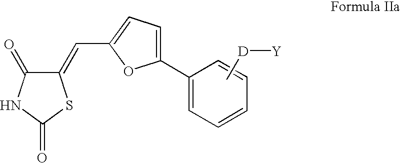
wherein D is alkylene, alkenylene, alkynylene, aryl, or heterocycle, and Y is OH, NHR₁₂, SH, COOH, SO₂OH, X, CN, N₃, CONH₂, CONHR₁₂, C≡CH, or CH═CH₂. R₁₂ is hydrogen, alkyl, alkenyl, alkynyl, aryl, or heterocycle.

In a second embodiment, the invention provides compounds and combinatorial libraries of compounds having formula IIb 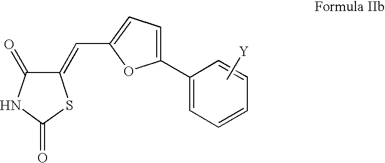
wherein Y is OH, NHR₁₂, SH, COOH, SO₂OH, X, CN, N₃, CONH₂, CONHR₁₂, C≡CH, or CH═CH₂. R₁₂ is hydrogen, alkyl, alkenyl, alkynyl, aryl, or heterocycle.

In another embodiment, the invention provides compounds and combinatorial libraries of compounds having formula IIc 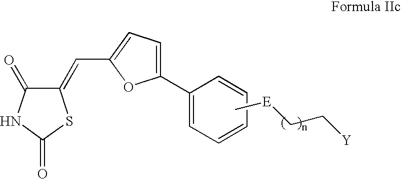
wherein E is O, S, NR₁₂, CR₁₁C₁₂, CONR₁₂, SO₂NR₁₂, NR₁₁CONR₁₂, NR₁₁CNHNR₁₂, NR₁₂COO, C≡C, or CH═CH. Y is OH, NHR₁₂, SH, COOH, SO₂OH, X, CN, N₃, CONH₂, CONHR₁₂, C≡CH, or CH═CH₂; and n is an integer between 0 and 5, inclusive. R₁₁ and R₁₂ each independently are hydrogen, alkyl, alkenyl, alkynyl, aryl, or heterocycle.

In yet another embodiment, the invention provides compounds and combinatorial libraries of compounds having formula IId 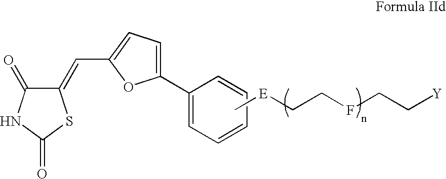
wherein E and F each independently are O, S, NR₁₂, CR₁₁C₁₂, CONR₁₂, SO₂NR₁₂, NR₁₁CONR₁₂, NR₁₁CNHNR₁₂, NR₁₂COO, C≡C, or CH═CH. Y is OH, NHR₁₂, SH, COOH, SO₂OH, X, CN, N₃, CONH₂, CONHR₁₂, C≡CH, or CH═CH₂; and n is an integer between 0 and 5, inclusive. R₁₁ and R₁₂ each independently are hydrogen, alkyl, alkenyl, alkynyl, aryl, or heterocycle.

In a further embodiment, the invention provides compounds and combinatorial libraries of compounds having formula IIe 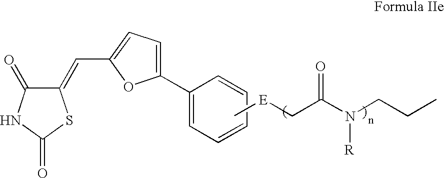
wherein E is O, S, NR₁₂, CR₁₁C₁₂, CONR₁₂, SO₂NR₁₂, NR₁₁CONR₁₂, NR₁₁CNHNR₁₂, NR₁₂COO, C≡C, or CH═CH. Y is OH, NHR₁₂, SH, COOH, SO₂OH, X, CN, N₃, CONH₂, CONHR₁₂, C≡CH, or CH═CH₂; and n is an integer between 0 and 5, inclusive. R, R₁₁, R₁₂, and R₁₃ each independently are hydrogen, alkyl, alkenyl, alkynyl, aryl, or heterocycle.

In another embodiment, the invention provides compounds and combinatorial libraries of compounds having formula IIf 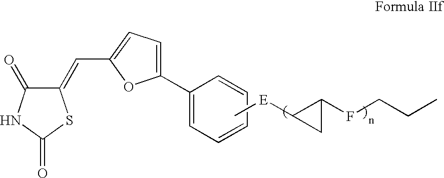
wherein E and F each independently are O, S, NR₁₂, CR₁₁C₁₂, CONR₁₂, SO₂NR₁₂, NR₁₁CONR₁₂, NR₁₁CNHNR₁₂, NR₁₂COO, C≡C, or CH═CH. Y is OH, NHR₁₂, SH, COOH, SO₂OH, X, CN, N₃, CONH₂, CONHR₁₂, C≡CH, or CH═CH₂; and n is an integer between 0 and 5, inclusive. R₁, and R₁₂ each independently are hydrogen, alkyl, alkenyl, alkynyl, aryl, or heterocycle.

In yet another embodiment, the invention provides compounds and combinatorial libraries of compounds having formula IIg 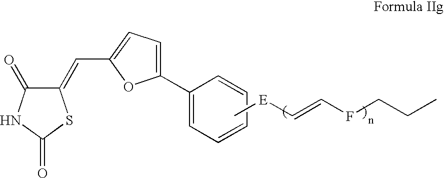
wherein E is O, S, NR₁₂, CR₁₁C₁₂, CONR₁₂, SO₂NR₁₂, NR₁₁CONR₁₂, NR₁₁CNHNR₁₂, NR₁₂COO, C≡C, or CH═CH. Each F independently is O, S, NR₁₂, CR₁₁R₁₂, CONR₁₂, NR₁₁CONR₁₂, NR₁₁CNHNR₁₂, NR₁₂COO, C═C, or CH═CH. Y is OH, NHR₁₂, SH, COOH, SO₂OH, X, CN, N₃, CONH₂, CONHR₁₂, C≡CH, or CH═CH₂; and n is an integer between 0 and 5, inclusive. R₁₁ and R₁₂ each independently are hydrogen, alkyl, alkenyl, alkynyl, aryl, or heterocycle.

In a further embodiment, the invention provides compounds and combinatorial libraries of compounds having formula IIh 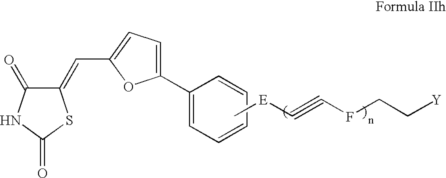
wherein E is O, S, NR₁₂, CR₁₁C₁₂, CONR₁₂, SO₂NR₁₂, NR₁₁CONR₁₂, NR₁₁CNHNR₁₂, NR₁₂COO, C≡C, or CH═CH. Each F independently is O, S, NR₁₂, CR₁₁R₁₂, CONR₁₂, NR₁₁CONR₁₂, NR₁₁CNHNR₁₂, NR₁₂COO, C═C, or CH═CH. Y is OH, NHR₁₂, SH, COOH, SO₂OH, X, CN, N₃, CONH₂, CONHR₁₂, C≡CH, or CH═CH₂; and n is an integer between 0 and 5, inclusive. R₁₁ and R₁₂ each independently are hydrogen, alkyl, alkenyl, alkynyl, aryl, or heterocycle.

In another embodiment, the invention provides compounds and combinatorial libraries of compounds having formula IIi 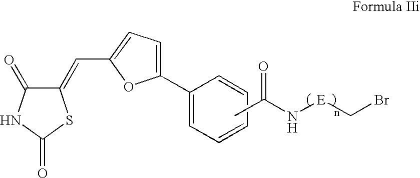
wherein E is CH₂, CH₂CH₂OCH, or CH₂CH₂SCH and n is an integer between 1 and 10, inclusive. In certain embodiments of the invention, when n is greater than 4, E is CH₂CH₂OCH or CH₂CH₂SCH.

In another embodiment, the invention provides compounds and combinatorial libraries of compounds having formula IIj 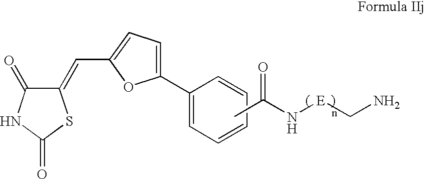
wherein E is CH₂, CH₂CH₂OCH, or CH₂CH₂SCH and n is an integer between 1 and 10, inclusive. In certain embodiments of the invention, when n is greater than 4, E is CH₂CH₂OCH or CH₂CH₂SCH.

In another embodiment, invention provides compounds and combinatorial libraries of compounds having formula IIk

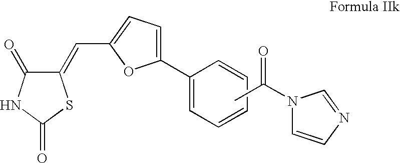

In another aspect, the invention provides 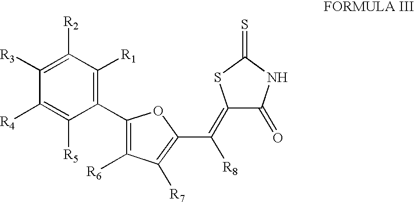
wherein R₁ to R₈ each independently are H, alkyl, alkenyl, alkynyl, aryl, heterocycle, COOH, COOAlkyl, CONR₁₀R₁₁, C(O)R₁₂, OH, OAlkyl, OAc, SH, SR₁₂, SO₃H, S(O)R₁₂, SO₂NR₁₀R₁₁, S(O)₂R₁₂, NH₂, NHR₁₂, NR₁₀R₁₁, NHCOR₁₂, NO₂, PH₃, PH₂R₁₂, H₂PO₄, H₂PO₃, H₂PO₂, HPO₄R₁₂, PO₂R₁₁R₁₂, CN, or X. R₁₀, R₁₁, and R₁₂ each independently are hydrogen, alkyl, alkenyl, alkynyl, aryl, or heterocycle, or R₁₀ and R₁₁ together with the nitrogen to which they are attached can be joined to form a heterocyclic ring. Such compounds include all manner of combinations for R₁ to R₈ as discussed above with regard to compounds of Formula I. Exemplified compounds of this formula include, but are not limited to, 4-[5-(4-oxo-2-thioxo-thiazolidin-5-ylidenemethyl)-furan-2-yl]benzoic acid; 3-[5-(4-oxo-2-thioxo-thiazolidin-5-ylidenemethyl)-furan-2-yl]benzoic acid; 5-[5-(4-hydroxy-phenyl)-furan-2-ylmethylene]-2-thioxo-thiazolidin-4-one; 2-hydroxy-5-[5-(4-oxo-2-thioxo-thizolidine-5-ylidenemethyl)-furan-2-yl]-2-benzoic acid methyl ester; 2-hydroxy-5-[5-(4-oxo-2-thioxo-thizolidine-5-ylidenemethyl)-furan-2-yl]-2-benzoic acid; N-{3-[5-(4-oxo-2-thioxo-thiazolidin-5-ylidenemethyl)-furan-2-yl]phenyl}acetamide; and 5-[5-(3,4-dimethoxy-phenyl)-furan-2-ylmethylene]-2-thioxo-thiazolidin-4-one.

In one embodiment, the invention provides compounds and combinatorial libraries of compounds having formula IIIa 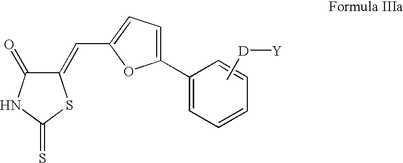
wherein D is alkylene, alkenylene, alkynylene, aryl, or heterocycle; and Y is OH, NHR₁₂, SH, COOH, SO₂OH, X, CN, N₃, CONH₂, C≡CH, or CH═CH₂. R₁₂ is hydrogen, alkyl, alkenyl, alkynyl, aryl, or heterocycle.

In a second embodiment, the invention provides compounds and combinatorial libraries of compounds having formula IIIb 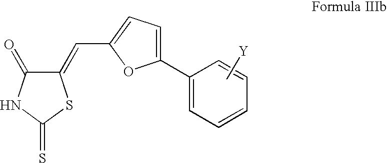
wherein, and Y is OH, NHR₁₂, SH, COOH, SO₂OH, X, CN, N₃, CONH₂, CONHR₁₂, C≡CH, or CH═CH₂. R₁₂ is hydrogen, alkyl, alkenyl, alkynyl, aryl, or heterocycle

In another embodiment, the invention provides compounds and combinatorial libraries of compounds having formula IIIc 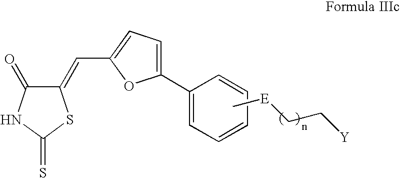
wherein E is O, S, NR₁₂, CR₁₁C₁₂, CONR₁₂, SO₂NR₁₂, NR₁₁CONR₁₂, NR₁₁CNHNR₁₂, NR₁₂COO, C≡C, or CH═CH. Y is OH, NHR₁₂, SH, COOH, SO₂OH, X, CN, N₃, CONH₂, CONHR₁₂, C≡CH, or CH═CH₂; and n is an integer between 0 and 5, inclusive. R₁₁ and R₁₂ each independently are hydrogen, alkyl, alkenyl, alkynyl, aryl, or heterocycle.

In yet another embodiment, the invention provides compounds and combinatorial libraries of compounds having formula IIId 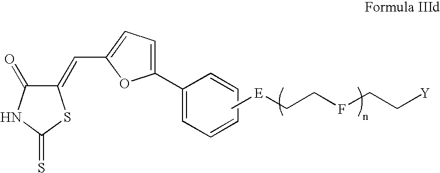
wherein E and F each independently are O, S, NR₁₂, CR₁₁C₁₂, CONR₁₂, SO₂NR₁₂, NR₁₁CONR₁₂, NR₁₁CNHNR₁₂, NR₁₂COO, C≡C, or CH═CH. Y is OH, NHR₁₂, SH, COOH, SO₂OH, X, CN, N₃, CONH₂, CONHR₁₂, C≡CH, or CH═CH₂; and n is an integer between 0 and 5, inclusive. R₁₁ and R₁₂ each independently are hydrogen, alkyl, alkenyl, alkynyl, aryl, or heterocycle.

In a further embodiment, the invention provides compounds and combinatorial libraries of compounds having formula IIIe 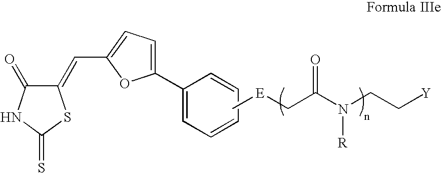
wherein E is O, S, NR₁₂, CR₁₁C₁₂, CONR₁₂, SO₂NR₁₂, NR₁₁CONR₁₂, NR₁₁CNHNR₁₂, NR₁₂COO, C≡C, or CH═CH. Y is OH, NHR₁₂, SH, COOH, SO₂OH, X, CN, N₃, CONH₂, CONHR₁₂, C≡CH, or CH═CH₂; and n is an integer between 0 and 5, inclusive. R, R₁₁, R₁₂, and R₁₃ each independently are hydrogen, alkyl, alkenyl, alkynyl, aryl, or heterocycle.

In another embodiment, the invention provides compounds and combinatorial libraries of compounds having formula IIIf 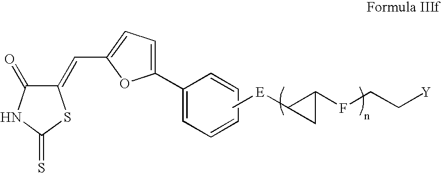
wherein E and F each independently are O, S, NR₁₂, CR₁₁C₁₂, CONR₁₂, SO₂NR₁₂, NR₁₁CONR₁₂, NR₁₁CNHNR₁₂, NR₁₂COO, C≡C, or CH═CH. Y is OH, NHR₁₂, SH, COOH, SO₂OH, X, CN, N₃, CONH₂, CONHR₁₂, C≡CH, or CH═CH₂; and n is an integer between 0 and 5, inclusive. R₁₁ and R₁₂ each independently are hydrogen, alkyl, alkenyl, alkynyl, aryl, or heterocycle.

In yet another embodiment, invention provides compounds and combinatorial libraries of compounds having formula IIIg 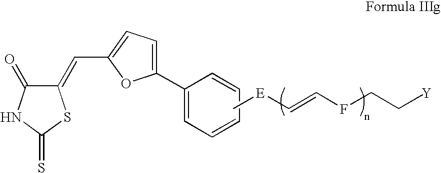
wherein E is O, S, NR₁₂, CR₁₁C₁₂, CONR₁₂, SO₂NR₁₂, NR₁₁CONR₁₂, NR₁₁CNHNR₁₂, NR₁₂COO, C≡C, or CH═CH. Each F independently is O, S, NR₁₂, CR₁₁R₁₂, CONR₁₂, NR₁₁CONR₁₂, NR₁₁CNHNR₁₂, NR₁₂COO, C═C, or CH═CH. Y is OH, NHR₁₂, SH, COOH, SO₂OH, X, CN, N₃, CONH₂, CONHR₁₂, C≡CH, or CH═CH₂; and n is an integer between 0 and 5, inclusive. R₁₁ and R₁₂ each independently are hydrogen, alkyl, alkenyl, alkynyl, aryl, or heterocycle.

In a further embodiment, invention provides compounds and combinatorial libraries of compounds having formula IIIh 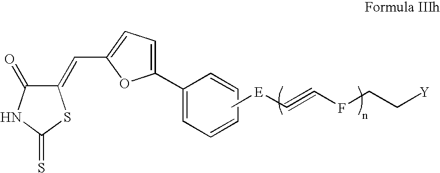
wherein E is O, S, NR₁₂, CR₁₁C₁₂, CONR₁₂, SO₂NR₁₂, NR₁₁CONR₁₂, NR₁₁CNHNR₁₂, NR₁₂COO, C≡C, or CH═CH. Each F independently is O, S, NR₁₂, CR₁₁R₁₂, CONR₁₂, NR₁₁CONR₁₂, NR₁₁CNHNR₁₂, NR₁₂COO, C═C, or CH═CH. Y is OH, NHR₁₂, SH, COOH, SO₂OH, X, CN, N₃, CONH₂, CONHR₁₂, C≡CH, or CH═CH₂; and n is an integer between 0 and 5, inclusive. R₁₁ and R₁₂ each independently are hydrogen, alkyl, alkenyl, alkynyl, aryl, or heterocycle.

In another embodiment, the invention provides compounds and combinatorial libraries of compounds having formula IIIi 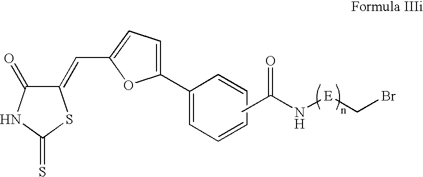
wherein E is CH₂, CH₂CH₂OCH, or CH₂CH₂SCH and n is an integer between 1 and 10, inclusive. In certain embodiments, when n is greater than 4, E is CH₂CH₂OCH or CH₂CH₂SCH.

In another embodiment, the invention provides compounds and combinatorial libraries of compounds having formula IIIj 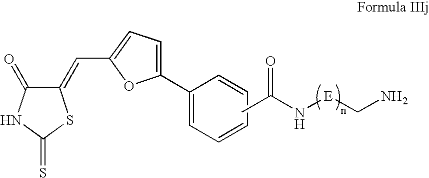
wherein E is CH₂, CH₂CH₂OCH, or CH₂CH₂SCH and n is an integer between 1 and 10, inclusive. In certain embodiments, when n is greater than 4, E is CH₂CH₂OCH or CH₂CH₂SCH.

In another embodiment, the invention provides compounds and combinatorial libraries of compounds having formula IIIk

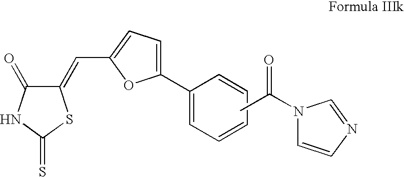

One or more of the compounds of the invention, even within a given library, can be present as a salt. The term “salt” encompasses those salts that form within the carboxylate anions and amine nitrogens and includes salts formed with the organic and inorganic anions and cations discussed below. Furthermore, the term includes salts that form by standard acid-based reactions with basic groups (such as amino groups) and organic or inorganic acids. Such acids include, hydrochloric, hydrofluoric, trifluoroacetic, sulfuric, phosphoric, acetic, succinic, citric, lactic, maleic, fumaric, glutaric, phthalic, tartaric, lauric, stearic, salicyclic, methanesulfonic, benzenesulfonic, sorbic, picric, benzoic, cinnamic, and like acids.

The term “organic or inorganic cation” refers to counter-ions for the carboxylate anion of a carboxylate salt. The counter-ions are chosen from the sodium, potassium, barium, aluminum, and calcium); ammonium and organic cations, such as mono-, di-, and tri-alkyl amines. Examples of suitable alkyl amines include, but are not limited to, trimethylamine, cyclohexylamine, dibenzylamine, bis(2-hydroxyethyl) amine, and the like. See for example “Pharmaceutical Salts,” Berge et al., J. Pharm. Sci., 66:1-19 (1977), which is incorporated herein by reference. Other cations encompassed by the above term include the protonated form of procaine, quinine, and N-methylglucosamine, and the protonated forms of basic amino acids such as glycine, ornithine, histidine, phenylglycine, lysine, and arginine. Furthermore, any zwitterionic form of the instant compounds formed by a carboxylic acid and an amino group is referred to by this term. For example, a cation for a carboxylate anion will exist when a position is substituted by a (quarternary ammonium)methyl group.

The compounds of the invention can also exist as solvates and hydrates. Thus, these compounds can crystallize with, for example, waters of hydration, or one, a number of, or any fraction thereof, of molecules of the mother liquor solvent. The solvates and hydrates of such compounds are included within the scope of this invention.

One or more compounds of the invention, even when in a library, can be in the biologically active ester form. Such as the non-toxic, metabolically-labile, ester-form. Such esters induce increased blood levels and prolong efficacy of the corresponding nonesterified forms of the compounds. Ester groups which can be used include the lower alkoxymethyl groups, for example, methoxymethyl, ethoxymethyl, isopropoxymethyl and the like; the —(C₁-C₁₂)alkoxyethyl groups, for example, methoxyethyl, ethoxyethyl, propoxyethyl, isopropoxyethyl and the like; the —(C₁-C₁₀)alkylthiomethyl groups, for example, methylthiomethyl, ethylthiomethyl, iso-propylmethyl and the like; and the acyloxymethyl groups, for example, pivaloyloxymethyl, pivaloyloxyethyl, acetoxymethyl, and acetoxyethyl. Salts, solvates, hydrates, biologically active esters of the compounds of the invention are common ligand variants of the compounds as defined above.

In another aspect, the present invention provides bi-ligands that contain a common ligand mimic as described above and a specificity ligand. In the bi-ligands of the invention, the common ligand mimic and the specificity ligand can be attached directly or indirectly. The common ligand mimic and specificity ligand are attached via a covalent bond formed from the reaction of one or more functional groups on the common ligand mimic with one or more functional groups on the specificity ligand. Direct attachment of the individual ligands in the bi-ligand can occur through reaction of simple functional groups on the ligands. Indirect attachment of the individual ligands in the bi-ligand can occur through a linker molecule. Such linkers include those provided in Tables 6 to 12. These linkers bind to each of the common ligand mimic and the specificity ligand through functional groups on the linker and the individual ligands. Some of the common ligand mimics of the present invention having substituents which include linker molecules, e.g. the common ligand mimics of Tables 6 to 12. Tailoring of the specific type and length of the linker attaching the common ligand mimic and specificity ligand allows tailoring of the bi-ligand to optimize binding of the common ligand mimic to a conservative site on the receptor and binding of the specificity ligand to a specificity site on the receptor.

The present invention provides specificity ligands that are specific for NAD receptors and combinatorial libraries containing these specificity ligands. For example, in one embodiment, compounds of the invention are ligands for specificity sites on dehydrogenases and reductases like those described above.

In another embodiment of the present invention, the specificity ligand is a compound having formula 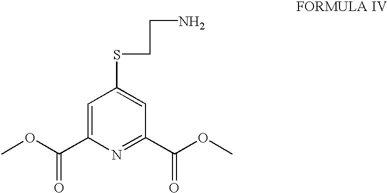
Specificity ligands, such as that of Formula IV can also exist as salts, or in other reactive forms.

Bi-ligands of the invention can be bi-ligands for any receptor. In one embodiment, the bi-ligand is a bi-ligand that binds an oxidoreductase. In another embodiment, bi-ligands of the present invention comprise a thiazolidinedione or rhodanine compound as a common ligand mimic and a specificity ligand. For example, bi-ligands of the invention can contain a common ligand mimic of Formula I coupled to a specificity ligand. Alternatively, bi-ligands of the invention can contain a common ligand mimic of Formula II or Formula III coupled to a specificity ligand. The specificity ligand can be any specificity ligand, for example a ligand that binds to a specificity site on an oxidoreductase. In such an embodiment, the specificity ligand can be a pyridine dicarboxylate. Examples of particular bi-ligands that fall within the invention are provided in FIG. 20.

The compounds of the present invention can be produced by any feasible method. For example, the compounds of the present invention can be produced by the following methods. Generally, these methods include the formation of an intermediate compound, followed by reaction of the intermediate with either 2,4-thiazolidinedione or rhodanine to form the final product.

The invention provides several methods for preparation of intermediates of the invention. Tailoring of each of these methods to produce a particular compound within the scope of the invention is within the level of skill of the ordinary artisan.

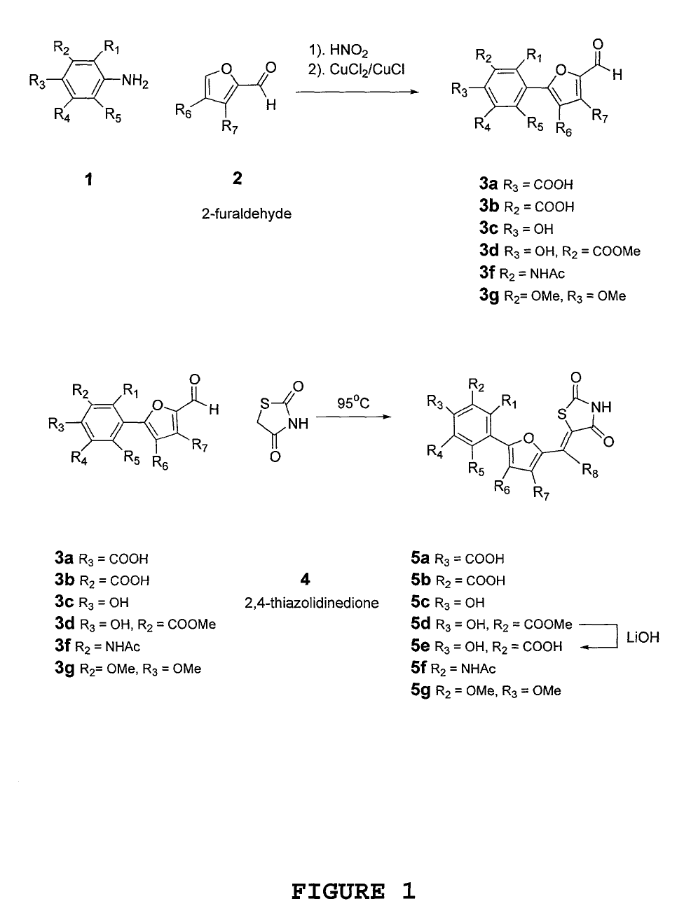

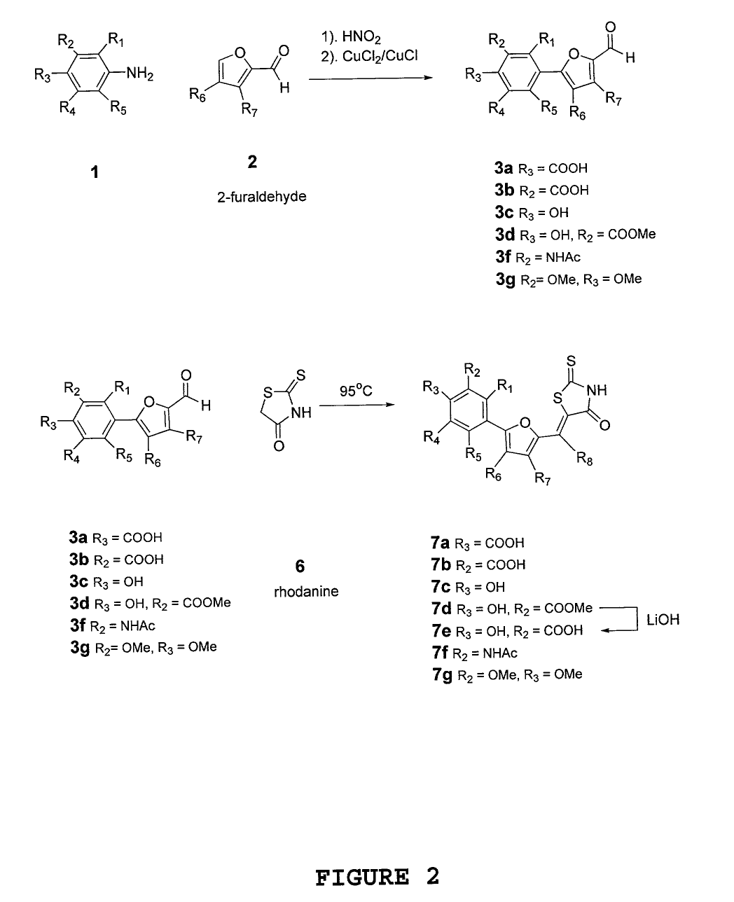

In one aspect, as shown in FIGS. 1 and 2, the present invention provides a method for the manufacture of an intermediate compound by reaction with 2-furaldehyde. For example, furanyl benzoic acid derivatives, such as 4-(5-formyl-furan-2-yl)-benzoic acid or 3-(5-formyl-furan-2-yl)-benzoic acid, can be prepared by this method.

Where the intermediate is a furanyl benzoic acid, the method provides reaction of an aminobenzoic acid, such as 4-aminobenzoic acid or 3-aminobenzoic acid, with a 2-furaldehyde in water or in acetone. The reaction is conducted in the presence of nitrous acid and a copper catalyst. In one embodiment, the nitrous acid is formed in situ from the reaction of HCl, such as 12M HCl, and a nitrate, such as sodium nitrate (NaNO₂). In such an embodiment, the HCl can be mixed with the aminobenzoic acid initially to form a suspension. This reaction is exothermic, and, thus, the suspension can be cooled to maintain a desirable reaction temperature. Once the suspension is cooled, for example, to a temperature of about 1° C., a solution of NaNO₂ in water can be added to the suspension in small amounts so that the temperature of the suspension is maintained, for example at a temperature of between about 5° C. and 10° C.

The copper catalyst employed in the reaction can be, for example, a CuCl₂/CuCl catalyst. In one embodiment, CuCl₂.2H₂O in water is added to the aminobenzoic acid/HCl suspension, followed by addition of a solution of 2-furaldehyde in acetone. The 2-furaldehyde can be pre-cooled, for instance by placing it in an ice bath, prior to addition to the suspension. CuCl is then added to the mixture in small portions, resulting in foaming of the mixture and precipitation of the desired intermediate compound. The CuCl can be added in small amounts over a period of time. For instance, the CuCl can be added over a period of time of about 10 to 60 minutes, for example, over a period of about 10 minutes. Because this reaction is exothermic, it is advantageous, but not necessary, to maintain the reaction mixture in an ice bath to control the reaction temperature.

The reaction mixture can be removed from the ice bath, and the internal temperature of the mixture allowed to rise. Additional amounts of CuCl can be added to the mixture. The mixture is then stirred at room temperature of a period of time, such as about 10 to 20 hours, for example, about 16 hours.

The resulting brown precipitate can then be filtered, washed with water, and dried. The product can be dried by conventional methods. For example, drying conveniently can be accomplished through lyophilization of the washed precipitate. The furaldehyde intermediate produced by this method can be used in subsequent reactions without further purification. However, if desired, purification can be carried out by any conventional means, for example, by recrystallization in ethanol.

In one embodiment of the invention, 4-aminobenzoic acid is employed in the present method to produce the compound 4-(5-formyl-furan-2-yl)benzoic acid which can subsequently be employed in the methods of the invention to form 4-[5-(2,4-dioxo-thiazolidin-5-ylidenemethyl)-furan-2-yl]benzoic acid or 4-[5-(4-oxo-2-thioxo-thiazolidin-5-ylidenemethyl)-furan-2-yl]benzoic acid. Examples 1 and 8 further describe preparation of these compounds.

In another embodiment, 3-aminobenzoic acid is employed in the present process to produce the compound 4(5-formyl-furan-2-yl)benzoic acid which can subsequently be employed in the methods of the invention to form 3-[5-(2,4-dioxo-thiazolidin-5-ylidenemethyl)-furan-2-yl]benzoic acid or 3-[5-(4-oxo-2-thioxo-thiazolidin-5-ylidenemethyl)-furan-2-yl]benzoic acid. Examples 2 and 9 further describe preparation of these compounds.

In another embodiment, this method of the invention can be employed to form additional intermediate compounds by reacting additional starting materials with 2-furaldehyde. One example of another group of intermediate compounds that can be formed by this method is furan-2-carbaldehydes. For example, when 4-hydroxybenzoic acid is employed as the starting material in the method, 5-(4-hydroxy-phenyl)-furan-2-carbaldehyde is produced. This intermediate can subsequently be employed to form 5-[5-(4-hydroxy-phenyl)-furan-2-ylmethylene]-thiazolidine-2,4-dione or 5-[5-(4-hydroxy-phenyl)-furan-2-ylmethylene]-2-thioxo-thiazolidin-4-one. Examples 3 and 10 further describe preparation of these compounds.

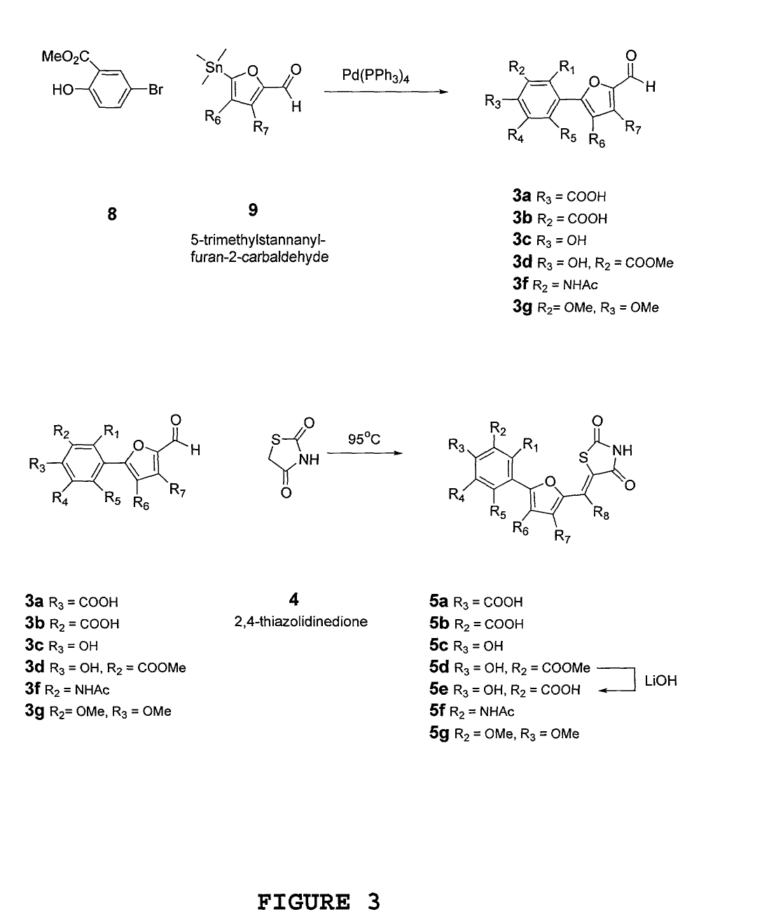

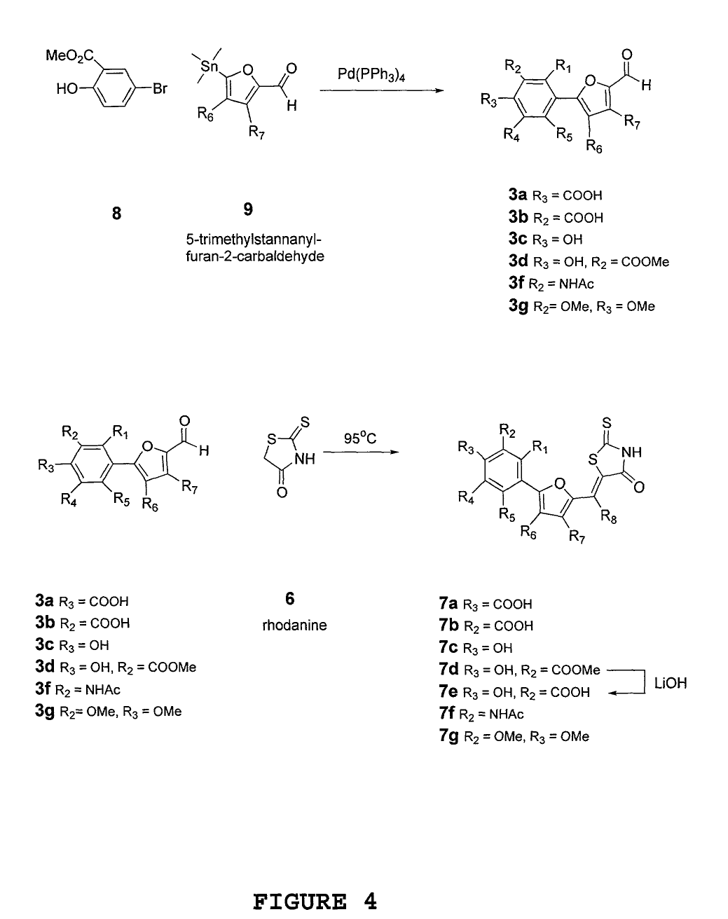

In another aspect, as shown in FIGS. 3 and 4, the present invention provides a method for the manufacture of methyl ester intermediates. In this method, a benzene derivative, such as a halobenzene, is reacted with 5-trimethylstannanyl-furan-2-carbaldehyde in the presence of tetrakis(triphenylphosphine)palladium (Pd(PPh₃)₄) in a solvent under an inert atmosphere. Suitable halobenzenes include, for example, bromobenzenes and iodobenzenes, such as 4-bromobenzoate. Suitable solvents for use in the reaction include, but are not limited to, tetrahydrofuran, dimethylformamide, dimethyl ether, and dioxane. For example, the reaction can be performed in dimethylformamide (DMF) under a nitrogen (N₂) atmosphere. The reaction mixture is heated to a temperature of between about 50 and 100° C. for a period of time of about 4 to 40 hours. For example, the reaction mixture can be heated to a temperature of about 600C for a period of about 30 hours.

The solution is then dried, for example, by evaporating under reduced pressure. If desired, the intermediate compound then can be purified by chromatography. Examples 4 and 11 further describe preparation of these compounds.

The 5-trimethylstannanyl-furan-2-carbaldehyde used in the above method can be prepared by any known method. In one embodiment of the present invention, this compound also can be prepared according to the following method.

A solution of 4-methylpiperidine in a solvent, such as THF, is formed at temperature of about −60 to about −100° C. under an inert atmosphere. For instance, the solution can be formed at a temperature of about −78° C. under a nitrogen atmosphere. Butyl lithium (BuLi) in hexane is then added to the solution, followed by the addition of 2-furaldehyde.

While maintaining the reaction temperature, another portion of BuLi is added to the reaction mixture. The mixture is then allowed to warm to a temperature of about −10 to −40° C. and stirred for a period of about 1 to 10 hours. For example, the reaction mixture can be warmed to a temperature of about −20° C. and stirred for a period of about 5 hours.

The reaction mixture is then cooled again to a temperature of about −60 to −100° C., for example −78° C., and added to a solution of Me₃SnCl in the same solvent. The reaction mixture is then allowed to warm gradually to room temperature and stirred overnight.

The reaction is then quenched, for example, by adding cold brine or cold water, followed by extraction with ethyl acetate or dichloromethane. The extracted organic phase then can be dried and concentrated using conventional methods. If desired, the product can be purified by chromatography or by any other suitable means. This process for the manufacture of 5-trimethylstannanyl-furan-2-carbaldehyde is further described in Examples 4 and 11.

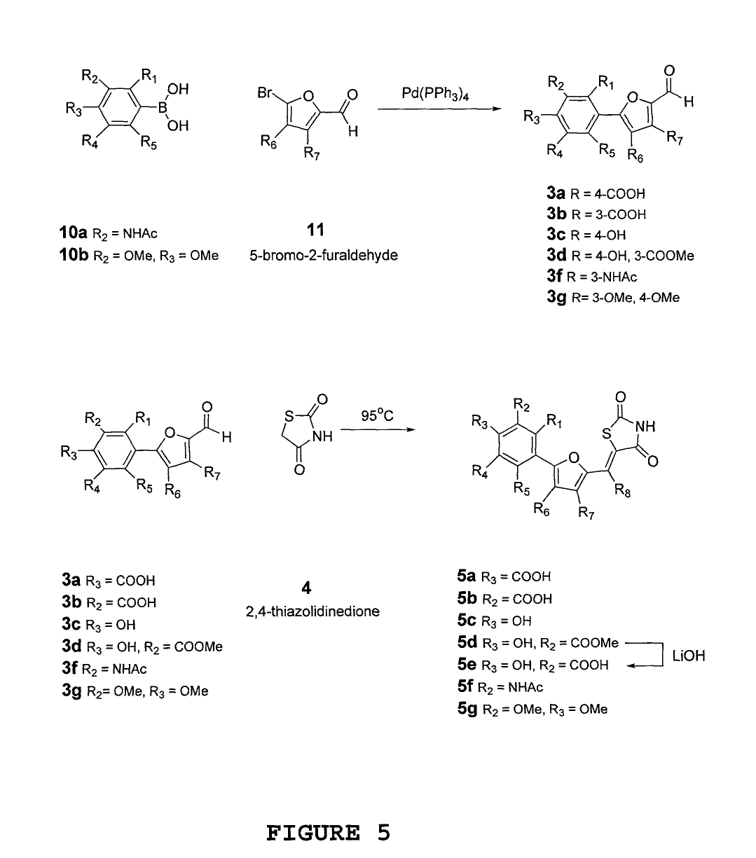

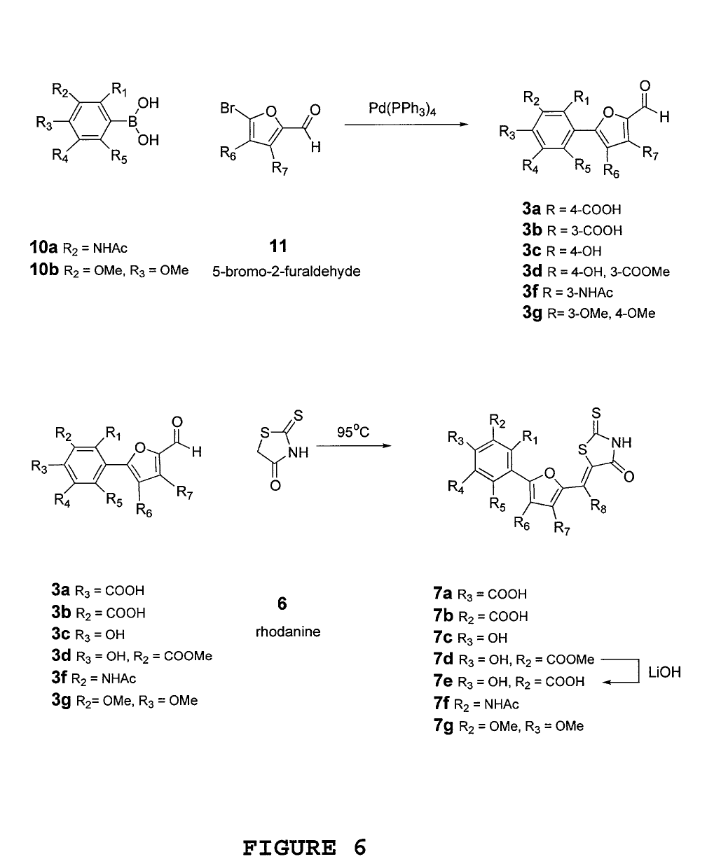

In an additional aspect, as shown in FIGS. 5 and 6, the present invention provides a method for the manufacture of intermediate compounds from a bromofuraldehyde and a phenylboronic acid. In accordance with this method, the bromofuraldehyde and the phenylboronic acid are mixed with tetrakis(triphenyl-phosphine)palladium, a salt, dioxane, and deionized water. Suitable salts for use in this reaction include, but are not limited to, sodium carbonate, potassium carbonate, and sodium bicarbonate. The solution is then deoxygenated, for example, with nitrogen. Following deoxygenation, the mixture is heated to a temperature of about 50 to 100° C. for a period of about 4 to 24 hours. For instance, the mixture can be heated to a temperature of about 90° C. for a period of about 10 hours.

The reaction mixture is then cooled to room temperature. The product then can be recovered by pouring the reaction mixture onto a silica gel column and eluting with a mixture of ethyl acetate and hexane.

In one embodiment, 4-bromofuraldehyde and 3-acetamidophenylboronic acid are employed in the present method to produce the compound N-[3-(5-formyl-furan-2-yl)phenyl]acetamide which can subsequently be employed in the methods of the invention to form N-{3-[5-(2,4-dioxo-thiazolidin-5-ylidenemethyl)-furan-2-yl]phenyl}acetamide or N-{3-[5-(4-oxo-2-thioxo-thiazolidin-5-ylidenemethyl)-furan-2-yl]phenyl}acetamide. Example 6 further describes preparation of these compounds.

In another embodiment, 3,4-dimethoxyphenyl-boronic acid and 5-bromo-2-furaldehyde are employed in the present method to produce the compound 5-(3,4-dimethoxyphenyl)-2-furaldehyde which can subsequently be employed in the methods of the invention to form 5-[5-(3,4-dimethoxy-phenyl)-furan-2-ylmethylene]-thiazolidine-2,4-dione or 5-[5-(3,4-dimethoxy-phenyl)furan-2-ylmethylene]-2-thioxo-thiazolidin-4-one. Examples 7 and 13 further describe preparation of these compounds.

Intermediate compounds formed by the methods of the invention described above can subsequently be used in the following methods of the invention to produce thiazolidinedione derivatives or rhodanine derivatives of the invention. In one aspect, as shown in FIGS. 1 to 7, the present invention provides methods for the preparation of thiazolidinedione compounds.

Such compounds can be formed by reacting the intermediate compound with 2,4-thiazolidinedione in a solvent, such as ethanol. The intermediate compound can be used in its crude form or can be purified, as by chromatography, prior to its use.

Piperidine is added to the mixture, and the resulting suspension is heated to a temperature of about 50 to 100° C., while stirring, for a period of about 1 to 12 hours. For example, the suspension can be heated to a temperature of about 70° C. for a period of about 5 hours.

The mixture is then cooled with ice, resulting in formation of a yellow precipitate. The precipitate can be filtered and washed, for example, with ethyl acetate and ether. To remove any residual piperidine, the crude product can be suspended in aqueous HCl and placed in an ultrasound bath for a period of about 10 minutes. The resulting product can be filtered and dried in a conventional manner, for example, by lyophilization. Examples 1 through 7 further describe preparation of thiazolidinedione compounds.

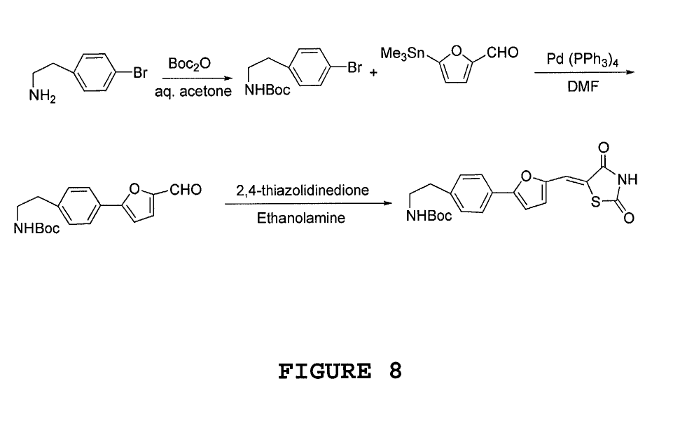

In another aspect, as shown in FIGS. 8 to 13, the present invention provides methods for the preparation of rhodanine compounds.

Such compounds can be formed by reacting an intermediate compound formed by the methods of the invention described above with rhodanine in a solvent, such as ethanol. It may be desirable to perform this reaction in the presence of a catalyst, for example, piperidine. The mixture can be stirred, under microwave irradiation, for a period of time of about 60 to 1000 seconds at a temperature of about 50 to 200° C. For instance, the mixture can be stirred for a period of time of about 300 seconds at 160° C., while stirring under microwave irradiation.

The reaction mixture is then cooled to room temperature, forming the product as a precipitate. The precipitate can be filtered, washed, for example, with ethyl acetate and ether, and dried, for example, in vacuo. Examples 8 through 13 further describe preparation of rhodanine compounds.

When the intermediate compound formed by the methods of the invention is a benzoic acid methyl ester, it may be desirable to convert the methyl ester to the corresponding benzoic acid. In such instances, the present invention provides a method by which this conversion can occur. The methyl ester intermediate is suspended in a solvent, such as methanol or a methanol/THF mixture. A solution of LiOH in water is then added to the solution. The reaction mixture is stirred at room temperature for a period of time of about 1 to 30 hours. For example, the reaction can be stirred at room temperature for a period of about 20 hours.

The solution is then acidified to a pH of about 1 and quickly extracted. The solution can be acidified, for example, with a solution of citric acid or 2N HCl. Extraction of the product can be accomplished with ethyl acetate or dichloromethane.

The extracted organic layers can then be dried, for example, over MgSO₄. If desired, the resulting benzoic acid can be filtered and concentrated in vacuo. Examples 5 and 12 further describe conversion of benzoic acid methyl esters to the corresponding benzoic acid.

The methods of the present invention now will be described in terms of specific embodiments for the preparation of a compound of formula I 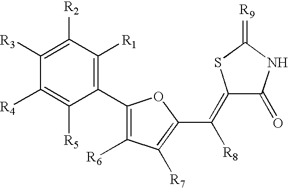
wherein R₁ to R₈ each independently are H, alkyl, alkenyl, alkynyl, aryl, heterocycle, COOH, COOAlkyl, CONR₁₀R₁₁, C(O)R₁₂, OH, OAlkyl, OAc, SH, SR₁₂, SO₃H, S(O)R₁₂, SO₂NR₁₀R₁₁, S(O)₂R₁₂, NH₂, NHR₁₂, NR₁₀R₁₁, NHCOR₁₂, N₃, NO₂, PH₃, PH₂R₁₂, H₂PO₄, H₂PO₃, H₂PO₂, HPO₄R₁₂, PO₂R₁₁R₁₂, CN, or X. R₉ is O, S, or NR₁₂; and R₁₀, R₁₁, and R₁₂ each independently are hydrogen, alkyl, alkenyl, alkynyl, aryl, or heterocycle, or R₁₀ and R₁₁ together with the nitrogen to which they are attached can be joined to form a heterocyclic ring. These embodiments exemplify the invention and do not limit the scope of the invention.

In one embodiment, the method involves reacting an aminobenzoic acid, such as 4-aminobenzoic acid or 3-aminobenzoic acid, with a 2-furaldehyde in the presence of nitrous acid and a copper catalyst to form a 5-formyl-furan-2-ylbenzonic acid intermediate. The 5-formyl-furan-2-yl-benzonic acid intermediate then is reacted with either 2,4-thiazolidinedione or rhodanine to form the corresponding thiazolidinedione or rhodanine derivative.

The nitrous acid employed in the reaction can be formed in situ by addition of a nitrate, such as sodium nitrate. The copper catalyst used in the invention can be, for example, a CuCl₂/CuCl catalyst. In some embodiments, the reaction mixture is heated to a temperature of about 70° C. to about 95° C., for example, to a temperature of about 70° C. Alternatively, the mixture can be heated to about 160° C. with irradiation.

In another embodiment, the method of the invention comprises reacting a bromobenzoate, such as 2-hydroxy-5-bromobenzoate, 5-trimethylstannanyl-furan-2-carbaldehyde, and Pd(PPh₃)₄ in a solvent, such as dimethylformamide, under an inert atmosphere, such as nitrogen, to form a 5-formyl-furan-2-ylbenzonic acid methyl ester intermediate. The 5-formyl-furan-2-ylbenzonic acid methyl ester intermediate formed in the reaction can be used to prepare the thiazolidinedione or rhodanine derivatives without additional manipulation. However, in some instances, it may be desirable to purify the intermediate. In such instances, the intermediate can be purified by chromotography.

The methyl ester intermediate is then heated with either 2,4-thiazolidinedione or rhodanine to form the corresponding thiazolidinedione or rhodanine derivative. The reaction mixture is heated, for example, to a temperature of about 70° C. to about 95° C., more particularly to a temperature of 90° C.

In one embodiment, the 5-trimethylstannanyl-furan-2-carbaldehyde employed in the reaction is formed by reacting 4-methylpiperidine and 2-furaldehyde in a solvent, such as tetrahydrofuran, under an inert atmosphere, such as nitrogen, in the presence of BuLi at a temperature of about −60 to −100° C. The mixture is stirred while allowing it to warm to a temperature of about −10 to −40° C. Then, the reaction mixture is cooled again to a temperature of about −60 to −100° C., followed by addition of a solution of Me₃SnCl and by warming of the reaction temperature under agitation. Next, the reaction is quenched with cold brine, and the 5-trimethylstannanyl-furan-2-carbaldehyde is extracted in the organic phase with EtOAc and, optionally, is dried.

The 5-trimethylstannanyl-furan-2-carbaldehyde can be used in the method of the invention without additional manipulation. However, in some instances, it may be desirable to purify the compound prior to use. In such instances, the 5-trimethylstannanyl-furan-2-carbaldehyde can be purified by, for example, chromatography.

In another embodiment, the method of the invention comprises reacting a bromobenzoate, such as 2-hydroxy-5-bromobenzoate, 5-trimethylstannanyl-furan-2-carbaldehyde, and Pd(PPh₃)₄ in a solvent, such as methanol or a mixture of methanol and tetrahydrofuran, under an inert atmosphere, such as nitrogen, to form a 5-formyl-furan-2-ylbenzonic acid methyl ester intermediate. The 5-formyl-furan-2-ylbenzonic acid methyl ester intermediate formed in the reaction can be used to prepare the thiazolidinedione or rhodanine derivatives without additional manipulation. However, in some instances, it may be desirable to purify the intermediate. In such instances, the intermediate can be purified by chromatography.

The 5-formyl-furan-2-ylbenzonic acid methyl ester intermediate is heated with either 2,4-thiazolidinedione or rhodanine to form the corresponding thiazolidinedione or rhodanine derivative. This derivative is then suspended in a solution of LiOH in a solvent. The suspension is stirred for a period of about 2 to 40 hours, and the pH of the mixture is adjusted to about pH 1, followed by extraction of the product with EtOAc. The product optionally is dried over MgSO₄. If desired, the final thiazolidinedione methyl ester or rhodanine methyl ester can be purified prior to conversion to the corresponding benzoic acid.

In yet another embodiment, the method of the invention comprises forming a mixture 4-bromofuraldehyde, a phenylboronic acid, such as 3-acetamidophenylboronic acid or 3,4-dimethoxy-phenylboronic acid, and Pd(PPh₃)₄ in the presence of dioxane, D.I. water, and sodium carbonate.

The mixture is then deoxygenated, for example with N₂, and heated for a period of about 5 to 12 hours to form a furaldehyde intermediate compound. The reaction mixture is then cooled to room temperature and poured over a silica gel column from which the furaldehyde intermediate compound is eluted, for example, with a 1:1 mixture of EtoAc/Hexane. The furaldehyde intermediate is then heated, for example, to a temperature of about 50 to 100° C. with either 2,4-thiazolidinedione or rhodanine to form the corresponding thiazolidinedione or rhodanine derivative.

Any of the thiazolidinedione or rhodanine compounds of the present invention can be made by the methods described above. Where it is necessary to add or modify substituents attached to the compounds, for example substituents on the phenyl or furan rings of the present invention, such modification are within the level of skill of an ordinary artisan in view of the present disclosure.

Common ligand mimics of the present invention containing linkers can be prepared from less complex common ligand mimics of the invention by conventional methods. These common ligand mimics can also be prepared by the following methods.

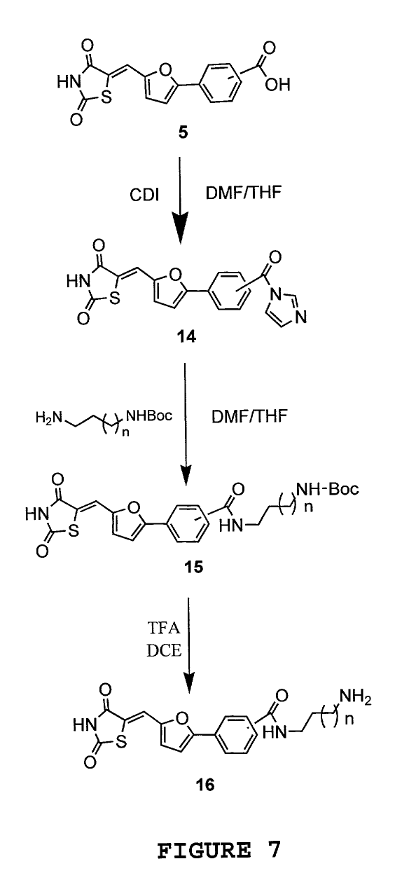

As shown in FIG. 7, a common ligand mimic of the present invention containing a carboxylic acid group is dissolved in a solvent, such as dimethylformamide or tetrahydrofuran. The compound is then reacted with 1,1′-carbonyldiimidazole in tetrahydrofuran at a temperature of about 40 to 80° C., for example, 40 to 50° C. The reaction mixture is agitated for a period of time of about 20 to 120 minutes, for example 20 minutes.

The mixture is then covered and refrigerated for a period of time at a temperature of about −20 to 10° C. For example the reaction mixture can be refrigerated overnight at a temperature of about −10° C. The precipitate can then be collected by filtration and washed with THF to form an intermediate compound.

The intermediate compound is then placed in a mixture of DMF and THF. Boc protected diamines (t-butyl carbamate protected diamines) are added to the mixture, and the mixture is heated to a temperature of about 40 to 80° C. for a period of about 1 to 5 hours, followed by evaporation of the solvent, for example, under reduced pressure. For example, the mixture can be heated at a temperature of about 65° C. for a period of about 1 hour.

Next, a solution of 50% trifluoacetic acid in dichloroethane (100 ml) is added to the precipitate and reacted for a period of about 10 to 40 minutes, followed by evaporation of the remaining solvent. For example, the mixture can be reacted for a period of about 10 minutes, followed by evaporation of extra solvent. The precipitate can then be dissolved in a solvent, such as DMF, by heating. The solution is cooled to room temperature, and a Na₂CO₃ solution added. When a precipitate forms, it is filtered. If necessary, additional solvent and water can be added. The final product can then be washed with a mixture of water and alcohol, such as water and MeOH, and then dried. This method is described further in Example 19.

As shown in FIG. 8, common ligand mimics of the invention also can be prepared by the following method. The compounds 4-bromophenethylamine and NaHCO₃ are suspended in aqueous acetone at a temperature of about −10 to 10° C., for example 0° C. A solution of di-tert-butyldicarbonate acetone then is added dropwise to the solution, which is stirred at room temperature for a period of time. For example, the solution can be stirred overnight at room temperature.

The reaction then can be poured into water and extracted with ethyl acetate. The extracts then can be dried by conventional means, for example with MgSO₄, and concentrated to provide a powder of an intermediate compound.

Next, a mixture of the intermediate product, 5-trimethylstannanyl-2-furaldehyde, and tetrakis(triphenylphosphine)palladium is formed in a solvent, such as DMF. The mixture is then heated to a temperature of about 50 to 90° C. for a period of about 20 to 30 hours. For example, the mixture can be heated to a temperature of about 60° C. for a period of about 24 hours. The reaction mixture then is concentrated under reduce pressure, and the residue purified by chromatography, for example using an extractant of EtOAc/Hexanes to provide an intermediate furaldehyde.

A solution of the intermediate furaldehyde, 2,4-thiazolidinedione, and ethanolamine is formed in a solvent, such as dioxane. The solution is then heated to reflux for a period of about 2 to 3 days. For example, the solution can be heated to reflux for a period of about 3 days. The reaction mixture is concentrated, and the resulting residue triturated several times with ethyl acetate. The precipitate is then collected by filtration to provide the desired common ligand mimic. This method is further described in Example 20.

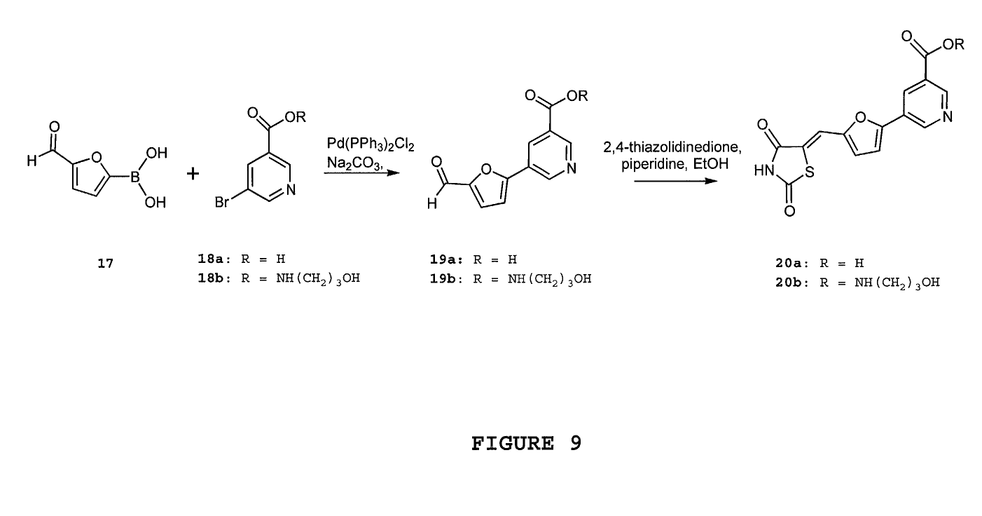

As shown in FIG. 9, common ligand mimics of the invention can also be prepared by the following method. The compounds 2-formylfuran-5-boronic acid, 5-bromonicotinic acid, and sodium carbonate (262 mg, 2.48 mmol) are added to a mixture of solvent and water, for example a mixture of dioxane, water, ethanol, and DMF. Dichlorobis(triphenylphosphine)palladium is added to the mixture, and the mixture heated to a temperature of about 80 to 100° C. for a period of about 12 to 18 hours. For example, the mixture can be heated to a temperature of about 90° C. for a period of about 15 hours. Another portion of dichlorobis(triphenyl-phosphine)palladium and 2-formylfuran-5-boronic acid can be added to the reaction mixture, if necessary, and the reaction again stirred, for example overnight at room temperature.

Volatiles then were removed in vacuo, and the residue diluted with water, followed by extraction with ethyl acetate. Combined organic layers then can be dried by conventional methods, for example over Mg₂SO₄, followed by filtration and concentration in vacuo. The crude product can be purified by flash chromatography, for example with a CH₂Cl₂/MeOH mixture, to provide an intermediate nicotinic acid.

The intermediate nicotinic acid and 2,4-thiazolidinedione then are mixed in ethanol. Piperidine is added dropwise, and the reaction mixture stirred at a temperature of about 60 to 80° C. for a period of about 1 to 6 hours. For example, 1 to 5 drops of piperidine can be added, and the reaction stirred at a temperature of is about 70° C. for a period of about 36 hours.

The resulting precipitate can be collected on filter paper using a Büchner funnel and washed with ethyl acetate, followed by ethyl ether to give the desired product. This method is further described in Examples 21 and 22.

Bi-ligands of the present invention can be produced by any feasible method. For example, the compounds of the present invention can be produced by the following methods. These methods are exemplified using a common ligand mimic or Formula I and a pyridine dicarboxylate specificity ligand. However, one having ordinary skill in the art will appreciate that variations in such methods can be employed to produce bi-ligands having other common ligand mimics or other specificity ligands.

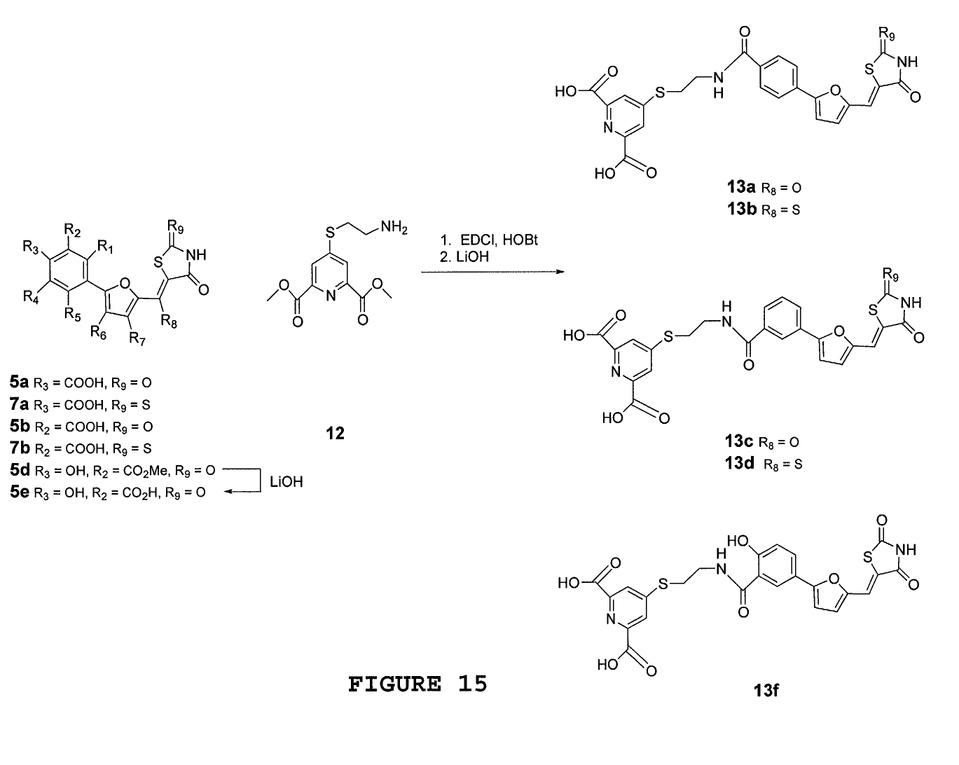

As shown in FIG. 15, a common ligand mimic of the invention, such as a thiazolidinedione or rhodanine compound of Formula I can be reacted in the presence of HOBt.H₂O. Suitable solvents include dimethylformamide, tetrahydrofuran, and dichloromethane. For example, the reaction of 4-(2-amino-ethylsulfanyl)-pyridine-2,6-dicarboxylic acid dimethyl ester can be performed in dimethylformamide with the addition of (HOBt.H₂O). Triethylamine and 1-dimethylaminopropyl-3-ethyl-carbodiimide (EDCI) are then added to the mixture. The reaction is then stirred at room temperature for a period of about 2 to 40 hours. For example, the reaction can be stirred at room temperature for a period of about 24 hours.

The reaction precipitate is collected and washed in a mixture of solvent, hydrochloric acid, and methanol. Then, the recovered solid can be suspended in a mixture of alcohol, base, and water, such as a methanol, LiOH, and water mixture. This solution is stirred at room temperature for a period of about 1 to 24 hours until it is homogenous. The solution is then acidified, for example with citric acid or aqueous 2N HCl. The resulting precipitated product can then be filtered, washed with water, and dried.

As used herein, a “combinatorial library” is an intentionally created collection of differing molecules that can be prepared by the means provided below or otherwise and screened for biological activity in a variety of formats (e.g., libraries of soluble molecules, libraries of compounds attached to resin beads, silica chips or other solid supports). A “combinatorial library,” as defined above, involves successive rounds of chemical syntheses based on a common starting structure. The combinatorial libraries can be screened in any variety of assays, such as those detailed below as well as others useful for assessing their biological activity. The combinatorial libraries will generally have at least one active compound and are generally prepared such that the compounds are in equimolar quantities.

Compounds described in previous work that are not taught as part of a collection of compounds or not taught as intended for use as part of such a collection are not part of a “combinatorial library” of the invention. In addition, compounds that are in an unintentional or undesired mixture are not part of a “combinatorial library” of the invention.

The present invention provides combinatorial libraries containing two or more compounds. The present invention also provides combinatorial libraries containing three, four, or five or more compounds. The present invention further provides combinatorial libraries that can contain ten or more compounds, for example, fifty or more compounds. If desired, the combinatorial libraries of the invention can contain 100,000 or more, or even 1,000,000 or more, compounds.

In one embodiment, the present invention provides combinatorial libraries containing common ligand variants of compounds of Formula I. These common ligand variants are active forms of the compounds of Formula I that are capable of binding to a specificity ligand to form a bi-ligand. For example, where one of R₁ to R₈ is a COOH or COOAlkyl group, the common ligand variant can be a compound containing the group COO⁻. Common ligand variants of the invention include common ligand mimics in which the subsituents on the compounds are complex ligands such as those attached to the compounds listed in Tables 6 to 12.

In another embodiment, the present invention provides combinatorial libraries containing bi-ligands of the invention. The bi-ligands are the reaction product of a common ligand mimic and a specificity ligand which interact with distinct sites on a single receptor. For example, the common ligand mimic can be one or more common ligand mimics for NAD which binds to a conserved site on a dehydrogenase, like ADH. In such a bi-ligand, the specificity ligand is one or more ligands which bind a specificity site on ADH.

Such combinatorial libraries can contain bi-ligands having a single common ligand mimic bonded to multiple specificity ligands. Alternatively, the combinatorial libraries can contain bi-ligands having a single specificity ligand bonded to multiple common ligand mimics. In another aspect, the combinatorial libraries can contain multiple common ligand mimics and multiple specificity ligands for one or more receptors.

The use of a common ligand mimic of the invention to produce the combinatorial library allows generation of combinatorial libraries having improved affinity and/or specificity. Selection and tailoring of the substituents on the common ligand mimic also allows for production of combinatorial libraries in a more efficient manner than heretofore possible.

Bi-ligand libraries of the invention can be prepared in a variety of different ways. For example, two methods employing a resin, such as HOBt resin, carbodiimide resin, or DIEA (diisopropyldiisoamine) resin, can be used to form bi-ligand libraries. In one such method, bi-ligand libraries can be prepared via direct coupling of amines to common ligand mimics of the invention having a carboxylic acid group.

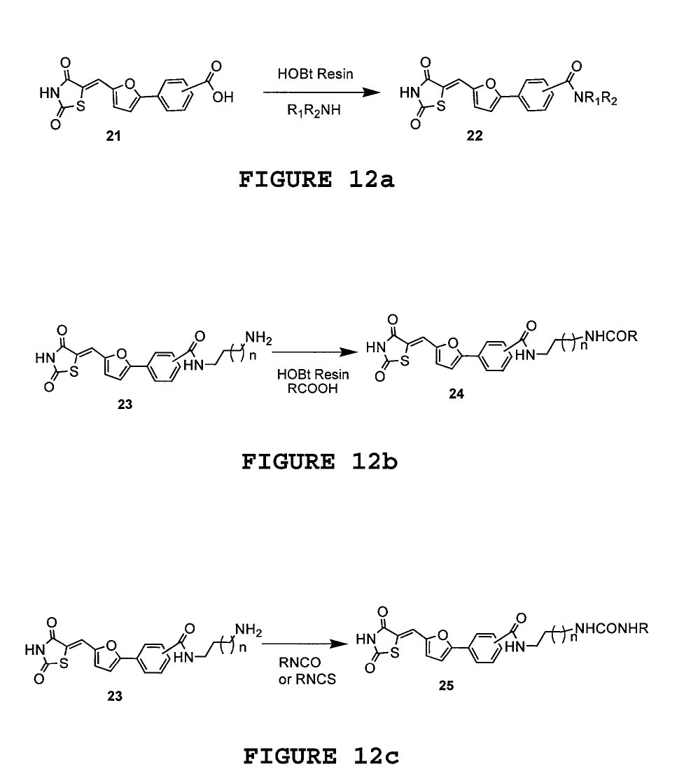

As shown in FIG. 12a, bi-ligand libraries can be prepared in the following manner. HOBt resin is swelled in a dry solvent, such as a mixture of dry THF and dry DMF, and added to a solution of a common ligand mimic of the invention that is dissolved in a solvent, such as a mixture of DMF and DIC. The solution is shaken at room temperature overnight and then washed with 3×dry DMF and 3×dry THF. The resin is added to a solution of an amine in a solvent, for example dry DMF. The mixture is shaken again at room temperature overnight. The resin then can be filtered and washed with solvent, and the filtrate can be collected and vacuum dried to provide bi-ligands of the invention. Nonlimiting examples of amines useful for the preparation of bi-ligand libraries include those in Table 1.

TABLE 1

| | | |
|---|---|---|
| cyclopropylamine | nipecotamide | 3-chloro-p-anisidine |
| isopropylamine | 1-(3-aminopropyl) | 5-amino-1-napthol |
| | pyrrolidine | |
| N,N-diethyl-N′- | 2-(2-aminoethyl)-1- | 2-amino-5,6-dimethyl- |
| methylethylenediamine | methylpyrrolidine | benzimidazole |
| N-(3-aminopropyl)-N- | 2-(aminomethyl)-1- | N,N-diethyl-p- |
| methylaniline | ethylpyrrolidine | phenylenediamine |
| hydroxylamine | N-(2-aminoethyl)- | 1-(2-pyridyl) |
| hydrochloride | piperidine | piperazine |
| cyclobutylamine | 4-(2-aminoethyl) | 4-pentylaniline |
| | morpholine | |
| N-methylallylamine | propylamine | pyrrolidine |
| 3-pyrroline | 2-(aminomethyl) | 1-phenylpiperazine |
| | benzimidazole | |
| diethylamine | ethyl 3-aminobutyrate | 4-butoxyaniline |
| isobutylamine | 5-aminoindan | 2,3-dimethoxybenzylamine |
| N-butylamine | trans-2- | 2,4- |
| | phenylcyclopropylamine | dimethoxybenzylamine |
| N-methylpropylamine | 3-phenyl-1-propylamine | 3,5-dimethoxybenzylamine |
| sec-butylamine | beta-methylphenethylamine | ethyl 4-aminobutyrate |
| 2-methoxyethylamine | N-methylphenethylamine | 1-cyclohexylpiperazine |
| 4-amino-1,2,4- | p-isopropylaniline | 4-piperidinopiperidine |
| triazole | | |
| cyclopentylamine | 3-aminobenzamide | 2-amino-5-chlorobenzoxazole |
| ethyl 4-amino-1- | N,N-dimethyl-1,4- | 2-amino-5- |
| piperidinecarboxylate | phenylenediamine | trifluoromethyl-1,3,4- |
| | | thiadiazole |
| morpholine | N-(4-pyridylmethyl)ethylamine | 2-aminobiphenyl |
| 1-ethylpropylamine | 4-aminobenzamide | 3-aminobiphenyl |
| neopentylamine | 3,4-(methylenedioxy)- | N-undecylamine |
| | aniline | |
| N-ethylisopropylamine | 4-hydroxybenzamide | piperidine |
| N-methylbutylamine | 6-aminonicotinamide | 4-cyclohexylaniline |
| 2-amino-1- | 4-fluorophenethylamine | 2- |
| methyloxypropane | hydrochloride | (trifluoromethyl)benzylamine |
| 3-methoxypropylamine | 3-amino-4-methylbenzyl | 2,4-dimethyl-6- |
| | alcohol | aminophenol |
| thiazolidine | 3-methoxybenzylamine | 2,4-dichlorobenzylamine |
| 3-amino-1,2,4-triazine | 4-ethoxyaniline | 3,4-dichlorobenzylamine |
| furfurylamine | 4-methoxy-2-methylaniline | 4-aminoquinaldine |
| diallylamine | 4-methoxybenzylamine | 4-(methylthio)aniline |
| 2-methylpiperidine | m-phenetidine | 1-benzylpiperazine |
| 3-methylpiperidine | 5-amino-2-methoxyphenol | 4-piperidino aniline |
| 4-methylpiperidine | tyramine | 4-(trifluoromethoxy)- |
| | | aniline |
| cyclohexylamine | 2-fluorophenethylamine | 4-hexylaniline |
| hexamethyleneimine | 3-fluorophenethylamine | 4-amino-2,6- |
| | | dichlorophenol |
| 1-aminopiperidine | 3-(methylthio)aniline | 4-morpholinoaniline |
| 2-amino-4-methoxy-6- | (3S)-(+)-1-benzyl-3- | N-(2-aminoethyl)-N- |
| methylpyrimidine | aminopyrrolidine | ethyl-m-toluidine |
| tetrahydrofurfurylamine | 1-methylpiperazine | 4-chlorobenzylamine |
| 1,3-dimethylbutylamine | 3,3,5- | 1-(2-furoyl)piperazine |
| | trimethylcyclohexylamine | |
| dipropylamine | 2-chlorobenzylamine | 1-(2- |
| | | fluorophenyl)piperazine |
| 4-aminomorpholine | 3-chlorobenzylamine | 1-(4- |
| | | fluorophenyl)piperazine |
| N-(3′-aminopropyl)-2- | 4-aminophenylacetic acid | 2-(3,4- |
| pyrrolidinone | ethyl ester | dimethoxyphenyl)ethylamine |
| 3- | N-acetylethylenediamine | 2-amino-fluorene |
| dimethylaminopropylamine | | |
| N- | 2,4-difluorobenzylamine | 3,4,5-trimethoxyaniline |
| isopropylethylenediamine | | |
| o-toluidine | N-phenyl-p-phenylenediamine | 4-aminodiphenylmethane |
| 1-aminonaphthalene | 2,6-difluorobenzylamine | aminodiphenylmethane |
| 5-amino-1-pentanol | 3,4-difluorobenzylamine | 2,5-difluorobenzylamine |
| 3-ethoxypropylamine | 2-(aminomethyl)-1,3- | 3-phenoxyaniline |
| | dioxolane | |
| 3- | 2-aminonaphthalene | 4-phenoxyaniline |
| (methylthio)propylamine | | |
| benzylamine | p-phenetidine hydrochloride | 1-(3- |
| | | chlorophenyl)piperazine |
| m-toluidine | 8-aminoquinoline | 4-amino-1- |
| | | benzylpiperidine |
| 3-fluoroaniline | N-(3-aminopropyl) | 4-aminohippuric acid |
| | morpholine | |
| p-toluidine | 7-amino-4-methylcoumarin | 2-amino-9-fluorenone |
| 1-amino-5,6,7,8- | 4-piperidone monohydrate | 2-methyl-1-(3- |
| tetrahydronaphthalene | hydrochloride | methylphenyl)piperazine |
| 2-(aminomethyl)pyridine | 2-amino-1- | 3,4,5- |
| | methylbenzimidazole | trimethoxybenzylamine |
| 3-(aminomethyl)pyridine | 4-phenylbutylamine | 2,2-diphenylethylamine |
| 4-(aminomethyl)pyridine | 4-amino-N-methylphthalimide | 3-benzyloxyaniline |
| 1,2,3,4-tetrahydro-1- | 4-(2-aminoethyl)benzene | 4-amino-4′- |
| naphthylamine | sulfonamide | methyldiphenylether |
| 2-amino-4- | N- | 1-methyl-3- |
| methylbenzothiazole | propylcyclopropanemethylamine | phenylpropylamine |
| 2-thiophenemethylamine | 4-tert-butylaniline | exo-2-aminonorbornane |
| 2-methylcyclohexylamine | 4′-aminoacetanilide | 1,4-benzodioxan-5-amine |
| 3,5-dimethylpiperidine | N-(4-aminobenzoyl)-beta- | piperonylamine |
| | alanine | |
| 4-methylcyclohexylamine | methyl 3-amino-benzoate | 5-phenoxy-o-anisidine |
| N-isopropyl-N-phenyl-p- | 2-methoxy-N-phenyl-1,4- | 4-amino-4′- |
| phenylenediamine | phenylenediamine | chlorodiphenylether |
| cyclohexanemethylamine | 2-ethoxybenzylamine | 1-piperonylpiperazine |
| heptamethyleneimine | 2-methoxyphenethylamine | 4-amino-4′- |
| | | methoxystilbene |
| 1-(4- | 4-isopropoxyaniline | cycloheptylamine |
| nitrophenyl)piperazine | | |
| 1- | 4-methoxyphenethylamine | (−)-cis-myrtanylamine |
| piperazinecarboxaldehyde | | |
| 2-amino-4- | 3,5-dimethoxyaniline | 4-(4-nitrophenoxy)- |
| methylthiazole | | aniline |
| 1,3,3-trimethyl-6- | alpha-(cyanoimino)-3,4- | 4-amino-4′- |
| azabicyclo [3,2,1] octane | dichlorophenethylamine | nitrodiphenylsulfide |
| 1-methylhomopiperazine | 1-ethylpiperazine | 2-amino-7-bromofluorene |
| N-(2- | 4-tert-butylcyclohexylamine | 2-(3- |
| aminoethyl)pyrrolidine | | chlorophenyl)ethylamine |
| 2-amino-5-phenyl-1,3,4- | 2-amino-4,5,6,7- | (1R,2S)-(+)-cis-1-amino- |
| thiadiazole sulfate | tetrahydrobenzo(b) | 2-indanol |
| | thiophene-3-carbonitrile | |
| 1-amino-4- | 2-(4- | n-undecylamine |
| methylpiperazine | chlorophenyl)ethylamine | |
| 2-heptylamine | 1-(3-aminopropyl)-2- | 2,6-dimethylmorpholine |
| | pipecoline | |
| N,N,N′-trimethyl-1,3- | 4-amino-2,2,6,6- | d(+)-alpha- |
| propanediamine | tetramethylpiperidine | methylbenzylamine |
| N-methylhexylamine | ethyl nipecotate | dl-1-amino-2-propanol |
| 1-(3-aminopropyl)-4- | N,N-dimethyl-N′- | dl-alpha- |
| methyl-piperazine | ethylethylenediamine | methylbenzylamine |
| 3-aminobenzyl alcohol | N,N-diethylethylenediamine | o-anisidine |
| (R)-(+)-2-amino-3- | 2-(furfurylthio) ethylamine | 3-amino-4-methylbenzyl |
| phenylpropanol | | alcohol |
| 2-(2-aminoethyl)-1,3- | 2,3-dimethyl | 3-amino5,5-dimethyl-2- |
| dioxolane | cyclohexylamine | cyclohexen-1-one |
| 6-amino-1-hexanol | N-methyl-b-alaninenitrile | 3-aminophenol |
| 3-isopropoxy | 1-methyl-4- | (R)-(+)-1- |
| propylamine | (methylamino)piperidine | phenylpropylamine |
| 2-methylbenzylamine | 1-amino-2-butanol | 2-piperidineethanol |
| (R)-1-(4- | 2-amino-2-methyl-1-propanol | 2,3-dimethyl-4- |
| methylphenyl)ethylamine | | aminophenol |
| 3-methylbenzylamine | 4-amino-1-butanol | 1-aminoindan |
| 4-methylbenzylamine | 3-(ethylamino)propionitrile | phenethylamine |
| N-methylbenzylamine | 4-hydroxypiperidine | 3,4-dimethylaniline |
| (+/−)-2-amino-1-butanol | N-(2-hydroxyethyl) | 1-naphthalene |
| | piperazine | methylamine |
| 2-(2-aminoethyl) | S(+)-1-cyclohexyl | 2-aminophenethyl alcohol |
| pyridine | ethylamine | |
| 6-amino-m-cresol | 4-aminophenol | decylamine |
| m-anisidine | 2-ethylpiperidine | 4-aminophenethyl alcohol |
| p-anisidine | N-methylcyclohexylamine | diethanolamine |
| methyl 4-aminobenzoate | 3-piperidinemethanol | 2-(methylthio)aniline |
| 5-amino-o-cresol | 2,4-dimethylaniline | 4-amino-2-chlorophenol |
| 4-fluorobenzylamine | 2,5-dimethylaniline | dibenzylamine |
| 1-(3-aminopropyl)- | 6′-amino-3′,4′(methylene- | 2-(aminomethyl)-5- |
| imidazole | dioxy)acetophenone | methylpyrazine |
| 2-(1-cyclohexenyl) | 3-amino-4-hydroxybenzoic | (R)-(+)-1-(4- |
| ethylamine | acid | methoxyphenyl)ethylamine |
| 2,(2-thienyl)ethylamine | (1R,2S)-1-amino-2-indanol | 4-ethynylaniline |
| 1-(3,4-dichlorophenyl) | N-(4-amino-2- | 1(−)-2amino-3-phenyl-1- |
| piperazine | chlorophenyl)morpholine | propanol |
| 1-acetylpiperazine | N-benzyl-2-phenylethylamine | 5-tert-butyl-o-anisidine |
| isonipecotamide | 5-phenyl-o-anisidine | 4-amino salicylic acid |
| 2-amino-m-cresol | cyclooctylamine | 2,4-dimethoxyaniline |
| 2-methoxy-6- | 3-hydroxytyramine | 4-amino-3-hydroxybenzoic |
| methylaniline | hydrobromide | acid |
| 2-aminonorbornane | 2-[2-(aminomethyl) | 1-amino-2- |
| hydrochloride | phenylthio] benzyl alcohol | methylnaphthalene |
| 5-aminoindazole | 2-amino-1,3-propanediol | 3-amino-5-phenylpyrazole |
| 5-aminobenzotriazole | 3-amino-1,2-propanediol | veratrylamine |
| methyl 4-aminobutyrate | 3-bromobenzylamine | 3-amino-1-phenyl-2- |
| hydrochloride | hydrochloride | pyrazolin-5-one |
| 2-chloro-4,6- | 1-(2-methoxyphenyl) | 5-amino-1-methyl-3- |
| dimethylaniline | piperazine hydrochloride | (thien-2-yl)pyrazole |
| (1S,2S)-(+)-2-amino-1- | 4-benzyloxyaniline | 3,5-bis (trifluoro- |
| phenyl-1,3-propanediol | hydrochloride | methyl)-benzylamine |
| 2-bromobenzylamine | (S)-(+)-2-amino-3- | 3-aminopyrrolidine |
| hydrochloride | cyclohexyl-1-propanol HCl | dihydrochloride |

| | |
|---|---|
| N-(4-methoxyphenyl)-p-phenylenediamine hydrochloride | 2-piperidinemethanol |

In another of such methods, bi-ligand libraries can be prepared by reacting carboxylic acids to common ligand mimics of the present invention having an amine or amide containing substituent.

As shown in FIG. 12b, bi-ligand libraries of the invention can also be prepared in the following manner. HOBt resin is swelled a dry solvent, such as dry THF, and added to a solution of a carboxylic acid in a solvent, such as a mixture of dry DMF and DIC. The solution is shaken at room temperature overnight and then washed with 3×dry DMF and 1×dry THF. The resin is added to a solution of a common ligand mimic of the invention in a solvent, for example dry DMF. The solution is again shaken at room temperature overnight. The resin then can be filtered and washed with solvent, followed by collection and vacuum drying of the filtrate to provide bi-ligands of the invention. Nonlimiting examples of carboxylic acids useful for the preparation of bi-ligand libraries include those in Table 2.

TABLE 2

| | | |
|---|---|---|
| acetic acid | 5-Bromonicotinic acid | 4-Chlorobenzoic acid |
| 4-Chloro-3-nitrobenzoic | 4-(3-Hydroxyphenoxy) | 4-Biphenylcarboxylic |
| acid | benzoic Acid | acid |
| N-Acetylglycine | 3,5-Dihydroxybenzoic acid | 2-Bromobenzoic acid |
| Propionic acid | 2,4-Dihydroxybenzoic acid | 3-Bromobenzoic acid |
| Crotonic acid | 2,3-Dihydroxybenzoic acid | 4-Bromobenzoic acid |
| 4-pentenoic acid | 2-Chloro-5-nitrobenzoic | 4-Phenoxybenzoic acid |
| | acid | |
| methacrylic acid | 6-Mercaptonicotinic acid | 4-Mercaptobezoic acid |
| Pyruvic acid | Cyclohexanepropionic acid | acrylic acid |
| 3-Hydroxy-2-methyl-4- | 1-(4-Chiorophenyl)-1- | 4-Hydroxy-3-(morpholino- |
| quinolinecarboxylic acid | cyclopropanecarboxylic acid | mehtyl)benzoic acid |
| n-butyric acid | 3-Chlorobenzoic acid | isobutyric acid |
| methoxyacetic acid | 2-Chlorobenzoic acid | 3-Indolebutyric acid |
| mercaptoacetic acid | 5-Nitro-2-furoic acid | 2,6-Difluorobenzoic acid |
| 2,3-Difluorobenzoic | 6-Chloronicotinic acid | Ethoxyacetic acid |
| acid | | |
| trans-2,3-dimethylacrylic acid | 1,4-Dihydroxy-2-napthoic | 3,7-Dihydroxy-2-napthoic |
| | acid | acid |
| Cyclobutanecarboxylic | 2-methylcyclopropane | 2-Chloro-4-nitrobenzoic |
| acid | carboxylic acid | acid |
| cyclopropanecarboxylic | 4-(4-Hydroxyphenoxy) | 9H-Fluorene-9-carboxylic acid |
| acid | benzoic Acid | |
| 2-ketobutyric acid | 3,5-Difluorobenzoic acid | Pentafluorobenzoic acid |
| Isovaleric acid | 2,4-Difluorobenzoic acid | Indole-5-carboxylic acid |
| Trimethylacetic acid | 3,4,5-Trimethoxybenzoic | 3-Nitrobenzoic acid |
| 99% | acid | |
| 3-methoxypropionic acid | Indole-2-carboxylic acid | 3-Phenoxybenzoic acid |
| 3-Hydroxybutyric acid | 2-benzofurancarboxylic acid | 4-Phenylbutyric acid |
| 4,8-Dihydroxyquinoline- | 2,3,4-Trimethoxybenzoic | 3-(3,4-Dimethoxyphenyl) |
| 2-carboxylic acid | acid | propionic acid |
| (Methylthio)acetic acid | indazole-3-carboxylic acid | 3-chloropropionic acid |
| Pyrrole-2-carboxylic | Benzotriazole-5-carboxylic | 3-bromo-4-methylbenzoic |
| acid | acid | acid |
| 4-Aminobenzoic acid | Indoline-2-carboxylic acid | 3-Bromophenylacetic acid |
| 5-Acetylsalicylic acid | Pentafluoropropionic acid | 4-bromophenylacetic acid |
| 2-Furoic acid | 4-acetylbenzoic acid | 2-Iodobenzonic acid |
| Cyclopentanecarboxylic | 5-Norbornene-2,3- | 9-Flourenone-2- |
| acid | dicarboxylic acid | carboxylic acid |
| | monomethyl ester | |
| trans-3-Hexenoic acid | 3-(5-Nitro-2-furyl)acrylic | xanthene-9-carboxylic |
| 97% | Acid | acid |
| Piperonylic acid | 4-Carboxyphenylboronic acid | 3-Benzoylbenzoic acid |
| 2-tetrahydrofuroic acid | 4-Dimethylaminobenzoic acid | 4-benzoylbenzoic acid |
| 2-Phenoxybenzoic acid | 3-Dimethylaminobenzoic acid | 2-Butynoic acid |
| Tetrahydro-3-furoic | 3-Methoxyphenylacetic acid | 2-Hydroxyisobutyric acid |
| acid | | |
| hexanoic acid | 4-Ethoxybenzoic acid | 2,4-Hexadienoic acid |
| 2-Ethylbutyric acid | 4-methoxyphenylacetic acid | (Ethylthio)acetic acid |
| DL-3-Methylvaleric | (alpha,alpha,alpha-tetra- | 1-Cyclohexene-1- |
| acid, 97% | fluoro-p-tolyl)acetic acid | carboxylic acid |
| Tert-Butylacetic acid, | 1,4-Benzodioxan-2- | 2-Phenoxymethylbenzoic |
| 98% | carboxylic acid | Acid |
| 1-Acetylpiperidine-4- | (R)-(−)-5-oxo-2- | 2-hydroxy-2- |
| carboxylic acid | tetrahydro-furancarboxylic | methylbutyric acid |
| | acid | |
| Vanillic acid | 2,6-Dichloronicotinic acid | 3-Allyloxypropionic acid |
| Benzoic acid | 5-Methoxysalicylic acid | 5-Methylhexanoic acid |
| Picolinic acid, 99% | (4-Pyridylthio)acetic acid | 2-Aminonicotinic acid |
| Nicotinic acid | 2-(Methylthio)nicotinic | 6-Methylpicolinic acid |
| | acid | |
| 2-Pyrazinecarboxylic | 1-Methyl-1- | 2-Ethyl-2-hydroxybutyric |
| acid | cyclohexanecarboxylic acid | acid |
| 1-methyl-2- | 2-Hydroxy-6-methylpyridine- | 3-Cyclohexenecarboxylic |
| pyrrolecarboxylic acid | 3-carboxylic acid | acid |
| 1- | (R)-(+)-3-Methylsuccinic | 2-Hydroxyphenylacetic |
| Isoquinolinecarboxylic | acid-1-monomethyl ester | acid |
| 4-butylbenzoic acid | Quinoline-4-carboxylic acid | 2,6-Dimethylbenzoic acid |
| 2-Thiophenecarboxylic | 1H-Indole-3-acetic acid | Thiophene-3-carboxylic |
| acid | | acid |
| 5-Fluoroindole-2- | 5-Hydroxy-2- | 2-(n-Propylthio) |
| carboxylic acid | indolecarboxylic acid | nicotinic acid |
| (S)-(−)-2-Pyrrolidone- | (R)-(−)-4-Methylglutaric | DL-2-Hydroxy-4- |
| 5-carboxylic acid | acid 1-monomethyl ester | (methylthio)butyric acid |
| Itaconic acid monoethyl | 5-methylisoxazole-4- | 2-Amino-6-fluorobenzoic |
| ester | carboxylic acid | acid |
| m-Toluic acid | 4-Acetamidobenzoic acid | 2-Mercaptonicotinic acid |
| p-Toluic acid | 4-Aminosalicylic acid | 6-Methylnicotinic acid |
| 2-Methylnicotinic acid | 3-Acetamidobenzoic acid | 2,5-Difluorobenzoic acid |
| 3-aminobenzoic acid | Succinamic acid | o-Toluic acid |
| 2-Chloroisonicotinic | 2-(4-Fluorobenzoyl)benzoic | 2-Fluorophenylacetic |
| acid | acid | acid |
| 3-Hydroxybenzoic acid | 3,4-Dimethoxybenzoic acid | 2-Acetylbenzoic acid |
| 4-Hydroxybenzoic acid | 3,5-Dimethoxybenzoic acid | 4-Chlorosalicylic acid |
| 2,5-Dimethoxybenzoic | 3-(3,4-Dihydroxyphenyl) | 1-Phenyl-1-cyclopropane |
| acid | propionic acid | carboxylic acid |
| 5-Norbornene-2- | 5-Methyl-2- | 2,5-Dimethylphenylacetic |
| carboxylic acid | pyrazinecarboxylic acid | acid |
| (2-n- | 3-Hydroxy-4-nitrobenzoic | 2,4,6-Trimethylbenzoic |
| Butoxyethoxy) acetic | acid | acid |
| Acid | | |
| 5-Bromofuroic acid | 5-Nitrosalicylic acid | 2-Ethoxybenzoic acid |
| 6-Hydroxynicotinic acid | 4-Chloro-o-anisic acid | Salicylic acid |
| 2-Methoxyphenylacetic | 3-Chloro-4- | 3-Methyl-2- |
| acid | hydroxyphenylacetic acid | thiophenecarboxylic acid |
| 2,4- | trans-4-n-propylcyclohexane | 2-Amino-5-chlorobenzoic |
| Difluorophenylacetic | carboxylic acid | acid |
| 2-Chloro-6-methyl-3- | 2-Hydroxyquinoline-4- | O-Chlorophenylacetic |
| pyridinecarboxylic acid | carboxylic acid | acid |
| 4-Fluorobenzoic acid | 3-indolepropionic acid | 4-Octyloxybenzoic acid |
| 3-Flurobenzoic acid | 2-Amino-4-chlorobenzoic | 5-Bromofuroic acid |
| | acid | |
| alpha, alpha,alpha- | Alpha,Alpha,Alpha- | Alpha, Alpha, Alpha- |
| trifluoro-p-toluic acid | Trifluoro-o-toluic acid | Trifluoro-m-toluic acid |
| 2-Thiopheneacetic acid | 2,5-Dimethyl-3-furoic acid | (+/−)-Citronellic acid |
| 3-Thiopheneacetic acid | Chromone-2-carboxylic acid | 2-Fluorobenzoic acid |
| 5-Bromo-2,4- | 2-[(4S)-2,2-Dimethyl-5-oxo- | 2,5-Difluorophenylacetic |
| dihydroxybenzoic acid | 1,3-dioxolane-4-yl]acetic | acid |
| monohydrate | acid | |
| (R )-(+)-2- | 3-Hydroxy-2- | 2,4,5-Trifluorobenzoic |
| Benzyloxypropionic acid | quinoxalinecarboxylic acid | acid |
| 4-cyanobenzoic acid | Coumarin-3-carboxylic acid | 2-Chloronicotinic acid |
| 3-Cyanobenzoic acid | 2,4-Dichlorobenzoic acid | 2-Chloro-6-fluorobenzoic |
| | | acid |
| phthalide-3-acetic acid | 2,5-Dichlorobenzoic acid | 3-indoleglyoxylic acid |
| 2,5-Dimethylphenoxy | 5-Methoxyindole-2- | 2,3,4-Trifluorobenzoic |
| acetic acid | carboxylic acid | acid |
| 2,5-Dimethylbenzoic | 2,6-Dichlorobenzoic acid | 4-Isobutylbenzoic acid |
| acid | | |
| 3,4-Dimethylbenzoic | 3,4-Dichlorobenzoic acid | 1-Naphthoic acid |
| acid | | |
| p-Tolylacetic acid | 2,3-Dichlorobenzoic acid | m-Tolylacetic acid |
| 4-acetylphenoxyacetic | 2,4-Dimethylphenoxyacetic | 2,4-Dimethoxybenzoic |
| acid | acid | acid |
| 2,4-Dimethylbenzoic | (−)-2-oxo-4- | 1-Adamantanecarboxylic |
| acid | thiazolidinecarboxylic acid | acid |
| 3,5-Dimethylbenzoic | 2,3-Dimethylphenoxyacetic | 2-Amino-5-nitrobenzoic |
| acid | acid | acid |
| 2-Bromoacrylic acid | 3-Methylhippuric acid | 3,5-Dichlorobenzoic acid |
| 3-(3-pyridyl)propionic | 4-(4-methoxyphenyl)butyric | 2,3-Dimethoxybenzoic |
| acid | acid | acid |
| 1-Hydroxy-2-naphthoic | 2-(4-Hydroxyphenoxy) | 2-(allylthio)nicotinic |
| acid | propionic acid | acid |
| 3-methylsalicylic acid | N,N-dimethylsuccinamic acid | 2-(Ethylthio)nicotinic |
| | | acid |
| P-Anisic acid | 2-Mehtylhippuric acid | 6-bromohexanoic acid |
| o-Anisic acid | 5-Chloroindole-2-carboxylic | Itaconic acid mono-n- |
| | acid | butyl ester |
| 4-Nitrophenoxyacetic | trans-4-n-Butylcyclohexane | 2-(4-Chlorophenyl)-2- |
| acid | carboxylic acid | methylpropionic acid |
| 5-methylsalicylic acid | Rhodanine-N-acetic acid | 2-Chloromandelic acid |
| 6-Hydroxy-1-napthoic | 2-Chloro-4,5- | 2-Biphenylcarboxylic |
| acid | difluorobenzoic acid | acid |
| 3,5-dimethoxy-4- | 2,3,4,5-Tetrafluorobenzoic | 4-Bromo-2-fluorocinnamic |
| methylbenzoic acid | acid | acid |
| 1-Adamantaneacetic acid | 2-Chloro-4- | 1-Naphthaleneacetic acid |
| | fluorophenylacetic acid | |
| Cyclopentylacetic acid | (2,5-Dimethoxyphenyl)acetic | 2-Chloro-4- |
| | acid | fluorocinnamic acid |
| 1-Phenylcyclopentane | 2-(4-Chlorophenoxy)-2- | Cyclohexanecarboxylic |
| carboxylic acid | methylpropionic acid | acid |
| 1-(p-Tolyl)-1- | (2S)-4-(1,3- | 2,6-Dichloro-5- |
| cyclopentanecarboxylic | Dioxoisoindolin-2-yl)-2- | fluoropyridine-3- |
| acid | hydroxy butanoic acid | carboxylic acid |
| 2,6- | (4-Chlorophenylthio) acetic | 3-Hydroxy-7-methoxy-2- |
| Dichlorophenylacetic | acid | naphthoic acid |
| acid | | |
| (−)-Camphanic acid | 2,3-Diphenylpropionic acid | DL-2-Methylbutyric acid |
| 2-Amino-5-bromobenzoic | Beta-(4-Methylbenzyl) | Rhodanine-3-propionic |
| acid | mercaptopropionic acid | acid |
| 2,5-Dimethoxy cinnamic | 2,5-Dichlorophenylthio | trans-2-Methyl-2- |
| acid | glycolic acid | pentenoic acid |
| trans-2-Pentenoic acid | (−)-Camphanic acid | 2-Methyl-3-furoic acid |
| Valeric acid | mono-Ethyl malonate | trans-2-hexenoic acid |
| 3-(2- | 2-Chloro-6- | 4-Benzyloxyphenylacetic |
| benzothiazolylthio) | fluorophenylacetic acid | acid |
| propionic acid | | |
| 2,4,Dichlorophenylacetic | 5-Bromo-2-fluorocinnamic | 4-(4-tert- |
| acid | acid | butylphenyl)benzoic acid |
| (+/−)-2-(6-Methoxy-2- | 2-(carboxymethylthio)-4,6- | 1-Piperidinepropionic |
| naphthyl)propionic acid | dimethylpyridine | acid |
| | monohydrate | |
| 3-Cyclopentylpropionic | (2- | Alpha-Methylcinnamic |
| acid | Benzothiazolylthio)acetic | acid |
| | acid | |
| 2-Ethoxynaphthoic acid | DL-Lactic acid | 2-Methylhexanoic acid |
| trans-3-Furanacrylic | 1-(4-Methoxyphenyl)-1- | 3-Hydroxy-2-pyridine- |
| acid | cyclopentanecarboxylic acid | carboxylic acid |
| 2,3-Dichlorophenoxy | 2,4-Dichlorophenoxy acetic | 3-Mercaptoisobutyric |
| acetic acid | acid | Acid |
| 5-Fluoro-2- | (3,4-Dimethoxyphenyl)acetic | 2-Thiopheneglyoxylic |
| methylbenzoic acid | acid | acid |
| (2-Napthoxy)-acetic | o-Tolylacetic acid | 2-Hydroxyoctanoic acid |
| acid | | |
| Urocanic acid | Hydrocinnamic acid | N-Acetyl-l-proline |
| Dl-Mandelic acid | DL-2-Phenylpropionic acid | N-Methyl-maleamic acid |
| Coumalic acid | 4-(Methylamino)benzoic acid | 3,4-Difluorobenzoic acid |
| 4-Methyl-1-cyclohexane | Tetrahydro-2,2-dimethyl-5- | DL-2-phenoxypropionic |
| carboxylic acid | oxo-3-furancarboxylic acid | acid |
| m-Anisic acid | 3-Hydroxyphenylacetic acid | Indole-3-carboxylic acid |
| Cyclohexylacetic acid | Phenoxyacetic acid | 3-Fluorocinnamic acid |
| Cycloheptanecarboxylic | 3-Amino-1H-1,2,4-triazole- | 3-Fluoro-4-methylbenzoic |
| acid | 5-carboxylic acid | acid |
| 2-Octynoic acid | trans-Styrylacetic acid | 2-Methylcinnamic acid |
| 2-Propylpentanoic acid | 3-Fluorophenylacetic acid | 4-Acetylbutyric acid |
| 2-Methylheptanoic acid | Furylacrylic acid | Phenylpyruvic acid |
| Octanoic acid | Thiosalicylic acid | mono-Ethyl succinate |
| 3-(2-Thienyl)acrylic | Alpha-Methylhydrocinnamic | Alpha-Fluorocinnamic |
| acid | acid | acid |
| mono-Methyl glutarate | 3-(2-Thienyl)propanoic acid | 3-Phenoxypropionic acid |
| trans-3-(3- | trans-3-(3-Thienyl)acrylic | 3,4-(Methylenedioxy) |
| Pyridyl)acrylic acid | acid | phenylacetic acid |
| 3-Noradamantane | 4-Acetyl-3,5-dimethyl-2- | 3-(2-Hydroxyphenyl) |
| carboxylic acid | pyrrolecarboxylic acid | propionic acid |
| 2-Nitrobenzoic acid | DL-Atrolactic acid | 4-Methylsalicylic acid |
| 4- | 2-Methyl-1H-benzimidazole- | 3-Fluoro-4- |
| (Dimethylamino)butyric | 5-carboxylic acid | methoxybenzoic acid |
| acid hydrochloride | | |
| 3-Chloro-4- | 4-(Dimethylamino) | 3,4-Difluorocinnamic |
| hydroxybenzoic acid | phenylacetic acid | acid |
| DL-3-Phenyllactic acid | 3-Benzoylpropionic acid | Homovanillic acid |
| 2-Methyl-terephthalic | 3-(Diethylamino) propionic | 3-(4-Methylbenzoyl) |
| acid | acid hydrochloride | propionic acid |
| 4-(2-Thienyl)butyric | 3,4-Dihydro-2,2-dimethyl-4- | Cyclohexanepentanoic |
| acid | oxo-2H-pyran-6-carboxylic | acid |
| | acid | |
| Cyclohexanebutyric acid | mono-Methyl phthalate | Undecanoic acid |
| 3-Chlorophenylacetic | 3,5-Difluorophenylacetic | 6-Hydroxy-2-naphthoic |
| acid | acid | acid |
| 3-Benzoylacrylic acid | 4-Amino-2-chlorobenzoic | 3-Indoleacrylic acid |
| | acid | |
| 3-Amino-4-chlorobenzoic | 4-(4-Methylphenyl)butyric | 3-Hydroxy-2-naphthoic |
| acid | acid | acid |
| 3,4- | 3-(4- | 2-Hydroxy-1-naphthoic |
| Difluorophenylacetic | Methoxyphenyl) propionic | acid |
| acid | acid | |
| 2,5-Dimethylphenoxy | trans-3-(4- | 5-Methyl-2-nitrobenzoic |
| acetic acid | Methylbenzoyl) acrylic acid | acid |
| 3-Quinolinecarboxylic | 3-(2- | 3,5-Dimethyl-p-anisic |
| acid | Methoxyphenyl)propionic | acid |
| | acid | |
| Decanoic acid | 2-Naphthoic acid | 4-Benzoylbutyric acid |
| 5-Chlorosalicylic acid | Quinaldic acid | N-Methylhippuric acid |
| 3-(3-Methoxyphenyl) | 5-Nitrothiophene-2- | 4-(Diethylamino) benzoic |
| propionic acid | carboxylic acid | acid |
| 2-Methyl-6-nitrobenzoic | Alpha,Alpha,Alpha-2- | N,N-Dimethyl-1- |
| acid | Tetrafluoro-p-toloic acid | phenylalanine |
| Ibuprofen | 2-Nitrophenylacetic acid | 4-Benzyloxybutyric acid |
| 3-Pyridylacetic acid | 2-Methyl-5-nitrobenzoic | Diethylphosphonoacetic |
| | acid | acid |
| 2-Oxo-6-pentyl-2H- | mono-Methyl cis-5- | 2-Methyl-3-nitrobenzoic |
| pyran-3-carboxylic acid | norbornene-endo-2,3- | acid |
| | dicarboxylate | |
| DL-2-(3-Chlorophenoxy) | 3,5-Dichloro-4- | trans-2-Chloro- |
| propionic acid | hydroxybenzoic acid | fluorocinnamic acid |
| 5-Bromo-2- | DL-4-Hydroxy-3- | |
| thiophenecarboxylic | methoxymandelic acid | |
| acid | | |
| 3,4-Diethoxybenzoic | Alpha-Phenyl-o-toluic acid | Diphenylacetic acid |
| acid | | |
| 5-Bromosalicylic Acid | Adipic acid monoethyl ester | Syringic acid |
| 3,5-Dichloroanthranilic | trans-2,4-Dimethoxycinnamic | 4-(4-Hydroxyphenyl) |
| acid | acid | benzoic Acid |
| Alpha-Phenylcinnamic | trans-2,3-dimethoxycinnamic | 3-(Phenylsulfonyl) |
| acid | acid | propionic acid |
| 3,3-Diphenylpropionic | (s)-(−)-2-[(Phenylamino) | 3-(Trifluoromethyl) |
| acid | carbonyloxy] propionic acid | cinnamic acid |
| Cyclohexylphenylacetic | 4-(3-Methyl-5-oxo-2- | 3,4-Dimethoxycinnamic |
| acid | pyrazoline-1-yl)benzoic | acid |
| | acid | |
| 4-(Trifluoromethyl) | Pentafluorophenoxyacetic | Trans-2,4- |
| mandelic acid | acid | Dichlorocinnamic acid |
| 2-Nitrophenylpyruvic | Alpha-Phenylcyclopentane | 3,4-Dichlorophenylacetic |
| acid | acetic acid | acid |
| 4-(Hexyloxy)benzoic | 4-Butoxyphenylacetic acid | 4-Bromocinnamic acid |
| acid | | |
| 7-Hydroxycoumarin-4- | 3-(3,4,5-Trimethoxyphenyl) | 2-Chloro-5- |
| acetic acid | propionic acid | (methylthio)benzoic acid |
| 1,3-dioxo-2- | 1,4-dihydro-1-ehtyl-7- | 2-phenylmercapto |
| isoindolineacetic acid | methyl-4-oxo-1,8- | methylbenzoic acid |
| | naphthyridine-3-carboxylic | |
| | acid | |
| Anthracene-9-carboxylic | 3,4,5- | 3-Bromo-4-fluorocinnamic |
| acid | Trimethoxyphenylacetic acid | acid |
| p-Bromophenoxyacetic | 4-Butoxyphenylacetic acid | N-Carbobenzyloxy-L- |
| acid | | proline |
| (Phenylthio)acetic acid | 4-Benzyloxybenzoic acid | 3-Phenylbutyric acid |
| 7-Chloro-4-hydroxy-3- | gamma-Oxo-(1,1′-biphenyl)- | 3,4,5-Triethoxybenzoic |
| quinolinecarboxylic | 4-butanoic aicd | acid |
| acid | | |
| Acridine-9-carboxylic | 2-Ethoxycarbonylamino-3- | 3,5-Di-tert-butyl-4- |
| acid hydrate | phenyl-propionic acid | hydroxybenzoic acid |
| 2-Cyclopentene-1-acetic | 3,4,5-Trimethoxycinnamic | 3-(BOC-amino)benzoic |
| acid | acid | acid |
| 4-Methoxysalicylic acid | 4-Fluorocinnamic acid | 4,5-Dibromo2-furoic acid |
| 2-Hydroxynicotinic acid | 4-Bromo-3,5- | 5-Phenylvaleric acid |
| | dihydroxybenzoic acid | |
| 4-Pentynoic acid | 4-Ethoxybenzoic acid | 4-Acetoxybenzoic acid |
| 3,3-Dimethylacrylic | Dicyclohexylacetic acid | 3-Acetoxybenzoic acid |
| acid | | |
| 4-Methoxy-2- | cis-2-(2- | 4-Methyl-3-nitrobenzoic |
| methylbenzoic acid | Thiophenecarbonyl)-1- | acid |
| | cyclohexanecarboxylic acid | |
| 4-Methylvaleric acid | (2-Methylphenoxy)acetic | 4-Isopropoxybenzoic acid |
| | acid | |
| 3,3,3- | (4-Methylphenoxy)acetic | 4-Nitrophenylacetic acid |
| Trifluoropropionic acid | acid | |
| 2-Methyl-1-cyclohexane | 2,2,3,3-Tetramethyl | 3-Methyl-1-cyclohexane |
| carboxylic acid | cyclopropanecarboxylic acid | carboxylic acid |
| 4-Amino-3-nitrobenzoic | 5-Methyl-2- | 4-Methoxyphenoxyacetic |
| acid | thiophenecarboxylic acid | acid |
| 3-Methoxysalicylic acid | 4-Fluorophenylacetic acid | 2-Phenoxybutyric acid |
| 3,5-Dimethoxy-4- | (R)-(−)-2,2-Dimethyl-5- | 4-Hydroxymandelic acid |
| hydroxycinnamic acid | oxo-1,3-dioxolane-4-acetic | monohydrate |
| | acid | |
| (2-Methoxyphenoxyl) | 2,2-Dichloro-1-methylcyclo- | 4-Hydroxyphenylacetic |
| acetic acid | propanecarboxylic acid | acid |
| 2-Ethylbenzoic acid | 4-Fluorophenoxyacetic acid | 4-tert-Butylbenzoic acid |
| 5-Fluoro-2- | (R)-(+)-2-(4-Hydroxy | 2,6-Dimethoxynicotinic |
| methoxybenzoic acid | phenoxy)-propionic acid | acid |
| 2- | 4-Hydroxy-3-nitrobenzoic | 3,4-Difluorohydro |
| Carboxyethylphosphonic | acid | cinnamic acid |
| acid | | |
| 4-Hydroxy-3-methoxy | 3-Chloro-2-methylbenzoic | 2-Chloro-4-fluorobenzoic |
| benzoic acid | acid | acid |
| 4-Fluoro-3- | 2-Chloro-6-methylnicotinic | 4-Chlorophenoxyacetic |
| methylbenzoic acid | acid | acid |
| 3-Fluoro-2- | 2,2-Bis(hydroxymethyl) | 5-Chloro-2- |
| methylbenzoic acid | butyric acid | methoxybenzoic acid |
| 5-Amino-4-methyl- | (2,2-Dimethyl-5-[2,5- | (Alpha, Alpha, Alpha- |
| cyclohexa-1,5-diene- | dimethylphenoxy]-pentanoic | Trifluoro-m-tolyl)acetic |
| 1,4-dicarboxylic acid | acid) | acid |
| 4-Methoxycyclohexane | 1-Methylindole-3-carboxylic | (R)-(−)-3- |
| carboxylic acid | acid | Chloromandelic acid |
| 4-Propylbenzoic acid | 4-Chlorophenylacetic acid | 4-Bromomandelic acid |
| 2-Methoxy-4- | 4-Oxo-4H-1-benzopyran-2- | 2-Mercapto-4-methyl-5- |
| (methylthio)-benzoic | carboxylic acid | thiazoleacetic acid |
| acid | | |
| 2-(Trifluoromethyl) | 4-Methoxy-3-nitrobenzoic | 3,4-Dichlorocinnamic |
| cinnamic acid | acid | acid |
| 3-Methylcyclohexane | 4-Methoxy-2- | 5-Methoxy-2-methyl-3- |
| carboxylic acid | quinolinecarboxylic acid | indoleacetic acid |
| 2-(4-Nitrophenyl) | 4-(4-Methoxyphenyl)butyric | 4-Carboxybenzene |
| propionic acid | acid | sulfonamide |
| 2-Hydroxy-5-(1H-pyrrol- | 3-Chloro-4- | 5-Chloro-2-nitrobenzoic |
| 1-yl)-benzoic acid | hydroxyphenylacetic acid | acid |
| 2-Methyl-3-indoleacetic | 2-Fluoro- | 4-Amino-5-chloro-2- |
| acid | 3(trifluoromethyl)-benzoic | methoxybenzoic acid |
| | acid | |
| 4-Chloro-2- | 2-(2-Nitrophenoxy)acetic | 3-Acetoxy-2- |
| fluorocinnamic acid | acid | methylbenzoic acid |
| 2,4,6-Trichlorobenzoic | 3,4-Dichlorophenoxyacetic | 2-Bibenzylcarboxylic |
| acid | acid | acid |
| 2-Chloro-5- | (S)-(+)-6-Methoxy-alpha- | 4-(3,4-Dimethoxyphenyl)- |
| (trifluoromethyl)benzoic | methyl-2-naphthalenacetic | butyric acid |
| acid | acid | |
| 4-Ethylbiphenyl-4′- | 2-Bromo-5-methoxybenzoic | 5-Bromo-2-chlorobenzoic |
| carboxylic acid | acid | acid |
| 3,5-Dinitro-p-toluic | 1-Methyl-2- | 1-Methyl-3-indoleacetic |
| acid | nitroterephthalate | acid |
| 4-Pentylbenzoic acid | 4-n-Heptyloxybenzoic acid | 4-Biphenylacetic acid |

Over 5450 compounds have been made using this process employing the amines and carboxylic acids listed in Tables 1 and 2.

Alternatively, bi-ligand libraries of the invention can be built through the direct reaction of isocyanates or thioisocyanates using a combination of solid phase chemistry and solution phase chemistry.

As shown in FIG. 12c, bi-ligand libraries of the invention can further be prepared in the following manner. A solution of an isocyanate or thioisocyanate and a common ligand mimic of the invention is formed in a solvent, such as DMSO. The isocyanate and common ligand mimic are allowed to react overnight, followed by the addition of aminomethylated polystyrene Resin (NovaBiochem, Cat. No. 01-64-0383). This mixture is then shaken at room temperature for a period of time, for example about 4 hours. The resin then can be filtered and dried under reduced pressure to yield the desired product. Nonlimiting examples of isocyanates and thioisocyanates are provided in Table 3.

TABLE 3

| | |
|---|---|
| allyl isocyanate | 3-chloro-4-methylphenyl isocyanate |
| N-propyl isocyanate | 1-naphthyl isocyanate |
| pentyl isocyanate | 3-chloro-4-fluorophenyl isocyanate |
| phenyl isocyanate | 2,6-diethylphenyl isocyanate |
| m-tolyl isocyanate | 1-adamantyl isocyanate |
| p-tolyl isocyanate | 2-methyl-4-nitrophenyl isocyanate |
| o-tolyl isocyanate | 2-methyl-5-nitrophenyl isocyanate |
| benzyl isocyanate | 2-methyl-3-nitrophenyl isocyanate |
| 4-fluorophenyl isocyanate | 4-methyl-2-nitrophenyl isocyanate |
| heptyl isocyanate | 4-methyl-3-nitrophenyl isocyanate |
| 3-cyanophenyl isocyanate | 2,4-dimethoxyphenyl isocyanate |
| 2,6-dimethylphenyl isocyanate | 2,5-dimethoxyphenyl isocyanate |
| 2-ethylphenyl isocyanate | 2-fluoro-5-nitrophenyl isocyanate |
| 2,5-dimethylphenyl isocyanate | 4-fluoro-3-nitrophenyl isocyanate |
| 2,4-dimethylphenyl isocyanate | 5-chloro-2-methoxyphenyl isocyanate |
| 3,4-dimethylphenyl isocyanate | ethyl-6-isocyanatohexanoate |
| 4-ethylphenyl isocyanate | 4-(trifluoromethyl)phenyl isocyanate |
| 3-ethylphenyl isocyanate | 3-(trifluoromethyl)phenyl isocyanate |
| 2,3-dimethylphenyl isocyanate | 2-(trifluoromethyl)phenyl isocyanate |
| 2-methoxyphenyl isocyanate | 3,4-dichlorophenyl isocyanate |
| 3-methoxyphenyl isocyanate | 2,4-dichlorophenyl isocyanate |
| 4-methoxyphenyl isocyanate | 3,5-dichlorophenyl isocyanate |
| 5-chloro-3-methylphenyl | 2,3-dichlorophenyl isocyanate |
| isocyanate | |
| 2-chlorophenyl isocyanate | trichloroacetyl isocyanate |
| 3-chlorophenyl isocyanate | ethyl-4-isocyanatobenzoate |
| 2,4-difluorophenyl isocyanate | Isopropyl isocyanate |
| 3,4-difluorophenyl isocyanate | Butyl isocyanate |
| 2,6-difluorophenyl isocyanate | cyclopentyl isocyanate |
| butyl isocyanatoacetate | cyclohexyl isocyanate |
| trans-2-phenylcyclopropyl | o-tolyl isocyanate |
| isocyanate | |
| trichloromethyl isocyanate | 3-fluorophenyl isocyanate |
| 3-acetylphenyl isocyanate | 2-fluorophenyl isocyanate |
| 4-acetylphenyl isocyanate | ethyl 3-isocyanatopropionate |
| 2-isopropylphenyl isocyanate | 4-methylbenzyl isocyanate |
| 2-ethyl-6-methylphenyl isocyanate | phenethyl isocyanate |
| 2,4,6-trimethylphenyl isocyanate | 3-fluorobenzyl isocyanate |
| 4-ethoxyphenyl isocyanate | 4-fluorobenzyl isocyanate |
| 2-methoxy-5-methylphenyl | 3-fluoro-4-methylphenyl isocyanate |
| isocyanate | |
| 2-ethoxyphenyl isocyanate | 2,4-difluorophenyl isocyanate |
| 4-methoxy-2-methylphenyl | 3,4-difluorophenyl isocyanate |
| isocyanate | |
| 4-methoxybenzyl isocyanate | 2,6-difluorophenyl isocyanate |
| 2-nitrophenyl isocyanate | 3,5-difluorophenyl isocyanate |
| 4-nitrophenyl isocyanate | octyl isocyanate |
| 3-nitrophenyl isocyanate | 1,1,3,3-tetramethylbutyl isocyanate |
| 4-(methylthio)phenyl isocyanate | trans-2-phenylcyclopropyl isocyanate |
| 2-(methylthio)phenyl isocyanate | trichloromethyl isocyanate |
| 5-chloro-2-methylphenyl | 4-isopropylphenyl isocyanate |
| isocyanate | |
| 4-chloro-2-methylphenyl | propyl isothiocyanate |
| isocyanate | |
| 2-isopropyl-6-methylphenyl | 3,4-(methylenedioxy)phenyl |
| isocyanate | isocyanate |
| 2-chloro-6-methylphenyl | 2-chloro-5-methylphenyl isocyanate |
| isocyanate | |
| 3-chloro-2-methylphenyl | 2-chlorobenzyl isocyanate |
| isocyanate | |
| isobutyl isothiocyanate | 3-chloro-4-fluorophenyl isocyanate |
| tert-butyl isothiocyanate | 2,6-diethylphenyl isocyanate |
| N-butyl isothiocyanate | 4-N-butylphenyl isocyanate |
| 2-methoxyethyl isothiocyanate | methyl-4-isocyanato-benzoate |
| N-amyl isothiocyanate | 3-carbomethoxyphenyl isocyanate |
| 3-methoxypropyl isothiocyanate | methyl-2-isocyanatobenzoate |
| phenyl isothiocyanate | 1-adamantyl isocyanate |
| cyclohexyl isothiocyanate | 2-methyl-4-nitrophenyl isocyanate |
| 2-tetrahydrofurfuryl isothiocyanate | 2-methyl-5-nitrophenyl isocyanate |
| o-tolyl isothiocyanate | 2-methyl-3-nitrophenyl isocyanate |
| benzyl isothiocyanate | 4-methyl-2-nitrophenyl isocyanate |
| m-tolyl isothiocyanate | 4-methyl-3-nitrophenyl isocyanate |
| 4-fluorophenyl isothiocyanate | diethoxyphosphinyl isocyanate |
| 2-fluorophenyl isothiocyanate | 2,4-dimethoxyphenyl isocyanate |
| 3-fluorophenyl isothiocyanate | 2,5-dimethoxyphenyl isocyanate |
| heptyl isothiocyanate | 3,4-dimethoxyphenyl isocyanate |
| ethyl 3-isothiocyanatopropionate | 2-fluoro-5-nitrophenyl isocyanate |
| ethyl 2-isothiocyanatopropionate | 4-fluoro-3-nitrophenyl isocyanate |
| 4-cyanophenyl isothiocyanate | benzenesulphonyl isocyanate |
| 2-ethylphenyl isothiocyanate | 5-chloro-2-methoxyphenyl isocyanate |
| 2,6-dimethylphenyl isothiocyanate | 3-chloro-4-methoxyphenyl isocyanate |
| 2-phenylethyl isothiocyanate | ethyl-6-isocyanatohexanoate |
| 2,4-dimethylphenyl isothiocyanate | 4-(trifluoromethyl)phenyl isocyanate |
| 4-methylbenzyl isothiocyanate | 3-(trifluoromethyl)phenyl isocyanate |
| 2-phenylethyl isothiocyanate | 2-(trifluoromethyl)phenyl isocyanate |
| 3-methoxyphenyl isothiocyanate | 2-(trifluoromethyl)phenyl isocyanate |
| 2-methoxyphenyl isothiocyanate | 3,4-dichlorophenyl isocyanate |
| 4-methoxyphenyl isothiocyanate | 2,6-dichlorophenyl isocyanate |
| 4-chlorophenyl isothiocyanate | 2,4-dichlorophenyl isocyanate |
| 2-chlorophenyl isothiocyanate | 2,5-dichlorophenyl isocyanate |
| 3-chlorophenyl isothiocyanate | 3,5-dichlorophenyl isocyanate |
| 2,4-difluorophenyl isothiocyanate | 2,3-dichlorophenyl isocyanate |
| 2-morpholinoethyl isothiocyanate | trichloroacetyl isocyanate |
| 3-acetylphenyl isothiocyanate | 2-ethyl-6-isopropylphenyl isocyanate |
| 4-isopropylphenyl isothiocyanate | ethyl-3-isocyanatobenzoate |
| 2-isopropylphenyl isothiocyanate | ethyl-4-isocyanatobenzoate |
| 4-(dimethylamino)phenyl | 2-isopropyl-6-methylphenyl |
| isothiocyanate | isocyanate |
| 4-ethoxyphenyl isothiocyanate | ethyl-2-isocyanatobenzoate |
| 4-methoxybenzyl isothiocyanate | 4-butoxyphenyl isocyanate |
| 3-nitrophenyl isothiocyanate | 2-methoxy-5-nitrophenyl isocyanate |
| 4-nitrophenyl isothiocyanate | 2-biphenylylisocyanate |
| 2-(methylthio)phenyl | 4-biphenyl isocyanate |
| isothiocyanate | |
| 3-(methylthio)phenyl | p-toluenesulphonyl isocyanate |
| isothiocyanate | |
| 4-(methylthio)phenyl | o-toluenesulphonyl isocyanate |
| isothiocyanate | |
| 1-naphthyl isothiocyanate | undecyl isocyanate |
| 2-chlorobenzyl isothiocyanate | 2-bromophenyl isocyanate |
| 4-chlorobenzyl isothiocyanate | 3-bromophenyl isocyanate |
| 3-chloro-4-methylphenyl | 4,5-dimethyl-2-nitrophenyl |
| isothiocyanate | isocyanate |
| 4-chloro-2-methylphenyl | 5-chloro-2-methylphenyl |
| isothiocyanate | isothiocyanate |
| 4-bromophenyl isocyanate | 2-chloro-4-nitrophenyl isocyanate |
| 3-morpholinopropyl isothiocyanate | 2-chloro-5-nitrophenyl isocyanate |
| 4-N-butylphenyl isothiocyanate | 4-chloro-2-nitrophenyl isocyanate |
| allyl isothiocyanate | ethyl isothiocyanate |
| 2-methoxycarbonylphenyl | 2-chloro-6-methylphenyl |
| isothiocyanate | isothiocyanate |
| 1-adamantyl isothiocyanate | isopropyl isothiocyanate |
| 4-methyl-2-nitrophenyl | 4-chloro-3-nitrophenyl |
| isothiocyanate | isothiocyanate |
| 3,4-dimethoxyphenyl | 3-bromophenyl isothiocyanate |
| isothiocyanate | |
| 2,5-dimethoxyphenyl | 2-bromophenyl isothiocyanate |
| isothiocyanate | |
| 2,4-dimethoxyphenyl | 2,6-diisopropylphenyl isothiocyanate |
| isothiocyanate | |
| 5-chloro-2-methoxyphenyl | 2-(3,4-dimethoxyphenyl)ethyl |
| isothiocyanate | isothiocyanate |
| 2-(trifluoromethyl)phenyl | 4-bromo-2-methylphenyl |
| isothiocyanate | isothiocyanate |
| 4-(trifluoromethyl)phenyl | 2-bromo-4-methylphenyl |
| isothiocyanate | isothiocyanate |
| 2,6-dichlorophenyl isothiocyanate | cyclododecyl isothiocyanate |
| 2,3-dichlorophenyl isothiocyanate | 4-phenylazophenyl |
| | isothiocyanate1111 |
| 3,5-dichlorophenyl isothiocyanate | 4-diethylaminophenyl isothiocyanate |
| 4-methoxy-2-nitrophenyl | |
| isothiocyanate | |

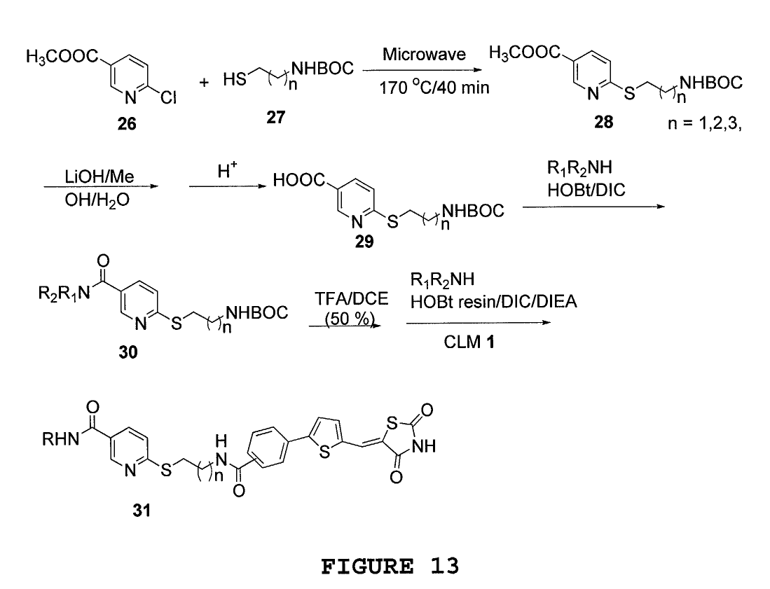

Bi-ligand libraries of the invention can also be made by the reaction sequence provided in FIG. 13, using Boc-protected amines. As shown in FIG. 13, bi-ligand libraries of the present invention can be prepared in the following manner. A mixture of DBU, a halopyridine and a thiol is formed in a solvent, such as dioxane. The reaction mixture then is agitated under microwave irradiation at a temperature of 150 to 170° C. for a period of about 30 to 40 minutes. For example, the reaction mixture is agitated under microwave irradiation at a temperature of about 170° C. for a period of about 40 minutes. The solvent can be removed from the mixture and the resultant oil residue subjected to a column to provide the desired intermediate compound.

The intermediate compound then can be suspended in a mixture of water and alcohol, for example a mixture of water and methanol. Lithium hydroxide is added to the solution, which then is refluxed for a period of about 1 to 2 hours, for example a period of about 2 hours. Solvent can be removed from the reaction mixture, and the residue dissolved in water. Dilute hydrochloric acid is added dropwise, forming a white precipitate.

The white precipitate is dissolved in a solvent, such as a mixture of dry DMF and DIC. HOBt resin, swelled in a solvent, such as dry THF, is then added to the solution, which is shaken at room temperature overnight. The resin then is washed with 3×dry DMF and 2×dry THF and added to a solution of an amine dissolved in a solvent, such as dry DMF. The mixture can be shaken at room temperature overnight, followed by filtration and washing in solvent of the Boc protected intermediate, which then can be collected and vacuum dried.

The Boc-protected intermediate is then dissolved in a solvent mixture, for example a mixture of TFA and dichloroethane. The mixture is then shaken at room temperature for a period of about 15 to 20 minutes, for example a period of about 20 minutes. Solvent can be removed from the mixture to form a deBoc intermediate.

HOBt resin, swelled in a solvent, such as a mixture of dry THF and dry DMF, is added to a solution of a common ligand mimic of the present invention, dissolved in a solvent, such as a mixture of dry DMF and DIC. This solution then is shaken at room temperature overnight and washed with 3×dry DMF and 3×dry THF.

The resin mixture then can be added to a solution of the deBoc intermediate in a solvent, such as dry THF. The mixture can be shaken at room temperature overnight, followed by filtration and washing of the resin in a solvent, such as dry DMF. The filtrate then can be collected and vacuum dried to provide bi-ligands of the invention. Nonlimiting examples of amines that are useful in this method include those provided in Table 4.

TABLE 4

| | |
|---|---|
| Cyclopropylamine | 2-methoxyethylamine |
| Isopropylamine | 3-amino-1-propanol |
| Propylamine | DL-1-amino-2-propanol |
| ethanolamine | N-Methyl-b-alaninenitrile |
| 3-pyrroline | 4-amino-4H-1,2,4-triazole |
| Hydroxylamine | cyclopentylamine |
| N-Methylallylamine | Piperidine |
| Cyclobutylamine | morpholine |
| Pyrrolidine | 1-Ethylpropylamine |
| Diethylamine | Neopentylamine |
| isobutylamine | N-ethylisopropylamine |
| N-butylamine | N-Methylbutylamine |
| N-Methylpropylamine | 2-Aminopyridine |
| sec-Butylamine | 3-Aminopyridine |
| Tert-butylamine | furfurylamine |
| 3-methoxypropylamine | 3-Amino5-methylpyrazole |
| (+/−)-2-amino-1-butanol | diallylamine |
| 2-amino-1-methyloxypropane | 3-(ethylamino)propionitrile |
| 4-amino-1-butanol | 2-methylpiperidine |
| 1-AMINO-2-BUTANOL | 3-methylpiperidine |
| 2-amino-2-methyl-1-propanol | 4-methylpiperidine |
| Thiazolidine | cyclohexylamine |
| 2-amino-1,3-propanediol | hexamethyleneimine |
| 3-amino-1,2-propanediol | Methylpiperazine |
| Aniline | 1-aminopiperidine |
| N-acetylethylenediamine | 4-hydroxypiperidine |
| 4-aminomorpholine | Tetrahydrofurfurylamine |
| 3-dimethylaminopropylamine | 1,3-Dimethylbutylamine |
| N-Isopropylethylenediamine | dipropylamine |
| 4-Amino Butyric Acid | cycloheptylamine |
| 5-Amino-1-pentanol | 3-Fluoroaniline |
| 3-ethoxypropylamine | 4-fluoroaniline |
| diethanolamine | exo-2-aminobornane |
| 3-(methylthio)propylamine | 2-thiophenemethylamine |
| m-toluidine | 2-ethylpiperidine |
| O-Toluidine | 2-methylcyclohexylamine |
| p-Toluidine | 3,5-dimethylpiperidine |
| 2-(Aminomethyl)pyridine | 4-methylcyclohexylamine |
| 3-(aminomethyl)pyridine | glycinamide hydrochloride |
| 4-(aminomethyl)pyridine | benzylamine |

Over 560 compounds have been made by this process employing the amines provided in Table 4.

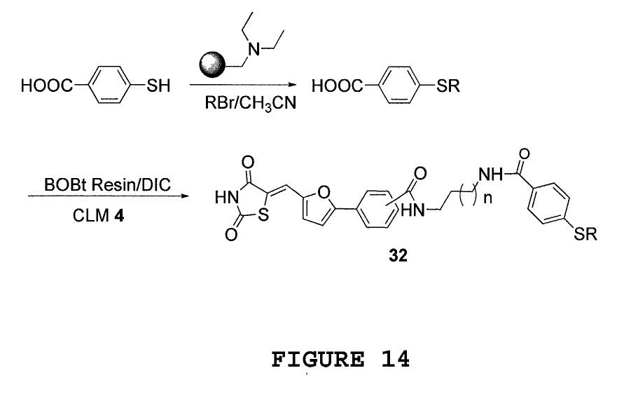

Bi-ligand libraries of the invention can also be built using alkyl halides following the reaction scheme depicted in FIG. 14. As shown in FIG. 14, bi-ligands libraries of the invention can be prepared in the following manner. A mixture of 4-mercaptobenzoic acid and an alkyl bromide is formed in a solvent, such as CH₃CN. Triethylamine resin (Fluka) then is added to the mixture, which is shaken at room temperature overnight. The resin can be filtered and washed with solvent, followed by collection and vacuum drying.

Next, the filtrate is dissolved in a solvent, such as a mixture of dry DMF and DIC. HOBt resin, swelled in a solvent, such as dry THF, is added to the solution. The solution then is shaken at room temperature overnight and washed with 3×dry DMF and 2×dry THF. The resin then is added to a common ligand mimic of the invention, which has been dissolved in a solvent, such as dry DMF. The solution is shaken at room temperature overnight. The resin then can be filtered and washed with solvent. The filtrate can be collected and vacuum dried to provide bi-ligands of the invention. Nonlimiting examples of alkylhalides useful in this method are provided in Table 5.

TABLE 5

| | |
|---|---|
| Bromoethane | 4-Bromo-1-butene |
| Propargyl bromide | 3-Bromo-2-methylpropene |
| Bromoacetonitrile | 1-Bromobutane |
| Allyl bromide | 2-Bromobutane |
| 2-Bromopropane | 2-Bromoacetamide |
| 1-Bromopropane | Cyclopentyl bromide |
| 1-Bromo-2-butyne | 4-Bromo-2-methyl-2-butene |
| 3-Bromopropionitrile | 5-Bromo-1-pentene |
| 2-Bromopropionitrile | Methyl 4-bromocrotonate |
| (Bromomethyl)cyclopropane | Methyl bromoacetate |
| Crotyl bromide remainder 3-bromo-1- | 2-(Bromomethyl)tetrahydro-2H- |
| butene | pyran |
| Bromomethyl acetate | 2-Bromopropionamide |
| 2-Bromo-1,1,1-trifluoroethane | Ethyl 3-bromopropionate |
| Cyclohexyl Bromide | Alpha-Bromo-p-xylene |
| 1-Bromohexane | alpha-Bromo-o-xylene |
| Methyl DL-2-bromopropionate | Alpha-Bromo-m-xylene |
| 2-Bromoethyl acetate | (2-Bromoethyl)benzene |
| 6-Bromohexanenitrile | 3-Bromo-1,1,1-trifluoroacetone |
| (Bromomethyl)cyclohexane | 4-Bromobutyl acetate |
| Alpha-Bromo-m-tolunitrile | tert-Butyl bromoacetate |

Over 240 compounds have been made using this process employing the alkyl halides listed in Table 5.

The present invention is based on the development of bi-ligands that bind to two independent sites on a receptor. The combination of two ligands into a single molecule allows both ligands to simultaneously bind to the receptor and thus can provide synergistically higher affinity than either ligand alone (Dempsey and Snell, Biochemistry 2:1414-1419 (1963); and Radzicka and Wolfenden, Methods Enzymol. 249:284-303 (1995), each of which is incorporated herein by reference). The generation of libraries of bi-ligands focused for binding to a receptor family or a particular receptor in a receptor family has been described previously (see WO 99/60404, which is incorporated herein by reference). The common ligand mimics of the present invention allow for increased diversity of bi-ligand libraries while simultaneously preserving the ability to focus a library for binding to a receptor family.

As described previously (see WO 99/60404), when developing bi-ligands having binding activity for a receptor family, it is generally desirable to use a common ligand having relatively modest binding activity, for example, mM to μM binding activity. This binding activity is increased when combined with a specificity ligand.

The common ligand mimic can be modified through the addition of substituents, which can also be called expansion linkers. Substitution of the common ligand mimic allows for tailoring of the bi-ligand by directing the attachment location of the specificity ligand on the common ligand mimic. Tailoring of the bi-ligand in this manner provides optimal binding of the common ligand mimic to the conserved site on the receptor and of the specificity ligand to the specificity site on the same receptor. Through such tailoring, libraries having improved diversity and improved receptor binding can be produced. The bi-ligands contained in such libraries also exhibit improved affinity and/or specificity.

A number of formats for generating combinatorial libraries are well known in the art, for example soluble libraries, compounds attached to resin beads, silica chips or other solid supports. As an example, the “split resin approach” can be used, as described in U.S. Pat. No. 5,010,175 to Rutter and in Gallop et al., J. Med. Chem., 37:1233-1251 (1994), incorporated by reference herein.

Methods for generating libraries of bi-ligands having diversity at the specificity ligand position have been described previously (see WO 99/60404, WO 00/75364, and U.S. Pat. No. 6,333,149 which issued Dec. 25, 2001). A library of bi-ligands is generated so that the binding affinity of the common ligand mimic and the specificity ligand can synergistically contribute to the binding interactions of the bi-ligand with a receptor having the respective conserved site and specificity site. Thus, the bi-ligands are generated with the specificity ligand and common ligand mimic oriented so that they can simultaneously bind to the specificity site and conserved site, respectively, of a receptor.

The present invention also provides methods of screening combinatorial libraries of bi-ligands comprising one or more common ligand mimic bound to a variety of specificity ligands and identification of bi-ligands having binding activity for the receptor. Thus, the present invention provides methods for generating a library of bi-ligands suitable for screening a particular member of a receptor family as well as other members of a receptor family.

Development of combinatorial libraries of bi-ligands of the invention begins with selection of a receptor family. Methods for determining that two receptors are in the same family, and thus constitute a receptor family, are well known in the art. For example, one method for determining if two receptors are related is BLAST, Basic Local Alignment Search Tool, available on the National Center for Biotechnology Information web page (www.ncbi.nlm.gov/BLAST/)(which is incorporated herein by reference) and modified BLAST protocols. A second resource for identifying members of a receptor family is PROSITE, available at ExPASy (www.expasy.ch/sprot/prosite.html)(which is incorporated herein by reference). A third resource for identifying members of a receptor family is Structural Classification of Proteins (SCOP) available at SCOP (scop.mrc-lmb.cam.ac.uk/scop/) (which is incorporated herein by reference).

Once a receptor family has been identified, the next step in development of bi-ligands involves determining whether there is a natural common ligand that binds at least two members of the receptor family, and preferably to several or most members of the receptor family. In some cases, a natural common ligand for the identified receptor family is already known. For example, it is known that dehydrogenases bind to dinucleotides such as NAD or NADP. Therefore, NAD or NADP are natural common ligands to a number of dehydrogenase family members. Similarly, all kinases bind ATP, and, thus, ATP is a natural common ligand to kinases.

After a receptor family has been selected, at least two receptors in the receptor family are selected as receptors for identifying useful common ligand mimics. Selection criteria depend upon the specific use of the bi-ligands to be produced. Once common ligand mimics are identified, these compounds are screened for binding affinity to the receptor family.

Those common ligand mimics having the most desirable binding activity then can be modified by adding substituents that are useful for the attachment and orientation of a specificity ligand. For example, in the present invention, thiazolidinedione and rhodanine were determined to be common ligand mimics for NAD. These compounds can be modified, for example, by the addition of substituents to the phenyl ring. For example, the phenyl ring can be substituted with a COOH group, two OMe groups, or an NHAc group. These groups provide attachment points for the specificity ligand. Substituents added to the phenyl ring can also act as blocking groups to prevent attachment of a specificity ligand at a particular site or can act to orient the specificity ligand in a particular manner to improve binding of the bi-ligand to the receptor.

Methods of screening for common ligand mimics and bi-ligands containing the common ligand mimics are well known in the art. For example, a receptor can be incubated in the presence of a known ligand and one or more potential common ligand mimics. In some cases, the natural common ligand has an intrinsic property that is useful for detecting whether the natural common ligand is bound. For example, the natural common ligand for dehydrogenases, NAD, has intrinsic fluorescence. Therefore, increased fluorescence in the presence of potential common ligand mimics due to displacement of NAD can be used to detect competition for binding of NAD to a target NAD binding receptor (Li and Lin, Eur. J. Biochem. 235:180-186 (1996); and Ambroziak and Pietruszko, Biochemistry 28:5367-5373 (1989), each of which is incorporated herein by reference).

In other cases, when the natural common ligand does not have an intrinsic property useful for detecting ligand binding, the known ligand can be labeled with a detectable moiety. For example, the natural common ligand for kinases, ATP, can be radiolabeled with ³²P, and the displacement of radioactive ATP from an ATP binding receptor in the presence of potential common ligand mimics can be used to detect additional common ligand mimics. Any detectable moiety, for example a radioactive or fluorescent label, can be added to the known ligand so long as the labeled known ligand can bind to a receptor having a conserved site. Similarly, a radioactive or fluorescent moiety can be added to NAD or a derivative thereof to facilitate screening of the NAD common ligand mimics and for bi-ligands of the invention.

The pool of potential common ligand mimics screened for competitive binding with a natural common ligand can be a broad range of compounds of various structures. However, the pool of potential ligands can also be focused on compounds that are more likely to bind to a conserved site in a receptor family. For example, a pool of candidate common ligand mimics can be chosen based on structural similarities to the natural common ligand.

Thiazolidinedione and rhodanine were identified as common ligand mimics of NAD by first determining the three-dimensional structure of NAD, the natural common ligand, and searching commercially available databases of commercially available molecules such as the Available Chemicals Directory (MDL Information Systems, Inc.; San Leandro CA) to identify potential common ligands having similar shape or electrochemical properties to NAD. Methods for identifying molecules having similar structure are well known in the art and are commercially available (Doucet and Weber, in Computer-Aided Molecular Design: Theory and Applications, Academic Press, San Diego Calif. (1996), which is incorporated herein by reference; software is available from Molecular Simulations, Inc., San Diego Calif.). Furthermore, if structural information is available for the conserved site in the receptor, particularly with a known ligand bound, compounds that fit the conserved site can be identified through computational methods (Blundell, Nature 384 Supp:23-26 (1996), which is incorporated herein by reference). These methods also can be used to screen for specificity ligands and bi-ligands of the invention.

Once a library of bi-ligands is generated, the library can be screened for binding activity to a receptor in a corresponding receptor family. Methods of screening for binding activity that are well known in the art can be used to test for binding activity.

The common ligand mimics and bi-ligands of the present invention can be screened, for example, by the following methods. Screening can be performed through kinetic assays that evaluate the ability of the common ligand mimic or bi-ligand to react with the receptor. For example, where the receptor is and reductase or dehydrogenase for which NAD is a natural common ligand, compounds of the invention can be assayed for their ability to oxidize NADH or NADPH or for their ability to reduce NAD⁺. Such assays are described more fully in Examples 23 through 25.

### EXAMPLES

Starting materials were obtained from commercial suppliers and used without further purification. ¹H NMR spectra were acquired on a Bruker Avance 300 spectrometer at 300 MHz for ¹H NMR and 75 MHz for ¹³C NMR. Chemical shifts are recorded in parts per million (δ) relative to TMS (δ=0.0 ppm) for 1H or to the residual signal of deuterated solvents (chloroform, δ=7.25 ppm for ¹H; δ=77.0 ppm for ¹³C). Coupling constant J is reported in Hz. Chromatography was performed on silica gel with ethyl acetate/hexane as elutant unless otherwise noted. Mass spectra were recorded on LCQ from Finnigan.

### Example 1

### Preparation of 4-[5-(2,4-dioxo-thiazolidin-5-ylidenemethyl)-furan-2-yl]-benzoic Acid (Compound 5a)

This example describes the synthesis of thiazolidinedione compounds following the scheme shown in FIG. 1. Compound numbers correspond to those in the figure.

Step a: Formation of 4-(5-formyl-furan-2-yl)-benzoic Acid (compound 3a)

The compound 4-aminobenzoic acid (compound 1, 60.0 g, 0.438 mol) was suspended in 100 ml of water. The solution was stirred while HCl 12M (225 ml) was added. The resulting suspension was stirred for about 10 minutes and then cooled to 1° C. A solution of NaNO₂ (30.2 g, 0.438 mol) in 200 ml of water was added to the mixture in small portions while maintaining the temperature between 5° C. and 10° C. Addition of the NaNO₂ was accomplished over a time period of approximately 30 minutes. The reaction mixture was stirred at 5° C. for an additional 30 minutes while adding another 300 ml of water. The mixture remained a suspension.

A solution of CuCl₂.2H₂O (7.5 g, 0.044 mol) in 300 ml of water was added, followed by a pre-cooled solution of 2-furaldehyde (compound 2, 36 ml, 0.435 mol) in 50 ml of acetone. While stirring, CuCl (1.8 g, 0.018 mol) was added in small portions over a period of time of 10 minutes, which resulted in foaming and precipitation of 4-(5-formyl-furan-2-yl)-benzoic acid (compound 3a).

The ice bath was removed and the mixture stirred for 30 minutes. During this period, the internal temperature rose from 5° C. to 15° C. An additional amount of CuCl (500 mg, 5 mmol) was added, and the mixture stirred for 20 minutes. This addition of CuCl resulted in a rise in the internal temperature of the suspension to 20° C.

An additional amount of CuCl (500 mg, 5 mmol) was then added, and the mixture stirred at room temperature for 16 hours. The resulting brown precipitate was filtered, thoroughly washed with water, and lyophilized. The compound 4-(5-formyl-furan-2-yl)-benzoic acid (compound 5a) was obtained as a brown powder (73.2 g, 77% mass yield). The purity of the material was about 70-80% according to NMR. The compound was employed in step b without further purification. However, a small amount of the compound was purified by recrystallization in ethanol. The results of the NMR analysis of the product follow.

¹H NMR (300 MHz, DMSO-d₆) δ 7.31 (d, J=3.5, 1H), 7.66 (d, J=3.5, 1H), 7.82 (d, J=8.0, 2H), 8.00 (d, J 8.0, 2H), 9.62 (s, 1H).

Step b: Formation of 4-[5-(2,4-dioxo-thiazolidin-5-ylidenemethyl)-furan-2-yl]-benzoic Acid (Compound 5a)

Crude 4-(5-formyl-furan-2-yl)-benzoic acid (compound 3a, 30.2 g, about 0.140 mol) and 2,4-thiazolidinedione (compound 4, 18.0 g, 0.154 mol) were mixed in 500 ml of ethanol in a 1L flask equipped with a magnetic stirring bar. Piperidine (2.8 ml, 0.028 mol) was added, and the resulting suspension was heated at 70° C. for 5 hours while stirring. The mixture was then cooled with ice, and the yellow precipitate was filtered off and washed with a mixture of ethyl acetate and ether.

The crude product was suspended in 100 ml of aqueous HCl 0.1N and placed in an ultrasound bath for 10 minutes to eliminate any residual piperidine (about 10%). The product was then filtered and dried by lyophilization to provide the compound 4-[5-(2,4-dioxo-thiazolidin-5-ylidenemethyl)-furan-2-yl]-benzoic acid (compound Sa) as a nice yellow orange powder (16.95 g, 38%). The product was analyzed by NMR with the following results.

¹H NMR (300 MHz, DMSO-d₆): δ 7.24 (d, J=3.6, 1H), 7.40 (d, J=3.6, 1H), 7.63 (s, 1H), 7.89 (d, J=8.2, 2H), 8.06 (d, J=8.3, 2H); ¹³C NMR (75.5 MHz, DMSO-d₆): δ 111.46, 117.67, 120.87, 121.06, 124.03, 130.18, 130.40, 132.36, 149.68, 155.58, 166.75, 166.92, 168.57; MS m/z 316 (M+1).

### Example 2

### Preparation of 3-[5-(2,4-dioxo-thiazolidin-5-ylidenemethyl)-furan-2-yl]-benzoic Acid (Compound 5b)

This example describes the synthesis of thiazolidinedione compounds following the reaction scheme shown in FIG. 1. Compound numbers correspond to those in the figure.

Step a: Formation of 3-(5-formyl-furan-2-yl)-benzoic Acid (Compound 3b)

The compound 3-(5-formyl-furan-2-yl)-benzoic acid (compound 3b) was prepared from 3-(5-formyl-furan-2-yl)-benzoic acid (compound 1) following the procedure in step a of Example 1. The compound was prepared in 69% yield and analyzed by NMR with the following results.

¹H NMR (300 MHz, DMSO-d₆): δ 7.42 (d, J=3.43, 1H), 7.63-7.69 (m, 2H), 8.01 (d, J=7.6, 1H), 8.13 (d, J=7.7, 1H), 8.40 (s, 1H), 9.66 (s, 1H); MS: m/z 217 (M+1).

Step b: Formation of 3-[5-(2,4-dioxo-thiazolidin-5-ylidenemethyl)-furan-2-yl]-benzoic Acid (Compound 5b)

Crude 3-(5-formyl-furan-2-yl)-benzoic acid (compound 3b, 35.0 g, 0.162 mol) and 2,4-thiazolidinedione (compound 4, 22.8 g, 0.195 mol) were mixed in 500 ml of ethanol in a 1L flask equipped with a magnetic stirring bar. Piperidine (1.6 ml, 0.0162 mol) was added to the mixture through syringe, and the suspension was heated at 70° C. for 5 hours while stirring.

The mixture was cooled with ice, and the yellow precipitate was collected and washed with a mixture of ethyl acetate and ether. The crude product was suspended in 100 ml of aqueous HCl (0.1N) and placed in an ultrasound bath for 10 minutes to eliminate residual piperidine (about 10%). The compound was filtered and lyophilized to obtain a yellow-orange powder (18.51 g, 36%). The product was analyzed by NMR with the following results.

¹H NMR (300 MHz, DMSO-d₆): δ 7.22 (d, J=3.4, 1H), 7.39 (d, J=3.4, 1H), 7.63 (s, 1H), 7.66 (t, J=7.8, 1H), 7.96 (d, J=7.3, 1H), 8.05 (d, J=7.7, 1H), 8.37 (s, 1H); ¹³C NMR (75.5 MHz, DMSO-d₆): δ 110.31, 117.72, 120.81, 120.86, 124.64, 128.22, 129.16, 129.39, 129.64, 131.82, 149.24, 155.68, 166.78, 167.26, 168.76; MS m/z 316 (M).

### Example 3

### Preparation of 5-[5-(4-hydroxy-phenyl)-furan-2-ylmethylene]-thiazolidine-2,4-dione (Compound 5c)

This example describes the synthesis of thiazolidinedione compounds following the reaction scheme shown in FIG. 1. Compound numbers correspond to those in the figure.

Step a: Formation of 5-(4-hydroxy-phenyl)-furan-2-carbaldehyde (Compound 3c)

The compound 5-(4-hydroxy-phenyl)-furan-2-carbaldehyde (compound 3c) was prepared following the procedure in step (a) of Example 1. The compound was prepared in 83% yield and analyzed with the following results.

¹H NMR (300 MHz, DMSO-d₆): δ 6.89 (d, J=8.5, 2H), 7.07 (d, J=3.6, 1H), 7.61 (d, J=3.6, 1H), 7.71 (d, J=8.5, 2H), 9.53 (s, 1H), 10.03 (br. s., 1H); MS m/z 189 (M+1).

Step b: Formation of 5-[5-(4-hydroxy-phenyl)-furan-2-ylmethylene]-thiazolidine-2,4-dione (Compound 5c)

The compound 5-[5-(4-hydroxy-phenyl)-furan-2-ylmethylene]-thiazolidine-2,4-dione (compound 5c) was prepared following the procedure in step b of Example 1. The compound was prepared in 78% yield and analyzed with NMR with the following results. ¹H NMR (300 MHz, CD₃OD): δ 6.85 (d, J=3.7, 1H), 6.89-6.92 (m, 2H), 7.03 (d, J=3.7, 1H), 7.58 (s, 1H), 7.64-7.68 (m, 1H).

### Example 4

### Preparation of 5-[5-(2,4-dioxo-thiazolidin-5-ylidenemethyl)-furan-2-yl]-2-hydroxy-benzoic Acid Methyl Ester (Compound 5d)

This example describes the synthesis of thiazolidinedione compounds following the reaction scheme shown in FIG. 3. Compound numbers correspond to those in the figure.

Step a: Formation of 5-trimethylstannanyl-furan-2-carbaldehyde (Compound 9)

A solution of butyl lithium (BuLi; 105 mmol, 2.5 M in hexanes) was added to a solution of 4-methylpiperidine (10.00 g, 100 mmol) in 50 ml of tetrahydrofuran (THF) under N₂ at −78° C., followed by the addition of 2-furaldehyde (8.73 g, 91 mmol). The solution was kept at −78° C. for 15 minutes, and then another portion of BuLi (105 mmol, 2.5 M solution in hexane) was added. The reaction mixture was allowed to warm to −20° C. and was stirred for 5 hours.

The solution was cooled to −78° C. and then added to a solution of Me₃SnCl (100 mmol, 1M solution in THF). The mixture was allowed to warm gradually to room temperature and then stirred overnight. The reaction was quenched by adding 150 ml of cold brine and extracted with EtOAc (3×100 ml). The combined organic phase was dried and concentrated.

Chromatography (EtOAc/Hexane 20:1) afforded 20.7 g (88.5%) of 5-trimethylstannanyl-furan-2-carbaldehyde. The product was analyzed by NMR as follows:

¹H NMR (300 MHz, CDCl₃) δ 0.41 (s, 9H), 6.74 (d, J=3.7, 1H), 7.25 (d, J=3.6, 1H), 9.67 (s, 1H); MS m/z 261 (M+1).

Step b: Formation of 5-(5-formyl-furan-2-yl)-2-hydroxy-benzoic Acid Methyl Ester (Compound 3d)

The 5-trimethylstannanyl-furan-2-carbaldehyde (compound 9, 2.60 g, 10 mmol), methyl 2-hydroxy-5-bromobenzoate (compound 8, 2.30 g, 10 mmol), and tetrakis(triphenylphosphine)palladium (Pd(PPh₃) 4; 0.577 g, 1 mmol) in 25 ml of dimethylformamide (DMF) was heated to 60° C. under N₂ atmosphere for 30 hours. The solution was evaporated to dryness under reduced pressure, and the residue was purified by chromatography (EtOAc/hexane 1:1) to give 2.13 g (86.2%) of methyl 5-(5-formyl-furan-2-yl)-2-hydroxy-benzoic acid methyl ester. NMR analysis of the product provided the following:

¹H NMR (300 MHz, CDCl₃) δ 4.03 (s, 3H), 6.78 (d, J=3.2, 1H), 7.10 (d, J=8.8, 1H), 7.27 (s, 1H), 7.34 (d, J=2.2, 1H), 7.92 (d, J=8.6, 1H), 8.36 (s, 1H), 9.64 (s, 1H), 11.03 (s, 1H); MS m/z 247 (M+1).

Step c: Formation of 5-[5-(2,4-dioxo-thiazolidin-5-ylidenemethyl)-furan-2-yl]-2-hydroxy-benzoic Acid Methyl Ester (Compound 5d)

The compound 5-(5-formyl-furan-2-yl)-2-hydroxy-benzoic acid methyl ester (compound 3d, 872 mg, 3.54 mmol) and 2,4-thiazolidinedione (compound 4, 539 mg, 4.60 mmol) were suspended in 25 ml of ethanol. Five drops of piperidine were added, and the mixture was heated to 70° C. for 5 hours. The mixture was then cooled to room temperature overnight. The bright orange precipitate that formed was collected on a fritted filter to give 1.1 g (90%) 5-[5-(2,4-dioxo-thiazolidin-5-ylidenemethyl)-furan-2-yl]-2-hydroxy-benzoic acid methyl ester (compound 5d). NMR analysis of the product provided the following data:

¹H NMR (300 MHz, DMSO-d₆): δ 3.93 (s, 3H), 7.14 (d, J=8.7, 1H), 7.19 (m, 2H), 7.61 (s, 1H), 7.92 (d, J=8.7, 2.3, 1H), 8.16 (d, J=2.3, 1H), 10.71 (s, 1H).

### Example 5

### Preparation of 5-[5-(2,4-dioxo-thiazolidin-5-ylidenemethyl)-furan-2-yl]-2-hydroxy-benzoic Acid (5e)

This example describes conversion of thiazolidinedione benzoic acid methyl esters to the corresponding thiazolidinedione benzoic acids following the reaction scheme shown in FIGS. 1 through 3.

The compound 5-[5-(2,4-dioxo-thiazolidin-5-ylidenemethyl)-furan-2-yl]-2-hydroxy-benzoic acid methyl ester (compound 5d, 500 mg, 1.45 mmol) was suspended in methanol. A solution of LiOH (800 mg, 16.7 mmol) in 8 ml of H₂O was added. The reaction mixture was stirred at room temperature for 20 hours. The clear solution was then acidified with 2N HCl to pH 1 and quickly extracted three times with EtOAc. The combined organic layers were dried over MgSO₄, filtered, and concentrated in vacuo to give 450 mg (94%) of 5-[5-(2,4-dioxo-thiazolidin-5-ylidenemethyl)-furan-2-yl]-2-hydroxy-benzoic acid (compound 5e). NMR analysis showed the following:

¹H NMR (300 MHz, DMSO-d₆): δ 6.76 (d, J=8.5, 1H), 6.96 (d, J=3.7, 1H), 7.14 (d, J=3.7, 1H), 7.54 (s, 1H), 7.63 (dd, J=8.5, 2.4, 1H), 8.14 (d, J=2.4, 1H).

### Example 6

### Preparation of N-{3-[5-(2,4-dioxo-thiazolidin-5-ylidenemethyl)-furan-2-yl]-phenyl}-acetamide (Compound 5f)

This example describes the synthesis of thiazolidinedione compounds following the reaction scheme shown in FIG. 5. Compound numbers correspond to those in the figures.

Step a: Formation of N-[3-(5-formyl-furan-2-yl)-phenyl]-acetamide (Compound 3f)

A mixture of 5-bromofuraldehyde (compound 11, 219 mg, 1.25 mmol), 3-acetamidophenylboronic acid (compound 10a, 291 mg, 1.63 mmol), Pd(PPh₃)₄ (72 mg, 0.062 mmol), sodium carbonate (345 mg, 3.25 mmol), dioxane (8 ml), and D. I. water (1 ml) was deoxygenated with nitrogen (N₂). The mixture was then heated at 90° C. for 10 hours and cooled to room temperature. The cooled mixture was poured onto a silica gel column and eluted with EtOAc/Hexane (1:1). The compound N-[3-(5-formyl-furan-2-yl)-phenyl]-acetamide (compound 3f, 290 mg, 1.26 mmol, 100%) was obtained as a white solid. NMR analysis of the product gave the following:

¹H NMR (300 MHz, Acetone-d₆): δ 2.13 (s, 3H), 7.10 (d, J=3.7, 1H), 7.39-7.44 (m, 1H), 7.53 (d, J=3.7, 1H), 7.53 -7.58 (m, 1H), 7.74-7.77 (m, 1H), 7.48 (d, J=1.7, 1H), 9.67 (s, 1H).

Step b: Formation of N-{3-[5-(2,4-dioxo-thiazolidin-5-ylidenemethyl)-furan-2-yl]-phenyl}-acetamide (Compound 5f)

The compound N-{3-[5-(2,4-dioxo-thiazolidin-5-ylidenemethyl)-furan-2-yl]-phenyl}-acetamide (compound 5f) from N-[3-(5-formyl-furan-2-yl)-phenyl]-acetamide (compound 3f) was prepared following the procedure in step b of Example 1. The compound was obtained in 90% yield, and NMR analysis gave the following:

¹H NMR (300 MHz, DMSO-d₆): δ 2.08 (s, 3H), 7.18 (d, J=3.7, 1H), 7.22 (d, J=3.7, 1H), 7.39-7.59 (m, 3H), 7.62 (s, 1H), 8.08 (s, 1H).

### Example 7

### Preparation of 5-[5-(3,4-dimethoxy-phenyl)-furan-2-yl-methylene]-thiazolidine-2,4-dione (compound 5g)

This example describes the synthesis of thiazolidinedione compounds following the reaction scheme show in FIG. 5. Compound numbers correspond to those in the figure.

Step a: Formation of 5-(3,4-Dimethoxyphenyl)-2-furaldehyde (Compound 3g)

The compound 5-(3,4-dimethoxyphenyl)-2-furaldehyde (compound 3g) was prepared from 3,4-dimethoxyphenylboronic acid (compound lob) and 5-bromo-2-furaldehyde following the procedure in step a of Example 6. The compound was obtained in 90% yield, and NMR analysis gave the following:

¹H NMR (300 MHz, CDCl₃) δ 3.92 (m, 3H), 3.95 (s, 3H), 6.73 (d, J=3.8, 1H), 6.92 (d, J=8.4, 1H), 7.30 (m, 2H), 7.40 (dd, J=2.0, 8.4, 1H), 9.59 (s, 1H); MS m/z 233 (M+1).

Step b: Formation of 5-[5-(3,4-dimethoxy-phenyl)-furan-2-yl-methylene]-thiazolidine-2,4-dione (Compound 5g)

The compound 5-[5-(3,4-dimethoxy-phenyl)-furan-2-ylmethylene]-thiazolidine-2,4-dione (compound 5g) was prepared from 5-(3,4-dimethoxyphenyl)-2-furaldehyde (compound 3g) following the procedure in step b of Example 1. The product was obtained in 94% yield, and NMR analysis showed the following:

¹H NMR (300 MHz, CDCl₃) δ 3.95 (s, 3H), 3.99 (s, 1H), 6.79 (d, J=3.9, 1H), 6.91 (d, J=3.8, 1H), 6.98 (d, J=8.4, 1H), 7.28 (s, 1H), 7.35 (dd, J=8.4, 1.9, 1H), 7.62 (s, 1H); MS m/z 332 (M+1).

### Example 8

### Preparation of 4-[5-(4-oxo-2-thioxo-thiazolidin-5-ylidenemethyl)-furan-2-yl]-benzoic Acid (compound 7a)

This example describes the synthesis of rhodanine compounds following the reaction scheme shown in FIG. 2. The compound numbers correspond to those in the figure.

The compound 4-(5-formyl-furan-2-yl)-benzoic acid (compound 3a, 412 mg, 1.91 mmol), rhodanine (compound 6, 279 mg, 2.09 mmol), and piperidine (38 μl, 0.384 mmol) were placed in 5 ml of ethanol in a vial. The mixture was stirred under microwave irradiation for 300 seconds at 160° C. The mixture was then cooled to room temperature, and the obtained orange precipitate was filtered, washed with a mixture of ethyl acetate and ether, and dried in vacuo to provide 4-[5-(4-oxo-2-thioxo-thiazolidin-5-ylidenemethyl)-furan-2-yl]-benzoic acid as an orange powder (compound 7a, 477 mg, 75% yield). NMR analysis of the product provided the following:

¹H NMR (300 MHz, DMSO-d₆): δ 7.34 (d, J=3.3, 1H), 7.45 (d, J=3.2, 1H), 7.52 (s, 1H), 7.93 (d, J=8.2, 2H) and 8.08 (d, J=8.0, 2H); MS: m/z 332 (M+1).

### Example 9

### Preparation of 3-[5-(4-oxo-2-thioxo-thiazolidin-5-ylidenemethyl)-furan-2-yl]-benzoic Acid (Compound 7b)

This example describes the synthesis of rhodanine compounds following the reaction scheme of FIG. 2. Compound numbers correspond to those in the figure.

The compound 3-(5-formyl-furan-2-yl)-benzoic acid (compound 3b, 3.45 mmol), rhodanine (compound 6, 460 mg, 3.45 mmol), water (15 ml), and ethanolamine (21 μl, 0.35 mmol) were placed in a flask. The suspension was stirred at 90° C. for 3 hours. After cooling to room temperature, the resulting orange precipitate was filtered and dried in vacuo to give 3-[5-(4-oxo-2-thioxo-thiazolidin-5-ylidenemethyl)-furan-2-yl]-benzoic acid (compound 7b, 573 mg, 50% yield). NMR analysis of the product revealed:

¹H NMR (300 MHz, DMSO-d₆): δ 7.31 (d, J=3.6, 1H), 7.43 (d, J=3.6, 1H), 7.50 (s, 1H), 7.69 (t, J=7.8, 1H), 7.97 (d, J=7.7, 1H), 8.07 (d, J=7.8, 1H), 8.38 (s, 1H).

### Example 10

### Preparation of 5-[5-(4-hydroxy-phenyl)-furan-2-ylmethylene]-2-thioxo-thiazolidin-4-one (Compound 7c)

This example describes the synthesis of rhodanine compounds following the reaction scheme of FIG. 2. Compound numbers correspond to those in the figure.

The compound 5-[5-(4-hydroxy-phenyl)-furan-2-ylmethylene]-2-thioxo-thiazolidin-4-one (compound 7c) was prepared from 5-(4-hydroxy-phenyl)-furan-2-carbaldehyde (compound 3c) following the procedure in step b of Example 1. The compound was prepared in 81% yield. NMR analysis provided the following:

¹H NMR (300 MHz, Acetone-d₆): δ 7.00-7.03 (m, 3H), 7.24-7.25 (m, 1H), 7.46 (s, 1H), 7.77-7.79 (m, 2H).

### Example 11

### Preparation of 2-hydroxy-5-[5-(4-oxo-2-thioxo-thiazolidin-5-ylidenemethyl)-furan-2-yl]-benzoic Acid Methyl Ester (Compound 7d)

This example describes the synthesis of rhodanine compounds following the reaction scheme shown in FIG. 4. Compound numbers correspond to those in the figure.

The compound 2-hydroxy-5-[5-(4-oxo-2-thioxo-thiazolidin-5-ylidenemethyl)-furan-2-yl]-benzoic acid methyl ester (compound 7d) was prepared from 5-(5-formyl-furan-2-yl)-2-hydroxy-benzoic acid methyl ester (compound 3d) following the procedure in Example 9. The compound was prepared in 83% yield. NMR analysis revealed the following:

¹H NMR (300 MHz, DMSO-d₆): δ 3.94 (s, 3H), 7.18 (d, J=8.7, 1H), 7.23 (d, J=3.5, 1H), 7.30 (d, J=3.5, 1H), 7.50 (s, 1H), 7.97 (dd, J=8.7, 1.9, 1H) and 8.26 (d, J 1.9, 1H).

### Example 12

### Preparation of 2-hydroxy-5-[5-(4-oxo-2-thioxo-thiazolidin-5-ylidenemethyl)-furan-2-yl]-benzoic Acid (Compound 7e)

This example describes conversion of rhodanine benzoic acid methyl esters to the corresponding rhodanine benzoic acids.

The compound 2-hydroxy-5-[5-(4-oxo-2-thioxo-thiazolidin-5-ylidenemethyl)-furan-2-yl]-benzoic acid methyl ester (compound 7d, 36 mg, 0.10 mmol) was suspended in methanol (0.5 ml) and THF (0.25 ml). A solution of LiOH (57 mg, 2.38 mmol) in H₂O (0.25 ml) was added. The reaction mixture was stirred at room temperature for 20 hours. The resulting clear solution was then acidified with 2N HCl to pH=1 and was quickly extracted three times with EtOAc. The combined organic layers were dried over MgSO₄, filtered, and concentrated in vacuo to give 2-hydroxy-5-[5-(4-oxo-2-thioxo-thiazolidin-5-ylidenemethyl)-furan-2-yl]-benzoic acid (compound 7e, 27 mg, 0.078 mmol, 78%). The product was analyzed by NMR to provide the following:

¹H NMR (300 MHz, CD₃OD): δ 6.88 (d, J=3.7, 1H), 6.96 (d, J=8.6, 1H), 7.07 (d, J=3.7, 1H), 7.37 (s, 1H), 7.79 (dd, J=8.6, 2.1, 1H), 8.33 (d, J 2.1, 1H). MS (ESI negative mode): m/z 346 (M−1).

### Example 13

### Preparation of 5-[5-(3,4-dimethoxy-phenyl)-furan-2-ylmethylene]-2-thioxo-thiazolidin-4-one (Compound 7f)

This example describes the synthesis of rhodanine compounds following the reaction scheme show in FIG. 6. Compound numbers correspond to those in the figure.

Step a: Formation of 5-(3,4-dimethoxyphenyl)-2-furaldehyde (7f)

A solution of 3,4-dimethoxyphenylboronic acid (compound 10b, 0.945 g, 5.2 mmol), 5-bromo-2-furaldehyde (0.696 g, 4 mmol), Pd(PPh₃)₄ (0.231 g, 0.2 mmol) and Na₂CO₃ (1.270 g, 12 mmol) in a mixture of 20 ml of water and dioxane (1:10) was heated under N₂ at reflux overnight. The reaction mixture was concentrated, and the residue was purified by chromatography (EtOAc/hexanes 1:3) to give 5-(3,4-Dimethoxyphenyl)-2-furaldehyde (0.823 g, 90%). The product was analyzed by NMR to give the following:

¹H NMR (300 MHz, CDCl₃) δ 3.92 (m, 3H), 3.95 (s, 3H), 6.73 (d, J=3.8, 1H), 6.92 (d, J=8.4, 1H), 7.30 (m, 2H), 7.40 (dd, J=8.4, 2.0, 1H), 9.59 (s, 1H); MS m/z 233 (M+1).

Step b: Formation of 5-[5-(3,4-dimethoxy-phenyl)-furan-2-ylmethylene]-2-thioxo-thiazolidin-4-one (Compound 7f)

A solution of 5-(3,4-dimethoxyphenyl)-2-furaldehyde (compound 3f, 100 mg, 0.43 mmol), rhodanine (compound 6, 75 mg, 0.64 mmol), and ethanolamine (26 μl, 0.43 mmol) in a mixture of 1 ml of AcOH and 5 ml of dioxane was heated at reflux for 3 hours. Concentration and recrystallization from ethanol afforded the coupling product 5-[5-(3,4-dimethoxy-phenyl)-furan-2-ylmethylene]-2-thioxo-thiazolidin-4-one (compound 7f, 81 mg, 93%). NMR analysis provided:

¹H NMR (300 MHz, CDCl₃) δ 3.93 (s, 3H), 4.01 (s, 3H), 6.77 (d, J=3.8, 1H), 6.99 (m, 2H), 7.28 (m, 2H), 7.42 (s, 1H); MS m/z 347 (M+1).

### Example 14

### Preparation of 4-(2-{4-[5-(2,4-dioxo-thiazolidin-5-ylidenemethyl)-furan-2-yl]-benzoylamino}-ethylsufanyl)-pyridine-2,6-dicarboxylic Acid (Compound 13a)

This example describes the synthesis of bi-ligands of the invention following the reaction scheme show in FIG. 15. Compound numbers correspond to those in the figure.

The compound 4-amino-pyridine-2,6-dicarboxylic acid dimethyl ester (compound 12, free base, 75 mg, 0.277 mmol), 4-[5-(2,4-dioxo-thiazolidin-5-ylidenemethyl)-furan-2-yl]-benzoic acid (compound 5a, 87 mg, 0.276 mg) and HOBt.H₂O (51 mg, 0.333 mmol) were dissolved in DMF (1 ml). Triethylamine (46 μl, 0.331 mmol) and 1-dimethylaminopropyl-3-ethyl-carbodiimide (EDCI) (70 mg, 0.333 mmol) were added to the mixture which was then stirred at room temperature for 24 hours. The resulting precipitate (52.4 mg) was collected on a funnel and washed with DMF, aqueous 0.5N HCl, and MeOH.

Next, 48.2 mg of the solid was suspended in a mixture of MeOH (0.5 ml) and water (0.5 ml), followed by the addition of LiOH (14 mg, 0.585 mmol). The solution was then stirred at room temperature for 1.5 hours until homogenous. The homogenous solution was acidified with aqueous 2N HCl, and the resulting precipitate was filtered, washed with water, and dried. The reaction afforded a bright yellow solid: 4-(2-{4-[5-(2,4-dioxo-thiazolidin-5-ylidenemethyl)-furan-2-yl]-benzoylamino}-ethylsufanyl)-pyridine-2,6-dicarboxylic acid (compound 13a, 41.5 mg, 30%).

¹H NMR (300 MHz, DMSO-d₆): δ 3.42 (m, 2H), 3.60 (m, 2H) 7.26 (d, J=3.6, 1H), 7.41 (d, J=3.5, 1H), 7.67 (s, 1H), 7.89 (d, J=8.3, 2H), 7.95 (d, J=8.4, 2H), 8.08 (s, 2H), 8.85 (br. t., 1H); MS m/z 540 (M+1).

### Example 15

### Preparation of 4-(2-{4-[5-(4-oxo-2-thioxo-thiazolidin-5-ylidenemethyl)-furan-2-yl]-benzoylamino}-ethylsulfanyl)-pyridine-2,6-dicarboxylic Acid (compound 13b)

This example describes the synthesis of bi-ligands of the invention following the reaction scheme shown in FIG. 15. Compound numbers correspond to those in the figure.

The compound 4-amino-pyridine-2,6-dicarboxylic acid dimethyl ester (compound 12, HCl salt, 84 mg, 0.275 mmol), 4-[5-(4-oxo-2-thioxo-thiazolidin-5-ylidenemethyl)-furan-2-yl]-benzoic acid (compound 7a, 91 mg, 0.275 mmol) and HOBt.H₂O (51 mg, 0.333 mmol) were dissolved in DMF (1 ml). Triethylamine (0.11 ml, 0.79 mmol) and EDCI (0.329 mmol) were added to the mixture, followed by stirring at room temperature for 24 hours.

Four drops of concentrated HCl were added to the mixture and induced formation of a precipitate (159 mg), which was filtered, washed with aqueous 0.1N HCl, and dried in vacuo. Then, 111 mg of this compound were placed in a mixture of water (0.5 ml) and MeOH (0.5 ml). LiOH (40 mg, 1.67 mmol) was added to the mixture which was stirred at room temperature for 2 hours.

The lithium salt of the expected compound precipitated from the solution and was isolated by filtration. The salt was dissolved in warm water (about 40° C.) and precipitated by addition of aqueous 2N HCl. The precipitate was filtered and dried in vacuo to give 4-(2-{4-[5-(4-oxo-2-thioxo-thiazolidin-5-ylidenemethyl)-furan-2-yl]-benzoylamino}-ethylsulfanyl)-pyridine-2,6-dicarboxylic acid as a red powder (compound 13b, 41 mg,

¹H NMR (300 MHz, DMSO-d₆): δ 3.54 (br. t., 2H), 3.60 (br. t., 2H), 7.35 (d, J=3.5, 1H), 7.44 (d, J=3.5, 1H), 7.54 (s, 1H), 7.91 (d, J=8.2, 2H), 7.99 (d, J=8.3, 2H), 8.08 (s, 2H), 8.87 (br. t., 1H); MS m/z 556 (M+1).

### Example 16

### Preparation of 4-(2-{3-[5-(2,4-dioxo-thiazolidin-5-ylidenemethyl)-furan-2-yl]-benzoylamino}-ethylsulfanyl)-pyridine-2,6-dicarboxylic Acid (13c)

This example describes the synthesis of bi-ligands of the invention following the reaction scheme shown in FIG. 15. Compound numbers correspond to the numbers in the figure.

The compound 4-amino-pyridine-2,6-dicarboxylic acid dimethyl ester (compound 12, HCl salt, 100 mg, 0.326 mmol), 3-[5-(2,4-dioxo-thiazolidin-5-ylidenemethyl)-furan-2-yl]-benzoic acid (compound 5b, 103 mg, 0.327 mmol) and HOBt-H₂O (60 mg, 0.392 mmol) were dissolved in DMF (1 ml). Triethylamine (0.14 ml, 1.01 mmol) and EDCI (75 mg, 0.391 mmol) were added to the mixture which was then stirred at room temperature for 2.5 days. The resulting solid (73 mg) was collected on a funnel, washed with aqueous 0.5N HCl and dried.

The product (63 mg) was suspended in a mixture of water (0.5 ml) and MeOH (0.5 ml), followed by the addition of LiOH (20 mg, 0.84 mmol). The mixture was then stirred at room temperature for 1.5 hours. Water was added, and the compound was precipitated by acidification with aqueous 2N HCl. After drying in vacuo, we obtained pure 4-(2-{3-[5-(2,4-dioxo-thiazolidin-5-ylidenemethyl)-furan-2-yl]-benzoylamino}-ethylsulfanyl)-pyridine-2,6-dicarboxylic acid was obtained as a yellow powder (compound 13c, 49 mg, 32%).

¹H NMR (300 MHz, DMSO-d₆): δ 3.62 (br. m., 2H) and one signal overlapped by water at 3.44, 7.25 (d, J=3.5, 1H), 7.33 (d, J=3.5, 1H), 7.62 (t, J=7.8, 1H), 7.67 (s, 1H), 7.81 (d, J=7.7, 1H), 7.95 (d, J=7.7, 1H), 8.08 (s, 2H), 8.24 (s, 1H), 8.91 (br. t., 1H); MS m/z 540 (M+1).

### Example 17

### Preparation of 4-(2-{3-[5-(4-oxo-2-thioxo-thiazolidin-5-ylidenemethyl)-furan-2-yl]-benzoylamino}-ethylsulfanyl)-pyridine-2,6-dicarboxylic Acid (13d)

This example describes the synthesis of bi-ligands of the invention following the reaction scheme of FIG. 15. Compound numbers correspond to those in the figure.

The compound 4-amino-pyridine-2,6-dicarboxylic acid dimethyl ester (compound 12, free base, 80 mg, 0.296 mmol), 3-[5-(4-oxo-2-thioxo-thiazolidin-5-ylidenemethyl-furan-2-yl]-benzoic acid (compound 7b, 98 mg, 0.296 mmol) and HOBt.H₂O (54 mg, 0.353 mmol) were dissolved in DMF (1 ml). Triethylamine (49 l, 0.352 mmol) and EDCI (72 mg, 0.375 mmol)were added to the solution which was then stirred at room temperature for 30 hours. The resulting orange precipitate (95 mg) was filtered, washed with DMF and aqueous 0.5N HCl, and dried.

The compound (88.2 mg) was suspended in a mixture of water (1 ml) and MeOH (1 ml), followed by the addition of LiOH (25 mg, 1.05 mmol). The solution was then stirred at room temperature for 2.5 hours, and the solution was acidified with aqueous 2N HCl. The resulting solid was filtered and washed with water. After drying 4-(2-{3-[5-(4-oxo-2-thioxo-thiazolidin-5-ylidenemethyl)-furan-2-yl]-benzoylamino}-ethylsulfanyl)-pyridine-2,6-dicarboxylic acid (compound 13d, 65 mg, 42%) was obtained as a red powder.

¹H NMR (300 MHz, DMSO-d₆): δ 3.63 (m, 2H) and one signal overlapped by water at 3.39, 7.35 (s, 2H), 7.55 (s, 1H), 7.63 (t, J=7.7, 1H), 7.82 (d, J=7.7, 1H), 7.97 (d, J=7.7, 1H), 8.08 (s, 2H), 8.27 (s, 1H), 8.93 (br. t., J=5.1, 1H); MS m/z 556 (M+1).

### Example 18

### Preparation of 4-(2-{5-[5-(2,4-dioxo-thiazolidin-5-ylidenemethyl)-furan-2-yl]-2-hydroxy-benzoylamino}-ethylsulfanyl)-pyridine-2,6-dicarboxylic Acid (Compound 13f)

This examples describes the synthesisi of bi-ligands of the invention following the reaction scheme shown in FIG. 15. Compound numbers correspond to those in the figure.

The compound 4-amino-pyridine-2,6-dicarboxylic acid dimethyl ester (compound 12, free base, 73 mg, 0.270 mmol), 5-[5-(2,4-dioxo-thiazolidin-5-ylidene methyl)-furan-2-yl]-2-hydroxy-benzoic acid (compound 5e, 89 mg, 0.269 mmol) and HOBt.H₂O (49 mg, 0.320 mmol) were dissolved in DMF (1 ml). Triethylamine (45 l, 0.324 mmol) and EDCI (62 mg, 0.323 mmol) were added to the mixture which was then stirred at room temperature for 30 hours. The reaction was acidified with HCl, inducing formation of an orange precipitate that was isolated by filtration.

The isolated compound was purified by flash chromatography (SiO₂, MeOH 5% to 7.5% in dichloromethane) and suspended in a mixture of MeOH (0.5 ml) and water (0.5 ml). LiOH (15 mg) was added to the mixture which was then stirred for 2 hours at room temperature to form a homogenous solution. The homogenous solution was then acidified by aqueous 2N HCl. The resulting compound was filtered and purified by preparative HPLC to give a reddish powder: 4-(2-{5-[5-(2,4-dioxo-thiazolidin-5-ylidenemethyl)-furan-2-yl]-2-hydroxy-benzoylamino}-ethylsulfanyl)-pyridine-2,6-dicarboxylic acid (compound 13f, 16.1 mg, 15% yield).

¹H NMR (300 MHz, DMSO-d₆): δ 3.66 (m, 2H) and signal overlapped by water at 3.37, 7.10 (m, 2H), 7.22 (d, J 3.0, 1H), 7.63 (s, 1H), 7.81 (d, J=8.1, 1H), 8.11 (s, 2H), 8.24 (s, 1H), 9.12 (br. t., 1H); MS m/z 468 (M+H−2CO₂).

### Example 19

### Preparation of Common Ligand Mimics having Amide Linkers

This example describes the synthesis of common ligand mimics of the invention containing a linker group following the reaction scheme shown in FIG. 7. Compound numbers correspond to the numbers in the figure.

In a 500 ml round-bottom flask, compound 5 (6.3 g, 20 mM) was dissolved in dry DMF (120 ml) by heating. The solution was cooled to a temperature of 40 to 50° C. THF (ca 150 ml) and 1,1′-carbonyldiimidazole (4.5 g) were added to the solution. After shaking for 20 minutes, the flask was capped and refrigerated overnight at −10° C. The precipitate was collected by filtration and washed with THF to provide intermediate compound 14 (5.3-6.0 g).

A mixture of dry DMF (30 ml) and dry THF (80 ml) was prepared in a 250 ml flask. Intermediate compound 14 (5.3-6.0 g) was added to the mixture. Boc protected diamines (1.2 eq) were added to the mixture which then was heated at a temperature of 65° C. for a period of 1 hour. By this time, the undissolved solid had dissolved, and a clear solution was obtained. The solvent was then evaporated under reduced pressure.

A solution of 50% trifluoacetic acid in dichloroethane (100 ml) was added and reacted for 10 minutes. Extra solvent was evaporated, resulting in a yellow solid. The yellow solid was then dissolved in 40 to 50 ml of DMF by heating. The solution was cooled to room temperature, and a Na₂CO₃ solution (150-200 ml, 5%) was added. When a yellow precipitate formed, it was filtered. Otherwise, more DMF solvent was evaporated, and more water was added. The yellow solid, compound 16, was washed with a mixture of water and MeOH and then dried to provide 5 to 5.5 g of product 16.
Compound 16a (CLM-3-COOH, n=0); MW calcd 357.38, found: MW 358.02;
Compound 16b (CLM-3-COOH, n=1), MW calcd 371.41, found: MW 372.05;
Compound 16c (CLM-3-COOH, n=2), MW calcd 385.44, found: MW 386.10;
Compound 16d (CLM-4-COOH, n=0); MW calcd 357.38, found: MW 358.02;
Compound 16e (CLM-4-COOH, n=1), MW calcd 371.41, found: MW 372.05;
Compound 16f (CLM-4-COOH, n=2), MW calcd 385.44, found: MW 386.10.

### Example 20

### Preparation of 5-(4-N-Boc-aminoethylphenyl)-2-((2,4-thiazolidinedion-5-yl)methylene)furan

This example describes the synthesis of common ligand mimics of the invention containing a linker group following the reaction scheme shown in FIG. 8.

Step a: Formation of N-Boc-4-bromophenethylamine

The compound 4-bromophenethylamine (50 g, 0.180 mol) and NaHCO₃ (15.12 g, 0.480 mol) were suspended in 300 ml of aqueous acetone (5% water) at a temperature of 0° C. A solution of di-tert-butyldicarbonate (38.80 g, 0.180 mol) in 50 ml of acetone was added dropwise to the solution. The solution was then stirred overnight at room temperature.

The reaction mixture was poured into 200 ml of water and extracted with ethyl acetate (2×250 ml). The extracts were dried with MgSO₄ and concentrated to give a white powder (53.8 g, 98.9%) that was pure enough for the next step.

¹H NMR (CDCl₃) δ 7.77 (d, J=8.9 Hz, 2H), 7.08 (d, J=8.5 Hz, 2H), 3.36 (m, 2H), 2.73 (m, 2H), 1.44 (s, 9H) ppm. MS (M+1⁺) 303.

Step b: Formation of 5-(4-N-Boc-aminoethylphenyl)-2-furaldehyde

A mixture of N-Boc-4-bromophenethylamine (95.0 g, 0.314 mol), 5-trimethylstannanyl-2-furaldehyde (94.3 g, 0.330 mol), and tetrakis(triphenylphosphine)palladium (17.3 g, 0.016 mol) in 300 ml of DMF was heated to a temperature of 60° C. for a period of 24 hours. The reaction mixture was concentrated under reduce pressure, and the residue was purified by chromatography (EtOAc/Hexanes 5:1) to give 83.0 (83.9%) of 5-(4-N-Boc-aminoethylphenyl)-2-furaldehyde.

¹H NMR (CDCl₃) δ 9.65 (s, 1H), 7.79 (d, J=8.1 Hz, 2H), 7.30 (m, 3H), 6.82 (d, J=3.5 Hz, 1H), 3.41 (m, 2H), 2.85 (m, 2H), 1.44 (s, 9H) ppm. MS (M+1⁺) 316.

Step c: Formation of 5-(4-N-Boc-aminoethylphenyl)-2-((2,4-thiazolidinedion-5-yl)methylene)furan

A solution of 5-(4-N-Boc-aminoethylphenyl)-2-furaldehyde (25.0 g, 0.079 mol), 2,4-thiazolidinedione (9.3 g, 0.079 mol), and ethanolamine (0.5 g, 0.005 mol) in 100 ml of dioxane was heated to reflux for 3 days. The reaction mixture was concentrated, and the resultant residue was triturated several times with ethyl acetate. The precipitates were collected by filtration to give 23.5 g (72.0%) of 5-(4-N-Boc-aminoethylphenyl)-2-((2,4-thiazolidinedion-5-yl)methylene)furan.

¹H NMR (CDCl₃) δ 7.74 (d, J=6.6 Hz, 2H), 7.63 (d, J=2.2 Hz, 1H), 7.35 (d, J=6.7 Hz, 2H), 7.22 (d, J=2.0 Hz, 2H), 6.90 (t, J=3.9 Hz, 1H), 3.13 (m, 2H), 2.73 (m, 2H), 1.35 (s, 9H) ppm. MS (M+1⁺) 314.

### Example 21

### Preparation of 5-[5-(2,4-dioxothiazolidin-5-ylidenemethyl)-furan-2-yl]-nicotinic Acid (Compound 20a)

This example describes the synthesis of common ligand mimics of the invention containing a linker group following the reaction scheme shown in FIG. 9. Compound numbers correspond to the numbers in the figure.

Step a: Preparation of 5-(5-formylfuran-2-yl)-nicotinic Acid (Compound 19a)

The compounds 2-formylfuran-5-boronic acid (compound 17, 289 mg, 2.06 mmol), 5-bromonicotinic acid (compound 18a, 500 mg, 2.48 mmol) and sodium carbonate (262 mg, 2.48 mmol) were added to a mixture of dioxane (10 ml), water (5 ml), ethanol (4 ml), and DMF (0.5 ml). Dichlorobis(triphenylphosphine)palladium (87 mg, 0.12 mmol) was added to the mixture, and the mixture was heated to a temperature of 90° C. for 15 hours. Volatiles were removed in vacuo, and the residue was diluted with water, followed by extraction with ethyl acetate. Combined organic layers were dried over Mg₂SO₄, filtered, and concentrated in vacuo. The crude product was purified by flash chromatography (CH₂Cl₂/MeOH, 10:1) to give 5-(5-formylfuran-2-yl)-nicotinic acid (compound 19a, 250 mg, 47%).

¹H NMR (300 MHz, DMSO-d₆) δ 7.70 (d, J=3.0, 1H), 7.57 (d, J=3.0, 1H), 8.59 (s, 1H), 9.06 (s, 1H), 9.28 (s, 1H), 9.67 (s, 1H); ¹³C NMR (300 MHz, DMSO-d₆) δ 110.9, 124.9, 127.4, 132.3, 149.4, 150.4, 152.4, 154.5, 165.8.

Step b: 5-[5-(2,4-dioxothiazolidin-5-ylidenemethyl)-furan-2-yl]-nicotinic Acid (Compound 20a)

The compounds 5-(5-formylfuran-2-yl)-nicotinic acid (compound 19a, 78.1 mg, 0.360 mmol) and 2,4-thiazolidinedione (63.2 mg, 0.539 mmol) were mixed in ethanol (5 ml). Piperidine (2 drops) was added, and the reaction was stirred at a temperature of 70° C. for a period of 36 hours. The resulting orange precipitate was collected on filter paper using a Büchner funnel. The solid was washed with ethyl acetate, followed by ethyl ether, to give pure 5-[5-(2,4-dioxo-thiazolidin-5-ylidenemethyl)-furan-2-yl]-nicotinic acid (compound 20a, 95 mg, 84%).

¹H NMR (300 MHz, DMSO-d₆) Y 7.18 (d, J=3.6, 1H), 7.54 (d, J=3.6, 1H), 7.56 (s, 1H), 8.56 (s, 1H), 9.02 (s, 1H), 9.22 (d, J=1.4, 1H); MS m/z 317.15 (M+1).

### Example 22

### Preparation of 5-[5-(2,4-dioxothiazolidin-5-ylidenemethyl)-furan-2-yl]-N-(3-hydroxypropyl)-nicotinamide (Compound 20b)

This example describes the synthesis of common ligand mimics of the invention containing a linker group following the reaction scheme shown in FIG. 9. Compound numbers correspond to the numbers in the figure.

Step a: Formation of 5-(5-formylfuran-2-yl)-N-(3-hydroxypropyl)-nicotinamide (Compound 19b)

The compounds 2-formylfuran-5-boronic acid (compound 17, 225 mg, 1.61 mmol), 5-bromo-N-(3-hydroxy-propyl)-nicotinamide (compound 18b, 530 mg, 1.93 mmol) and sodium carbonate (205 mg, 1.93 mmol) were added to a mixture of dioxane (7 ml), water (3 ml), ethanol (2 ml) and DMF (0.4 ml). Dichlorobis(triphenylphosphine) palladium (67.8 mg, 0.0966 mmol) was added, and the reaction was heated to a temperature of 80° C. for 5 hours.

Another portion of dichlorobis(triphenyl-phosphine)palladium (67.8 mg, 0.0966 mmol) and 2-formylfuran-5-boronic acid (compound 17, 23 mg, 0.19 mmol) was added to the reaction mixture, which was then stirred overnight at room temperature. Volatiles were removed in vacuo, and the residue was diluted with saturated NaHCO₃ solution, followed by extraction with ethyl acetate.

Combined organic layers were dried over Mg₂SO₄, filtered, and concentrated in vacuo. The crude product was purified by flash chromatography (EtOAc/MeOH, 9:1) to give 5-(5-formylfuran-2-yl)-N-(3-hydroxypropyl)-nicotinamide (compound 19b, 358 mg, 81.2%).

¹H NMR (300 MHz, MeOH-d₃) δ 1.88 (m, 2H), 3.52 (m, 2H) 3.69 (m, 2H), 7.24 (d, J=3.8, 1H), 7.51 (d, J=3.8, 1H), 8.53 (m, 1H), 8.91 (d, J=1.7, 1H), 9.06 (d, J=1.7, 1H), 9.62 (s, 1H); ¹³C NMR (300 MHz, MeOH-d₃) δ 33.2, 38.5, 60.7, 111.5, 125.3, 126.9, 132.1, 132.5, 139.3, 149.1, 149.5, 154.4, 156.4, 167.1; MS m/z 374.2 (M+1).

Step b: Formation of 5-[5-(2,4-dioxothiazolidin-5-ylidenemethyl)-furan-2-yl]-N-(3-hydroxypropyl)-nicotinamide (Compound 20b)

The compounds 5-(5-formylfuran-2-yl)-N-(3-hydroxypropyl)-nicotinamide (compound 19b, 123 mg, 0.448 mmol) and 2,4-thiazolidinedione (64.2 mg, 0.493 mmol) were mixed in ethanol (5 ml). Piperidine (1 drop) was added, and the reaction was stirred at a temperature of 70° C. for a period of 2 hours. The resulting orange precipitate was collected on filter paper using a Büchner funnel. The solid was washed with ethyl acetate, followed by ethyl ether, to give pure 5-[5-(2,4-Dioxothiazolidin-5-ylidenemethyl)-furan-2-yl]-N-(3-hydroxypropyl)-nicotinamide (compound 20b, 115 mg, 76%).

¹H NMR (300 MHz, DMSO-d₆) δ 1.71 (dt, J=6.7, 6.7, 2H) 3.37 (m, 2H), 3.48 (m, 2H), 4.49 (bs, 1H), 7.28 (d, J=3.7, 1H), 7.48 (d, J=3.7, 1H), 7.68 (s, 1H), 8.50 (m, 1H), 8.76 (m, 1H), 8.96 (d, J=1.8, 1H), 9.13 (d, J=2.0, 1H); ¹³C NMR (300 MHz, DMSO-d₆) δ 32.3, 36.7, 58.5, 111.6, 117.6, 120.6, 121.4, 124.7, 130.1, 130.5, 149.9, 153.3, 164.2, 167.0, 168.4.

Examples of compounds which can be produced by the methods described in Examples 19 to 22 include those in Tables 6 to 12.

TABLE 6

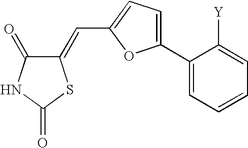

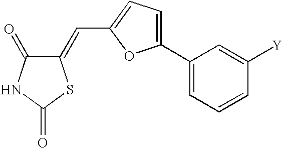

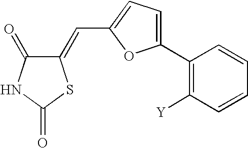

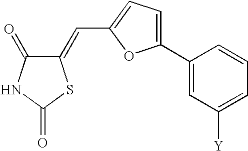

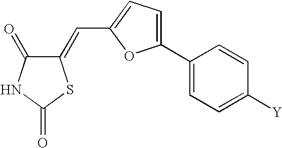

| | Y | | Y | | Y | | Y | | Y |
|---|---|---|---|---|---|---|---|---|---|
| 1 | OH | 2 | OH | 3 | OH | 4 | OH | 5 | OH |
| 1 | SH | 2 | SH | 3 | SH | 4 | SH | 5 | SH |
| 1 | COOH | 2 | COOH | 3 | COOH | 4 | COOH | 5 | COOH |
| 1 | SO₂H | 2 | SO₂H | 3 | SO₂H | 4 | SO₂H | 5 | SO₂H |
| 1 | Cl | 2 | Cl | 3 | Cl | 4 | Cl | 5 | Cl |
| 1 | Br | 2 | Br | 3 | Br | 4 | Br | 5 | Br |
| 1 | I | 2 | I | 3 | I | 4 | I | 5 | I |
| 1 | F | 2 | F | 3 | F | 4 | F | 5 | F |
| 1 | CN | 2 | CN | 3 | CN | 4 | CN | 5 | CN |
| 1 | N₃ | 2 | N₃ | 3 | N₃ | 4 | N₃ | 5 | N₃ |
| 1 | CONH₂ | 2 | CONH₂ | 3 | CONH₂ | 4 | CONH₂ | 5 | CONH₂ |
| 1 | CH═CH₂ | 2 | CH═CH₂ | 3 | CH═CH₂ | 4 | CH═CH₂ | 5 | CH═CH₂ |
| 1 | C≡CH | 2 | C≡CH | 3 | C≡CH | 4 | C≡CH | 5 | C≡CH |
| 1 | NH₂ | 2 | NH₂ | 3 | NH₂ | 4 | NH₂ | 5 | NH₂ |
| 1 | NHR | 2 | NHR | 3 | NHR | 4 | NHR | 5 | NHR |
| 1 | COH | 2 | COH | 3 | COH | 4 | COH | 5 | COH |
| 1 | COR | 2 | COR | 3 | COR | 4 | COR | 5 | COR |

R = alkyl, alkenyl, alkynyl, aryl, or heterocycle

TABLE 7

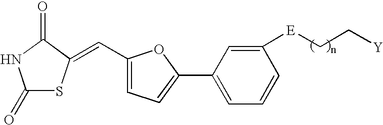

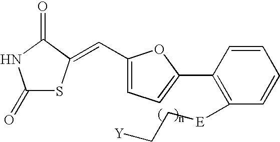

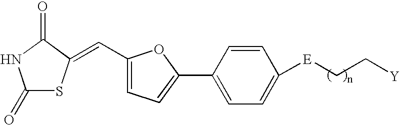

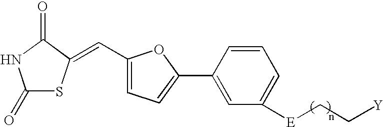

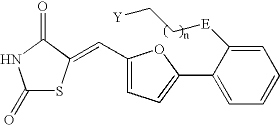

| n | E | Y | n | E | Y | n | E | Y | n | E | Y |
|---|---|---|---|---|---|---|---|---|---|---|---|
| 0 | O | OH | 0 | S | OH | 0 | NH | OH | 0 | NR | OH |
| 0 | O | SH | 0 | S | SH | 0 | NH | SH | 0 | NR | SH |
| 0 | O | COOH | 0 | S | COOH | 0 | NH | COOH | 0 | NR | COOH |
| 0 | O | SO₂H | 0 | S | SO₂H | 0 | NH | SO₂H | 0 | NR | SO₂H |
| 0 | O | Cl | 0 | S | Cl | 0 | NH | Cl | 0 | NR | Cl |
| 0 | O | Br | 0 | S | Br | 0 | NH | Br | 0 | NR | Br |
| 0 | O | I | 0 | S | I | 0 | NH | I | 0 | NR | I |
| 0 | O | F | 0 | S | F | 0 | NH | F | 0 | NR | F |
| 0 | O | CN | 0 | S | CN | 0 | NH | CN | 0 | NR | CN |
| 0 | O | N₃ | 0 | S | N₃ | 0 | NH | N₃ | 0 | NR | N₃ |
| 0 | O | CONH₂ | 0 | S | CONH₂ | 0 | NH | CONH₂ | 0 | NR | CONH₂ |
| 0 | O | CH═CH₂ | 0 | S | CH═CH₂ | 0 | NH | CH═CH₂ | 0 | NR | CH═CH₂ |
| 0 | O | C≡CH | 0 | S | CH ≡CH | 0 | NH | C≡CH | 0 | NR | C≡CH |
| 0 | O | NH₂ | 0 | S | NH₂ | 0 | NH | NH₂ | 0 | NR | NH₂ |
| 0 | O | NHR | 0 | S | NHR | 0 | NH | NHR | 0 | NR | NHR |
| 0 | O | COH | 0 | S | COH | 0 | NH | COH | 0 | NR | COH |
| 0 | O | COR | 0 | S | COR | 0 | NH | COR | 0 | NR | COR |
| 0 | CH₂ | OH | 0 | COR₁R₂ | OH | 0 | CONH | OH | 0 | CONR | OH |
| 0 | CH₂ | SH | 0 | COR₁R₂ | SH | 0 | CONH | SH | 0 | CONR | SH |
| 0 | CH₂ | COOH | 0 | COR₁R₂ | COOH | 0 | CONH | COOH | 0 | CONR | COOH |
| 0 | CH₂ | SO₂H | 0 | COR₁R₂ | SO₂H | 0 | CONH | SO₂H | 0 | CONR | SO₂H |
| 0 | CH₂ | Cl | 0 | COR₁R₂ | Cl | 0 | CONH | Cl | 0 | CONR | Cl |
| 0 | CH₂ | Br | 0 | COR₁R₂ | Br | 0 | CONH | Br | 0 | CONR | Br |
| 0 | CH₂ | I | 0 | COR₁R₂ | I | 0 | CONH | I | 0 | CONR | I |
| 0 | CH₂ | F | 0 | COR₁R₂ | F | 0 | CONH | F | 0 | CONR | F |
| 0 | CH₂ | CN | 0 | COR₁R₂ | CN | 0 | CONH | CN | 0 | CONR | CN |
| 0 | CH₂ | N₃ | 0 | COR₁R₂ | N₃ | 0 | CONH | N₃ | 0 | CONR | N₃ |
| 0 | CH₂ | CONH₂ | 0 | COR₁R₂ | CONH₂ | 0 | CONH | CONH₂ | 0 | CONR | CONH₂ |
| 0 | CH₂ | CH═CH₂ | 0 | COR₁R₂ | CH═CH₂ | 0 | CONH | CH═CH₂ | 0 | CONR | CH═CH₂ |
| 0 | CH₂ | C≡CH | 0 | COR₁R₂ | C≡CH | 0 | CONH | C≡CH | 0 | CONR | C≡CH |
| 0 | CH₂ | NH₂ | 0 | COR₁R₂ | NH₂ | 0 | CONH | NH₂ | 0 | CONR | NH₂ |
| 0 | CH₂ | NHR | 0 | COR₁R₂ | NHR | 0 | CONH | NHR | 0 | CONR | NHR |
| 0 | CH₂ | COH | 0 | COR₁R₂ | COH | 0 | CONH | COH | 0 | CONR | COH |
| 0 | CH₂ | COR | 0 | COR₁R₂ | COR | 0 | CONH | COR | 0 | CONR | COR |
| 0 | SO₂NH | OH | 0 | SO₂NR | OH | 0 | NHCONH | OH | 0 | NRCONR | OH |
| 0 | SO₂NH | SH | 0 | SO₂NR | SH | 0 | NHCONH | SH | 0 | NRCONR | SH |
| 0 | SO₂NH | COOH | 0 | SO₂NR | COOH | 0 | NHCONH | COOH | 0 | NRCONR | COOH |
| 0 | SO₂NH | SO₂H | 0 | SO₂NR | SO₂H | 0 | NRCONR | SO₂H | 0 | NRCONR | SO₂H |
| 0 | SO₂NH | Cl | 0 | SO₂NR | Cl | 0 | NHCONH | Cl | 0 | NRCONR | Cl |
| 0 | SO₂NH | Br | 0 | SO₂NR | Br | 0 | NHCONH | Br | 0 | NRCONR | Br |
| 0 | SO₂NH | I | 0 | SO₂NR | I | 0 | NHCONH | I | 0 | NRCONR | I |
| 0 | SO₂NH | F | 0 | SO₂NR | F | 0 | NHCONH | F | 0 | NRCONR | F |
| 0 | SO₂NH | CN | 0 | SO₂NR | CN | 0 | NHCONH | CN | 0 | NRCONR | CN |
| 0 | SO₂NH | N₃ | 0 | SO₂NR | N₃ | 0 | NHCONH | N₃ | 0 | NRCONR | N₃ |
| 0 | SO₂NH | CONH₂ | 0 | SO₂NR | CONH₂ | 0 | NHCONH | CONH₂ | 0 | NRCONR | CONH₂ |
| 0 | SO₂NH | CH═CH₂ | 0 | SO₂NR | CH═CH₂ | 0 | NHCONH | CH═CH₂ | 0 | NRCONR | CH═CH₂ |
| 0 | SO₂NH | C≡CH | 0 | SO₂NR | C≡CH | 0 | NHCONH | C≡CH | 0 | NRCONR | C≡CH |
| 0 | SO₂NH | NH₂ | 0 | SO₂NR | NH₂ | 0 | NHCONH | NH₂ | 0 | NRCONR | NH₂ |
| 0 | SO₂NH | NHR | 0 | SO₂NR | NHR | 0 | NHCONH | NHR | 0 | NRCONR | NHR |
| 0 | SO₂NH | COH | 0 | SO₂NR | COH | 0 | NHCONH | COH | 0 | NRCONR | COH |
| 0 | SO₂NH | COR | 0 | SO₂NR | COR | 0 | NHCONH | COR | 0 | NRCONR | COR |
| 0 | NHCNHNH | OH | 0 | NRCNHNR | OH | 0 | NHCOO | OH | 0 | NRCOO | OH |
| 0 | NHCNHNH | SH | 0 | NRCNHNR | SH | 0 | NHCOO | SH | 0 | NRCOO | SH |
| 0 | NHCNHNH | COOH | 0 | NRCNHNR | COOH | 0 | NRCOO | COOH | 0 | NRCOO | COOH |
| 0 | NHCNHNH | SO₂H | 0 | NRCNHNR | SO₂H | 0 | NRCOO | SO₂H | 0 | NRCOO | SO₂H |
| 0 | NHCNHNH | Cl | 0 | NRCNHNR | Cl | 0 | NRCOO | Cl | 0 | NRCOO | Cl |
| 0 | NHCNHNH | Br | 0 | NRCNHNR | Br | 0 | NRCOO | Br | 0 | NRCOO | Br |
| 0 | NHCNHNH | I | 0 | NRCNHHR | I | 0 | NRCOO | I | 0 | NRCOO | I |
| 0 | NHCNHNH | F | 0 | NRCNHNR | F | 0 | NHCOO | F | 0 | NRCOO | F |
| 0 | NHCNHNH | CN | 0 | NRCNHNR | CN | 0 | NHCOO | CN | 0 | NRCOO | CN |
| 0 | NHCNHNH | N₃ | 0 | NRCNHNR | N₃ | 0 | NHCOO | N₃ | 0 | NRCOO | N₃ |
| 0 | NHCNHNH | CONH₂ | 0 | NRCNHNR | CONH₂ | 0 | NHCOO | CONH₂ | 0 | NRCOO | CONH₂ |
| 0 | NHCNHNH | CH═CH₂ | 0 | NRCNHNR | CH═CH₂ | 0 | NHCOO | CH═CN | 0 | NRCOO | CH═CH₂ |
| 0 | NHCNHNH | CC≡H | 0 | NRCNHNR | C≡CH | 0 | NHCOO | C≡CH | 0 | NRCOO | C≡Cu |
| 0 | NHCNHNH | NH₂ | 0 | NRCNHNR | NH₂ | 0 | NHCOO | NE₂ | 0 | NRCOO | NE₂ |
| 0 | NHCNHNH | NHR | 0 | NRCNHNR | NHR | 0 | NHCOO | NHR | 0 | NRCOO | NHR |
| 0 | NHCNHNH | COH | 0 | NRCNHNR | COH | 0 | NHCOO | COH | 0 | NRCOO | COH |
| 0 | NHCNHNH | COR | 0 | NRCNHNR | COR | 0 | NHCOO | COR | 0 | NRCOO | COR |
| 0 | C≡C | OH | 0 | CH₂═CH₂ | OH | 1 | O | OH | 1 | S | OH |
| 0 | C≡C | SH | 0 | CH₂═CH₂ | SH | 1 | O | SH | 1 | S | SH |
| 0 | C≡C | COOH | 0 | CH₂═CH₂ | COOH | 1 | O | COOH | 1 | S | COON |
| 0 | C≡C | SO₂H | 0 | CH₂═CH₂ | SO₂H | 1 | O | SO₂H | 1 | S | SO₂H |
| 0 | C≡C | Cl | 0 | CH₂═CH₂ | Cl | 1 | O | Cl | 1 | S | Cl |
| 0 | C≡C | Br | 0 | CH₂═CH₂ | Br | 1 | O | Br | 1 | S | Br |
| 0 | C≡C | I | 0 | CH₂═CH₂ | I | 1 | O | I | 1 | S | I |
| 0 | C≡C | F | 0 | CH₂═CH₂ | F | 1 | O | F | 1 | S | F |
| 0 | C≡C | CN | 0 | CH₂═CH₂ | CN | 1 | O | CN | 1 | S | CN |
| 0 | C≡C | N₃ | 0 | CH₂═CH₂ | N₃ | 1 | O | N₃ | 1 | S | N₃ |
| 0 | C≡C | CONH₂ | 0 | CH₂═CH₂ | CONH₂ | 1 | O | CONH₂ | 1 | S | CONH₂ |
| 0 | C≡C | CH═CH₂ | 0 | CH₂═CH₂ | CH═CH₂ | 1 | O | CH═CH₂ | 1 | S | CH═CH₂ |
| 0 | C≡C | C≡CH | 0 | CH₂═CH₂ | C≡CH | 1 | O | C≡CH | 1 | S | C≡CH |
| 0 | C≡C | NH₂ | 0 | CH₂═CH₂ | NH₂ | 1 | O | NH₂ | 1 | S | NH₂ |
| 0 | C≡C | NHR | 0 | CN₂═CH₂ | NHR | 1 | O | NHR | 1 | S | NHR |
| 0 | C≡C | COH | 0 | CN₂═CH₂ | COH | 1 | O | COH | 1 | S | COH |
| 0 | C≡C | COR | 0 | CH₂═CH₂ | COR | 1 | O | COR | 1 | S | COR |
| 1 | NH | OH | 1 | NR | OH | 1 | CH₂ | OH | 1 | COR₁R₂ | OH |
| 1 | NH | SH | 1 | NR | SH | 1 | CH₂ | SH | 1 | COR₁R₂ | SH |
| 1 | NH | COOH | 1 | NR | COOH | 1 | CH₂ | COOH | 1 | COR₁R₂ | COOH |
| 1 | NH | SO₂H | 1 | NR | SO₂H | 1 | CH₂ | SO₂H | 1 | COR₁R₂ | SO₂H |
| 1 | NH | Cl | 1 | NR | Cl | 1 | CH₂ | Cl | 1 | COR₁R₂ | Cl |
| 1 | NH | Br | 1 | NR | Br | 1 | CH₂ | Br | 1 | COR₁R₂ | Br |
| 1 | NH | I | 1 | NR | I | 1 | CH₂ | I | 1 | COR₁R₂ | I |
| 1 | NH | F | 1 | NR | F | 1 | CH₂ | F | 1 | COR₁R₂ | F |
| 1 | NH | CN | 1 | NR | CN | 1 | CH₂ | CN | 1 | COR₁R₂ | CN |
| 1 | NH | N₃ | 1 | NR | N₃ | 1 | CH₂ | N₃ | 1 | COR₁R₂ | N₃ |
| 1 | NH | CONH₂ | 1 | NR | CONH₂ | 1 | CH₂ | CONH₂ | 1 | COR₁R₂ | CONH₂ |
| 1 | NH | CH═CH₂ | 1 | NR | CH═CH₂ | 1 | CH₂ | CH═CH₂ | 1 | COR₁R₂ | CH═CH₂ |
| 1 | NH | C≡CH | 1 | NR | C≡CH | 1 | CH₂ | CH≡CH | 1 | COR₁R₂ | CH≡CH |
| 1 | NH | NH₂ | 1 | NR | NH₂ | 1 | CH₂ | NH₂ | 1 | COR₁R₂ | NH₂ |
| 1 | NH | NHR | 1 | NR | NHR | 1 | CH₂ | NHR | 1 | COR₁R₂ | NHR |
| 1 | NH | COH | 1 | NR | COH | 1 | CH₂ | COH | 1 | COR₁R₂ | COH |
| 1 | NH | COR | 1 | NR | COR | 1 | CH₂ | COR | 1 | COR₁R₂ | COR |
| 1 | CONH | OH | 1 | CONR | OH | 1 | SO₂NH | OH | 1 | SO₂NR | OH |
| 1 | CONH | SH | 1 | CONR | SH | 1 | SO₂NH | SH | 1 | SO₂NR | SH |
| 1 | CONH | COOH | 1 | CONR | COOH | 1 | SO₂NH | COOH | 1 | SO₂NR | COOH |
| 1 | CONH | SO₂H | 1 | CONR | SO₂H | 1 | SO₂NH | SO₂H | 1 | SO₂NR | SO₂H |
| 1 | CONH | Cl | 1 | CONR | Cl | 1 | SO₂NH | Cl | 1 | SO₂NR | Cl |
| 1 | CONH | Br | 1 | CONR | Br | 1 | SO₂NH | Br | 1 | SO₂NR | Br |
| 1 | CONH | I | 1 | CONR | I | 1 | SO₂NH | I | 1 | SO₂NR | I |
| 1 | CONH | F | 1 | CONR | F | 1 | SO₂NH | F | 1 | SO₂NR | F |
| 1 | CONH | CN | 1 | CONR | CN | 1 | SO₂NH | CN | 1 | SO₂NR | CN |
| 1 | CONH | N₃ | 1 | CONR | N₃ | 1 | SO₂NH | N₃ | 1 | SO₁NR | N₃ |
| 1 | CONH | CONH₂ | 1 | CONR | CONH₂ | 1 | SO₂NH | CONH₂ | 1 | SO₂NR | CONH₂ |
| 1 | CONH | CH═CH₂ | 1 | CONR | CH═CH₂ | 1 | SO₂NH | CH═CH₂ | 1 | SO₂NR | CH═CH₂ |
| 1 | CONH | C≡CH | 1 | CONR | C≡CH | 1 | SO₂NH | C≡CH | 1 | SO₂NR | C≡CH |
| 1 | CONH | NH₂ | 1 | CONR | NH₂ | 1 | SO₂NH | NH₂ | 1 | SO₂NR | NH₂ |
| 1 | CONH | NHR | 1 | CONR | NHR | 1 | SO₂NH | NHR | 1 | SO₂NR | NHR |
| 1 | CONH | COH | 1 | CONR | COH | 1 | SO₂NH | COH | 1 | SO₂NR | COH |
| 1 | CONH | COR | 1 | CONR | COR | 1 | SO₂NH | COR | 1 | SO₂NR | COR |
| 1 | NHCONH | OH | 1 | NRCONR | OH | 1 | NHCNHNH | OH | 1 | NRCNHNR | OH |
| 1 | NHCONH | SH | 1 | NRCONR | SH | 1 | NHCNHNH | SH | 1 | NRCNHNR | SH |
| 1 | NHCONH | COOH | 1 | NRCONR | COOH | 1 | NHCNHNH | COOH | 1 | NRCNHNR | COOH |
| 1 | NHCONH | SO₂H | 1 | NRCONR | SO₂H | 1 | NHCNHNH | SO₂H | 1 | NHCNHNR | SO₂H |
| 1 | NHCONH | Cl | 1 | NRCONR | Cl | 1 | NHCNHNH | Cl | 1 | NRCNHNR | Cl |
| 1 | NHCONH | Br | 1 | NRCONR | Br | 1 | NHCNHNH | Br | 1 | NRCNHNR | Br |
| 1 | NHCONH | I | 1 | NRCONR | I | 1 | NHCNHNH | I | 1 | NRCNHNR | I |
| 1 | NHCONH | F | 1 | NBCONR | F | 1 | NHCNHNH | F | 1 | NRCNHNR | F |
| 1 | NHCONH | CN | 1 | NRCONR | CN | 1 | NHCNHNH | CN | 1 | NRCNHNR | CN |
| 1 | NHCONH | N₃ | 1 | NRCONR | N₃ | 1 | NHCNHNH | N₃ | 1 | NRCNHNR | N₃ |
| 1 | NHCONH | CONH₂ | 1 | NRCONR | CONH₂ | 1 | NHCNHNH | CONH₂ | 1 | NRCNHNR | CONH₂ |
| 1 | NHCONH | CH═CH₂ | 1 | NRCONR | CH═CH₂ | 1 | NHCNHNH | CH═CH₂ | 1 | NRCNHNR | CH═CH₂ |
| 1 | NHCONH | C≡CH | 1 | NRCONR | C≡CH | 1 | NHCNHNH | C≡CH | 1 | NRCNHNR | C≡CH |
| 1 | NHCONH | NH₂ | 1 | NRCONR | NH₂ | 1 | NHCNHNH | NH₂ | 1 | NRCNHNR | NH₂ |
| 1 | NHCONH | NHR | 1 | NRCONR | NHR | 1 | NHCNHNH | NHR | 1 | NRCNHNR | NHR |
| 1 | NHCONH | COH | 1 | NRCONR | COH | 1 | NHCNHNH | COH | 1 | NRCNHNR | COH |
| 1 | NHCONH | COR | 1 | NHCONR | COR | 1 | NHCNHNH | COR | 1 | NRCNHNR | COR |
| 1 | NHCOO | OH | 1 | NRCOO | OH | 1 | C≡C | OH | 1 | CH═CH₂ | OH |
| 1 | NHCOO | SH | 1 | NRCOO | SH | 1 | C≡C | SH | 1 | CH═CH₂ | SH |
| 1 | NHCOO | COOH | 1 | NRCOO | COOH | 1 | C≡C | COOH | 1 | CH═CH₂ | COOH |
| 1 | NHCOO | SO₂H | 1 | NRCOO | SO₂H | 1 | C≡C | SO₂H | 1 | CH═CH₂ | SO₂H |
| 1 | NHCOO | Cl | 1 | NRCOO | Cl | 1 | C≡C | Cl | 1 | CH≡CH₂ | Cl |
| 1 | NHCOO | Br | 1 | NRCOO | Br | 1 | C≡C | Br | 1 | CH═CH₂ | Br |
| 1 | NHCOO | I | 1 | NRCOO | I | 1 | C≡C | I | 1 | CH═CH₂ | I |
| 1 | NHCOO | F | 1 | NRCOO | F | 1 | C≡C | F | 1 | CH═CH₂ | F |
| 1 | NHCOO | CN | 1 | NHCOO | OH | 1 | C≡C | CN | 1 | CH═CH₂ | CN |
| 1 | NHCOO | N₃ | 1 | NRCOO | N₃ | 1 | C≡C | N₃ | 1 | CH═CH₂ | N₃ |
| 1 | NHCOO | CONH₂ | 1 | NRCOO | CONH₂ | 1 | C≡C | CONH₂ | 1 | CH═CH₂ | CONH₂ |
| 1 | NHCOO | CH═CH₂ | 1 | NRCOO | CH≡CH₂ | 1 | C≡C | CH═CH₂ | 1 | CH═CH₂ | CH═CH₂ |
| 1 | NHCOO | C≡CH | 1 | NRCOO | C≡CH | 1 | C≡C | CCH | 1 | CH═CH₂ | CCH |
| 1 | NHCOO | NH₂ | 1 | NRCOO | NH₂ | 1 | C≡C | NH₂ | 1 | CH═CH₂ | NH₂ |
| 1 | NHCOO | NHR | 1 | NHCOO | NHR | 1 | C≡C | NHR | 1 | CH═CH₂ | NHR |
| 1 | NHCOO | COH | 1 | NRCOO | COH | 1 | C≡C | COH | 1 | CH═CH₂ | COH |
| 1 | NHCOO | COR | 1 | NRCOO | COR | 1 | C≡C | COR | 1 | CH═CH₂ | COR |
| 2 | O | OH | 2 | S | OH | 2 | NH | OH | 2 | NR | OH |
| 2 | O | SH | 2 | S | SH | 2 | NH | SH | 2 | NR | SH |
| 2 | O | COOH | 2 | S | COOH | 2 | NH | COON | 2 | NR | COOH |
| 2 | O | SO₂H | 2 | S | SO₂H | 2 | NH | SO₂H | 2 | NR | SO₂H |
| 2 | O | Cl | 2 | S | Cl | 2 | NH | Cl | 2 | NR | Cl |
| 2 | O | Br | 2 | S | Br | 2 | NH | Br | 2 | NR | Br |
| 2 | O | I | 2 | S | I | 2 | NH | I | 2 | NR | I |
| 2 | O | F | 2 | S | F | 2 | NH | F | 2 | NR | F |
| 2 | O | CN | 2 | S | CN | 2 | NH | CN | 2 | NR | CN |
| 2 | O | N₃ | 2 | S | N₃ | 2 | NH | N₃ | 2 | NR | N₃ |
| 2 | O | CONH₂ | 2 | S | CONH₂ | 2 | NH | CONH₂ | 2 | NR | CONH₂ |
| 2 | O | CH═CH₂ | 2 | S | CH═CH₂ | 2 | NH | CH≡CH₂ | 2 | NR | CH═CH₂ |
| 2 | O | C≡CH | 2 | S | C≡CH | 2 | NH | C≡CH | 2 | NR | C≡CH |
| 2 | O | NH₂ | 2 | S | NH₂ | 2 | NH | NH₂ | 2 | NR | NH₂ |
| 2 | O | NHR | 2 | S | NHR | 2 | NH | NHR | 2 | NR | NHR |
| 2 | O | COH | 2 | S | COH | 2 | NH | COH | 2 | NR | COH |
| 2 | O | COR | 2 | S | COR | 2 | NH | COR | 2 | NR | COR |
| 2 | CH₂ | OH | 2 | COR₁R₂ | OH | 2 | CONH | OH | 2 | CONR | OH |
| 2 | CH₂ | SH | 2 | COR₁R₂ | SH | 2 | CONH | SH | 2 | CONR | SH |
| 2 | CH₂ | COOH | 2 | COR₁R₂ | COOH | 2 | CONH | COOH | 2 | CONR | COOH |
| 2 | CH₂ | SO₂H | 2 | COR₁R₂ | SO₂H | 2 | CONH | SO₂H | 2 | CONR | SO₂H |
| 2 | CH₂ | Cl | 2 | COR₁R₂ | Cl | 2 | CONH | Cl | 2 | CONR | Cl |
| 2 | CH₂ | Br | 2 | COR₁R₂ | Br | 2 | CONH | Br | 2 | CONR | Br |
| 2 | CH₂ | I | 2 | COR₂R₂ | I | 2 | CONH | I | 2 | CONR | I |
| 2 | CH₂ | F | 2 | COR₁R₂ | F | 2 | CONH | F | 2 | CONR | F |
| 2 | CH₂ | CN | 2 | COR₁R₂ | CN | 2 | CONH | CN | 2 | CONR | CN |
| 2 | CH₂ | N₃ | 2 | COR₁R₂ | N₃ | 2 | CONH | N₃ | 2 | CONR | N₃ |
| 2 | CH₂ | CONH₂ | 2 | COR₁R₂ | CONH₂ | 2 | CONH | CONH₂ | 2 | CONR | CONH₂ |
| 2 | CH₂ | CH═CH₂ | 2 | COR₁R₂ | CH≡CH₂ | 2 | CONH | CH═CH₂ | 2 | CONR | CH═CH₂ |
| 2 | CH₂ | C≡CH | 2 | COR₂R₂ | C≡CH | 2 | CONH | C≡CH | 2 | CONR | C≡CH |
| 2 | CH₂ | NH₂ | 2 | COR₁R₂ | NH₂ | 2 | CONH | NH₂ | 2 | CONR | NH₂ |
| 2 | CH₂ | NHR | 2 | COR₂R₂ | NHR | 2 | CONH | NHR | 2 | CONR | NHR |
| 2 | CH₂ | COH | 2 | COR₁R₂ | COH | 2 | CONH | COH | 2 | CONR | COH |
| 2 | CH₂ | COR | 2 | COR₁R₂ | COR | 2 | CONH | COR | 2 | CONR | COR |
| 2 | SO₂NH | OH | 2 | SO₂NR | OH | 2 | NHCONH | OH | 2 | NRCONR | OH |
| 2 | SO₂NH | SH | 2 | SO₂NR | SH | 2 | NHCONH | SH | 2 | NRCONR | SH |
| 2 | SO₂NH | COOH | 2 | SO₂NR | COOH | 2 | NHCONH | COOH | 2 | NRCONR | COOH |
| 2 | SO₂NH | SO₂NH | 2 | SO₂NR | SO₂H | 2 | NHCONH | SO₂H | 2 | NRCONR | SO_{2B} |
| 2 | SO₂NH | Cl | 2 | SO₂NR | Cl | 2 | NHCONH | Cl | 2 | NRCONR | Cl |
| 2 | SO₂NH | Br | 2 | SO₂NR | Br | 2 | NHCONH | Br | 2 | NRCONR | Br |
| 2 | SO₂NH | I | 2 | SO₂NR | I | 2 | NHCONH | I | 2 | NRCONR | I |
| 2 | SO₂NH | F | 2 | SO₂NR | F | 2 | NHCONH | F | 2 | NRCONR | F |
| 2 | SO₂NH | CN | 2 | SO₂NR | CN | 2 | NHCONH | CN | 2 | NRCONR | CN |
| 2 | SO₂NH | N₃ | 2 | SO₂NR | N₃ | 2 | NHCONH | N₃ | 2 | NRCONR | N₃ |
| 2 | SO₂NH | CONH₂ | 2 | SO₂NR | CONH₂ | 2 | NHCONH | CONH₂ | 2 | NRCONR | CONH₂ |
| 2 | SO₂NH | CH≡CH₂ | 2 | SO₂NR | CH═CH₂ | 2 | NHCONH | CH═CH₂ | 2 | NRCONR | CH═CH₂ |
| 2 | SO₂NH | C≡CH | 2 | SO_{2 NR} | CCH | 2 | NHCONH | CCH | 2 | NRCONR | CCH |
| 2 | SO₂NH | NH₂ | 2 | SO₂NR | NH₂ | 2 | NHCONH | NH₂ | 2 | NRCONR | NH₂ |
| 2 | SO₂NH | NHR | 2 | SO_{2 NR} | NHR | 2 | NHCONH | NHR | 2 | NRCONR | NHR |
| 2 | SO₂NH | COH | 2 | SO_{2 NR} | COH | 2 | NHCONH | COH | 2 | NRCONR | COH |
| 2 | SO₂NH | COR | 2 | SO_{2 NR} | COR | 2 | NHCONH | COR | 2 | NRCONR | COR |
| 2 | NHCNHNH | OH | 2 | NRCNHNR | OH | 2 | NHCOO | OH | 2 | NRCOO | OH |
| 2 | NHCNHNH | SH | 2 | NRCNHNR | SH | 2 | NHCOO | SH | 2 | NRCOO | SH |
| 2 | NHCNHNH | COOH | 2 | NRCNHNR | COOH | 2 | NHCOO | COOH | 2 | NRCOO | COOH |
| 2 | NRCNHNH | SO₂H | 2 | NRCNHNR | SO₂H | 2 | NHCOO | SO₂H | 2 | NRCOO | SO₂H |
| 2 | NHCNHNH | Cl | 2 | NRCNHNR | Cl | 2 | NHCOO | Cl | 2 | NRCOO | Cl |
| 2 | NHCNHNH | Br | 2 | NRCNHNR | Br | 2 | NHCOO | Br | 2 | NRCOO | Br |
| 2 | NHCNHNH | I | 2 | NRCNNHR | I | 2 | NHCOO | I | 2 | NRCOO | I |
| 2 | NHCNHNH | F | 2 | NRCNNHR | F | 2 | NHCOO | F | 2 | NRCOO | F |
| 2 | NHCNHNH | CN | 2 | NRCNHNR | CN | 2 | NHCOO | CN | 2 | NRCOO | CN |
| 2 | NHCNHNH | N₃ | 2 | NRCNHNR | N₃ | 2 | NHCOO | N₃ | 2 | NRCOO | N₃ |
| 2 | NHCNHNH | CONH₂ | 2 | NRCNHNR | CONH₂ | 2 | NHCOO | CONH₂ | 2 | NRCOO | CONH₂ |
| 2 | NHCNHNH | CH═CH₂ | 2 | NRCNHNR | CH═CH₂ | 2 | NHCOO | CH≡CH₂ | 2 | NRCOO | CH═CH₂ |
| 2 | NHCNHNH | CCH | 2 | NRCNHNR | C≡CH | 2 | NHCOO | C≡CH | 2 | NRCOO | C≡CH |
| 2 | NHCNHNH | NH₂ | 2 | NRCNHNR | NH₂ | 2 | NHCOO | NH₂ | 2 | NRCOO | NH₂ |
| 2 | NHCNHNH | NHR | 2 | NRCNHNR | NHR | 2 | NHCOO | NHR | 2 | NRCOO | NHR |
| 2 | NHCNHNH | COH | 2 | NRCNHNR | COH | 2 | NHCOO | COH | 2 | NRCOO | COH |
| 2 | NHCNHNH | COR | 2 | NRCNHNR | COR | 2 | NHCOO | COR | 2 | NRCOO | COR |
| 2 | C≡C | OH | 2 | CH₂═CH₂ | OH | 3 | O | OH | 3 | S | OH |
| 2 | C≡C | SH | 2 | CH₂═CH₂ | SH | 3 | O | SH | 3 | S | SH |
| 2 | C≡C | COOH | 2 | CH₂═CH₂ | COOH | 3 | O | COOH | 3 | S | COOH |
| 2 | C≡C | SO₂H | 2 | CH₂═CH₂ | SO₂H | 3 | O | SO₂H | 3 | S | SO₂H |
| 2 | C≡C | Cl | 2 | CH₂═CH₂ | Cl | 3 | O | Cl | 3 | S | Cl |
| 2 | C≡C | Br | 2 | CH₂═CH₂ | Br | 3 | O | Br | 3 | S | Br |
| 2 | C≡C | I | 2 | CH₂═CH₂ | I | 3 | O | I | 3 | S | I |
| 2 | C≡C | F | 2 | CH₂═CH₂ | F | 3 | O | F | 3 | S | F |
| 2 | C≡C | CN | 2 | CH₂═CH₂ | CN | 3 | O | CN | 3 | S | CN |
| 2 | C≡C | N₃ | 2 | CH₂═CH₂ | N₃ | 3 | O | N₃ | 3 | S | N₃ |
| 2 | C≡C | CONH₂ | 2 | CH₂═CH₂ | CONH₂ | 3 | O | CONH₂ | 3 | S | CONH₂ |
| 2 | C≡C | CH═CH₂ | 2 | CH₂═CH₂ | CH═CH₂ | 3 | O | CH═CH₂ | 3 | S | CH═CH₂ |
| 2 | C≡C | C≡CH | 2 | CH₂═CH₂ | C≡CH | 3 | O | C≡CH | 3 | S | C≡CH |
| 2 | C≡C | NH₂ | 2 | CH₂═CH₂ | NH₂ | 3 | O | NH₂ | 3 | S | NH₂ |
| 2 | C≡C | NHR | 2 | CH₂═CH₂ | NHR | 3 | O | NHR | 3 | S | NHR |
| 2 | C≡C | COH | 2 | CH₂═CH₂ | COH | 3 | O | COH | 3 | S | COH |
| 2 | C≡C | COR | 2 | CH₂═CH₂ | COR | 3 | O | COR | 3 | S | COR |
| 3 | NH | OH | 3 | NR | OH | 3 | CH₂ | OH | 3 | COR₁R₂ | OH |
| 3 | NH | SH | 3 | NR | SH | 3 | CH₂ | SH | 3 | COR₁R₂ | SH |
| 3 | NH | COOH | 3 | NR | COOH | 3 | CH₂ | COOH | 3 | COR₁R₂ | COOH |
| 3 | NH | SO₂H | 3 | NR | SO₂H | 3 | CH₂ | SO₂H | 3 | COR₁R₂ | SO₂H |
| 3 | NH | Cl | 3 | NR | Cl | 3 | CH₂ | Cl | 3 | COR₁R₂ | Cl |
| 3 | NH | Br | 3 | NR | Br | 3 | CH₂ | Br | 3 | COR₂R₂ | Br |
| 3 | NH | I | 3 | NR | I | 3 | CH₂ | I | 3 | COR₁R₂ | I |
| 3 | NH | F | 3 | NR | F | 3 | CH₂ | F | 3 | COR₁R₂ | F |
| 3 | NH | CN | 3 | NR | CN | 3 | CH₂ | CN | 3 | COR₁R₂ | CN |
| 3 | NH | N₃ | 3 | NR | N₃ | 3 | CH₂ | N₃ | 3 | COR₁R₂ | N₃ |
| 3 | NH | CONH₂ | 3 | NR | CONH₂ | 3 | CH₂ | CONH₂ | 3 | COR₁R₂ | CONH₂ |
| 3 | NH | CH═CH₂ | 3 | NR | CH═CH₂ | 3 | CH₂ | CH═CH₂ | 3 | COR₁R₂ | CH═CH₂ |
| 3 | NH | C≡CH | 3 | NR | C≡CH | 3 | CH₂ | C≡CH | 3 | COR₁R₂ | C≡CH |
| 3 | NH | NH₂ | 3 | NR | NH₂ | 3 | CH₂ | NH₂ | 3 | COR₁R₂ | NH₂ |
| 3 | NH | NHR | 3 | NR | NHR | 3 | CH₂ | NHR | 3 | COR₁R₂ | NHR |
| 3 | NH | COH | 3 | NR | COH | 3 | CH₂ | COH | 3 | COR₁R₂ | COH |
| 3 | NH | COR | 3 | NR | COR | 3 | CH₂ | COR | 3 | COR₁R₂ | COR |
| 3 | CONH | OH | 3 | CONR | OH | 3 | SO₂NH | OH | 3 | SO₂NR | OH |
| 3 | CONH | SH | 3 | CONR | SH | 3 | SO₂NH | SH | 3 | SO₂NR | SH |
| 3 | CONH | COOH | 3 | CONR | COOH | 3 | SO₂NH | COOH | 3 | SO₂NR | COOH |
| 3 | CONH | SO₂H | 3 | CONR | SO₂H | 3 | SO₂NH | SO₂H | 3 | SO₂NR | SO₂H |
| 3 | CONH | Cl | 3 | CONR | Cl | 3 | SO₂NH | Cl | 3 | SO₂NR | Cl |
| 3 | CONH | Br | 3 | CONR | Br | 3 | SO₂NH | Br | 3 | SO₂NR | Br |
| 3 | CONH | I | 3 | CONR | I | 3 | SO₂NH | I | 3 | SO₂NR | I |
| 3 | CONH | F | 3 | CONR | F | 3 | SO₂NH | F | 3 | SO₂NR | F |
| 3 | CONH | CN | 3 | CONR | CN | 3 | SO₂NH | CN | 3 | SO₂NR | CN |
| 3 | CONH | N₃ | 3 | CONR | N₃ | 3 | SO₂NH | N₃ | 3 | SO₂NR | N₃ |
| 3 | CONH | CONH₂ | 3 | CONR | CONH₂ | 3 | SO₂NH | CONH₂ | 3 | SO₂NR | CONH₂ |
| 3 | CONH | CH≡CH₂ | 3 | CONR | CH═CH₂ | 3 | SO₂NH | CH═CH₂ | 3 | SO₂NR | CH═CH₂ |
| 3 | CONH | C≡CH | 3 | CONR | C≡CH | 3 | SO₂NH | C≡CH | 3 | SO₂NR | C≡CH |
| 3 | CONH | NH₂ | 3 | CONR | NH₂ | 3 | SO₂NH | NH₂ | 3 | SO₂NR | NH₂ |
| 3 | CONH | NHR | 3 | CONR | NHR | 3 | SO₂NH | NHR | 3 | SO₂NR | NHR |
| 3 | CONH | COH | 3 | CONR | COH | 3 | SO₂NH | COH | 3 | SO₂NR | COH |
| 3 | CONH | COR | 3 | CONR | COR | 3 | SO₂NH | COR | 3 | SO₂NR | COR |
| 3 | NHCONH | OH | 3 | NRCONR | OH | 3 | NHCNHNH | OH | 3 | NRCNHNR | OH |
| 3 | NHCONH | SH | 3 | NRCONR | SH | 3 | NHCNHNH | SH | 3 | NRCNHNR | SH |
| 3 | NHCONH | COOH | 3 | NRCONR | COOH | 3 | NHCNHNH | COOH | 3 | NRCNHNR | COOH |
| 3 | NHCONH | SO₂H | 3 | NRCONR | SO₂H | 3 | NHCNHNH | SO₂H | 3 | NRCNHNR | SO₂H |
| 3 | NHCONH | Cl | 3 | NRCONR | Cl | 3 | NHCNHNH | Cl | 3 | NRCNHNR | Cl |
| 3 | NHCONH | Br | 3 | NRCONR | Br | 3 | NHCNHNH | Br | 3 | NRCNHNR | Br |
| 3 | NHCONH | I | 3 | NRCONR | I | 3 | NHCNHNH | I | 3 | NRCNHNR | I |
| 3 | NHCONH | F | 3 | NRCONR | F | 3 | NHCNHNH | F | 3 | NRCNHNR | F |
| 3 | NHCONH | CN | 3 | NRCONR | CN | 3 | NHCNHNH | CN | 3 | NRCNHNR | CN |
| 3 | NHCONH | N₃ | 3 | NRCONR | N₃ | 3 | NHCNHNH | N₃ | 3 | NRCNHNR | N₃ |
| 3 | NHCONH | CONH₂ | 3 | NRCONR | CONH₂ | 3 | NHCNHNH | CONH₂ | 3 | NRCNHNR | CONH₂ |
| 3 | NHCONH | CH═CH₂ | 3 | NRCONR | CH═CH₂ | 3 | NHCNHNH | CH═CH₂ | 3 | NRCNHNR | CH═CH₂ |
| 3 | NHCONH | C≡CH | 3 | NRCONR | C≡C | 3 | NHCNHNH | C≡CH | 3 | NRCNHNR | C≡CH |
| 3 | NHCONH | NH₂ | 3 | NRCONR | NH₂ | 3 | NHCNHNH | NH₂ | 3 | NRCNHNR | NH₂ |
| 3 | NHCONH | NHR | 3 | NRCONR | NHR | 3 | NHCNHNH | NHR | 3 | NRCNHNR | NHR |
| 3 | NHCONH | COH | 3 | NRCONR | COH | 3 | NHCNHNH | COH | 3 | NRCNHNR | COH |
| 3 | NHCONH | COR | 3 | NRCONR | COR | 3 | NHCNHNH | COR | 3 | NRCNHNR | COR |
| 3 | NHCOO | OH | 3 | NRCOO | OH | 3 | C≡C | OH | 3 | CH₂═CH₂ | OH |
| 3 | NHCOO | SH | 3 | NRCOO | SH | 3 | C≡C | SH | 3 | CH₂═CH₂ | SH |
| 3 | NHCOO | COOH | 3 | NRCOO | COOH | 3 | C≡C | COOH | 3 | CH₂═CH₂ | COOH |
| 3 | NHCOO | SO₂H | 3 | NRCOO | SO₂H | 3 | C≡C | SO₂H | 3 | CH₂═CH₂ | SO₂H |
| 3 | NHCOO | Cl | 3 | NRCOO | Cl | 3 | C≡C | Cl | 3 | CH₂═CH₂ | Cl |
| 3 | NHCOO | Br | 3 | NRCOO | Br | 3 | C≡C | Br | 3 | CH₂═CH₂ | Br |
| 3 | NHCOO | I | 3 | NRCOO | I | 3 | C≡C | I | 3 | CH₂═CH₂ | I |
| 3 | NHCOO | F | 3 | NRCOO | F | 3 | C≡C | F | 3 | CH₂═CH₂ | F |
| 3 | NHCOO | CN | 3 | NRCOO | CN | 3 | C≡C | CN | 3 | CH₂═CH₂ | CN |
| 3 | NHCOO | N₃ | 3 | NRCOO | N₃ | 3 | C≡C | N₃ | 3 | CH₂═CH₂ | N₃ |
| 3 | NHCOO | CONH₂ | 3 | NRCOO | CONH₂ | 3 | C≡C | CONH₂ | 3 | CH₂═CH₂ | CONH₂ |
| 3 | NHCOO | CH═CH₂ | 3 | NRCOO | CH═CH₂ | 3 | C≡C | CH═CH₂ | 3 | CH₂═CH₂ | CH═CH₂ |
| 3 | NHCOO | C≡CH | 3 | NRCOO | C≡CH | 3 | C≡C | C≡CH | 3 | CH₂═CH₂ | C≡CH |
| 3 | NHCOO | NH₂ | 3 | NRCOO | NH₂ | 3 | C≡C | NH₂ | 3 | CH₂═CH₂ | NH₂ |
| 3 | NHCOO | NHR | 3 | NRCOO | NHR | 3 | C≡C | NHR | 3 | CH₂═CH₂ | NHR |
| 3 | NHCOO | COH | 3 | NRCOO | COH | 3 | C≡C | COH | 3 | CH₂═CH₂ | COH |
| 3 | NHCOO | COR | 3 | NRCOO | COR | 3 | C≡C | COR | 3 | CH₂═CH₂ | COR |
| 4 | O | OH | 4 | S | OH | 4 | NH | OH | 4 | NR | OH |
| 4 | O | SH | 4 | S | SH | 4 | NH | SH | 4 | NR | SH |
| 4 | O | COOH | 4 | S | COOH | 4 | NH | COOH | 4 | NR | COOH |
| 4 | O | SO₂H | 4 | S | SO₂H | 4 | NH | SO₂H | 4 | NR | SO₂H |
| 4 | O | Cl | 4 | S | Cl | 4 | NH | Cl | 4 | NR | Cl |
| 4 | O | Br | 4 | S | Br | 4 | NH | Br | 4 | NR | Br |
| 4 | O | I | 4 | S | I | 4 | NH | I | 4 | NR | I |
| 4 | O | F | 4 | S | F | 4 | NH | F | 4 | NR | F |
| 4 | O | CN | 4 | S | CN | 4 | NH | CN | 4 | NR | CN |
| 4 | O | N₃ | 4 | S | N₃ | 4 | NH | N₃ | 4 | NR | N₃ |
| 4 | O | CONH₂ | 4 | S | CONH₂ | 4 | NH | CONH₂ | 4 | NR | CONH₂ |
| 4 | O | CH═CH₂ | 4 | S | CH═CH₂ | 4 | NH | CH═CH₂ | 4 | NR | CH═CH₂ |
| 4 | O | C≡CH | 4 | S | C≡CH | 4 | NH | C≡CH | 4 | NR | C≡CH |
| 4 | O | NH₂ | 4 | S | NH₂ | 4 | NH | NH₂ | 4 | NR | NH₂ |
| 4 | O | NHR | 4 | S | NHR | 4 | NH | NHR | 4 | NR | NHR |
| 4 | O | COH | 4 | S | COH | 4 | NH | COH | 4 | NR | COH |
| 4 | O | COR | 4 | S | COR | 4 | NH | COR | 4 | NR | COR |
| 4 | CH₂ | OH | 4 | COR₁R₂ | OH | 4 | CONH | OH | 4 | CONR | OH |
| 4 | CH₂ | SH | 4 | COR₁R₂ | SH | 4 | CONH | SH | 4 | CONR | SH |
| 4 | CH₂ | COOH | 4 | COR₁R₂ | COOH | 4 | CONH | COOH | 4 | CONR | COOH |
| 4 | CH₂ | SO₂H | 4 | COR₁R₂ | SO₂H | 4 | CONH | SO₂H | 4 | CONR | SO₂H |
| 4 | CH₂ | Cl | 4 | COR₁R₂ | Cl | 4 | CONH | Cl | 4 | CONR | Cl |
| 4 | CH₂ | Br | 4 | COR₁R₂ | Br | 4 | CONH | Br | 4 | CONR | Br |
| 4 | CH₂ | I | 4 | COR₁R₂ | I | 4 | CONH | I | 4 | CONR | I |
| 4 | CH₂ | F | 4 | COR₁R₂ | F | 4 | CONH | F | 4 | CONR | F |
| 4 | CH₂ | CN | 4 | COR₁R₂ | CN | 4 | CONH | CN | 4 | CONR | CN |
| 4 | CH₂ | N₃ | 4 | COR₁R₂ | N₃ | 4 | CONH | N₃ | 4 | CONR | N₃ |
| 4 | CH₂ | CONH₂ | 4 | COR₁R₂ | CONH₂ | 4 | CONH | CONH₂ | 4 | CONR | CONH₂ |
| 4 | CH₂ | CH═CH₂ | 4 | COR₁R₂ | CH═CH₂ | 4 | CONH | CH≡CH₂ | 4 | CONR | CH═CH₂ |
| 4 | CH₂ | C≡CH | 4 | COR₁R₂ | C≡CH | 4 | CONH | C≡CH | 4 | CONR | C≡CH |
| 4 | CH₂ | NH₂ | 4 | COR₁R₂ | NH₂ | 4 | CONH | NH₂ | 4 | CONR | NH₂ |
| 4 | CH₂ | NHR | 4 | COR₁R₂ | NHR | 4 | CONH | NHR | 4 | CONR | NHR |
| 4 | CH₂ | COH | 4 | COR₁R₂ | COH | 4 | CONH | COH | 4 | CONR | COH |
| 4 | CH₂ | COR | 4 | COR₁R₂ | COR | 4 | CONH | COR | 4 | CONR | COR |
| 4 | SO₂NH | OH | 4 | SO₂NR | OH | 4 | NHCONH | OH | 4 | NRCONR | OH |
| 4 | SO₂NH | SH | 4 | SO₂NR | SH | 4 | NHCONH | SH | 4 | NRCONR | SH |
| 4 | SO₂NH | COOH | 4 | SO₂NR | COOH | 4 | NHCONH | COOH | 4 | NRCONR | COOH |
| 4 | SO₂NH | SO₂H | 4 | SO₂NR | SO₂H | 4 | NHCONH | SO₂H | 4 | NRCONR | SO₂H |
| 4 | SO₂NH | Cl | 4 | SO₂NR | Cl | 4 | NHCONH | Cl | 4 | NRCONR | Cl |
| 4 | SO₂NH | Br | 4 | SO₂NR | Br | 4 | NHCONH | Br | 4 | NRCONR | Br |
| 4 | SO₂NH | I | 4 | SO₂NR | I | 4 | NHCONH | I | 4 | NRCONR | I |
| 4 | SO₂NH | F | 4 | SO₂NR | F | 4 | NHCONH | F | 4 | NRCONR | F |
| 4 | SO₂NH | CN | 4 | SO₂NR | CN | 4 | NHCONH | CN | 4 | NRCONR | CN |
| 4 | SO₂NH | N₃ | 4 | SO₂NR | N₃ | 4 | NHCONH | N₃ | 4 | NRCONR | N₃ |
| 4 | SO₂NH | CONH₂ | 4 | SO₂NR | CONH₂ | 4 | NHCONH | CONH₂ | 4 | NRCONR | CONH₂ |
| 4 | SO₂NH | CH═CH₂ | 4 | SO₂NR | CH═CH₂ | 4 | NHCONH | CH═CH₂ | 4 | NRCONR | CH═CH₂ |
| 4 | SO₂NH | C≡CH | 4 | SO₂NR | C≡CH | 4 | NHCONH | C≡CH | 4 | NRCONR | C≡CH |
| 4 | SO₂NH | NH₂ | 4 | SO₂NR | NH₂ | 4 | NHCONH | NH₂ | 4 | NRCONR | NH₂ |
| 4 | SO₂NH | NHR | 4 | SO₂NR | NHR | 4 | NHCONH | NHR | 4 | NRCONR | NHR |
| 4 | SO₂NH | COH | 4 | SO₂NR | COH | 4 | NHCONH | COH | 4 | NRCONR | COH |
| 4 | SO₂NH | COR | 4 | SO₂NR | COR | 4 | NHCONH | COR | 4 | NRCONR | COR |
| 4 | NHCNHNH | OH | 4 | NRCNHNR | OH | 4 | NHCOO | OH | 4 | NRCOO | OH |
| 4 | NHCNHNH | SH | 4 | NRCNHNR | SH | 4 | NHCOO | SH | 4 | NRCOO | SH |
| 4 | NHCNHNH | COOH | 4 | NRCNHNR | COOH | 4 | NHCOO | COOH | 4 | NRCOO | COOH |
| 4 | NHCNHNH | SO₂H | 4 | NRCNHNR | SO₂H | 4 | NHCOO | SO₂H | 4 | NRCOO | SO₂H |
| 4 | NHCNHNH | Cl | 4 | NRCNHNR | Cl | 4 | NHCOO | Cl | 4 | NRCOO | Cl |
| 4 | NHCNHNH | Br | 4 | NRCNHNR | Br | 4 | NHCOO | Br | 4 | NRCOO | Br |
| 4 | NHCNHNH | I | 4 | NRCNHNR | I | 4 | NHCOO | I | 4 | NRCOO | I |
| 4 | NHCNHNH | F | 4 | NRCNHNR | F | 4 | NHCOO | F | 4 | NRCOO | F |
| 4 | NHCNHNH | CN | 4 | NRCNHNR | CN | 4 | NHCOO | CN | 4 | NRCOO | CN |
| 4 | NHCNHNH | N₃ | 4 | NRCNHNR | N₃ | 4 | NHCOO | N₃ | 4 | NRCOO | N₃ |
| 4 | NHCNHNH | CONH₂ | 4 | NRCNHNR | CONH₂ | 4 | NHCOO | CONH₂ | 4 | NRCOO | CONH₂ |
| 4 | NHCNHNH | CH═CH₂ | 4 | NRCNHNR | CH═CH₂ | 4 | NHCOO | CH═CH₂ | 4 | NRCOO | CH═CH₂ |
| 4 | NHCNHNH | C≡CH | 4 | NRCNHNR | C≡CH | 4 | NHCOO | C≡CH | 4 | NRCOO | C≡CH |
| 4 | NHCNHNH | NH₂ | 4 | NRCNHNR | NH₂ | 4 | NHCOO | NH₂ | 4 | NRCOO | NH₂ |
| 4 | NHCNHNH | NHR | 4 | NRCNHNR | NHR | 4 | NHCOO | NBR | 4 | NRCOO | NBR |
| 4 | NHCNHNH | COH | 4 | NRCNHNR | COH | 4 | NHCOO | COH | 4 | NRCOO | COH |
| 4 | NHCNHNH | COR | 4 | NRCNHNR | COR | 4 | NHCOO | COR | 4 | NRCOO | COR |
| 4 | C≡C | OH | 4 | CH₂═CH₂ | OH | 5 | O | OH | 5 | S | OH |
| 4 | C≡C | SH | 4 | CH₂═CH₂ | SH | 5 | O | SH | 5 | S | SH |
| 4 | C≡C | COOH | 4 | CH₂═CH₂ | COOH | 5 | O | COOH | 5 | S | COOH |
| 4 | C≡C | SO₂H | 4 | CH₂═CH₂ | SO₂H | 5 | O | SO₂H | 5 | S | SO₂H |
| 4 | C≡C | Cl | 4 | CH₂═CH₂ | Cl | 5 | O | Cl | 5 | S | Cl |
| 4 | C≡C | Br | 4 | CH₂═CH₂ | Br | 5 | O | Br | 5 | S | Br |
| 4 | C≡C | I | 4 | CH₂═CH₂ | I | 5 | O | I | 5 | S | I |
| 4 | C≡C | F | 4 | CH₂═CH₂ | F | 5 | O | F | 5 | S | F |
| 4 | C≡C | CN | 4 | CH₂═CH₂ | CN | 5 | O | CN | 5 | S | CN |
| 4 | C≡C | N₃ | 4 | CH₂═CH₂ | N₃ | 5 | O | N₃ | 5 | S | N₃ |
| 4 | C≡C | CONH₂ | 4 | CH₂═CH₂ | CONH₂ | 5 | O | CONH₂ | 5 | S | CONH₂ |
| 4 | C≡C | CH═CH₂ | 4 | CH₂═CH₂ | CH═CH₂ | 5 | O | CH═CH₂ | 5 | S | CH═CH₂ |
| 4 | C≡C | C≡CH | 4 | CH₂═CH₂ | C≡CH | 5 | O | C≡CH | 5 | S | C≡CH |
| 4 | C≡C | NH₂ | 4 | CH₂═CH₂ | NH₂ | 5 | O | NH₂ | 5 | S | NH₂ |
| 4 | C≡C | NHR | 4 | CH₂═CH₂ | NHR | 5 | O | NHR | 5 | S | NHR |
| 4 | C≡C | COH | 4 | CH₂═CH₂ | COH | 5 | O | COH | 5 | S | COH |
| 4 | C≡C | COR | 4 | CH₂═CH₂ | COR | 5 | O | COR | 5 | S | COR |
| 5 | NH | OH | 5 | NR | OH | 5 | CH₂ | OH | 5 | COR₁R₂ | OH |
| 5 | NH | SH | 5 | NR | SH | 5 | CH₂ | SH | 5 | COR₁R₂ | SH |
| 5 | NH | COOH | 5 | NR | COOH | 5 | CH₂ | COOH | 5 | COR₁R₂ | COOH |
| 5 | NH | SO₂H | 5 | NR | SO₂H | 5 | CH₂ | SO₂H | 5 | COR₁R₂ | SO₂H |
| 5 | NH | Cl | 5 | NR | Cl | 5 | CH₂ | Cl | 5 | COR₁R₂ | Cl |
| 5 | NH | Br | 5 | NR | Br | 5 | CH₂ | Br | 5 | COR₁R₂ | Br |
| 5 | NH | I | 5 | NR | I | 5 | CH₂ | I | 5 | COR₁R₂ | I |
| 5 | NH | F | 5 | NR | F | 5 | CH₂ | F | 5 | COR₁R₂ | F |
| 5 | NH | CN | 5 | NR | CN | 5 | CH₂ | CN | 5 | COR₁R₂ | CN |
| 5 | NH | N₃ | 5 | NR | N₃ | 5 | CH₂ | N₃ | 5 | COR₁R₂ | N₃ |
| 5 | NH | CONH₂ | 5 | NR | CONH₂ | 5 | CH₂ | CONH₂ | 5 | COR₁R₂ | CONH₂ |
| 5 | NH | CH═CH₂ | 5 | NR | CH═CH₂ | 5 | CH₂ | CH═CH₂ | 5 | COR₁R₂ | CH═CH₂ |
| 5 | NH | C≡CH | 5 | NR | C≡CH | 5 | CH₂ | C≡CH | 5 | COR₁R₂ | C≡CH |
| 5 | NH | NH₂ | 5 | NR | NH₂ | 5 | CH₂ | NH₂ | 5 | COR₁R₂ | NH₂ |
| 5 | NH | NHR | 5 | NR | NHR | 5 | CH₂ | NHR | 5 | COR₁R₂ | NHR |
| 5 | NH | COH | 5 | NR | COH | 5 | CH₂ | COH | 5 | COR₁R₂ | COH |
| 5 | NH | COR | 5 | NR | COR | 5 | CH₂ | COR | 5 | COR₁R₂ | COR |
| 5 | CONH | OH | 5 | CONR | OH | 5 | SO₂NH | OH | 5 | SO₂NR | OH |
| 5 | CONH | SH | 5 | CONR | SH | 5 | SO₂NH | SH | 5 | SO₂NR | SH |
| 5 | CONH | COOH | 5 | CONR | COOH | 5 | SO₂NH | COOH | 5 | SO₂NR | COOH |
| 5 | CONH | SO₂H | 5 | CONR | SO₂H | 5 | SO₂NH | SO₂H | 5 | SO₂NR | SO₂H |
| 5 | CONH | Cl | 5 | CONR | Cl | 5 | SO₂NH | Cl | 5 | SO₂NR | Cl |
| 5 | CONH | Br | 5 | CONR | Br | 5 | SO₂NH | Br | 5 | SO₂NR | Br |
| 5 | CONH | I | 5 | CONR | I | 5 | SO₂NH | I | 5 | SO₂NR | I |
| 5 | CONH | F | 5 | CONR | F | 5 | SO₂NH | F | 5 | SO₂NR | F |
| 5 | CONH | CN | 5 | CONR | CN | 5 | SO₂NH | CN | 5 | SO₂NR | CN |
| 5 | CONH | N₃ | 5 | CONR | N₃ | 5 | SO₂NH | N₃ | 5 | SO₂NR | N₃ |
| 5 | CONH | CONH₂ | 5 | CONR | CONH₂ | 5 | SO₂NH | CONH₂ | 5 | SO₂NR | CONH₂ |
| 5 | CONH | CH═CH₂ | 5 | CONR | CH═CH₂ | 5 | SO₂NH | CH═CH₂ | 5 | SO₂NR | CB═CH₂ |
| 5 | CONH | C≡CH | 5 | CONR | C≡CH | 5 | SO₂NH | C≡CH | 5 | SO₂NR | C≡CB |
| 5 | CONH | NH₂ | 5 | CONR | NH₂ | 5 | SO₂NH | NH₂ | 5 | SO₂NR | NH₂ |
| 5 | CONH | NHR | 5 | CONR | NHR | 5 | SO₂NH | NHR | 5 | SO₂NR | NHR |
| 5 | CONH | COH | 5 | CONR | COH | 5 | SO₂NH | COH | 5 | SO₂NR | COH |
| 5 | CONH | COR | 5 | CONR | COR | 5 | SO₂NH | COH | 5 | SO₂NR | COH |
| 5 | NHCONH | OH | 5 | NHCONR | OH | 5 | NHCNHNH | OH | 5 | NRCNHNR | OH |
| 5 | NHCONH | SH | 5 | NRCONR | SH | 5 | NHCNHNH | SH | 5 | NRCNHNR | SH |
| 5 | NHCONH | COOH | 5 | NRCONR | COOH | 5 | NHCNHNH | COOH | 5 | NRCNHNR | COOH |
| 5 | NHCONH | SO₂H | 5 | NRCONR | SO₂H | 5 | NHCNHNH | SO₂H | 5 | NRCNHNR | SO₂H |
| 5 | NHCONH | Cl | 5 | NRCONR | Cl | 5 | NHCNHNH | Cl | 5 | NRCNHNR | Cl |
| 5 | NHCONH | Br | 5 | NRCONR | Br | 5 | NHCNHNH | Br | 5 | NRCNHNR | Br |
| 5 | NHCONH | I | 5 | NRCONR | I | 5 | NHCNHNH | I | 5 | NRCNHNR | I |
| 5 | NHCONH | F | 5 | NRCONR | F | 5 | NHCNHNH | F | 5 | NRCNHNR | F |
| 5 | NHCONH | CN | 5 | NRCONR | CN | 5 | NHCNHNH | CN | 5 | NRCNHNR | CN |
| 5 | NHCONH | N₃ | 5 | NRCONR | N₃ | 5 | NHCNHNH | N₃ | 5 | NRCNHNR | N₃ |
| 5 | NHCONH | CONH₂ | 5 | NRCONR | CONH₂ | 5 | NHCNHNH | CONH₂ | 5 | NRCNHNR | CONH₂ |
| 5 | NHCONH | CB═CH3 | 5 | NRCONR | CH═CH₂ | 5 | NHCNHNH | CH═CH₂ | 5 | NRCNHNR | CH═CH₂ |
| 5 | NHCONH | C≡Cu | 5 | NRCONR | C≡Cu | 5 | NHCNHNH | C≡CH | 5 | NRCNHNR | C≡CH |
| 5 | NHCONH | NH₂ | 5 | NRCONR | NH₂ | 5 | NHCNHNH | NH₂ | 5 | NRCNHNR | NH₂ |
| 5 | NHCONH | NHR | 5 | NRCONR | NHR | 5 | NHCNHNH | NHR | 5 | NRCNBNR | NHR |
| 5 | NHCONH | COH | 5 | NRCONR | COH | 5 | NHCNHNH | COH | 5 | NRCNHNR | COH |
| 5 | NHCONH | COR | 5 | NRCONR | COR | 5 | NHCNHNH | COR | 5 | NRCNHNR | COR |
| 5 | NRCNHNR | OH | 5 | NHCOO | OH | 5 | NRCOO | OH | 5 | C≡C | OH |
| 5 | NRCNHNR | SH | 5 | NHCOO | SH | 5 | NRCOO | SH | 5 | C≡C | SH |
| 5 | NRCNHNR | COOH | 5 | NHCOO | COOH | 5 | NRCOO | COOH | 5 | C≡C | COOH |
| 5 | NRCNHNR | SO₂H | 5 | NHCOO | SO₂H | 5 | NRCOO | SO₂H | 5 | C≡C | SO₂H |
| 5 | NRCNHNR | Cl | 5 | NHCOO | Cl | 5 | NRCOO | Cl | 5 | C≡C | Cl |
| 5 | NRCNHNR | Br | 5 | NHCOO | Br | 5 | NRCOO | Br | 5 | C≡C | Br |
| 5 | NRCNHNR | I | 5 | NHCOO | I | 5 | NRCOO | I | 5 | C≡C | I |
| 5 | NRCNHNR | F | 5 | NHCOO | F | 5 | NRCOO | F | 5 | C≡C | F |
| 5 | NRCNHNR | CN | 5 | NHCOO | CN | 5 | NRCOO | CN | 5 | C≡C | CN |
| 5 | NRCNHNR | N₃ | 5 | NHCOO | N₃ | 5 | NRCOO | N₃ | 5 | C≡C | N₃ |
| 5 | NRCNHNR | CONH₂ | 5 | NHCOO | CONH₂ | 5 | NRCOO | CONH₃ | 5 | C≡C | CONH₂ |
| 5 | NRCNHNR | CH═CH₂ | 5 | NHCOO | CH═CH₂ | 5 | NRCOO | CH═CH₂ | 5 | C≡C | CH≡CH₂ |
| 5 | NRCNHNR | C≡CH | 5 | NHCOO | C≡CH | 5 | NRCOO | C≡CH | 5 | C≡C | C≡CH |
| 5 | NRCNHNR | NH₂ | 5 | NHCOO | NH₂ | 5 | NRCOO | NH₂ | 5 | C≡C | NH₂ |
| 5 | NRCNHNR | NHR | 5 | NHCOO | NHR | 5 | NRCOO | NHR | 5 | C≡C | NHR |
| 5 | NRCNHNR | COH | 5 | NHCOO | COH | 5 | NRCOO | COH | 5 | C≡C | COH |
| 5 | NRCNHNR | COR | 5 | NHCOO | COR | 5 | NRCOO | COR | 5 | C≡C | COR |
| 5 | CH₂═CH₂ | OH | 5 | CH₂═CH₂ | Br | 5 | CH₂═CH₂ | N₃ | 5 | CH₂═CH₂ | NH₂ |
| 5 | CH₂═CH₂ | SH | 5 | CH₂═CH₂ | I | 5 | CH₂═CH₂ | CONH₂ | 5 | CH₂═CH₂ | NHR |
| 5 | CH₂═CH₂ | COOH | 5 | CH₂═CH₂ | F | 5 | CH₂═CH₂ | CH═CH₂ | 5 | CH₂═CH₂ | COH |
| 5 | CH₂═CH₂ | SO₂H | 5 | CH₂═CH₂ | CN | 5 | CH₂═CH₂ | C≡CH | 5 | CH₂═CH₂ | COR |

| | | | |
|---|---|---|---|
| 5 | CH₂═CH₂ | Cl | R, R₁, and R₂ =H, alkyl, alkenyl, alkynyl, aryl, and |
| | | | heterocycle |

TABLE 8

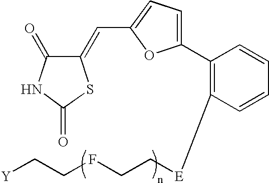

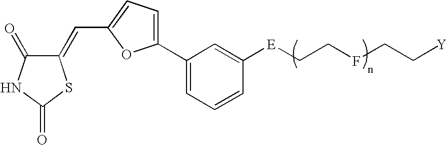

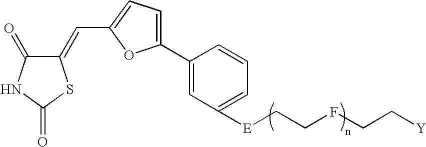

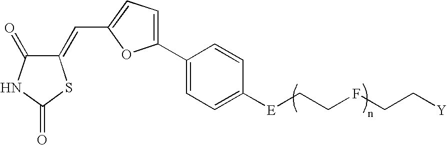

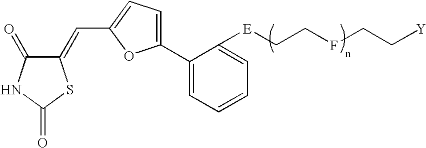

| n | E | F | Y | n | E | F | Y |
|---|---|---|---|---|---|---|---|
| 0 | O | O | OH | 0 | O | S | OH |
| 0 | O | O | NH₂ | 0 | O | S | NH₂ |
| 0 | O | CONR | I | 0 | O | SO₂NR | I |
| 0 | O | NRCONR | COH | 0 | O | NRCNHNR | COH |
| 0 | O | NRCONR | COR | 0 | O | NRCNHNR | COR |
| 0 | O | NRCOO | CH═CH₂ | 0 | O | C≡C | CH═CH₂ |
| 0 | O | CH═CH | NHR | 0 | S | O | NHR |
| 0 | O | CH═CH | COH | 0 | S | O | COH |
| 0 | S | S | NHR | 0 | S | NR | NHR |
| 0 | S | S | COH | 0 | S | NR | COH |
| 0 | S | S | COR | 0 | S | NR | COR |
| 0 | S | CR₁R₂ | COH | 0 | S | CONR | COH |
| 0 | S | CR₁R₂ | COR | 0 | S | CONR | COR |
| 0 | S | SO₂NR | OH | 0 | S | NRCONR | OH |
| 0 | S | SO₂NR | SO₂H | 0 | S | NRCONR | SO₂H |
| 0 | S | NRCNHNR | CONH₂ | 0 | S | NRCOO | CONH₂ |
| 0 | S | NRCNHNR | CH═CH₂ | 0 | S | NRCOO | CH═CH₂ |
| 0 | NR | O | C≡CH | 0 | NR | S | C≡CH |
| 0 | NR | CONR | Cl | 0 | NR | SO₂NR | Cl |
| 0 | NR | CONR | COR | 0 | NR | SO₂NR | COR |
| 0 | NR | NRCONR | OH | 0 | NR | NRCNHNR | OH |
| 0 | NR | NRCONR | SH | 0 | NR | NRCNHNR | SH |
| 0 | NR | NRCONR | CONH₂ | 0 | NR | NRCNHNR | CONH₂ |
| 0 | NR | NRCOO | COR | 0 | NR | | COR |
| 0 | NR | CH═CH | OH | 0 | CR₁R₂ | O | OH |
| 0 | NR | CH═CH | N₃ | 0 | CR₁R₂ | O | N₃ |
| 0 | NR | CH═CH | CONH₂ | 0 | CR₁R₂ | O | CONH₂ |
| 0 | NR | CH═CH | CH═CH₂ | 0 | CR₁R₂ | O | CH═CH₂ |
| 0 | CR₁R₂ | S | COH | 0 | CR₁R₂ | NR | COH |
| 0 | CR₁R₂ | S | COR | 0 | CR₁R₂ | NR | COR |
| 0 | CR₁R₂ | CR₁R₂ | SH | 0 | CR₁R₂ | CONR | SH |
| 0 | CR₁R₂ | CR₁R₂ | COOH | 0 | CR₁R₂ | CONR | COOH |
| 0 | CR₁R₂ | CR₁R₂ | NH₂ | 0 | CR₁R₂ | CONR | NH₂ |
| 0 | CR₁R₂ | SO₂NR | Cl | 0 | CR₁R₂ | NRCONR | Cl |
| 0 | CR₁R₂ | SO₂NR | CN | 0 | CR₁R₂ | NRCONR | CN |
| 0 | CR₁R₂ | SO₂NR | N₃ | 0 | CR₁R₂ | NRCONR | N₃ |
| 0 | CR₁R₂ | NRCNHNR | NHR | 0 | CR₁R₂ | NRCOO | NHR |
| 0 | CR₁R₂ | NRCNHNR | COR | 0 | CR₁R₂ | NRCOO | COR |
| 0 | CR₁R₂ | C≡C | OH | 0 | CR₁R₂ | CH═CH | OH |
| 0 | CR₁R₂ | C≡C | Br | 0 | CR₁R₂ | CH═CH | Br |
| 0 | CONR | O | OH | 0 | CONR | S | OH |
| 0 | CONR | O | SH | 0 | CONR | S | SH |
| 0 | CONR | O | COR | 0 | CONR | S | COR |
| 0 | CONR | NR | OH | 0 | CONR | CR₁R₂ | OH |
| 0 | CONR | NR | COR | 0 | CONR | CR₁R₂ | COR |
| 0 | CONR | CONR | OH | 0 | CONR | SO₂NR | OH |
| 0 | CONR | CONR | SH | 0 | CONR | SO₂NR | SH |
| 0 | CONR | CONR | COOH | 0 | CONR | SO₂NR | COOH |
| 0 | CONR | NRCOO | Br | 0 | CONR | C≡C | Br |
| 0 | CONR | NRCOO | CONH₂ | 0 | CONR | C≡C | CONH₂ |
| 0 | CONR | CH═CH | CONH₂ | 0 | SO₂NR | O | CONH₂ |
| 0 | CONR | CH═CH | CH═CH₂ | 0 | SO₂NR | O | CH═CH₂ |
| 0 | CONR | CH═CH | NH₂ | 0 | SO₂NR | O | NH₂ |
| 0 | SO₂NR | S | SH | 0 | SO₂NR | NR | SH |
| 0 | SO₂NR | S | COOH | 0 | SO₂NR | NR | COOH |
| 0 | SO₂NR | S | F | 0 | SO₂NR | NR | F |
| 0 | SO₂NR | CR₁R₂ | CONH₂ | 0 | SO₂NR | CONR | CONH₂ |
| 0 | SO₂NR | SO₂NR | F | 0 | SO₂NR | NRCONR | F |
| 0 | SO₂NR | SO₂NR | N₃ | 0 | SO₂NR | NRCONR | N₃ |
| 0 | SO₂NR | SO₂NR | CH═CH₂ | 0 | SO₂NR | NRCONR | CH═CH₂ |
| 0 | SO₂NR | NRCNHNR | SH | 0 | SO₂NR | NRCOO | SH |
| 0 | SO₂NR | NRCNHNR | SO₂H | 0 | SO₂NR | NRCOO | SO₂H |
| 0 | SO₂NR | NRCNHNR | Cl | 0 | SO₂NR | NRCOO | Cl |
| 0 | SO₂NR | C≡C | NHR | 0 | SO₂NR | CH═CH | NHR |
| 0 | SO₂NR | C≡C | COR | 0 | SO₂NR | CH═CH | COR |
| 0 | NRCONR | O | OH | 0 | NRCONR | S | OH |
| 0 | NRCONR | O | SH | 0 | NRCONR | S | SH |
| 0 | NRCONR | O | COOH | 0 | NRCONR | S | COOH |
| 0 | NRCONR | NR | SO₂H | 0 | NRCONR | CR₁R₂ | SO₂H |
| 0 | NRCONR | NR | COH | 0 | NRCONR | CR₁R₂ | COH |
| 0 | NRCONR | NR | COR | 0 | NRCONR | CR₁R₂ | COR |
| 0 | NRCONR | CONR | F | 0 | NRCONR | SO₂NR | F |
| 0 | NRCONR | CONR | CH═CH₂ | 0 | NRCONR | SO₂NR | CH═CH₂ |
| 0 | NRCONR | CONR | C≡CH | 0 | NRCONR | SO₂NR | C≡CH |
| 0 | NRCONR | NRCONR | COR | 0 | NRCONR | NRCNHNR | COR |
| 0 | NRCONR | NRCOO | OH | 0 | NRCONR | C≡C | OH |
| 0 | NRCONR | NRCOO | COH | 0 | NRCONR | C≡C | COH |
| 0 | NRCONR | NRCOO | COR | 0 | NRCONR | | COR |
| 0 | NRCONR | CH═CH | OH | 0 | NRCNHNR | O | OH |
| 0 | NRCONR | CH═CH | SH | 0 | NRCNHNR | O | SH |
| 0 | NRCONR | CH═CH | COOH | 0 | NRCNHNR | O | COOH |
| 0 | NRCNHNR | S | C≡CH | 0 | NRCNHNR | NR | C≡CH |
| 0 | NRCNHNR | S | NH₂ | 0 | NRCNHNR | NR | NH₂ |
| 0 | NRCNHNR | S | NHR | 0 | NRCNHNR | NR | NHR |
| 0 | NRCNHNR | CR₁R₂ | Br | 0 | NRCNHNR | CONR | Br |
| 0 | NRCNHNR | CR₁R₂ | NH₂ | 0 | NRCNHNR | CONR | NH₂ |
| 0 | NRCNHNR | CR₁R₂ | NHR | 0 | NRCNHNR | CONR | NHR |
| 0 | NRCNHNR | SO₂NR | SH | 0 | NRCNHNR | NRCONR | SH |
| 0 | NRCNHNR | SO₂NR | COOH | 0 | NRCNHNR | NRCONR | COOH |
| 0 | NRCNHNR | NRCNHNR | CN | 0 | NRCNHNR | NRCOO | CN |
| 0 | NRCNHNR | NRCNHNR | N₃ | 0 | NRCNHNR | NRCOO | N₃ |
| 0 | NRCNHNR | NRCNHNR | CONH₂ | 0 | NRCNHNR | NRCOO | CONH₂ |
| 0 | NRCNHNR | C≡C | SH | 0 | NRCNHNR | CH═CH | SH |
| 0 | NRCNHNR | C≡C | COOH | 0 | NRCNHNR | CH═CH | COOH |
| 0 | NRCOO | O | CN | 0 | NRCOO | S | CN |
| 0 | NRCOO | O | N₃ | 0 | NRCOO | S | N₃ |
| 0 | NRCOO | O | CONH₂ | 0 | NRCOO | S | CONH₂ |
| 0 | NRCOO | CONR | CN | 0 | NRCOO | SO₂NR | CN |
| 0 | NRCOO | CONR | N₃ | 0 | NRCOO | SO₂NR | N₃ |
| 0 | NRCOO | NRCONR | COH | 0 | NRCOO | NRCNHNR | COH |
| 0 | NRCOO | NRCONR | COR | 0 | NRCOO | NRCNHNR | COR |
| 0 | NRCOO | NRCOO | OH | 0 | NRCOO | C≡C | OH |
| 0 | NRCOO | NRCOO | SH | 0 | NRCOO | C≡C | SH |
| 0 | NRCOO | CH═CH | F | 0 | C≡C | O | F |
| 0 | C≡C | S | COOH | 0 | C≡C | NR | COOH |
| 0 | C≡C | S | SO₂H | 0 | C≡C | NR | SO₂H |
| 0 | C≡C | CR₁R₂ | NH₂ | 0 | C≡C | CONR | NH₂ |
| 0 | C≡C | CR₁R₂ | NHR | 0 | C≡C | CONR | NHR |
| 0 | C≡C | CR₁R₂ | COH | 0 | C≡C | CONR | COH |
| 0 | C≡C | SO₂NR | COH | 0 | C≡C | NRCONR | COH |
| 0 | C≡C | SO₂NR | COR | 0 | C≡C | NRCONR | COR |
| 0 | C≡C | NRCNHNR | OH | 0 | C≡C | NRCOO | OH |
| 0 | C≡C | NRCNHNR | SO₂H | 0 | C≡C | NRCOO | SO₂H |
| 0 | C≡C | NRCNHNR | Cl | 0 | C≡C | NRCOO | Cl |
| 0 | C≡C | C≡C | OH | 0 | C≡C | CH═CH | OH |
| 0 | C≡C | C≡C | CN | 0 | C≡C | CH═CH | CN |
| 0 | CH═CH | O | CH═CH₂ | 0 | CH═CH | S | CH═CH₂ |
| 0 | CH═CH | O | C≡CH | 0 | CH═CH | S | C≡CH |
| 0 | CH═CH | O | COR | 0 | CH═CH | S | COR |
| 0 | CH═CH | NR | OH | 0 | CH═CH | CR₁R₂ | OH |
| 0 | CH═CH | NR | SH | 0 | CH═CH | CR₁R₂ | SH |
| 0 | CH═CH | NRCONR | COH | 0 | CH═CH | NRCNHNR | COH |
| 0 | CH═CH | NRCONR | COR | 0 | CH═CH | NRCNHNR | COR |
| 0 | CH═CH | NRCOO | SH | 0 | CH═CH | C≡C | SH |
| 0 | CH═CH | NRCOO | NHR | 0 | CH═CH | C≡C | NHR |
| 0 | CH═CH | NRCOO | COH | 0 | CH═CH | C≡C | COH |
| 0 | CH═CH | CH═CH | OH | 0 | CH═CH | CH═CH | N₃ |
| 0 | CH═CH | CH═CH | SH | 0 | CH═CH | CH═CH | CONH₂ |
| 1 | O | O | C≡CH | 1 | O | S | C≡CH |
| 1 | O | O | NH₂ | 1 | O | S | NH₂ |
| 1 | O | O | NHR | 1 | O | S | NHR |
| 1 | O | NR | NHR | 1 | O | CR₁R₂ | NHR |
| 1 | O | NR | COH | 1 | O | CR₁R₂ | COH |
| 1 | O | CONR | SH | 1 | O | SO₂NR | SH |
| 1 | O | CONR | SO₂H | 1 | O | SO₂NR | SO₂H |
| 1 | O | NRCONR | OH | 1 | O | NRCNHNR | OH |
| 1 | O | NRCONR | SH | 1 | O | NRCNHNR | SH |
| 1 | O | NRCOO | SH | 1 | O | C≡C | SH |
| 1 | O | NRCOO | COOH | 1 | O | C≡C | COOH |
| 1 | O | CH═CH | OH | 1 | S | O | OH |
| 1 | O | CH═CH | COH | 1 | S | O | COH |
| 1 | O | CH═CH | COR | 1 | S | O | COR |
| 1 | S | S | OH | 1 | S | NR | OH |
| 1 | S | S | CH═CH₂ | 1 | S | NR | CH═CH₂ |
| 1 | S | S | NH₂ | 1 | S | NR | NH₂ |
| 1 | S | CR₁R₂ | Cl | 1 | S | CONR | Cl |
| 1 | S | CR₁R₂ | Br | 1 | S | CONR | Br |
| 1 | S | SO₂NR | Br | 1 | S | NRCONR | Br |
| 1 | S | SO₂NR | COH | 1 | S | NRCONR | COH |
| 1 | S | NRCNHNR | COOH | 1 | S | NRCOO | COOH |
| 1 | S | NRCNHNR | F | 1 | S | NRCOO | F |
| 1 | S | C≡C | OH | 1 | S | CH═CH | OH |
| 1 | S | C≡C | SH | 1 | S | CH═CH | SH |
| 1 | S | C≡C | COOH | 1 | S | CH═CH | COOH |
| 1 | S | C≡C | C≡CH | 1 | S | CH═CH | C≡CH |
| 1 | NR | O | SO₂H | 1 | NR | S | SO₂H |
| 1 | NR | O | Cl | 1 | NR | S | Cl |
| 1 | NR | O | CN | 1 | NR | S | CN |
| 1 | NR | NR | CONH₂ | 1 | NR | CR₁R₂ | CONH₂ |
| 1 | NR | NR | CH═CH₂ | 1 | NR | CR₁R₂ | CH═CH₂ |
| 1 | NR | CONR | CONH₂ | 1 | NR | SO₂NR | CONH₂ |
| 1 | NR | CONR | COR | 1 | NR | SO₂NR | COR |
| 1 | NR | NRCONR | NHR | 1 | NR | NRCNHNR | NHR |
| 1 | NR | NRCONR | COH | 1 | NR | NRCNHNR | COH |
| 1 | NR | NRCOO | OH | 1 | NR | C≡C | OH |
| 1 | NR | NRCOO | N₃ | 1 | NR | C≡C | N₃ |
| 1 | NR | NRCOO | CONH₂ | 1 | NR | C≡C | CONH₂ |
| 1 | NR | CH═CH | N₃ | 1 | CR₁R₂ | O | N₃ |
| 1 | NR | CH═CH | CONH₂ | 1 | CR₁R₂ | O | CONH₂ |
| 1 | NR | CH═CH | CH═CH₂ | 1 | CR₁R₂ | O | CH═CH₂ |
| 1 | CR₁R₂ | S | Br | 1 | CR₁R₂ | NR | Br |
| 1 | CR₁R₂ | S | N₃ | 1 | CR₁R₂ | NR | N₃ |
| 1 | CR₁R₂ | S | NHR | 1 | CR₁R₂ | NR | NHR |
| 1 | CR₁R₂ | S | COH | 1 | CR₁R₂ | NR | COH |
| 1 | CR₁R₂ | CR₁R₂ | SO₂H | 1 | CR₁R₂ | CONR | SO₂H |
| 1 | CR₁R₂ | SO₂NR | COOH | 1 | CR₁R₂ | NRCONR | COOH |
| 1 | CR₁R₂ | SO₂NR | SO₂H | 1 | CR₁R₂ | NRCONR | SO₂H |
| 1 | CR₁R₂ | NRCNHNR | CN | 1 | CR₁R₂ | NRCOO | CN |
| 1 | CR₁R₂ | NRCNHNR | COH | 1 | CR₁R₂ | NRCOO | COH |
| 1 | CR₁R₂ | NRCNHNR | COR | 1 | CR₁R₂ | NRCOO | COR |
| 1 | CR₁R₂ | C≡C | SH | 1 | CR₁R₂ | CH═CH | SH |
| 1 | CR₁R₂ | C≡C | COOH | 1 | CR₁R₂ | CH═CH | COOH |
| 1 | CONR | O | OH | 1 | CONR | S | OH |
| 1 | CONR | O | SH | 1 | CONR | S | SH |
| 1 | CONR | O | COOH | 1 | CONR | S | COOH |
| 1 | CONR | NR | CN | 1 | CONR | CR₁R₂ | CN |
| 1 | CONR | NR | N₃ | 1 | CONR | CR₁R₂ | N₃ |
| 1 | CONR | NR | COH | 1 | CONR | CR₁R₂ | COH |
| 1 | CONR | NR | COR | 1 | CONR | CR₁R₂ | COR |
| 1 | CONR | CONR | OH | 1 | CONR | SO₂NR | OH |
| 1 | CONR | CONR | F | 1 | CONR | SO₂NR | F |
| 1 | CONR | CONR | NHR | 1 | CONR | SO₂NR | NHR |
| 1 | CONR | CONR | COR | 1 | CONR | SO₂NR | COR |
| 1 | CONR | NRCONR | OH | 1 | CONR | NRCNHNR | OH |
| 1 | CONR | NRCONR | SO₂H | 1 | CONR | NRCNHNR | SO₂H |
| 1 | CONR | NRCOO | SH | 1 | CONR | C≡C | SH |
| 1 | CONR | NRCOO | COOH | 1 | CONR | C≡C | COOH |
| 1 | CONR | NRCOO | COH | 1 | CONR | C≡C | COH |
| 1 | CONR | CH═CH | Cl | 1 | SO₂NR | O | Cl |
| 1 | CONR | CH═CH | Br | 1 | SO₂NR | O | Br |
| 1 | SO₂NR | S | N₃ | 1 | SO₂NR | NR | N₃ |
| 1 | SO₂NR | S | CONH₂ | 1 | SO₂NR | NR | CONH₂ |
| 1 | SO₂NR | S | COR | 1 | SO₂NR | NR | COR |
| 1 | SO₂NR | CR₁R₂ | SH | 1 | SO₂NR | CONR | SH |
| 1 | SO₂NR | CR₁R₂ | COOH | 1 | SO₂NR | CONR | COOH |
| 1 | SO₂NR | SO₂NR | SO₂H | 1 | SO₂NR | NRCONR | SO₂H |
| 1 | SO₂NR | SO₂NR | Cl | 1 | SO₂NR | NRCONR | Cl |
| 1 | SO₂NR | SO₂NR | Br | 1 | SO₂NR | NRCONR | Br |
| 1 | SO₂NR | SO₂NR | COH | 1 | SO₂NR | NRCONR | COH |
| 1 | SO₂NR | NRCNHNR | OH | 1 | SO₂NR | NRCOO | OH |
| 1 | SO₂NR | NRCNHNR | NH₂ | 1 | SO₂NR | NRCOO | NH₂ |
| 1 | SO₂NR | C≡C | Br | 1 | SO₂NR | CH═CH | Br |
| 1 | SO₂NR | C≡C | COR | 1 | SO₂NR | CH═CH | COR |
| 1 | NRCONR | O | SH | 1 | NRCONR | S | SH |
| 1 | NRCONR | O | NH₂ | 1 | NRCONR | S | NH₂ |
| 1 | NRCONR | NR | Cl | 1 | NRCONR | CR₁R₂ | Cl |
| 1 | NRCONR | NR | I | 1 | NRCONR | CR₁R₂ | I |
| 1 | NRCONR | CONR | F | 1 | NRCONR | SO₂NR | F |
| 1 | NRCONR | CONR | N₃ | 1 | NRCONR | SO₂NR | N₃ |
| 1 | NRCONR | NRCONR | OH | 1 | NRCONR | NRCNHNR | OH |
| 1 | NRCONR | NRCONR | COR | 1 | NRCONR | NRCNHNR | COR |
| 1 | NRCONR | NRCOO | OH | 1 | NRCONR | C≡C | OH |
| 1 | NRCONR | NRCOO | COR | 1 | NRCONR | | COR |
| 1 | NRCONR | CH═CH | OH | 1 | NRCNHNR | O | OH |
| 1 | NRCONR | CH═CH | COOH | 1 | NRCNHNR | O | COOH |
| 1 | NRCNHNR | S | NH₂ | 1 | NRCNHNR | NR | NH₂ |
| 1 | NRCNHNR | S | NHR | 1 | NRCNHNR | NR | NHR |
| 1 | NRCNHNR | S | COH | 1 | NRCNHNR | NR | COH |
| 1 | NRCNHNR | CR₁R₂ | F | 1 | NRCNHNR | CONR | F |
| 1 | NRCNHNR | CR₁R₂ | CN | 1 | NRCNHNR | CONR | CN |
| 1 | NRCNHNR | SO₂NR | CN | 1 | NRCNHNR | NRCONR | CN |
| 1 | NRCNHNR | SO₂NR | NHR | 1 | NRCNHNR | NRCONR | NHR |
| 1 | NRCNHNR | SO₂NR | COH | 1 | NRCNHNR | NRCONR | COH |
| 1 | NRCNHNR | NRCNHNR | Cl | 1 | NRCNHNR | NRCOO | Cl |
| 1 | NRCNHNR | NRCNHNR | Br | 1 | NRCNHNR | NRCOO | Br |
| 1 | NRCNHNR | NRCNHNR | CH═CH₂ | 1 | NRCNHNR | NRCOO | CH═CH₂ |
| 1 | NRCNHNR | C≡C | OH | 1 | NRCNHNR | CH═CH | OH |
| 1 | NRCNHNR | C≡C | SO₂H | 1 | NRCNHNR | CH═CH | SO₂H |
| 1 | NRCNHNR | C≡C | COR | 1 | NRCNHNR | CH═CH | COR |
| 1 | NRCOO | O | F | 1 | NRCOO | S | F |
| 1 | NRCOO | O | N₃ | 1 | NRCOO | S | N₃ |
| 1 | NRCOO | O | CONH₂ | 1 | NRCOO | S | CONH₂ |
| 1 | NRCOO | NR | OH | 1 | NRCOO | CR₁R₂ | OH |
| 1 | NRCOO | NR | SH | 1 | NRCOO | CR₁R₂ | SH |
| 1 | NRCOO | NR | I | 1 | NRCOO | CR₁R₂ | I |
| 1 | NRCOO | CONR | OH | 1 | NRCOO | SO₂NR | OH |
| 1 | NRCOO | CONR | N₃ | 1 | NRCOO | SO₂NR | N₃ |
| 1 | NRCOO | CONR | COR | 1 | NRCOO | SO₂NR | COR |
| 1 | NRCOO | NRCONR | OH | 1 | NRCOO | NRCNHNR | OH |
| 1 | NRCOO | NRCONR | N₃ | 1 | NRCOO | NRCNHNR | N₃ |
| 1 | NRCOO | NRCOO | SH | 1 | NRCOO | C≡C | SH |
| 1 | NRCOO | NRCOO | CH═CH₂ | 1 | NRCOO | C≡C | CH═CH₂ |
| 1 | NRCOO | CH═CH | I | 1 | C≡C | O | I |
| 1 | NRCOO | CH═CH | F | 1 | C≡C | O | F |
| 1 | NRCOO | CH═CH | C≡CH | 1 | C≡C | O | C≡CH |
| 1 | C≡C | S | I | 1 | C≡C | NR | I |
| 1 | C≡C | S | F | 1 | C≡C | NR | F |
| 1 | C≡C | S | CH═CH₂ | 1 | C≡C | NR | CH═CH₂ |
| 1 | C≡C | CR₁R₂ | OH | 1 | C≡C | CONR | OH |
| 1 | C≡C | CR₁R₂ | SH | 1 | C≡C | CONR | SH |
| 1 | C≡C | CR₁R₂ | COOH | 1 | C≡C | CONR | COOH |
| 1 | C≡C | CR₁R₂ | SO₂H | 1 | C≡C | CONR | SO₂H |
| 1 | C≡C | SO₂NR | NHR | 1 | C≡C | NRCONR | NHR |
| 1 | C≡C | NRCNHNR | SH | 1 | C≡C | NRCOO | SH |
| 1 | C≡C | NRCNHNR | SO₂H | 1 | C≡C | NRCOO | SO₂H |
| 1 | C≡C | NRCNHNR | COR | 1 | C≡C | NRCOO | COR |
| 1 | C≡C | C≡C | OH | 1 | C≡C | CH═CH | OH |
| 1 | C≡C | C≡C | COH | 1 | C≡C | CH═CH | COH |
| 1 | C≡C | C≡C | COR | 1 | C≡C | CH═CH | COR |
| 1 | CH═CH | O | OH | 1 | CH═CH | S | OH |
| 1 | CH═CH | O | COOH | 1 | CH═CH | S | COOH |
| 1 | CH═CH | O | COH | 1 | CH═CH | S | COH |
| 1 | CH═CH | NR | SO₂H | 1 | CH═CH | CR₁R₂ | SO₂H |
| 1 | CH═CH | NR | F | 1 | CH═CH | CR₁R₂ | F |
| 1 | CH═CH | NR | COH | 1 | CH═CH | CR₁R₂ | COH |
| 1 | CH═CH | CONR | SH | 1 | CH═CH | SO₂NR | SH |
| 1 | CH═CH | CONR | I | 1 | CH═CH | SO₂NR | I |
| 1 | CH═CH | CONR | F | 1 | CH═CH | SO₂NR | F |
| 1 | CH═CH | NRCONR | CH═CH_{2 1} | CH═CH | NRCNHNR | CH═CH₂ | |
| 1 | CH═CH | NRCONR | C≡CH | 1 | CH═CH | NRCNHNR | C≡CH |
| 1 | CH═CH | NRCONR | NH₂ | 1 | CH═CH | NRCNHNR | NH₂ |
| 1 | CH═CH | NRCOO | COH | 1 | CH═CH | C≡C | COH |
| 1 | CH═CH | NRCOO | COR | 1 | CH═CH | C≡C | COR |
| 1 | CH═CH | CH═CH | OH | 1 | CH═CH | CH═CH | N₃ |
| 1 | CH═CH | CH═CH | Br | 1 | CH═CH | CH═CH | NHR |
| 1 | CH═CH | CH═CH | I | 1 | CH═CH | CH═CH | COH |
| 2 | O | O | F | 2 | O | S | F |
| 2 | O | O | CN | 2 | O | S | CN |
| 2 | O | O | N₃ | 2 | O | S | N₃ |
| 2 | O | NR | Br | 2 | O | CR₁R₂ | Br |
| 2 | O | NR | F | 2 | O | CR₁R₂ | F |
| 2 | O | NR | COR | 2 | O | CR₁R₂ | COR |
| 2 | O | CONR | OH | 2 | O | SO₂NR | OH |
| 2 | O | CONR | SH | 2 | O | SO₂NR | SH |
| 2 | O | CONR | COOH | 2 | O | SO₂NR | COOH |
| 2 | O | NRCONR | N₃ | 2 | O | NRCNHNR | N₃ |
| 2 | O | NRCONR | CONH₂ | 2 | O | NRCNHNR | CONH₂ |
| 2 | O | NRCOO | Cl | 2 | O | C≡C | Cl |
| 2 | O | NRCOO | CH═CH₂ | 2 | O | C≡C | CH═CH₂ |
| 2 | O | CH═CH | SH | 2 | S | O | SH |
| 2 | O | CH═CH | COOH | 2 | S | O | COOH |
| 2 | O | CH═CH | COH | 2 | S | O | COH |
| 2 | S | S | COOH | 2 | S | NR | COOH |
| 2 | S | S | SO₂H | 2 | S | NR | SO₂H |
| 2 | S | S | Cl | 2 | S | NR | Cl |
| 2 | S | S | NHR | 2 | S | NR | NHR |
| 2 | S | CR₂R₂ | CN | 2 | S | CONR | CN |
| 2 | S | CR₂R₂ | C≡CH | 2 | S | CONR | C≡CH |
| 2 | S | CR₂R₂ | NH₂ | 2 | S | CONR | NH₂ |
| 2 | S | SO₂NR | Cl | 2 | S | NRCONR | Cl |
| 2 | S | SO₂NR | Br | 2 | S | NRCONR | Br |
| 2 | S | SO₂NR | N₃ | 2 | S | NRCONR | N₃ |
| 2 | S | NRCNHNR | Br | 2 | S | NRCOO | Br |
| 2 | S | NRCNHNR | I | 2 | S | NRCOO | I |
| 2 | S | NRCNHNR | COR | 2 | S | NRCOO | COR |
| 2 | S | C≡C | OH | 2 | S | CH═CH | OH |
| 2 | S | C≡C | SH | 2 | S | CH═CH | SH |
| 2 | S | C≡C | CH═CH₂ | 2 | S | CH═CH | CH═CH₂ |
| 2 | NR | O | C≡CH | 2 | NR | S | C≡CH |
| 2 | NR | O | NH₂ | 2 | NR | S | NH₂ |
| 2 | NR | O | NHR | 2 | NR | S | NHR |
| 2 | NR | NR | Br | 2 | NR | CR₂R₂ | Br |
| 2 | NR | NR | F | 2 | NR | CR₂R₂ | F |
| 2 | NR | NR | NH₂ | 2 | NR | CR₂R₂ | NH₂ |
| 2 | NR | NR | NHR | 2 | NR | CR₂R₂ | NHR |
| 2 | NR | CONR | CN | 2 | NR | SO₂NR | CN |
| 2 | NR | CONR | COR | 2 | NR | SO₂NR | COR |
| 2 | NR | NRCONR | OH | 2 | NR | NRCNHNR | OH |
| 2 | NR | NRCONR | SH | 2 | NR | NRCNHNR | SH |
| 2 | NR | NRCOO | CH═CH₂ | 2 | NR | C≡C | CH═CH₂ |
| 2 | NR | NRCOO | C≡CH | 2 | NR | C≡C | C≡CH |
| 2 | NR | NRCOO | NH₂ | 2 | NR | C≡C | NH₂ |
| 2 | NR | CH═CH | Br | 2 | CR₂R₂ | O | Br |
| 2 | NR | CH═CH | NH₂ | 2 | CR₂R₂ | OO | NH₂ |
| 2 | NR | CH═CH | COH | 2 | CR₂R₂ | O | COH |
| 2 | NR | CH═CH | COR | 2 | CR₂R₂ | O | COR |
| 2 | CR₂R₂ | S | OH | 2 | CR₂R₂ | NR | OH |
| 2 | CR₂R₂ | S | SH | 2 | CR₂R₂ | NR | SH |
| 2 | CR₂R₂ | S | NH₂ | 2 | CR₂R₂ | NR | NH₂ |
| 2 | CR₂R₂ | CR₂R₂ | CN | 2 | CR₂R₂ | CONR | CN |
| 2 | CR₂R₂ | CR₂R₂ | N₃ | 2 | CR₂R₂ | CONR | N₃ |
| 2 | CR₂R₂ | CR₂R₂ | CONH₂ | 2 | CR₂R₂ | CONR | CONH₂ |
| 2 | CR₂R₂ | CR₂R₂ | CH═CH₂ | 2 | CR₂R₂ | CONR | CH═CH₂ |
| 2 | CR₂R₂ | SO₂NR | OH | 2 | CR₂R₂ | NRCONR | OH |
| 2 | CR₂R₂ | SO₂NR | Br | 2 | CR₂R₂ | NRCONR | Br |
| 2 | CR₂R₂ | SO₂NR | I | 2 | CR₂R₂ | NHCONR | I |
| 2 | CR₂R₂ | SO₂NR | F | 2 | CR₂R₂ | NRCONR | F |
| 2 | CR₂R₂ | NRCNHNR | SH | 2 | CR₂R₂ | NRCOO | SH |
| 2 | CR₂R₂ | NRCNHNR | COOH | 2 | CR₂R₂ | NRCOO | COOH |
| 2 | CR₂R₂ | NRCNHNR | SO₂H | 2 | CR₂R₂ | NRCOO | SO₂H |
| 2 | CR₂R₂ | C≡C | Cl | 2 | CR₂R₂ | CH═CH | Cl |
| 2 | CR₂R₂ | C≡C | NH₂ | 2 | CR₂R₂ | CH═CH | NH₂ |
| 2 | CR₂R₂ | C≡C | COH | 2 | CR₂R₂ | CH═CH | COH |
| 2 | CONR | O | SO₂H | 2 | CONR | S | SO₂H |
| 2 | CONR | O | N₃ | 2 | CONR | S | N₃ |
| 2 | CONR | NR | COOH | 2 | CONR | CR₂R₂ | COOH |
| 2 | CONR | NR | SO₂H | 2 | CONR | CR₂R₂ | SO₂H |
| 2 | CONR | NR | Cl | 2 | CONR | CR₂R₂ | Cl |
| 2 | CONR | CONR | CH═CH₂ | 2 | CONR | SO₂NR | CH═CH₂ |
| 2 | CONR | CONR | C≡CH | 2 | CONR | SO₂NR | C≡CH |
| 2 | CONR | CONR | NH₂ | 2 | CONR | SO₂NR | NH₂ |
| 2 | CONR | NRCONR | NH₂ | 2 | CONR | NRCNHNR | HR₂ |
| 2 | CONR | NRCONR | NHR | 2 | CONR | NRCNHNR | NHR |
| 2 | CONR | NRCOO | CN | 2 | CONR | C≡C | CN |
| 2 | CONR | NRCOO | COR | 2 | CONR | C≡C | COR |
| 2 | CONR | CH═CH | OH | 2 | SO₂NR | O | OH |
| 2 | CONR | CH═CH | Br | 2 | SO₂NR | O | Br |
| 2 | CONR | CH═CH | I | 2 | SO₂NR | O | I |
| 2 | SO₂NR | S | OH | 2 | SO₂NR | NR | OH |
| 2 | SO₂NR | S | SH | 2 | SO₂NR | NR | SH |
| 2 | SO₂NR | S | COH | 2 | SO₂NR | NR | COH |
| 2 | SO₂NR | CR₂R₂ | COOH | 2 | SO₂NR | CONR | COOH |
| 2 | SO₂NR | CR₂R₂ | COR | 2 | SO₂NR | CONR | COR |
| 2 | SO₂NR | SO₂NR | OH | 2 | SO₂NR | NRCONR | OH |
| 2 | SO₂NR | SO₂NR | SH | 2 | SO₂NR | NRCONR | SH |
| 2 | SO₂NR | SO₂NR | COOH | 2 | SO₂NR | NRCONR | COOH |
| 2 | SO₂NR | NRCNHNR | CH═CH₂ | 2 | SO₂NR | NRCOO | CH═CH₂ |
| 2 | SO₂NR | NRCNHNR | COH | 2 | SO₂NR | NRCOO | COH |
| 2 | SO₂NR | NRCNHNR | COR | 2 | SO₂NR | NRCOO | COR |
| 2 | SO₂NR | C≡C | NHR | 2 | SO₂NR | CH═CH | NHR |
| 2 | SO₂NR | C≡C | COH | 2 | SO₂NR | CH═CH | COH |
| 2 | NRCONR | O | COOH | 2 | NRCOHR | S | COOH |
| 2 | NRCONR | O | CONH₂ | 2 | NRCONR | S | CONH₂ |
| 2 | NRCONR | O | CH═CH₂ | 2 | NRCONR | S | CH═CH₂ |
| 2 | NRCONR | NR | Cl | 2 | NRCONR | CR₂R₂ | Cl |
| 2 | NRCONR | NR | Br | 2 | NRCONR | CR₂R₂ | Br |
| 2 | NRCONR | CONR | COH | 2 | NRCONR | SO₂NR | COH |
| 2 | NRCONR | CONR | COR | 2 | NRCONR | SO₂NR | COR |
| 2 | NRCONR | NRCONR | SH | 2 | NRCONR | NRCNHNR | SH |
| 2 | NRCONR | NRCONR | CN | 2 | NRCONR | NRCNHNR | CN |
| 2 | NRCONR | NRCOO | F | 2 | NRCONR | C≡C | F |
| 2 | NRCONR | NRCOO | CN | 2 | NRCONR | C≡C | CN |
| 2 | NRCONR | CH═CH | I | 2 | NRCNHNR | O | I |
| 2 | NRCONR | CH═CH | F | 2 | NRCNHNR | O | F |
| 2 | NRCONR | CH═CH | CN | 2 | NRCNHNR | O | CN |
| 2 | NRCNHNR | S | F | 2 | NRCNHNR | NR | F |
| 2 | NRCNHNR | S | COH | 2 | NRCNHNR | NR | COH |
| 2 | NRCNHNR | S | COR | 2 | NRCNHNR | NR | COR |
| 2 | NRCNHNR | CR₂R₂ | COR | 2 | NRCNHNR | CONR | COR |
| 2 | NRCNHNR | SO₂NR | OH | 2 | NRCNHNR | NRCONR | OH |
| 2 | NRCNHNR | SO₂NR | N₃ | 2 | NRCNHNR | NRCONR | N₃ |
| 2 | NRCNHNR | NRCHHNR | CONH₂ | 2 | NRCNHNR | NRCOO | CONH₂ |
| 2 | NRCNHNR | NRCNHNR | COH | 2 | NRCNHNR | NRCOO | COH |
| 2 | NRCNHNR | NRCNHNR | COR | 2 | NRCNHNR | NRCOO | COR |
| 2 | NRCNHNR | C≡C | OH | 2 | NRCNHNR | CH═CH OH | |
| 2 | NRCNHNR | C≡C | SH | 2 | NRCNHNR | C≡CH | SH |
| 2 | NRCNHNR | C≡C | NH₂ | 2 | NRCNHNR | CH═CH | NH₂ |
| 2 | NRCOO | O | I | 2 | NRCOO | S | I |
| 2 | NRCOO | O | C≡CH | 2 | NRCOO | S | C≡CH |
| 2 | NRCOO | O | COR | 2 | NRCOO | S | COR |
| 2 | NRCOO | NR | SH | 2 | NRCOO | CR₂R₂ | SH |
| 2 | NRCOO | NR | COOH | 2 | NRCOO | CR₂R₂ | COOH |
| 2 | NRCOO | CONR | I | 2 | NRCOO | SO₂NR | I |
| 2 | NRCOO | CONR | CN | 2 | NRCOO | SO₂NR | CN |
| 2 | NRCOO | NRCONR | OH | 2 | NRCOO | NRCNHNR | OH |
| 2 | NRCOO | NRCONR | SH | 2 | NRCOO | NRCNHNR | SH |
| 2 | NRCOO | NRCOO | Br | 2 | NRCOO | C≡C | Br |
| 2 | NRCOO | NRCOO | F | 2 | NRCOO | C≡C | F |
| 2 | NRCOO | NRCOO | N₃ | 2 | NRCOO | C≡C | N₃ |
| 2 | NRCOO | CH═CH | CN | 2 | C≡C | O | CN |
| 2 | NRCOO | CH═CH | C≡CH | 2 | C≡C | O | C≡CH |
| 2 | NRCOO | CH═CH | NH₂ | 2 | C≡C | O | NH₂ |
| 2 | C≡C | S | COOH | 2 | C≡C | NR | COOH |
| 2 | C≡C | S | CONH₂ | 2 | C≡C | NR | CONH₂ |
| 2 | C≡C | S | NHR | 2 | C≡C | NR | NHR |
| 2 | C≡C | CR₂R₂ | COOH | 2 | C≡C | CONR | COOH |
| 2 | C≡C | SO₂NR | SH | 2 | C≡C | NRCONR | SH |
| 2 | C≡C | SO₂NR | N₃ | 2 | C≡C | NRCONR | N₃ |
| 2 | C≡C | SO₂NR | CONH₂ | 2 | C≡C | NRCONR | CONH₂ |
| 2 | C≡C | SO₂NR | CH═CH₂ | 2 | C≡C | NRCONR | CH═CH₂ |
| 2 | C≡C | NRCNHNR | I | 2 | C≡C | NRCOO | I |
| 2 | C≡C | NRCNHNR | F | 2 | C≡C | NRCOO | F |
| 2 | C≡C | NRCNHNR | NHR | 2 | C≡C | NRCOO | NHR |
| 2 | C≡C | C≡C | CH═CH₂ | 2 | C≡C | CH═CH | CH═CH₂ |
| 2 | C≡C | C≡C | C≡CH | 2 | C≡C | CH═CH | C≡CH |
| 2 | CH═CH | O | CONH₂ | 2 | CH═CH | S | CONH₂ |
| 2 | CH═CH | O | NHR | 2 | CH═CH | S | NHR |
| 2 | CH═CH | O | COR | 2 | CH═CH | S | COR |
| 2 | CH═CH | NR | I | 2 | CH═CH | CR₂R₂ | I |
| 2 | CH═CH | NR | F | 2 | CH═CH | CR₂R₂ | F |
| 2 | CH═CH | NR | CN | 2 | CH═CH | CR₂R₂ | CN |
| 2 | CH═CH | NR | CH═CH₂ | 2 | CH═CH | CR₂R₂ | CH═CH₂ |
| 2 | CH═CH | CONR | C≡CH | 2 | CH═CH | SO₂NR | C≡CH |
| 2 | CH═CH | CONR | NH₂ | 2 | CH═CH | SO₂NR | NH₂ |
| 2 | CH═CH | NRCONR | Cl | 2 | CH═CH | NRCNHNR | Cl |
| 2 | CH═CH | NRCONR | N₃ | 2 | CH═CH | NRCNHNR | N₃ |
| 2 | CH═CH | NRCOO | SH | 2 | CH═CH | C≡C | SH |
| 2 | CH═CH | NRCOO | CONH₂ | 2 | CH═CH | C≡C | CONH₂ |
| 2 | CH═CH | NRCOO | CH═CH₂ | 2 | CH═CH | C≡C | CH═CH₂ |
| 2 | CH═CH | NRCOO | C≡CH | 2 | CH═CH | C≡C | C≡CH |
| 2 | CH═CH | CH═CH | SO₂H | 2 | CH═CH | CH═CH | C≡CH |
| 2 | CH═CH | CH═CH | Cl | 2 | CH═CH | CH═CH | NH₂ |
| 2 | CH═CH | CH═CH | Br | 2 | CH═CH | CH═CH | NHR |
| 3 | O | O | Cl | 3 | O | S | Cl |
| 3 | O | O | I | 3 | O | S | I |
| 3 | O | NR | CONH₂ | 3 | O | CR₃R₂ | CONH₂ |
| 3 | O | NR | CH═CH₂ | 3 | O | CR₃R₂ | CH═CH₂ |
| 3 | O | NR | NH₂ | 3 | O | CR₃R₂ | NH₂ |
| 3 | O | CONR | NH₂ | 3 | O | SO₂NR | NH₂ |
| 3 | O | CONR | NHR | 3 | O | SO₂NR | NHR |
| 3 | O | NRCONR | N₃ | 3 | O | NRCNHNR | N₃ |
| 3 | O | NRCONR | CONH₂ | 3 | O | NRCNHNR | CONH₂ |
| 3 | O | NRCOO | SH | 3 | O | C≡C | SH |
| 3 | O | NRCOO | F | 3 | O | C≡C | F |
| 3 | O | NRCOO | N₃ | 3 | O | C═C | N₃ |
| 3 | O | NRCOO | C≡CH | 3 | O | C≡C | C≡CH |
| 3 | O | NRCOO | NH₂ | 3 | O | C≡C | NH₂ |
| 3 | O | CH═CH | NH₂ | 3 | S | O | NH₂ |
| 3 | O | CH═CH | COH | 3 | S | O | COH |
| 3 | O | CH═CH | COR | 3 | S | O | COR |
| 3 | S | S | OH | 3 | S | NR | OH |
| 3 | S | S | SH | 3 | S | NR | SH |
| 3 | S | S | NHR | 3 | S | NR | NHR |
| 3 | S | S | COH | 3 | S | NR | COH |
| 3 | S | CH₃H₂ | NH₂ | 3 | S | CONR | NH₂ |
| 3 | S | SO₂NR | SH | 3 | S | NRCONR | SH |
| 3 | S | SO₂NR | COOH | 3 | S | NRCONR | COOH |
| 3 | S | NRCNHNR | I | 3 | S | NRCOO | I |
| 3 | S | NRCNHNR | CONH₂ | 3 | S | NRCOO | CONH₂ |
| 3 | S | NRCNHNR | COR | 3 | S | NHCOO | COR |
| 3 | S | C≡C | OH | 3 | S | CH═CH | OH |
| 3 | S | C≡C | SH | 3 | S | CH═CH | SH |
| 3 | NR | O | CH═CH₂ | 3 | NR | S | CH═CH₂ |
| 3 | NR | O | C≡CH | 3 | NR | S | C≡CH |
| 3 | NR | O | COH | 3 | NR | S | COH |
| 3 | NR | NR | SH | 3 | NR | CR₃R₂ | SH |
| 3 | NR | NR | COOH | 3 | NR | CR₃R₂ | COOH |
| 3 | NR | NR | SO₂H | 3 | NR | CR₃R₂ | SO₂H |
| 3 | NR | CONR | NH₂ | 3 | NR | SO₂NR | NH₂ |
| 3 | NR | CONR | NHR | 3 | NR | SO₂NR | NHR |
| 3 | NR | CONR | COH | 3 | NR | SO₂NR | COH |
| 3 | NR | NRCONR | COOH | 3 | NR | NRCNHNR | COOH |
| 3 | NR | NRCONR | C≡CH | 3 | NR | NRCNHNR | C≡CH |
| 3 | NR | NRCONR | NH₂ | 3 | NR | NRCNHNR | NH₂ |
| 3 | NR | NRCOO | OH | 3 | NR | C≡C | OH |
| 3 | NR | NRCOO | NHR | 3 | NR | C≡C | NHR |
| 3 | NR | CH═CH | COOH | 3 | CH₃H₂ | O | COOH |
| 3 | NR | CH═CH | I | 3 | CH₃H₂ | O | I |
| 3 | CR₃H₂ | S | Br | 3 | CH₃H₂ | NR | Br |
| 3 | CR₃H₂ | CH₃H₂ | CH═CH₂ | 3 | CH₃H₂ | CONR | CH═CH₂ |
| 3 | CR₃H₂ | CH₃H₂ | C≡CH | 3 | CH₃H₂ | CONR | C≡CH |
| 3 | CR₃H₂ | SO₂NR | NH₂ | 3 | CH₃H₂ | NRCONR | NH₂ |
| 3 | CR₃H₂ | SO₂NR | NHR | 3 | CH₃H₂ | NRCONR | NHR |
| 3 | CR₃H₂ | SO₂NR | COH | 3 | CH₃H₂ | NRCONR | COH |
| 3 | CR₃H₂ | NRCNHNR | COOH | 3 | CH₃H₂ | NHCOO | COOH |
| 3 | CR₃H₂ | NRCNHNR | SO₂H | 3 | CH₃H₂ | NHCOO | SO₂H |
| 3 | CR₃H₂ | NRCNHNR | COH | 3 | CH₃H₂ | NHCOO | COH |
| 3 | CR₃H₂ | C≡C | SO₂H | 3 | CH₃H₂ | CH═CH | SO₂H |
| 3 | CR₃H₂ | C≡C | CN | 3 | CH₃H₂ | CH═CH | CN |
| 3 | CONR | O | SO₂H | 3 | CONR | S | SO₂H |
| 3 | CONR | O | Cl | 3 | CONR | S | Cl |
| 3 | CONR | O | Br | 3 | CONR | S | Br |
| 3 | CONR | NR | N₃ | 3 | CONR | CH₃R₂ | N₃ |
| 3 | CONR | NR | CONH₂ | 3 | CONR | CH₃H₂ | CONH₂ |
| 3 | CONR | NR | CH═CH₂ | 3 | CONR | CH₃H₂ | CH═CH₂ |
| 3 | CONR | CONR | C≡CH | 3 | CONR | SO₂NR | C≡CH |
| 3 | CONR | CONR | NH₂ | 3 | CONR | SO₂NR | NH₂ |
| 3 | CONR | NRCONR | I | 3 | CONR | NRCNHNR | I |
| 3 | CONR | NRCONR | N₃ | 3 | CONR | NRCNHNR | N₃ |
| 3 | CONR | NRCOO | COH | 3 | CONR | C≡C | COH |
| 3 | CONR | NRCOO | COR | 3 | CONR | C≡C | COR |
| 3 | CONR | CH═CH | OH | 3 | SO₂NR | O | OH |
| 3 | CONR | CH═CH | SH | 3 | SO₂NR | O | SH |
| 3 | SO₂NR | S | SO₂H | 3 | SO₂NR | NR | SO₂N |
| 3 | SO₂NR | S | COH | 3 | SO₂NR | NR | COH |
| 3 | SO₂NR | S | COR | 3 | SO₂NR | NR | COR |
| 3 | SO₂NR | CR₃R₂ | OH | 3 | SO₂NR | CONR | OH |
| 3 | SO₂NR | CR₃R₂ | SH | 3 | SO₂NR | CONR | SH |
| 3 | SO₂NR | CR₃R₂ | CONH₂ | 3 | SO₂NR | CONR | CONH₂ |
| 3 | SO₂NR | CR₃R₂ | CH═CH₂ | 3 | SO₂NR | CONR | CH═CH₂ |
| 3 | SO₂NR | SO₂NR | SH | 3 | SO₂NR | NRCONR | SH |
| 3 | SO₂NR | SO₂NR | COH | 3 | SO₂NR | NRCONR | COH |
| 3 | SO₂NR | SO₂NR | COR | 3 | SO₂NR | NRCONR | COR |
| 3 | SO₂NR | NRCNHNR | OH | 3 | SO₂NR | NRCOO | OH |
| 3 | SO₂NR | NRCNHNR | SH | 3 | SO₂NR | NRCOO | SH |
| 3 | SO₂NR | C≡C | CH═CH₂ | 3 | SO₂NR | CH═CH | CH═CH₂ |
| 3 | SO₂NR | C≡C | NH₂ | 3 | SO₂NR | CH═CH | NH₂ |
| 3 | SO₂NR | C≡C | NHR | 3 | SO₂NR | CH═CH | NHR |
| 3 | NRCONR | O | Br | 3 | NRCONR | S | Br |
| 3 | NRCONR | O | I | 3 | NRCONR | S | I |
| 3 | NRCONR | NR | F | 3 | NRCONR | CR₃R₂ | F |
| 3 | NRCONR | NR | CN | 3 | NRCONR | CR₃R₂ | CN |
| 3 | NRCONR | CONR | SO₂N | 3 | NRCONR | SO₂NR | SO₂N |
| 3 | NRCONR | CONR | Cl | 3 | NRCONR | SO₂NR | Cl |
| 3 | NRCONR | NRCONR | SH | 3 | NRCONR | NRCNHNR | SH |
| 3 | NRCONR | NRCONR | CONH₂ | 3 | NRCONR | NRCNHNR | CONH₂ |
| 3 | NRCONR | NRCONR | CH═CH₂ | 3 | NRCONR | NRCNHNR | CH═CH₂ |
| 3 | NRCONR | NRCOO | NH₂ | 3 | NRCONR | C≡C | NH₂ |
| 3 | NRCONR | NRCOO | COH | 3 | NRCONR | C≡C | COH |
| 3 | NRCONR | CH═CH | OH | 3 | NRCNHNR | O | OH |
| 3 | NRCONR | CH═CH | CONH₂ | 3 | NRCNHNR | O | CONH₂ |
| 3 | NRCONR | CH═CH | CH═CH₂ | 3 | NRCNHNR | O | CH═CH₂ |
| 3 | NRCNHNR | S | SH | 3 | NRCNHNR | NR | SH |
| 3 | NRCNHNR | S | COOH | 3 | NRCNHNR | NR | COOH |
| 3 | NRCNHNR | S | SO₂N | 3 | NRCNHNR | NR | SO₂N |
| 3 | NRCNHNR | SO₂NR | Br | 3 | NRCNHNR | NRCONR | Br |
| 3 | NRCNHNR | SO₂NR | C≡CH | 3 | NRCNHNR | NRCONR | C≡CH |
| 3 | NRCNHNR | SO₂NR | NH₂ | 3 | NRCNHNR | NRCONR | NH₂ |
| 3 | NRCNHNR | NRCNHNR | COOH | 3 | NRCNHNR | NRCOO | COOH |
| 3 | NRCNHNR | NRCNHNR | SO₂H | 3 | NRCNHNR | NRCOO | SO₂H |
| 3 | NRCNHNR | C≡C | Cl | 3 | NRCNHNR | CH═CH | Cl |
| 3 | NRCNHNR | C≡C | Br | 3 | NRCNHNR | CH═CH | Br |
| a3 | NRCOO | O | SH | 3 | NRCOO | S | SH |
| 3 | NRCOO | O | COOH | 3 | NRCOO | S | COOH |
| 3 | NRCOO | O | SO₂N | 3 | NRCOO | S | SO₂N |
| 3 | NRCOO | NR | F | 3 | NRCOO | CR₃R₂ | F |
| 3 | NRCOO | NR | CN | 3 | NRCOO | CR₃R₂ | CN |
| 3 | NRCOO | NR | COR | 3 | NRCOO | CR₃R₂ | COR |
| 3 | NRCOO | CONR | C≡CH | 3 | NRCOO | SO₂NR | C≡CH |
| 3 | NRCOO | CONR | COH | 3 | NRCOO | SO₂NR | COH |
| 3 | NRCOO | CONR | COR | 3 | NRCOO | SO₂NR | COR |
| 3 | NRCOO | NRCONR | OH | 3 | NRCOO | NRCNHNR | OH |
| 3 | NRCOO | NRCONR | COR | 3 | NRCOO | NRCNHNR | COR |
| 3 | NRCOO | NRCOO | Br | 3 | NRCOO | C≡C | Br |
| 3 | NRCOO | CH═CH | CONH₂ | 3 | C≡C | O | CONH₂ |
| 3 | NRCOO | CH═CH | CH═CH₂ | 3 | C≡C | O | CH═CH₂ |
| 3 | C≡C | S | OH | 3 | C≡C | NR | OH |
| 3 | C≡C | CR₃R₂ | I | 3 | C≡C | CONR | I |
| 3 | C≡C | CR₃R₂ | F | 3 | C≡C | CONR | F |
| 3 | C≡C | CR₃R₂ | NH₂ | 3 | C≡C | CONR | NH₂ |
| 3 | C≡C | SO₂NR | N₃ | 3 | C≡C | NRCONR | N₃ |
| 3 | C≡C | SO₂NR | CONH₂ | 3 | C≡C | NRCONR | CONH₂ |
| 3 | C≡C | SO₂NR | CH═CH₂ | 3 | C≡C | NRCONR | CH═CH₂ |
| 3 | C≡C | NRCNHNR | CH═CH₂ | 3 | C≡C | NRCOO | CH═CH₂ |
| 3 | C≡C | NRCNHNR | C≡CH | 3 | C≡C | NRCOO | C≡CH |
| 3 | C≡C | C≡C | I | 3 | C≡C | CH═CH | I |
| 3 | C≡C | C≡C | C≡CH | 3 | C≡C | CH═CH | C≡CH |
| 3 | C≡C | C≡C | NH₂ | 3 | C≡C | CH═CH | NH₂ |
| 3 | C≡C | C≡C | NHR | 3 | CH═CH | CH═CH | NHR |
| 3 | CH═CH | O | COOH | 3 | CH═CH | S | COOH |
| 3 | CH═CH | O | CN | 3 | CH═CH | S | CN |
| 3 | CH═CH | NR | I | 3 | CH═CH | CR₃R₂ | I |
| 3 | CH═CH | NR | F | 3 | CH═CH | CR₃R₂ | F |
| 3 | CH═CH | CONR | CN | 3 | CH═CH | SO₂NR | CN |
| 3 | CH═CH | CONR | N₃ | 3 | CH═CH | SO₂NR | N₃ |
| 3 | CH═CH | CONR | C≡CH | 3 | CH═CH | SO₂NR | C≡CH |
| 3 | CH═CH | NRCONR | NHR | 3 | CH═CH | NRCNHNR | NHR |
| 3 | CH═CH | NRCOO | Br | 3 | CH═CH | C≡C | Br |
| 3 | CH═CH | NRCOO | I | 3 | CH═CH | C≡C | I |
| 3 | CH═CH | CH═CH | Cl | 3 | CH═CH | CH═CH | NH₂ |
| 3 | O | O | OH | 3 | O | S | OH |
| 3 | O | O | SH | 3 | O | S | SH |
| 3 | O | NR | CH═CH₂ | 3 | O | CR₃R₂ | CH═CH₂ |
| 3 | O | NR | C≡CH | 3 | O | CR₃R₂ | C≡CH |
| 3 | O | NR | NH₂ | 3 | O | CR₃R₂ | NH₂ |
| 3 | O | CONR | Br | 3 | O | SO₂NR | Br |
| 3 | O | NRCONR | Br | 3 | O | NRCNHNR | Br |
| 3 | O | NRCONR | CONH₂ | 3 | O | NRCNHNR | CONH₂ |
| 3 | O | NRCOO | COH | 3 | O | C≡C | COH |
| 3 | O | NRCOO | COR | 3 | O | C≡C | COR |
| 3 | O | CH═CH | CONH₂ | 3 | S | O | CONH₂ |
| 3 | O | CH═CH | CH═CH₂ | 3 | S | O | CH═CH₂ |
| 3 | O | CH═CH | C≡CH | 3 | S | O | C≡CH |
| 3 | S | S | CONH₂ | 3 | S | NR | CONH₂ |
| 3 | S | S | CH═CH₂ | 3 | S | NR | CH≡CH₂ |
| 3 | S | S | C≡CH | 3 | S | NR | C≡CH |
| 3 | S | S | NH₂ | 3 | S | NR | NH₂ |
| 3 | S | CR₃R₂ | N₃ | 3 | S | CONR | N₃ |
| 3 | S | CR₃R₂ | C≡CH | 3 | S | CONR | C≡CH |
| 3 | S | SO₂NR | Br | 3 | S | NRCONR | Br |
| 3 | S | SO₂NR | NHR | 3 | S | NRCONR | NHR |
| 3 | S | SO₂NR | COH | 3 | S | NRCONR | COH |
| 3 | S | NRCNHNR | N₃ | 3 | S | NRCOO | N₃ |
| 3 | S | NRCNHNR | COR | 3 | S | NRCOO | COR |
| 3 | S | C≡C | OH | 3 | S | CH═CH | ON |
| 3 | S | C≡C | SH | 3 | S | CH═CH | SH |
| 3 | S | C≡C | Br | 3 | S | CH═CH | Br |
| 3 | NR | O | SH | 3 | NR | S | SH |
| 3 | NR | O | COOH | 3 | NR | S | COOH |
| 3 | NR | O | CONH₂ | 3 | NR | S | CONH₂ |
| 3 | NR | O | COR | 3 | NR | S | COR |
| 3 | NR | NR | OH | 3 | NR | CR₃R₂ | OH |
| 3 | NR | NR | I | 3 | NR | CR₃R₂ | I |
| 3 | NR | NR | F | 3 | NR | CR₃R₂ | F |
| 3 | NR | CONR | F | 3 | NR | SO₂NR | F |
| 3 | NR | CONR | CONH₂ | 3 | NR | SO₂NR | CONH₂ |
| 3 | NR | NRCONR | Br | 3 | NR | NRCNHNR | Br |
| | NR | NRCONR | I | 3 | NR | NRCNHNR | I |
| 3 | NR | NRCOO | CN | 3 | NR | C≡C | CN |
| 3 | NR | NRCOO | N₃ | 3 | NR | C≡C | N₃ |
| 3 | NR | NRCOO | CONH₂ | 3 | NR | C≡C | CONH₂ |
| 3 | NR | CH═CH | Cl | 3 | CR₃R₂ | O | Cl |
| 3 | NR | CH═CH | Br | 3 | CR₃R₂ | O | Br |
| 3 | CR₃R₂ | S | COOH | 3 | CR₃R₂ | NR | COOH |
| 3 | CR₃R₂ | S | SO₂H | 3 | CR₃R₂ | NR | SO₂H |
| 3 | CR₃R₂ | S | Cl | 3 | CR₃R₂ | NR | Cl |
| 3 | CR₃R₂ | CR₃R₂ | COOH | 3 | CR₃R₂ | CONR | COOH |
| 3 | CR₃R₂ | CR₃R₂ | I | 3 | CR₃R₂ | CONR | I |
| 3 | CR₃R₂ | CR₃R₂ | CH═CH₂ | 3 | CR₃R₂ | CONR | CH═CH₂ |
| 3 | CR₃R₂ | CR₃R₂ | C≡CH | 3 | CR₃R₂ | CONR | C≡CH |
| 3 | CR₃R₂ | SO₂NR | F | 3 | CR₃R₂ | NRCONR | F |
| 3 | CR₃R₂ | SO₂NR | CH═CH₂ | 3 | CR₃R₂ | NRCONR | CH═CH₂ |
| 3 | CR₃R₂ | SO₂NR | C≡CH | 3 | CR₃R₂ | NRCONR | C≡CH |
| 3 | CR₃R₂ | SO₂NR | NH₂ | 3 | CR₃R₂ | NRCONR | NH₂ |
| 3 | CR₃R₂ | NRCNHNR | OH | 3 | CR₃R₂ | NRCOO | OH |
| 3 | CR₃R₂ | NRCNHNR | SH | 3 | CR₃R₂ | NRCOO | SH |
| 3 | CR₃R₂ | C≡C | C≡CH | 3 | CR₃R₂ | CH═CH | C≡CH |
| 3 | CR₃R₂ | C≡C | NH₂ | 3 | CR₃R₂ | CH═CH | NH₂ |
| 3 | CONR | O | SH | 3 | CONR | S | SH |
| 3 | CONR | O | COOH | 3 | CONR | S | COOH |
| 3 | CONR | O | CONH₂ | 3 | CONR | S | CONH₂ |
| 3 | CONR | NR | I | 3 | CONR | CR₃R₂ | I |
| 3 | CONR | NR | F | 3 | CONR | CR₃R₂ | F |
| 3 | CONR | CONR | OH | 3 | CONR | SO₂NR | OH |
| 3 | CONR | CONR | SH | 3 | CONR | SO₂NR | SH |
| 3 | CONR | CONR | COOH | 3 | CONR | SO₂NR | COOH |
| 3 | CONR | NRCONR | NHR | 3 | CONR | NRCNHNR | NHR |
| 3 | CONR | NRCONR | COH | 3 | CONR | NRCNHNR | COH |
| 3 | CONR | NRCOO | I | 3 | CONR | C≡C | I |
| 3 | CONR | NRCOO | F | 3 | CONR | C≡C | F |
| 3 | CONR | CH═CH | F | 3 | SO₂NR | O | F |
| 3 | CONR | CH═CH | COR | 3 | SO₂NR | O | COR |
| 3 | SO₂NR | S | OH | 3 | SO₂NR | NR | OH |
| 3 | SO₂NR | S | SH | 3 | SO₂NR | NR | SH |
| 3 | SO₂NR | CR₃R₂ | N₃ | 3 | SO₂NR | COHR | N₃ |
| 3 | SO₂NR | CR₃R₂ | CONH₂ | 3 | SO₂NR | CONR | CONH₂ |
| 3 | SO₂NR | SO₂NR | COOH | 3 | SO₂NR | NRCONR | COOH |
| 3 | SO₂NR | SO₂NR | CN | 3 | SO₂NR | NRCONR | CN |
| 3 | SO₂NR | SO₂NR | N₃ | 3 | SO₂NR | NRCONR | N₃ |
| 3 | SO₂NR | SO₂NR | CONH₂ | 3 | SO₂NR | NRCONR | CONH₂ |
| 3 | SO₂NR | NRCNHNR | CN | 3 | SO₂NR | NRCOO | CN |
| 3 | SO₂NR | NRCNHNR | CH═CH₂ | 3 | SO₂NR | NRCOO | CH═CH₂ |
| 3 | SO₂NR | C≡C | SO₂H | 3 | SO₂NR | CH═CH | SO₂H |
| 3 | SO₂NR | C≡C | Cl | 3 | SO₂NR | CH═CH | Cl |
| 3 | SO₂NR | C≡C | Br | 3 | SO₂NR | CH═CH | Br |
| 3 | NRCONR | O | C≡CH | 3 | NRCONR | S | C≡CH |
| 3 | NRCONR | O | NH₂ | 3 | NRCONR | S | NH₂ |
| 3 | NRCONR | NR | Cl | 3 | NRCONR | CR₃R₂ | Cl |
| 3 | NRCONR | NR | Br | 3 | NRCONR | CR₃R₂ | Br |
| 3 | NRCONR | NR | CONH₂ | 3 | NRCONR | CR₃R₂ | CONH₂ |
| 3 | NRCONR | CONR | OH | 3 | NRCONR | SO₂NR | OH |
| 3 | NRCONR | CONR | F | 3 | NRCONR | SO₂NR | F |
| 3 | NRCONR | CONR | CN | 3 | NRCONR | SO₂NR | CN |
| 3 | NRCONR | NRCONR | CONH₂ | 3 | NRCONR | NRCNHNR | CONH₂ |
| 3 | NRCONR | NRCONR | CH═CH₂ | 3 | NRCONR | NRCNHNR | CH═CH₂ |
| 3 | NRCONR | NRCOO | CONH₂ | 3 | NRCONR | C≡C | CONH₂ |
| 3 | NRCONR | NRCOO | COH | 3 | NRCONR | C≡C | COH |
| 3 | NRCONR | CH═CH | SO₂H | 3 | NRCNHNR | O | SO₂H |
| 3 | NRCONR | CH═CH | Cl | 3 | NRCNHNR | O | Cl |
| 3 | NRCONR | CH═CH | F | 3 | NRCNHNR | O | F |
| 3 | NRCNHNR | S | OH | 3 | NRCNHNR | NR | OH |
| 3 | NRCNHNR | S | Br | 3 | NRCNHNR | NR | Br |
| 3 | NRCNHNR | CR₃R₂ | OH | 3 | NRCNHNR | CONR | OH |
| 3 | NRCNHNR | CR₃R₂ | SH | 3 | NRCNHNR | CONR | SH |
| 3 | NRCNHNR | CR₃R₂ | CH═CH₂ | 3 | NRCNHNR | CONR | CH═CH₂ |
| 3 | NRCNHNR | SO₂NR | I | 3 | NRCNHNR | NRCONR | I |
| 3 | NRCNHNR | SO₂NR | NHR | 3 | NRCNHNR | NRCONR | NHR |
| 3 | NRCNHNR | SO₂NR | COH | 3 | NRCNHNR | NRCONR | COH |
| 3 | NRCNHNR | SO₂NR | COR | 3 | NRCNHNR | NRCONR | COR |
| 3 | NRCNHNR | NRCNHNR | N₃ | 3 | NRCNHNR | NRCOO | N₃ |
| 3 | NRCNHNR | NRCNHNR | CONH₂ | 3 | NRCNHNR | NRCOO | CONH₂ |
| 3 | NRCNHNR | NRCNHNR | COR | 3 | NRCNHNR | NRCOO | COR |
| 3 | NRCNHNR | C≡C | OH | 3 | NRCNHNR | CH═CH | OH |
| 3 | NRCNHNR | C≡C | COR | 3 | NRCNHNR | CH═CH | COR |
| 3 | NRCOO | O | OH | 3 | NRCOO | S | OH |
| 3 | NRCOO | O | SH | 3 | NRCOO | S | SH |
| a3 | NRCOO | O | COR | 3 | NRCOO | S | COR |
| 3 | NRCOO | NR | OH | 3 | NRCOO | CR₃R₂ | OH |
| 3 | NRCOO | NR | SH | 3 | NRCOO | CR₃R₂ | SH |
| 3 | NRCOO | NR | COOH | 3 | NRCOO | CR₃R₂ | COOH |
| 3 | NRCOO | CONR | NH₂ | 3 | NRCOO | SO₂NR | NH₂ |
| 3 | NRCOO | CONR | NHR | 3 | NRCOO | SO₂NR | NHR |
| 3 | NRCOO | NRCONR | CH═CH₂ | 3 | NRCOO | NRCNHNR | CH═CH₂ |
| 3 | NRCOO | NRCONR | NHR | 3 | NRCOO | NRCNHNR | NHR |
| 3 | NRCOO | NRCOO | I | 3 | NRCOO | C≡C | I |
| 3 | NRCOO | CH═CH | OH | 3 | C≡C | O | OH |
| 3 | NRCOO | CH═CH | SH | 3 | C≡C | O | SH |
| 3 | NRCOO | CH═CH | COOH | 3 | C≡C | O | COOH |
| 3 | C≡C | S | C≡CH | 3 | C≡C | NR | C≡CH |
| 3 | C≡C | S | NH₂ | 3 | C≡C | NR | NH₂ |
| 3 | C≡C | S | NHR | 3 | C≡C | NR | NHR |
| 3 | C≡C | CR₃R₂ | SO₂H | 3 | C≡C | CONR | SO₂H |
| 3 | C≡C | CR₃R₂ | Cl | 3 | C≡C | CONR | Cl |
| 3 | C≡C | CR₃R₂ | Br | 3 | C≡C | CONR | Br |
| 3 | C≡C | SO₂NR | OH | 3 | C≡C | NRCONR | OH |
| 3 | C≡C | SO₂NR | SH | 3 | C≡C | NRCONR | SH |
| 3 | C≡C | SO₂NR | Br | 3 | C≡C | NRCONR | Br |
| 3 | C≡C | NRCNHNR | CONH₂ | 3 | C≡C | NRCOO | CONH₂ |
| 3 | C≡C | NRCNHNR | NHR | 3 | C≡C | NRCOO | NHR |
| 3 | C≡C | C≡C | C≡CH | 3 | C≡C | CH═CH | C≡CH |
| 3 | C≡C | C≡C | NH₂ | 3 | C≡C | CH═CH | NH₂ |
| 3 | C≡C | C≡C | COR | 3 | C≡C | CH═CH | COR |
| 3 | CH═CH | O | OH | 3 | CH═CH | S | OH |
| 3 | CH═CH | O | SH | 3 | CH═CH | S | SH |
| 3 | CH═CH | O | COOH | 3 | CH═CH | S | COOH |
| 3 | CH═CH | O | SO₂H | 3 | CH═CH | S | SO₂H |
| 3 | CH═CH | O | Cl | 3 | CH═CH | S | Cl |
| 3 | CH═CH | NR | OH | 3 | CH═CH | CR₃R₂ | OH |
| 3 | CH═CH | NR | COOH | 3 | CH═CH | CR₃R₂ | COOH |
| 3 | CH═CH | NR | F | 3 | CH═CH | CR₃R₂ | F |
| 3 | CH═CH | CONR | NH₂ | 3 | CH═CH | SO₂NR | NH₂ |
| 3 | CH═CH | CONR | NHR | 3 | CH═CH | SO₂NR | NHR |
| 3 | CH═CH | CONR | COH | 3 | CH═CH | SO₂NR | COH |
| 3 | CH═CH | CONR | COR | 3 | CH═CH | SO₂NR | COR |
| 3 | CH═CH | NRCONR | OH | 3 | CH═CH | NRCNHNR | OH |
| 3 | CH═CH | NRCOO | CH═CH₂ | 3 | CH═CH | C≡C | CH═CH₂ |
| 3 | CH═CH | NRCOO | NHR | 3 | CH═CH | C≡C | NHR |
| 3 | CH═CH | CH═CH | I | 3 | CH═CH | CH═CH | COH |
| 3 | CH═CH | CH═CH | F | 3 | CH═CH | CH═CH | COR |
| 3 | CH═CH | CH═CH | CN | | | | |
| 3 | O | O | OH | 3 | O | S | OH |
| 3 | O | O | SH | 3 | O | S | SH |
| 3 | O | O | COOH | 3 | O | S | COOH |
| 3 | O | NR | CONH₂ | 3 | O | CR₃R₂ | CONH₂ |
| 3 | O | NR | CH═CH₂ | 3 | O | CR₃R₂ | CH═CH₂ |
| 3 | O | NR | C≡CH | 3 | O | CR₃R₂ | C≡CH |
| 3 | O | CONR | CONH₂ | 3 | O | SO₂NR | CONH₂ |
| 3 | O | CONR | CH═CH₂ | 3 | O | SO₂NR | CH═CH₂ |
| 3 | O | NRCONR | CONH₂ | 3 | O | NRCNHNR | CONH₂ |
| 3 | O | NRCONR | CH═CH₂ | 3 | O | NRCNHNR | CH═CH₂ |
| 3 | O | NRCOO | COOH | 3 | O | C≡C | COOH |
| 3 | O | NRCOO | SO₂H | 3 | O | C≡C | SO₂H |
| 3 | O | NRCOO | Cl | 3 | O | C≡C | Cl |
| 3 | O | CH═CH | SO₂H | 3 | S | O | SO₂H |
| 3 | O | CH═CH | Cl | 3 | S | O | Cl |
| 3 | O | CH═CH | COR | 3 | S | O | COR |
| 3 | S | S | OH | 3 | S | NR | OH |
| 3 | S | S | SH | 3 | S | NR | SH |
| 3 | S | S | COOH | 3 | S | NR | COOH |
| 3 | S | S | SO₂H | 3 | S | NR | SO₂H |
| 3 | S | CR₃R₂ | CONH₂ | 3 | S | CONR | CONH₂ |
| 3 | S | CR₃R₂ | CH═CH₂ | 3 | S | CONR | CH═CH₂ |
| 3 | S | CR₃R₂ | NHR | 3 | S | CONR | NHR |
| 3 | S | SO₂NR | NHR | 3 | S | NRCONR | NHR |
| 3 | S | SO₂NR | COH | 3 | S | NRCONR | COH |
| 3 | S | SO₂NR | COR | 3 | S | NRCONR | COR |
| 3 | S | NRCNHNR | OH | 3 | S | NRCOO | OH |
| 3 | S | NRCNHNR | NH₂ | 3 | S | NRCOO | NH₂ |
| 3 | S | NRCNHNR | NHR | 3 | S | NRCOO | NHR |
| 3 | S | C≡C | I | 3 | S | CH═CH | I |
| 3 | S | C≡C | NH₂ | 3 | S | CH═CH | NH₂ |
| 3 | NR | O | SO₂H | 3 | NR | S | SO₂H |
| 3 | NR | O | F | 3 | NR | S | F |
| 3 | NR | O | CN | 3 | NR | S | CN |
| 3 | NR | O | N₃ | 3 | NR | S | N₃ |
| 3 | NR | O | NH₂ | 3 | NR | S | NH₂ |
| 3 | NR | NR | SH | 3 | NR | CR₃R₂ | SH |
| 3 | NR | NR | COOH | 3 | NR | CR₃R₂ | COOH |
| 3 | NR | CONR | CN | 3 | NR | SO₂NR | CN |
| 3 | NR | CONR | COR | 3 | NR | SO₂NR | COR |
| 3 | NR | NRCONR | OH | 3 | NR | NRCNHNR | OH |
| 3 | NR | NRCONR | NHR | 3 | NR | NRCNHNR | NHR |
| 3 | NR | NRCOO | SO₂H | 3 | NR | C≡C | SO₂H |
| 3 | NR | NRCOO | C≡CH | 3 | NR | C≡C | C≡CH |
| 3 | NR | NRCOO | NH₂ | 3 | NR | C≡C | NH₂ |
| 3 | NR | NRCOO | NHR | 3 | NR | C≡C | NHR |
| 3 | NR | CH═CH | COR | 3 | CR₃R₂ | O | COR |
| 3 | CR₃R₂ | S | OH | 3 | CR₃R₂ | NR | OH |
| 3 | CR₃R₂ | S | SH | 3 | CR₃R₂ | NR | SH |
| 3 | CR₃R₂ | CR₃R₂ | SO₂H | 3 | CR₃R₂ | CONR | SO₂H |
| 3 | CR₃R₂ | CR₃R₂ | Cl | 3 | CR₃R₂ | CONR | Cl |
| 3 | CR₃R₂ | SO₂NR | OH | 3 | CR₃R₂ | NRCONR | OH |
| 3 | CR₃R₂ | SO₂NR | C≡CH | 3 | CR₃R₂ | NRCONR | C≡CH |
| 3 | CR₃R₂ | SO₂NR | NH₂ | 3 | CR₃R₂ | NRCONR | NH₂ |
| 3 | CR₃R₂ | SO₂NR | NHR | 3 | CR₃R₂ | NRCONR | NHR |
| 3 | CR₃R₂ | NRCNHNR | Cl | 3 | CR₃R₂ | NRCOO | Cl |
| 3 | CR₃R₂ | NRCNHNR | COR | 3 | CR₃R₂ | NRCOO | COR |
| 3 | CR₃R₂ | C≡C | Cl | 3 | CR₃R₂ | CH═CH | Cl |
| 3 | CR₃R₂ | C≡C | Br | 3 | CR₃R₂ | CH═CH | Br |
| 3 | CR₃R₂ | C≡C | NHR | 3 | CR₃R₂ | CH═CH | NHR |
| 3 | CONR | O | COR | 3 | CONR | S | COR |
| 3 | CONR | NR | OH | 3 | CONR | CR₃R₂ | OH |
| 3 | CONR | NR | SH | 3 | CONR | CR₃R₂ | SH |
| 3 | CONR | NR | C≡CH | 3 | CONR | CR₃R₂ | C≡CH |
| 3 | CONR | CONR | Br | 3 | CONR | SO₂NR | Br |
| 3 | CONR | CONR | I | 3 | CONR | SO₂NR | I |
| 3 | CONR | CONR | F | 3 | CONR | SO₂NR | F |
| 3 | CONR | NRCONR | OH | 3 | CONR | NRCNHNR | OH |
| 3 | CONR | NRCOO | COOH | 3 | CONR | C≡C | COOH |
| 3 | CONR | NRCOO | SO₂H | 3 | CONR | C≡C | SO₂H |
| 3 | CONR | NRCOO | F | 3 | CONR | C≡C | F |
| 3 | CONR | CH═CH | Cl | 3 | SO₂NR | O | Cl |
| 3 | CONR | CH═CH | NHR | 3 | SO₂NR | O | NHR |
| 3 | SO₂NR | S | OH | 3 | SO₂NR | NR | OH |
| 3 | SO₂NR | S | SH | 3 | SO₂NR | NR | SH |
| 3 | SO₂NR | S | NH₂ | 3 | SO₂NR | NR | NH₂ |
| 3 | SO₂NR | S | NHR | 3 | SO₂NR | NR | NHR |
| 3 | SO₂NR | CR₃R₂ | Cl | 3 | SO₂NR | CONR | Cl |
| 3 | SO₂NR | CR₃R₂ | Br | 3 | SO₂NR | CONR | Br |
| 3 | SO₂NR | SO₂NR | Br | 3 | SO₂NR | NRCONR | Br |
| 3 | SO₂NR | SO₂NR | I | 3 | SO₂NR | NRCONR | I |
| 3 | SO₂NR | NRCNHNR | OH | 3 | SO₂NR | NRCOO | OH |
| 3 | SO₂NR | NRCNHNR | SH | 3 | SO₂NR | NRCOO | SH |
| 3 | SO₂NR | NRCNHNR | COR | 3 | SO₂NR | NRCOO | COR |
| 3 | SO₂NR | C≡C | OH | 3 | SO₂NR | CH═CH | OH |
| 3 | SO₂NR | C≡C | CN | 3 | SO₂NR | CH═CH | CN |
| 3 | NRCONR | O | I | 3 | NRCONR | S | I |
| 3 | NRCONR | O | COH | 3 | NRCONR | S | COH |
| 3 | NRCONR | O | COR | 3 | NRCONR | S | COR |
| 3 | NRCONR | NR | OH | 3 | NRCONR | CR₃R₂ | OH |
| 3 | NRCONR | NR | SH | 3 | NRCONR | CR₃R₂ | SH |
| 3 | NRCONR | CONR | OH | 3 | NRCONR | SO₂NR | OH |
| 3 | NRCONR | CONR | SH | 3 | NRCONR | SO₂NR | SH |
| 3 | NRC0NR | CONR | SO₂H | 3 | NRCONR | SO₂NR | SO₂H |
| 3 | NRCONR | NRCONR | I | 3 | NRCONR | NRCNHNR | I |
| 3 | NRCONR | NRCONR | N₃ | 3 | NRCONR | NRCNHNR | N₃ |
| 3 | NRCONR | NRCONR | CONH₂ | 3 | NRCONR | NRCNHNR | CONH₂ |
| 3 | NRCONR | NRCOO | SH | 3 | NRCONR | C≡C | SH |
| 3 | NRCONR | NRCOO | COOH | 3 | NRCONR | C≡C | COOH |
| 3 | NRCONR | CH═CH | CN | 3 | NRCNHNR | O | CN |
| 3 | NRCONR | CH═CH | N₃ | 3 | NRCNHNR | O | N₃ |
| 3 | NRCONR | CH═CH | COR | 3 | NRCNHNR | O | COR |
| 3 | NRCNHNR | S | OH | 3 | NRCNHNR | NR | OH |
| 3 | NRCNHNR | S | COH | 3 | NRCNHNR | NR | COH |
| 3 | NRCNHNR | S | COR | 3 | NRCNHNR | NR | COR |
| 3 | NRCNHNR | CR₃R₂ | Br | 3 | NRCNHNR | CONR | Br |
| 3 | NRCNHNR | CR₃R₂ | N₃ | 3 | NRCNHNR | CONR | N₃ |
| 3 | NRCNHNR | SO₂NR | C≡CH | 3 | NRCNHNR | NRCONR | C≡CH |
| 3 | NRCNHNR | SO₂NR | COH | 3 | NRCNHNR | NRCONR | COH |
| 3 | NRCNHNR | NRCNHNR | NHR | 3 | NRCNHNR | NRCOO | NHR |
| 3 | NRCNHNR | NRCNHNR | COH | 3 | NRCNHNR | NRCOO | COH |
| 3 | NRCNHNR | NRCNHNR | COR | 3 | NRCNHNR | NRCOO | COR |
| 3 | NRCNHNR | C≡C | OH | 3 | NRCNHNR | CH═CH | OH |
| 3 | NRCNHNR | C≡C | Br | 3 | NRCNHNR | CH═CH | Br |
| 3 | NRCNHNR | C≡C | I | 3 | NRCNHNR | CH═CH | I |
| 3 | NRCOO | O | COH | 3 | NRCOO | S | COH |
| 3 | NRCOO | O | COR | 3 | NRCOO | S | COR |
| 3 | NRCOO | NR | CONH₂ | 3 | NRCOO | CR₃R₂ | CONH₂ |
| 3 | NRCOO | NR | CH═CH₂ | 3 | NRCOO | CR₃R₂ | CH═CH₂ |
| 3 | NRCOO | NR | COH | 3 | NRCOO | CR₃R₂ | COH |
| 3 | NRCOO | NR | COR | 3 | NRCOO | CR₃R₂ | COR |
| 3 | NRCOO | CONR | OH | 3 | NRCOO | SO₂NR | OH |
| 3 | NRCOO | CONR | Cl | 3 | NRCOO | SO₂NR | Cl |
| 3 | NRCOO | CONR | CONH₂ | 3 | NRCOO | SO₂NR | CONH₂ |
| 3 | NRCOO | NRCONR | Cl | 3 | NRCOO | NRCNHNR | Cl |
| 3 | NRCOO | NRCONR | N₃ | 3 | NRCOO | NRCNHNR | N₃ |
| 3 | NRCOO | NRCONR | CONH₂ | 3 | NRCOO | NRCNHNR | CONH₂ |
| 3 | NRCOO | NRCONR | CH═CH₂ | 3 | NRCOO | NRCNHNR | CH═CH₂ |
| 3 | NRCOO | NRCOO | Cl | 3 | NRCOO | C≡C | Cl |
| 3 | NRCOO | NRCOO | NH₂ | 3 | NRCOO | C≡C | NH₂ |
| 3 | NRCOO | CH═CH | I | 3 | C≡C | O | I |
| 3 | NRCOO | CH═CH | F | 3 | C≡C | O | F |
| 3 | C≡C | S | CN | 3 | C≡C | NR | CN |
| 3 | C≡C | S | NHR | 3 | C≡C | NR | NHR |
| 3 | C≡C | CR₃R₂ | COOH | 3 | C≡C | CONR | COOH |
| 3 | C≡C | CR₃R₂ | SO₂H | 3 | C≡C | CONR | SO₂H |
| 3 | C≡C | CR₃R₂ | CN | 3 | C≡C | CONR | CN |
| 3 | C≡C | SO₂NR | Cl | 3 | C≡C | NRCONR | Cl |
| 3 | C≡C | SO₂NR | COR | 3 | C≡C | NRCONR | COR |
| 3 | C≡C | NRCNHNR | OH | 3 | C≡C | NRCOO | OH |
| 3 | C≡C | NRCNHNR | F | 3 | C≡C | NRCOO | F |
| 3 | C≡C | NRCNHNR | NH₂ | 3 | C≡C | NRCOO | NH₂ |
| 3 | C≡C | C≡C | I | 3 | C≡C | CH═CH | I |
| 3 | C≡C | C≡C | F | 3 | C≡C | CH═CH | F |
| 3 | C≡C | C≡C | CN | 3 | C≡C | CH═CH | CN |
| 3 | CH═CH | O | F | 3 | CH═CH | S | F |
| 3 | CH═CH | O | CN | 3 | CH═CH | S | CN |
| 3 | CH═CH | NR | CONH₂ | 3 | CH═CH | CR₃R₂ | CONH₂ |
| 3 | CH═CH | NR | CH═CH₂ | 3 | CH═CH | CR₃R₂ | CH═CH₂ |
| 3 | CH═CH | NR | C≡CH | 3 | CH═CH | CR₃R₂ | C≡CH |
| 3 | CH═CH | NR | NH₂ | 3 | CH═CH | CR₃R₂ | NH₂ |
| 3 | CH═CH | CONR | C≡CH | 3 | CH═CH | SO₂NR | C≡CH |
| 3 | CH═CH | CONR | NH₂ | 3 | CH═CH | SO₂NR | NH₂ |
| 3 | CH═CH | NRCONR | I | 3 | CH═CH | NRCNHNR | I |
| 3 | CH═CH | NRCONR | F | 3 | CH═CH | NRCNHNR | F |
| 3 | CH═CH | NRCOO | OH | 3 | CH═CH | C≡C | OH |
| 3 | CH═CH | NRCOO | COOH | 3 | CH═CH | C≡C | COOH |
| 3 | CH═CH | NRCOO | SO₂H | 3 | CH═CH | C≡C | SO₂H |
| 3 | CH═CH | CH═CH | OH | 3 | CH═CH | CH═CH | N₃ |
| 3 | CH═CH | CH═CH | COOH | 3 | CH═CH | CH═CH | CH═CH₂ |
| 3 | CH═CH | CH═CH | CN | | | | |
| 4 | O | O | OH | 4 | O | S | OH |
| 4 | O | O | SH | 4 | O | S | SH |
| 4 | O | O | CONH₂ | 4 | O | S | CONH₂ |
| 4 | O | NR | SH | 4 | O | CR₄R₂ | SH |
| 4 | O | NR | Cl | 4 | O | CR₄R₂ | Cl |
| 4 | O | NR | NHR | 4 | O | CR₄R₂ | NHR |
| 4 | O | CONR | F | 4 | O | SO₂NR | F |
| 4 | O | CONR | CH═CH₂ | 4 | O | SO₂NR | CH═CH₂ |
| 4 | O | CONR | COR | 4 | O | SO₂NR | COR |
| 4 | O | NRCONR | OH | 4 | O | NRCNHNR | OH |
| 4 | O | NRCONR | NHR | 4 | O | NRCNHNR | NHR |
| 4 | O | NRCOO | CN | 4 | O | C≡C | CN |
| 4 | O | NRCOO | NHR | 4 | O | C≡C | NHR |
| 4 | O | CH═CH | Br | 4 | S | O | Br |
| 4 | O | CH═CH | C≡CH | 4 | S | O | C≡CH |
| 4 | O | CH═CH | NH₂ | 4 | S | O | NH₂ |
| 4 | S | S | Br | 4 | S | NR | Br |
| 4 | S | S | N₃ | 4 | S | NR | N₃ |
| 4 | S | S | NH₂ | 4 | S | NR | NH₂ |
| 4 | S | S | NHR | 4 | S | NR | NHR |
| 4 | S | CR₄R₂ | OH | 4 | S | CONR | OH |
| 4 | S | CR₄R₂ | COR | 4 | S | CONR | COR |
| 4 | S | SO₂NR | COOH | 4 | S | NRCONR | COOH |
| 4 | S | SO₂NR | I | 4 | S | NRCONR | I |
| 4 | S | SO₂NR | F | 4 | S | NRCONR | F |
| 4 | S | SO₂NR | COR | 4 | S | NRCONR | COR |
| 4 | S | NRCNHNR | OH | 4 | S | NRCOO | OH |
| 4 | S | NRCNHNR | I | 4 | S | NRCOO | I |
| 4 | S | NRCNHNR | F | 4 | S | NRCOO | F |
| 4 | S | C≡C | SH | 4 | S | CH═CH | SH |
| 4 | NR | O | OH | 4 | NR | S | OH |
| 4 | NR | O | SH | 4 | NR | S | SH |
| 4 | NR | O | NH₂ | 4 | NR | S | NH₂ |
| 4 | NR | NR | SO₂H | 4 | NR | CR₄R₂ | SO₂H |
| 4 | NR | NR | Cl | 4 | NR | CR₄R₂ | Cl |
| 4 | NR | NR | NHR | 4 | NR | CR₄R₂ | NHR |
| 4 | NR | NR | COR | 4 | NR | CR₄R₂ | COR |
| 4 | NR | CONR | OH | 4 | NR | SO₂NR | OH |
| 4 | NR | CONR | NH₂ | 4 | NR | SO₂NR | NH₂ |
| 4 | NR | CONR | NHR | 4 | NR | SO₂NR | NHR |
| 4 | NR | NRCONR | I | 4 | NR | NRCNHNR | I |
| 4 | NR | NRCONR | F | 4 | NR | NRCNHNR | F |
| 4 | NR | NRCOO | OH | 4 | NR | C≡C | OH |
| 4 | NR | NRCOO | CONH₂ | 4 | NR | C≡C | CONH₂ |
| 4 | NR | CH═CH | NH₂ | 4 | CR₄R₂ | OO | NH₂ |
| 4 | NR | CH═CH | NHR | 4 | CR₄R₂ | O | NHR |
| 4 | NR | CH═CH | COR | 4 | CR₄R₂ | O | COR |
| 4 | CR₄R₂ | S | OH | 4 | CR₄R₂ | NR | OH |
| 4 | CR₄R₂ | S | Br | 4 | CR₄R₂ | NR | Br |
| 4 | CR₄R₂ | CR₄R₂ | SO₂H | 4 | CR₄R₂ | CONR | SO₂H |
| 4 | CR₄R₂ | CR₄R₂ | CH═CH₂ | 4 | CR₄R₂ | CONR | CH═CH₂ |
| 4 | CR₄R₂ | CR₄R₂ | C≡CH | 4 | CR₄R₂ | CONR | C≡CH |
| 4 | CR₄R₂ | SO₂NR | F | 4 | CR₄R₂ | NRCONR | F |
| 4 | CR₄R₂ | SO₂NR | CN | 4 | CR₄R₂ | NRCONR | CN |
| 4 | CR₄R₂ | SO₂NR | N₃ | 4 | CR₄R₂ | NRCONR | N₃ |
| 4 | CR₄R₂ | NRCNHNR | CONH₂ | 4 | CR₄R₂ | NRCOO | CONH₂ |
| 4 | CR₄R₂ | NRCNHNR | CH═CH₂ | 4 | CR₄R₂ | NRCOO | CH═CH₂ |
| 4 | CR₄R₂ | NRCNHNR | C≡CH | 4 | CR₄R₂ | NRCOO | C≡CH |
| 4 | CR₄R₂ | C≡C | Cl | 4 | CR₄R₂ | CH═CH | Cl |
| 4 | CR₄R₂ | C≡C | Br | 4 | CR₄R₂ | CH═CH | Br |
| 4 | CR₄R₂ | C≡C | I | 4 | CR₄R₂ | CH═CH | I |
| 4 | CONR | O | COH | 4 | CONR | S | COH |
| 4 | CONR | O | COR | 4 | CONR | S | COR |
| 4 | CONR | NR | OH | 4 | CONR | CR₄R₂ | OH |
| 4 | CONR | NR | Br | 4 | CONR | CR₄R₂ | Br |
| 4 | CONR | NR | N₃ | 4 | CONR | CR₄R₂ | N₃ |
| 4 | CONR | CONR | Br | 4 | CONR | SO₂NR | Br |
| 4 | CONR | CONR | N₃ | 4 | CONR | SO₂NR | N₃ |
| 4 | CONR | CONR | C≡CH | 4 | CONR | SO₂NR | C≡CH |
| 4 | CONR | NRCONR | OH | 4 | CONR | NRCNHNR | OH |
| 4 | CONR | NRCONR | SH | 4 | CONR | NRCNHNR | SH |
| 4 | CONR | NRCONR | COH | 4 | CONR | NRCNHNR | COH |
| 4 | CONR | NRCOO | F | 4 | CONR | C≡C | F |
| 4 | CONR | NRCOO | CN | 4 | CONR | C≡C | CN |
| 4 | CONR | NRCOO | COR | 4 | CONR | C≡C | COR |
| 4 | CONR | CH═CH | OH | 4 | SO₂NR | O | OH |
| 4 | CONR | CH═CH | CN | 4 | SO₂NR | O | CN |
| 4 | CONR | CH═CH | COR | 4 | SO₂NR | O | COR |
| 4 | SO₂NR | S | OH | 4 | SO₂NR | NR | OH |
| 4 | SO₂NR | S | SH | 4 | SO₂NR | NR | SH |
| 4 | SO₂NR | CR₄R₂ | N₃ | 4 | SO₂NR | CONR | N₃ |
| 4 | SO₂NR | CR₄R₂ | NHR | 4 | SO₂NR | CONR | NHR |
| 4 | SO₂NR | CR₄R₂ | COH | 4 | SO₂NR | CONR | COH |
| 4 | SO₂NR | SO₂NR | COOH | 4 | SO₂NR | NRCONR | COOH |
| 4 | SO₂NR | SO₂NR | NHR | 4 | SO₂NR | NRCONR | NHR |
| 4 | SO₂NR | SO₂NR | COH | 4 | SO₂NR | NRCONR | COH |
| 4 | SO₂NR | NRCNHNR | SH | 4 | SO₂NR | NRCOO | SH |
| 4 | SO₂NR | NRCNHNR | COOH | 4 | SO₂NR | NRCOO | COOH |
| 4 | SO₂NR | NRCNHNR | SO₂H | 4 | SO₂NR | NRCOO | SO₂H |
| 4 | SO₂NR | NRCNHNR | Cl | 4 | SO₂NR | NRCOO | Cl |
| 4 | SO₂NR | C≡C | I | 4 | SO₂NR | CH═CH | I |
| 4 | SO₂NR | C≡C | F | 4 | SO₂NR | CH═CH | F |
| 4 | SO₂NR | C≡C | CN | 4 | SO₂NR | CH═CH | CN |
| 4 | NRCONR | O | F | 4 | NRCONR | S | F |
| 4 | NRCONR | O | CN | 4 | NRCONR | S | CN |
| 4 | NRCONR | O | N₃ | 4 | NRCONR | S | N₃ |
| 4 | NRCONR | NR | CONH₂ | 4 | NRCONR | CR₄R₂ | CONH₂ |
| 4 | NRCONR | NR | CH═CH₂ | 4 | NRCONR | CR₄R₂ | CH═CH₂ |
| 4 | NRCONR | NR | C≡CH | 4 | NRCONR | CR₄R₂ | C≡CH |
| 4 | NRCONR | CONR | SH | 4 | NRCONR | SO₂NR | SH |
| 4 | NRCONR | CONR | COOH | 4 | NRCONR | SO₂NR | COOH |
| 4 | NRCONR | NRCONR | CH═CH₂ | 4 | NRCONR | NRCNHNR | CH═CH₂ |
| 4 | NRCONR | NRCOO | SH | 4 | NRCONR | C≡C | SH |
| 4 | NRCONR | NRCOO | COOH | 4 | NRCONR | C≡C | COOH |
| 4 | NRCONR | CH═CH | SO₂H | 4 | NRCNHNR | O | SO₂H |
| 4 | NRCONR | CH═CH | Cl | 4 | NRCNHNR | O | Cl |
| 4 | NRCNHNR | S | Br | 4 | NRCNHNR | NR | Br |
| 4 | NRCNHNR | S | I | 4 | NRCNHNR | NR | I |
| 4 | NRCNHNR | CR₄R₂ | N₃ | 4 | NRCNHNR | CONR | N₃ |
| 4 | NRCNHNR | CR₄R₂ | CONH₂ | 4 | NRCNHNR | CONR | CONH₂ |
| 4 | NRCNHNR | SO₂NR | SO₂H | 4 | NRCNHNR | NRCONR | SO₂H |
| 4 | NRCNHNR | SO₂NR | Cl | 4 | NRCNHNR | NRCONR | Cl |
| 4 | NRCNHNR | SO₂NR | Br | 4 | NRCNHNR | NRCONR | Br |
| 4 | NRCNHNR | NRCNHNR | COR | 4 | NRCNHNR | NRCOO | COR |
| 4 | NRCNHNR | C≡C | Br | 4 | NRCNHNR | CH═CH | Br |
| 4 | NRCOO | O | COH | 4 | NRCOO | S | COH |
| 4 | NRCOO | O | COR | 4 | NRCOO | S | COR |
| 4 | NRCOO | NR | OH | 4 | NRCOO | CR₄R₂ | OH |
| 4 | NRCOO | NR | COH | 4 | NRCOO | CR₄R₂ | COH |
| 4 | NRCOO | NR | COR | 4 | NRCOO | CR₄R₂ | COR |
| 4 | NRCOO | CONR | OH | 4 | NRCOO | SO₂NR | OH |
| 4 | NRCOO | CONR | SH | 4 | NRCOO | SO₂NR | SH |
| 4 | NRCOO | NRCONR | NH₂ | 4 | NRCOO | NRCNHNR | NH₂ |
| 4 | NRCOO | NRCOO | SH | 4 | NRCOO | C≡C | SH |
| 4 | NRCOO | NRCOO | COOH | 4 | NRCOO | C≡C | COOH |
| 4 | NRCOO | CH═CH | COH | 4 | C≡C | O | COH |
| 4 | NRCOO | CH═CH | COR | 4 | C≡C | O | COR |
| 4 | C≡C | S | OH | 4 | C≡C | NR | OH |
| 4 | C≡C | CR₄R₂ | COOH | 4 | C≡C | CONR | COOH |
| 4 | C≡C | CR₄R₂ | SO₂H | 4 | C≡C | CONR | SO₂H |
| 4 | C≡C | SO₂NR | SO₂H | 4 | C≡C | NRCONR | SO₂H |
| 4 | C≡C | SO₂NR | COR | 4 | C≡C | NRCONR | COR |
| 4 | C≡C | NRCNHNR | OH | 4 | C≡C | NRCOO | OH |
| 4 | C≡C | NRCNHNR | SH | 4 | C≡C | NRCOO | SH |
| 4 | C≡C | C≡C | CONH₂ | 4 | CH═CH | CONH₂ | |
| 4 | C≡C | C≡C | COR | 4 | CH═CH | COR | |
| 4 | CH═CH | O | OH | 4 | CH═CH | S | OH |
| 4 | CH═CH | O | NH₂ | 4 | CH═CH | S | NH₂ |
| 4 | CH═CH | O | COR | 4 | CH═CH | S | COR |
| 4 | CH═CH | NR | OH | 4 | CH═CH | CR₄R₂ | OH |
| 4 | CH═CH | NR | COH | 4 | CH═CH | CR₄R₂ | COH |
| 4 | CH═CH | CONR | OH | 4 | CH═CH | SO₂NR | OH |
| 4 | CH═CH | CONR | CH═CH₂ | 4 | CH═CH | SO₂NR | CH═CH₂ |
| 4 | CH═CH | CONR | C≡CH | 4 | CH═CH | SO₂NR | C≡CH |
| 4 | CH═CH | CONR | NH₂ | 4 | CH═CH | SO₂NR | NH₂ |
| 4 | CH═CH | NRCONR | C≡CH | 4 | CH═CH | NRCNHNR | C≡CH |
| 4 | CH═CH | NRCONR | NH₂ | 4 | CH═CH | NRCNHNR | NH₂ |
| 4 | CH═CH | NRCOO | I | 4 | CH═CH | C≡C | I |
| 4 | CH═CH | NRCOO | C≡CH | 4 | CH═CH | C≡C | C≡CH |
| 4 | CH═CH | CH═CH | OH | 4 | CH═CH | CH═CH | N₃ |
| 4 | CH═CH | CH═CH | SH | 4 | CH═CH | CH═CH | CONH₂ |
| 4 | CH═CH | CH═CH | Br | 4 | CH═CH | CH═CH | NHR |
| 5 | O | O | CN | 5 | O | 5 | CN |
| 5 | O | O | N₃ | 5 | O | 5 | N₃ |
| 5 | O | NR | Br | 5 | O | CR₅R₂ | Br |
| 5 | O | NR | I | 5 | O | CR₅R₂ | I |
| 5 | O | CONR | CONH₂ | 5 | O | SO₂NR | CONH₂ |
| 5 | O | CONR | CH═CH₂ | 5 | O | SO₂NR | CH═CH₂ |
| 5 | O | NRCONR | NHR | 5 | O | NRCNHNR | NHR |
| 5 | O | NRCONR | COH | 5 | O | NRCNHNR | COH |
| 5 | O | NRCOO | OH | 5 | O | C≡C | OH |
| 5 | O | NRCOO | COOH | 5 | O | C≡C | COOH |
| 5 | O | CH═CH | OH | 5 | S | O | OH |
| 5 | O | CH═CH | C≡CH | 5 | S | O | C≡CH |
| 5 | S | S | Cl | 5 | S | NR | Cl |
| 5 | S | S | Br | 5 | S | NR | Br |
| 5 | S | S | I | 5 | S | NR | I |
| 5 | S | S | NH₂ | 5 | S | NR | NH₂ |
| 5 | S | CR₅R₂ | COOH | 5 | S | CONR | COOH |
| 5 | S | CR₅R₂ | NHR | 5 | S | CONR | NHR |
| 5 | S | CR₅R₂ | COH | 5 | S | CONR | COH |
| 5 | S | CR₅R₂ | COR | 5 | S | CONR | COR |
| 5 | S | SO₂NR | Cl | 5 | S | NRCONR | Cl |
| 5 | S | SO₂NR | CN | 5 | S | NRCONR | CN |
| 5 | S | SO₂NR | N₃ | 5 | S | NRCONR | N₃ |
| 5 | S | SO₂NR | COR | 5 | S | NRCONR | COR |
| 5 | S | NRCNHNR | OH | 5 | S | NRCOO | OH |
| 5 | S | NRCNHNR | COR | 5 | S | NRCOO | COR |
| 5 | S | C≡C | OH | 5 | S | CH═CH | OH |
| 5 | S | C≡C | SH | 5 | S | CH═CH | SH |
| 5 | NR | O | SH | 5 | NR | S | SH |
| 5 | NR | O | COOH | 5 | NR | S | COOH |
| 5 | NR | O | SO₂H | 5 | NR | S | SO₂H |
| 5 | NR | NR | OH | 5 | NR | CR₅R₂ | OH |
| 5 | NR | NR | SH | 5 | NR | CR₅R₂ | SH |
| 5 | NR | CONR | OH | 5 | NR | SO₂NR | OH |
| 5 | NR | CONR | COR | 5 | NR | SO₂NR | COR |
| 5 | NR | NRCONR | OH | 5 | NR | NRCNHNR | OH |
| 5 | NR | NRCONR | SH | 5 | NR | NRCNHNR | SH |
| 5 | NR | NRCOO | NH₂ | 5 | NR | C≡C | NH₂ |
| 5 | NR | NRCOO | NHR | 5 | NR | C≡C | NHR |
| 5 | NR | CH═CH | COOH | 5 | CR₅R₂ | O | COOH |
| 5 | NR | CH═CH | SO₂H | 5 | CR₅R₂ | O | SO₂H |
| 5 | CR₅R₂ | S | SO₂H | 5 | CR₅R₂ | NR | SO₂H |
| 5 | CR₅R₂ | S | NH₂ | 5 | CR₅R₂ | NR | NH₂ |
| 5 | CR₅R₂ | S | NHR | 5 | CR₅R₂ | NR | NHR |
| 5 | CR₅R₂ | S | COH | 5 | CR₅R₂ | NR | COH |
| 5 | CR₅R₂ | CR₅R₂ | COOH | 5 | CR₅R₂ | CONR | COOH |
| 5 | CR₅R₂ | CR₅R₂ | F | 5 | CR₅R₂ | CONR | F |
| 5 | CR₅R₂ | SO₂NR | NH₂ | 5 | CR₅R₂ | NRCONR | NH₂ |
| 5 | CR₅R₂ | SO₂NR | NHR | 5 | CR₅R₂ | NRCONR | NHR |
| 5 | CR₅R₂ | SO₂NR | COH | 5 | CR₅R₂ | NRCONR | CON |
| 5 | CR₅R₂ | NRCNHNR | COH | 5 | CR₅R₂ | NRCOO | COH |
| 5 | CR₅R₂ | NRCNHNR | COR | 5 | CR₅R₂ | NRCOO | COR |
| 5 | CR₅R₂ | C≡C | OH | 5 | CR₅R₂ | CH═CH | OH |
| 5 | CR₅R₂ | C≡C | Cl | 5 | CR₅R₂ | CH═CH | Cl |
| 5 | CONR | O | N₃ | 5 | CONR | S | N₃ |
| 5 | CONR | O | COH | 5 | CONR | S | COH |
| 5 | CONR | O | COR | 5 | CONR | S | COR |
| 5 | CONR | NR | OH | 5 | CONR | CR₅R₂ | OH |
| 5 | CONR | NR | NHR | 5 | CONR | CR₅R₂ | NHR |
| 5 | CONR | CONH | COOH | 5 | CONR | SO₂NR | COOH |
| 5 | CONR | CONR | NHR | 5 | CONR | SO₂NR | NHR |
| 5 | CONR | NRCONR | F | 5 | CONR | NRCNHNR | F |
| 5 | CONR | NRCONR | CN | 5 | CONR | NRCNHNR | CN |
| 5 | CONR | NRCOO | OH | 5 | CONR | C≡C | OH |
| 5 | CONR | NRCOO | COH | 5 | CONR | C≡C | COH |
| 5 | CONR | CH═CH | I | 5 | SO₂NR | O | I |
| 5 | CONR | CH═CH | F | 5 | SO₂NR | O | F |
| 5 | CONR | CH═CH | COR | 5 | SO₂NR | O | COR |
| 5 | SO₂NR | S | OH | 5 | SO₂NR | NR | OH |
| 5 | SO₂NR | S | SO₂H | 5 | SO₂NR | NR | SO₂H |
| 5 | SO₂NR | S | Cl | 5 | SO₂NR | NR | Cl |
| 5 | SO₂NR | CR₅R₂ | F | 5 | SO₂NR | CONR | F |
| 5 | SO₂NR | CR₅R_{2 NHR} | 5 | SO₂NR | CONR | NHR | |
| 5 | SO₂NR | SO₂NR | COOH | 5 | SO₂NR | NRCONR | COOH |
| 5 | SO₂NR | SO₂NR | SO₂H | 5 | SO₂NR | NRCONR | SO₂H |
| 5 | SO₂NR | SO₂NR | Cl | 5 | SO₂NR | NRCONR | Cl |
| 5 | SO₂NR | SO₂NR | Br | 5 | SO₂NR | NRCONR | Br |
| 5 | SO₂NR | NRCNHNR | NH₂ | 5 | SO₂NR | NRCOO | NH₂ |
| 5 | SO₂NR | NRCNHNR | NHR | 5 | SO₂NR | NRCOO | NHR |
| 5 | SO₂NR | C≡C | COOH | 5 | SO₂NR | CH═CH | COOH |
| 5 | SO₂NR | C≡C | COH | 5 | SO₂NR | CH═CH | COH |
| 5 | SO₂NR | C≡C | COR | 5 | SO₂NR | CH═CH | COR |
| 5 | NRCONR | O | OH | 5 | NRCONR | S | OH |
| 5 | NRCONR | O | SH | 5 | NRCONR | S | SH |
| 5 | NRCONR | O | COOH | 5 | NRCONR | S | COOH |
| 5 | NRCONR | O | CONH₂ | 5 | NRCONR | S | CONH₂ |
| 5 | NRCONR | NR | CN | 5 | NRCONR | CR₅R₂ | CN |
| 5 | NRCONR | NR | NHR | 5 | NRCONR | CR₅R₂ | NHR |
| 5 | NRCONR | NR | COH | 5 | NRCONR | CR₅R₂ | COH |
| 5 | NRCONR | CONR | CONH₂ | 5 | NRCONR | SO₂NR | CONH₂ |
| 5 | NRCONR | CONR | COH | 5 | NRCONR | SO₂NR | COH |
| 5 | NRCONR | CONR | COR | 5 | NRCONR | SO₂NR | COR |
| 5 | NRCONR | NRCONR | OH | 5 | NRCONR | NRCNHNR | OH |
| 5 | NRCONR | NRCONR | SH | 5 | NRCONR | NRCNHNR | SH |
| 5 | NRCONR | NRCONR | COOH | 5 | NRCONR | NRCNHNR | COOH |
| 5 | NRCONR | NRCOO | F | 5 | NRCONR | C≡C | F |
| 5 | NRCONR | NRCOO | CN | 5 | NRCONR | C≡C | CN |
| 5 | NRCONR | CH═CH | Cl | 5 | NRCNHNR | O | Cl |
| 5 | NRCONR | CH═CH | Br | 5 | NRCNHNR | O | Br |
| 5 | NRCONR | CH═CH | NH₂ | 5 | NRCNHNR | OO | NH₂ |
| 5 | NRCNHNR | S | CONH₂ | 5 | NRCNHNR | NR | CONH₂ |
| 5 | NRCNHNR | S | CH═CH₂ | 5 | NRCNHNR | NR | CH═CH₂ |
| 5 | NRCNHNR | S | C≡CH | 5 | NRCNHNR | NR | C≡CH |
| 5 | NRCNHNR | S | NH₂ | 5 | NRCNHNR | NR | NH₂ |
| 5 | NRCNHNR | S | NHR | 5 | NRCNHNR | NR | NHR |
| 5 | NRCNHNR | S | COH | 5 | NRCNHNR | NR | COH |
| 5 | NRCNHNR | CR₅R₂ | SO₂H | 5 | NRCNHNR | CONR | SO₂H |
| 5 | NRCNHNR | CR₅R₂ | Cl | 5 | NRCNHNR | CONR | Cl |
| 5 | NRCNHNR | SO₂NR | SO₂H | 5 | NRCNHNR | NRCONR | SO₂H |
| 5 | NRCNHNR | SO₂NR | Cl | 5 | NRCNHNR | NRCONR | Cl |
| 5 | NRCNHNR | SO₂NR | Br | 5 | NRCNHNR | NRCONR | Br |
| 5 | NRCNHNR | SO₂NR | I | 5 | NRCNHNR | NRCONR | I |
| 5 | NRCNHNR | SO₂NR | F | 5 | NRCNHNR | NRCONR | F |
| 5 | NRCNHNR | SO₂NR | CN | 5 | NRCNHNR | NRCONR | CN |
| 5 | NRCNHNR | NRCNHNR | NH₂ | 5 | NRCNHNR | NRCOO | NH₂ |
| 5 | NRCNHNR | NRCNHNR | NHR | 5 | NRCNHNR | NRCOO | NHR |
| 5 | NRCNHNR | NRCNHNR | COH | 5 | NRCNHNR | NRCOO | COH |
| 5 | NRCNHNR | NRCNHNR | COR | 5 | NRCNHNR | NRCOO | COR |
| 5 | NRCNHNR | C≡C | OH | 5 | NRCNHNR | CH═CH | OH |
| 5 | NRCNHNR | C≡C | SH | 5 | NRCNHNR | CH═CH | SH |
| 5 | NRCNHNR | C≡C | I | 5 | NRCNHNR | CH═CH | I |
| 5 | NRCNHNR | C≡C | NHR | 5 | NRCNHNR | CH═CH | NHR |
| 5 | NRCOO | O | COOH | 5 | NRCOO | S | COOH |
| 5 | NRCOO | O | SO₂H | 5 | NRCOO | S | SO₂H |
| 5 | NRCOO | O | NHR | 5 | NRCOO | S | NHR |
| 5 | NRCOO | O | COH | 5 | NRCOO | S | COH |
| 5 | NRCOO | O | COR | 5 | NRCOO | S | COR |
| 5 | NRCOO | NR | OH | 5 | NRCOO | CR₅R₂ | OH |
| 5 | NRCOO | NR | SH | 5 | NRCOO | CR₅R₂ | SH |
| 5 | NRCOO | NR | COOH | 5 | NRCOO | CR₅R₂ | COOH |
| 5 | NRCOO | NR | SO₂H | 5 | NRCOO | CR₅R₂ | SO₂H |
| 5 | NRCOO | CONR | NHR | 5 | NRCOO | SO₂NR | NHR |
| 5 | NRCOO | CONR | COH | 5 | NRCOO | SO₂NR | COH |
| 5 | NRCOO | CONR | COR | 5 | NRCOO | SO₂NR | COR |
| 5 | NRCOO | NRCONR | OH | 5 | NRCOO | NRCNHNR | OH |
| 5 | NRCOO | NRCONR | SH | 5 | NRCOO | NRCNHNR | SH |
| 5 | NRCOO | NRCONR | COOH | 5 | NRCOO | NRCNHNR | COOH |
| 5 | NRCOO | NRCONR | COR | 5 | NRCOO | NRCNHNR | COR |
| 5 | NRCOO | NRCOO | OH | 5 | NRCOO | C≡C | OH |
| 5 | NRCOO | NRCOO | SH | 5 | NRCOO | C≡C | SH |
| 5 | NRCOO | NRCOO | COH | 5 | NRCOO | C≡C | COH |
| 5 | NRCOO | NRCOO | COR | 5 | NRCOO | C≡C | COR |
| 5 | NRCOO | CH═CH | N₃ | 5 | C≡C | O | N₃ |
| 5 | NRCOO | CH═CH | CONH₂ | 5 | C≡C | O | CONH₂ |
| 5 | NRCOO | CH═CH | COH | 5 | C≡C | O | COH |
| 5 | NRCOO | CH═CH | COR | 5 | C≡C | O | COR |
| 5 | C≡C | S | OH | 5 | C≡C | NR | OH |
| 5 | C≡C | S | SH | 5 | C≡C | NR | SH |
| 5 | C≡C | S | COOH | 5 | C≡C | NR | COOH |
| 5 | C≡C | S | NH₂ | 5 | C≡C | NR | NH₂ |
| 5 | C≡C | CR₅R₂ | SH | 5 | C≡C | CONR | SH |
| 5 | C≡C | CR₅R₂ | SO₂H | 5 | C≡C | CONR | SO₂H |
| 5 | C≡C | CR₅R₂ | N₃ | 5 | C≡C | CONR | N₃ |
| 5 | C≡C | CR₅R₂ | COR | 5 | C≡C | CONR | COR |
| 5 | C≡C | SO₂NR | NHR | 5 | C≡C | NRCONR | NHR |
| 5 | C≡C | SO₂NR | COH | 5 | C≡C | NRCONR | COH |
| 5 | C≡C | SO₂NR | COR | 5 | C≡C | NRCONR | COR |
| 5 | C≡C | NRCNHNR | CN | 5 | C≡C | NRCOO | CN |
| 5 | C≡C | NRCNHNR | CH═CH₂ | 5 | C≡C | NRCOO | CH═CH₂ |
| 5 | C≡C | NRCNHNR | C≡CH | 5 | C≡C | NRCOO | C≡CH |
| 5 | C≡C | C≡C | COOH | 5 | C≡C | CH═CH | COOH |
| 5 | CH═CH | O | OH | 5 | CH═CH | S | OH |
| 5 | CH═CH | O | C≡CH | 5 | CH═CH | S | C≡CH |
| 5 | CH═CH | O | NH₂ | 5 | CH═CH | S | NH₂ |
| 5 | CH═CH | O | NHR | 5 | CH═CH | S | NHR |
| 5 | CH═CH | NR | NHR | 5 | CH═CH | CR₅R₂ | NHR |
| 5 | CH═CH | NR | COH | 5 | CH═CH | CR₅R₂ | COH |
| 5 | CH═CH | NR | COR | 5 | CH═CH | CR₅R₂ | COR |
| 5 | CH═CH | CONR | Br | 5 | CH═CH | SO₂NR | Br |
| 5 | CH═CH | CONR | COR | 5 | CH═CH | SO₂NR | COR |
| 5 | CH═CH | NRCONR | Br | 5 | CH═CH | NRCNHNR | Br |
| 5 | CH═CH | NRCOO | OH | 5 | CH═CH | C≡C | OH |
| 5 | CH═CH | CH═CH | COOH | 5 | CH═CH | CH═CH | CH═CH₂ |
| 5 | CH═CH | CH═CH | SO₂H | 5 | CH═CH | CH═CH | C≡CH |

R, R₂, and R₂ = hydrogen, alkyl, alkenyl, alkynyl, aryl, and heterocyclic

TABLE 9

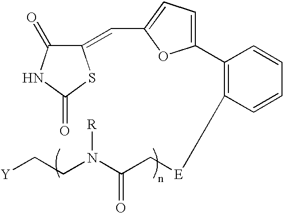

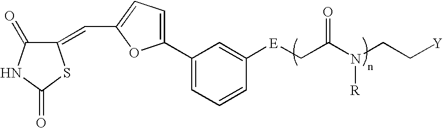

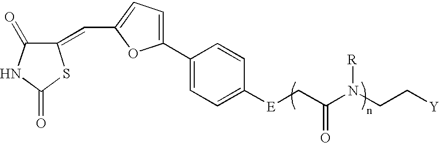

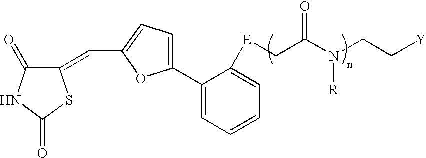

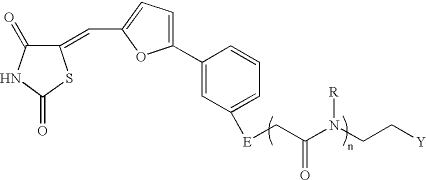

The variables E, Y, and n can have the values provided in Table 7 above. R in the compounds is alky, alkenyl, alkynyl, aromatic, or heterocyclic.

TABLE 10

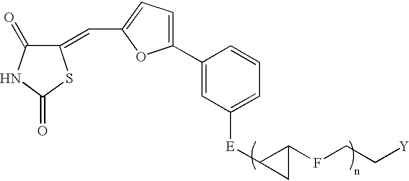

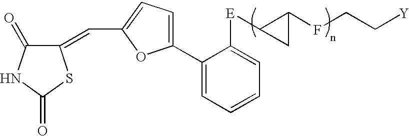

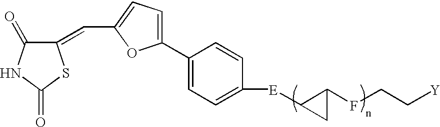

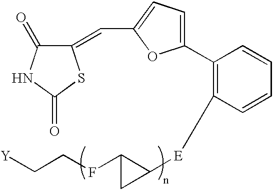

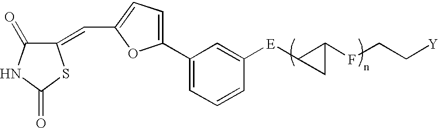

The variables E, F, Y, and n can have the values provided in Table 8 above.

TABLE 11

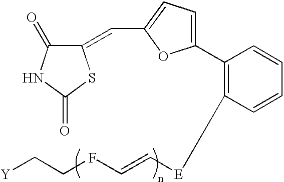

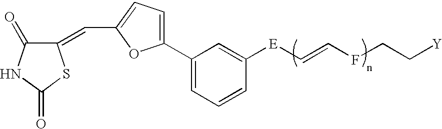

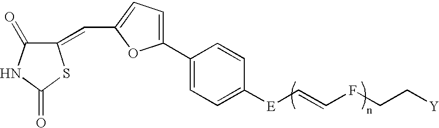

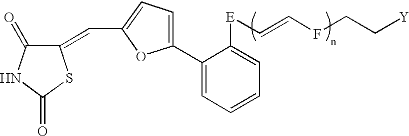

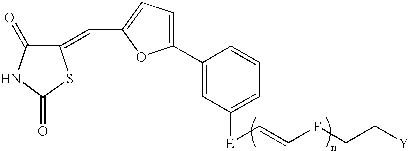

The variables E, F, Y, and n can have the values provided in Table 8 above.

TABLE 12

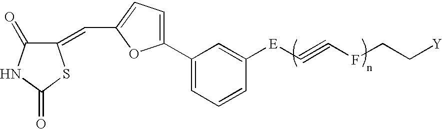

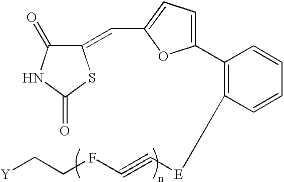

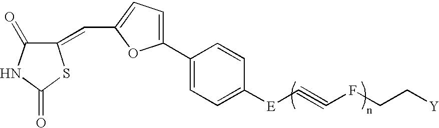

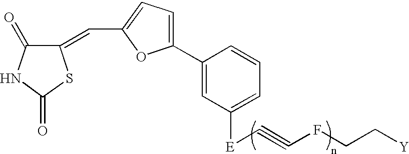

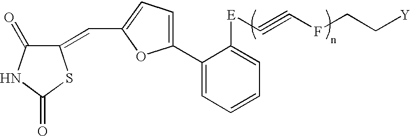

The variables E, F, Y, and n can have the values provided in Table 8 above.

### Example 23

### Preparation of [2-(4-oxo-2-thioxo-thiazolidin-3-yl)-ethyl]-carbamic Acid Tert-Butyl Ester (Compound 35)

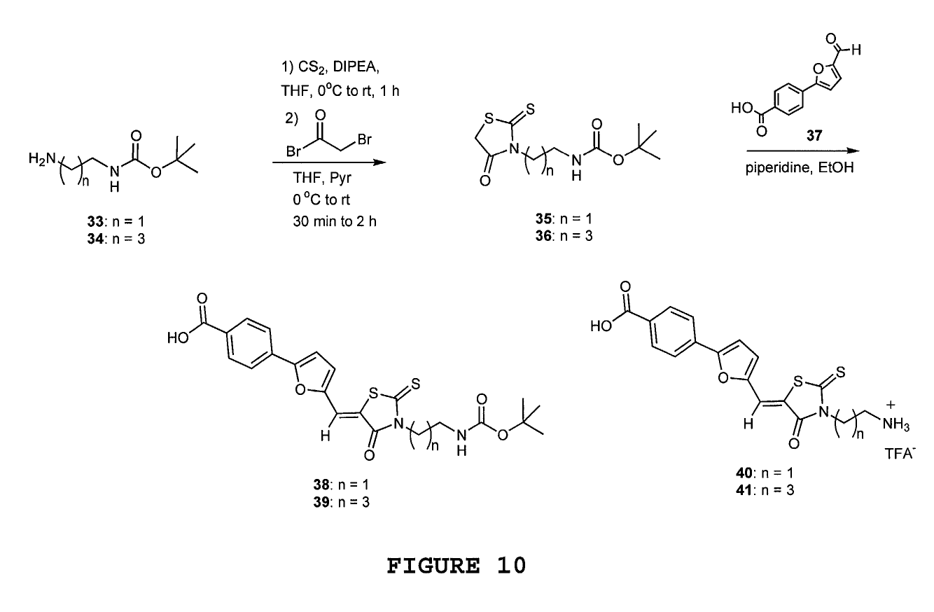

This example describes the synthesis of common ligand mimics of the invention containing a linker group following the reaction scheme shown in FIG. 10. Compound numbers correspond to the numbers in the figure.

The compound N-(2-aminoethyl)carbamic acid tert-butyl ester (compound 33, 5.03 g, 31.4 mmol) was dissolved in THF (120 ml), followed by the addition of diisopropylethylamine (5.47 ml, 31.4 mmol). Carbon disulfide (2.08 ml, 34.5 mmol) in THF (10 ml) was added to the reaction mixture at a temperature of 0° C. The reaction mixture was stirred at room temperature for 1 hour. The reaction then was cooled to a temperature of −78° C. Pyridine (5.08 ml, 62.8 mmol) and bromoacetyl bromide (3.01 ml, 34.5 mmol) were added successively to the reaction mixture, which then was stirred at −78° C. for 30 minutes, followed by stirring at room temperature for an additional 2 hours. The precipitate formed was filtered and washed with ethyl acetate.

The filtrate was concentrated in vacuo, and was quickly diluted with saturated sodium bicarbonate solution, followed by extraction with ethyl acetate. The combined organic layers were quickly washed twice with 0.4 N HCl and then once with brine. The organic layer was dried over MgSO₄, filtered, and concentrated in vacuo. The crude product was purified by flash chromatography (gradient 9:1 to 2:1 hexane/ethyl acetate) to give [2-(4-oxo-2-thioxo-thiazolidin-3-yl)-ethyl]-carbamic acid tert-butyl ester (Compound 35, 2.45 g, 29%).

¹H NMR (300 MHz, CDCl₃) δ 1.39 (s, 9H), 3.42 (m, 2H), 3.95 (s, 2H), 4.15 (s, J=5.4, 2H); ¹³C NMR (300 MHz, CDCl₃) δ 28.2, 35.1, 37.9, 44.4, 79.5, 156.0, 174.2, 201.8.

### Example 24

### Preparation of 4-{5-[3-(2-tert-butoxycarbonylamino-ethyl)-4-oxo-2-thioxo-thiazolidin-5-ylidenemethyl]-furan-2-yl}-benzoic Acid (Compound 38)

This example describes the synthesis of common ligand mimics of the invention containing a linker group following the reaction scheme shown in FIG. 10. Compound numbers correspond to the numbers in the figure.

The compounds [2-(4-oxo-2-thioxo-thiazolidin-3-yl)-ethyl]-carbamic acid tert-butyl ester (compound 35, 652 mg, 3.02 mmol) and 4-(5-formyl-furan-2-yl)-benzoic acid (compound 37, 1.0 g, 3.62 mmol) were mixed in ethanol (10 ml). Piperidine (2 drops) was added, and the reaction was stirred at 75° C. for 1 hour, followed by stirring at room temperature for an additional 18 hours. The resulting orange precipitate was collected on a fritted filter funnel. The solid was washed with ethyl acetate and then with ethyl ether to give pure 4-{5-[3-(2-tert-butoxycarbonylamino-ethyl)-4-oxo-2-thioxo-thiazolidin-5-ylidenemethyl]-furan-2-yl}-benzoic acid (compound 38, 1.05 g, 73%).

¹H NMR (300 MHz, DMSO-d₆) δ 1.34 (s, 9H), 3.29 (m, 2H), 4.12 (t, J=5.0, 2H), 6.94 (t, J=5.8, 1H), 7.39 (d, J=3.7, 1H), 7.48 (d, J=3.7, 1H), 7.69 (s, 1H), 7.95 (d, J=8.3, 2H), 8.10 (d, J=8.3, 2H)

### Example 25

### Preparation of 2-{5-[5-(4-carboxy-phenyl)-furan-2-ylmethylene]-4-oxo-2-thioxo-thiazolidin-3-yl}-ethyl-ammonium Trifluoroacetate (Compound 40)

This example describes the synthesis of common ligand mimics of the invention containing a linker group following the reaction scheme shown in FIG. 10. Compound numbers correspond to the numbers in the figure.

The compound 4-{5-[3-(2-tert-butoxycarbonylamino-ethyl)-4-oxo-2-thioxo-thiazolidin-5-ylidenemethyl]-furan-2-yl}-benzoic acid (compound 38, 500 mg, 1.05 mmol) was dissolved in a mixture of dichloromethane (7 ml) and trifluoroacetic acid (3 ml) at room temperature. The reaction mixture was stirred at room temperature for 1 hour, and the volatiles were removed in vacuo. The residue was washed with ethyl acetate and then with ethyl ether on a fritted filter funnel to give pure 2-{5-[5-(4-carboxy-phenyl)-furan-2-ylmethylene]-4-oxo-2-thioxo-thiazolidin-3-yl}-ethyl-ammonium trifluoroacetate (compound 40, 475 mg, 92%). MS m/z 374.97 (M+1).

### Example 26

### Preparation of [4-(4-oxo-2-thioxo-thiazolidin-3-yl)-butyl]-carbamic Acid Tert-Butyl Ester (Compound 36)

This example describes the synthesis of common ligand mimics of the invention containing a linker group following the reaction scheme shown in FIG. 10. Compound numbers correspond to the numbers in the figure.

The compound (4-amino-butyl)-carbamic acid tert-butyl ester (compound 34, 12.5 g, 66.3 mmol) was dissolved in THF (180 ml), followed by the addition of diisopropylethylamine (11.6 ml, 66.3 mmol). Carbon disulfide (4.4 ml, 73 mmol) in THF (20 ml) was added dropwise to the reaction mixture over 10 minutes at a temperature of 0° C. The reaction mixture was stirred at room temperature for 1 hour and then cooled to a temperature of 0° C. Pyridine (10.7 ml, 133 mmol) and bromoacetyl bromide (6.94 ml, 79.7 mmol) were added successively to the reaction mixture, which was then stirred at room temperature for 6 hours.

The precipitate formed was filtered and washed with ethyl acetate. The filtrate was concentrated in vacuo and was quickly diluted with saturated sodium bicarbonate solution, followed by extraction with ethyl ether. The combined organic layers were dried over MgSO₄, filtered, and concentrated in vacuo. The crude product was purified by flash chromatography (gradient 5:1 to 2:1 hexane/ethyl acetate) to give [2-(4-oxo-2-thioxo-thiazolidin-3-yl)-ethyl]-carbamic acid tert-butyl ester (Compound 36, 7.53 g, 37%).

### Example 27

### Preparation of 4-{5-[3-(2-tert-butoxycarbonylamino-ethyl)-4-oxo-2-thioxo-thiazolidin-5-ylidenemethyl]-furan-2-yl}-benzoic Acid (Compound 39)

This example describes the synthesis of common ligand mimics of the invention containing a linker group following the reaction scheme shown in FIG. 10. Compound numbers correspond to the numbers in the figure.

The compounds [2-(4-oxo-2-thioxo-thiazolidin-3-yl)-ethyl]-carbamic acid tert-butyl ester (Compound 36, 387 mg, 1.27 mmol) and 4-(5-formyl-furan-2-yl)-benzoic acid (compound 37, 250 mg, 1.16 mmol) were mixed in ethanol (5 ml). Piperidine (2 drops) was added and the reaction was stirred at 75° C. for 1 hour, followed by stirring at room temperature for an additional 18 hours. The resulting orange precipitate was collected on a fritted filter funnel and washed with ethyl acetate, followed by ethyl ether to give pure 4-{5-[3-(4-tert-Butoxycarbonylamino-butyl)-4-oxo-2-thioxo-thiazolidin-5-ylidenemethyl]-furan-2-yl}-benzoic acid (compound 39, 410 mg, 71%).

¹H NMR (300 MHz, DMSO-d₆) δ 1.37 (s, 9H), 1.37 (m, 2H), 1.61 (m, 2H), 2.93 (m, 2H), 4.02 (t, J=6.7, 2H), 6.79 (m, 1H), 7.38 (d, J=3.6, 1H), 7.46 (d, J=3.6, 1H), 7.66 (s, 1H), 7.93 (d, J=8.2, 2H), 8.08 (d, J=8.2, 2H).

### Example 28

### Preparation of 4-{5-[5-(4-carboxy-phenyl)-furan-2-ylmethylene]-4-oxo-2-thioxo-thiazolidin-3-yl}-butyl-ammonium trifluoroacetate (Compound 41)

This example describes the synthesis of common ligand mimics of the invention containing a linker group following the reaction scheme shown in FIG. 10. Compound numbers correspond to the numbers in the figure.

The compound 4-{5-[3-(4-tert-butoxycarbonylamino-butyl)-4-oxo-2-thioxo-thiazolidin-5-ylidenemethyl]-furan-2-yl}-benzoic acid (compound 39, 380 mg, 0.756 mmol) was dissolved in a mixture of dichloromethane (7 ml) and trifluoroacetic acid (3 ml) at room temperature. The reaction was stirred at room temperature for 1 hour, and then the volatiles were removed in vacuo. The residue was washed with ethyl acetate and then with ethyl ether on a fritted filter funnel to give pure 4-{5-[5-(4-carboxy-phenyl)-furan-2-ylmethylene]-4-oxo-2-thioxo-thiazolidin-3-yl}-butyl-ammonium trifluoroacetate (compound 41, 147 mg, 38%).

### Example 29

### Preparation of 5-(4-{3-[3-(4-fluoro-phenyl)-5-oxo-1,5-dihydro-[1,2,4]triazol-4-yl]-propenyl}-phenyl)-furan-2-carbaldehyde (compound 44)

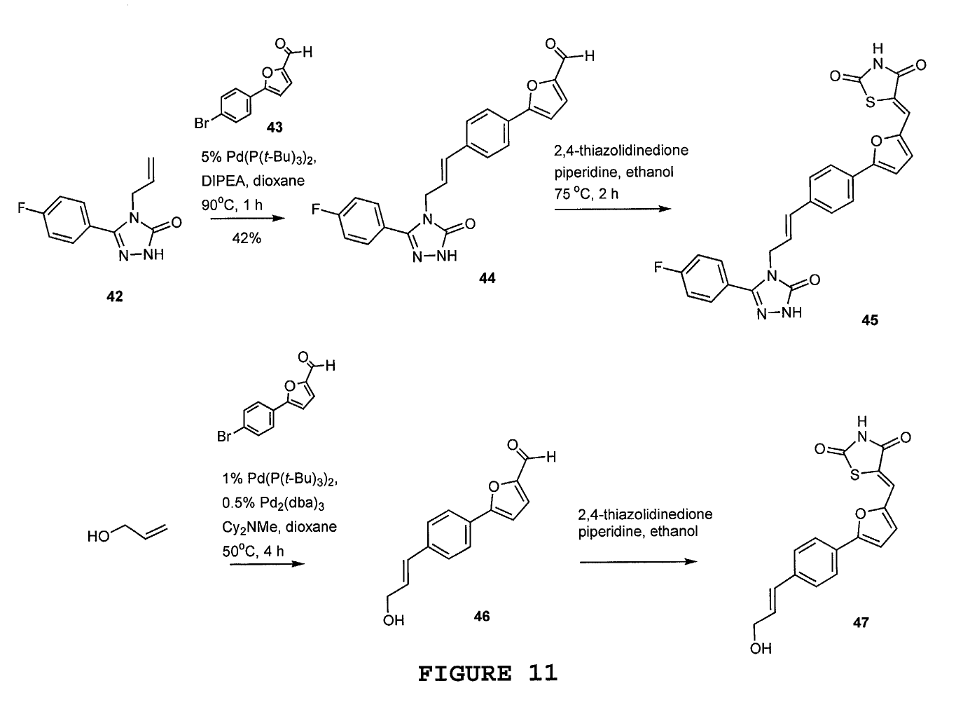

This example describes the synthesis of common ligand mimics of the invention containing a linker group following the reaction scheme shown in FIG. 11. Compound numbers correspond to the numbers in the figure.

The compounds 4-allyl-5-(4-fluoro-phenyl)-2,4-dihydro-[1,2,4]triazol-3-one (compound 42, 500 mg, 2.28 mmol) and 5-(4-bromo-phenyl)-furfural were mixed in dioxane (10 ml), followed by the addition of diisopropylethylamine (0.795 ml, 4.56 mmol). Bis(tri-tert-butylphosphine) palladium (56 mg, 0.109 mmol) was added to the reaction mixture, which then was stirred at a temperature of 90° C. for a period of 1 hour. Volatiles were removed in vacuo, and the residue was diluted in 0.2 N HCl solution, followed by extraction with ethyl acetate. Combined organic layers were dried over MgSO₄, filtered, and concentrated in vacuo. The crude product was purified by flash chromatography (gradient 7:3 to 9:1 ethyl acetate/hexanes+0.5% MeOH) to give 5-(4-{3-[3-(4-fluoro-phenyl)-5-oxo-1,5-dihydro-[1,2,4]triazol-4-yl]-propenyl}-phenyl)-furan-2-carbaldehyde (compound 44, 375 mg, 42%).

¹H NMR (300 MHz, CDCl₃) δ 4.55 (d, J=4.7, 2H), 6.31 (td, J=3.2, 16.0, 1H), 6.44 (d, J=16.0, 1H), 6.84 (d, J=3.7, 1H), 7.18 (dd, J=8.5, J_{HF}=8.5, 2H), 7.32 (d, J=3.7, 1H), 7.40 (d, J=8.3, 2H), 7.61 (dd, J=8.5, J_{HF}=5.2, 2H), 7.76 (d, J=8.3, 2H), 9.64 (s, 1H), 10.56 (s, 1H); ¹³C NMR (300 MHz, CDCl₃) δ 43.8, 107.9, 116.3 (d, J_{CF}=22), 123.2, 124.4, 125.6, 127.1, 128.7, 130.3 (d, J_{CF}=9), 132.3, 137.1, 147.0, 152.2, 155.7, 158.9, 164.1 (d, J_{CF}=250), 206.6; MS m/s 389.96 (M+1).

### Example 30

### Preparation of 5-[5-(4-{3-[3-(4-fluoro-phenyl)-5-oxo-1,5-dihydro-[1,2,4]triazol-4-yl]-propenyl}-phenyl)-furan-2-ylmethylene]-thiazolidine-2,4-dione (Compound 45)

This example describes the synthesis of common ligand mimics of the invention containing a linker group following the reaction scheme shown in FIG. 10. Compound numbers correspond to the numbers in the figure.

The compounds 5-(4-{3-[3-(4-fluoro-phenyl)-5-oxo-1,5-dihydro-[1,2,4]triazol-4-yl]-propenyl}-phenyl)-furan-2-carbaldehyde (compound 44, 70 mg, 0.181 mmol) and 2,4-thiazolidinedione (23 mg, 0.199 mmol) were mixed in ethanol (2 ml). Piperidine (0.20 ml) was added, and the reaction was stirred at 75° C. for 2 hours, followed by stirring at room temperature for an additional 18 hours. The resulting yellow precipitate was collected on a fritted filter funnel. The solid was washed with cold ethanol and then with ethyl ether to give pure 5-[5-(4-{3-[3-(4-fluoro-phenyl)-5-oxo-1,5-dihydro-[1,2,4]triazol-4-yl]-propenyl}-phenyl)-furan-2-ylmethylene]-thiazolidine-2,4-dione (compound 45, 10.6 mg, 12%).

¹H NMR (300 MHz, DMSO-d₆) δ 4.48 (bs, 2H), 6.35 (bs, 2H), 6.44 (d, J=16.0, 1H), 7.21 (d, 1H), 7.27 (d, 1H), 7.32 (dd, J=8.5, J_{HF}=8.5, 2H), 7.53 (d, J=8.3, 2H), 7.61 (s, 1H), 7.73 (m, 4H), 12.05 (s, 1H); ¹³C NMR (300 MHz, DMSO-d₆) δ 43.8, 107.9, 116.3 (d, J_{CF}=22), 123.2, 124.4, 125.6, 127.1, 128.7, 130.3 (d, J_{CF}=9), 132.3, 137.1, 147.0, 152.2, 155.7, 158.9, 164.1 (d, J_{CF}=250), 206.6; MS m/s 389.96 (M+1).

### Example 31

### Preparation of Bi-ligand Libraries of the Present Invention

This example provides a general procedure for preparing bi-ligand libraries from common ligand mimics of the invention according to the reaction scheme presented in FIG. 12a. Compound numbers correspond to the numbers in the figure.

HOBt resin (40 mg, 1.41 mmol/g, Argonaut) was swelled in a mixture of 150 μl dry THF and 50 μl of dry DMF. The resin then was added to a solution of compound 21 (2 eq, 0.226 mmol) dissolved in a mixture of 153 μl of dry DMF and 10 eq, 0.564 mmol, of DIC (N,N′-diisopropylcarbodiimide). The solution was shaken at room temperature overnight and then washed three times with dry DMF and three times with dry THF.

The resin was added to a solution of the amine (0.4 eq, 0.0226 mmol) dissolved in 200 μl dry DMF. The mixture was again shaken at room temperature overnight. The resin was filtered and washed once with 500 μl of dry DMF. The filtrate was collected and vacuum dried. Amines that have been used for the development of bi-ligand libraries of the invention using this reaction are provided in Table 1.

### Example 32

### Preparation of Bi-ligand Libraries of the Present Invention

This example provides a general procedure for preparing bi-ligand libraries from common ligand mimics of the invention according to the reaction scheme presented in FIG. 12b. Compound numbers correspond to the numbers in the figure.

HOBt resin (40 mg; 1.41 mmol/g, Argonaut) was swelled in 200 μl dry THF. The resin (4 eq, 0.226 mmol) was added to a solution of carboxylic acid (1-naphthaleneacetic acid) dissolved in a mixture of 153 μl of dry DMF and 10 eq, 0.564 mmol, of DIC. The solution was shaken at room temperature overnight and washed with 3×dry DMF and 1×dry THF.

The resin was added to a solution of compound 23 (0.4 eq, 0.0226 mmol) dissolved in 200 μl dry DMF. The solution was again shaken at room temperature overnight. The resin was filtered and washed once with 500 μl of dry DMF. The filtrate was collected and vacuum dried. Carboxylic acids that have been used for the development of bi-ligand libraries of the invention using this reaction are provided in Table 2.

### Example 33

### Preparation of Bi-ligand Libraries of the Present Invention

This example provides a general procedure for preparing bi-ligand libraries from common ligand mimics of the invention according to the reaction scheme presented in FIG. 12c. Compound numbers correspond to the numbers in the figure.

Three equivalents of an isocyanate (0.070 ml, 0.49 M in DMSO) were added to a solution of compound 23 (4 mg, 0.0112 mmol) in 0.200 ml of DMSO. The reaction was allowed to proceed overnight. Then, 20 to 30 mg of aminomethylated polystyrene Resin (NovaBiochem, Cat. No. 01-64-0383) was added to the solution. The mixture was shaken for 4 hours at room temperature. The resin was filtered off, and the solution was dried under reduced pressure to yield the desired product. Isocyanates that have been used for the development of bi-ligand libraries of the invention using this reaction are provided in Table 3.

### Example 34

### Preparation of Bi-ligand Libraries of the Present Invention

This example provides a general procedure for preparing bi-ligand libraries from common ligand mimics of the invention according to the reaction scheme presented in FIG. 13. Compound numbers correspond to the numbers in the figure.

In a 10 ml vial, DBU (1,8-diazabicyclo [5.4.0]undec-7-ene (760 mg, 5 mmol) was added to a mixture of compound 26 (860 mg, 5 mmol) and compound 27 (7.5 mmol) in dioxane. The reaction mixture was agitated under microwave irradiation at a temperature of 170° C. for a period of 40 minutes. The solvent was removed from the mixture, and the resultant oil residue was subjected to flash chromatography to provide desired compound 28 (65% yield).

Compound 28 (6.4 mmol) was suspended in a mixture of water (5 ml) and MeOH (15 ml). LiOH (307 mg, 12.8 mmol) was added, and the solution was refluxed for 2 hours. Solvent was removed from the reaction mixture, and the residue was dissolved in water. Dilute hydrochloric acid was added dropwise, forming a white precipitate that then was collected.

HOBt resin (20 mg, 1.41 mmol/g, Argonaut) was swelled in 100 μl dry THF. The resin was added to a solution of compound 29 (2 eq, 0.056 mmol) dissolved in a mixture 100 μl of dry DMF and 6 eq (0.168 mmol) of DIC. The solution was shaken at room temperature overnight and washed with 3×dry DMF and 2×dry THF.

The resin then was added to a solution of the amine (0.5 eq, 0.014 mmol), dissolved in 200 μl dry DMF. The mixture was shaken at room temperature overnight. The resin was filtered and washed twice with 100 μl of dry DMF to provide compound 30. The filtrate of compound 30 was collected and vacuum dried.

Compound 30 was dissolved in a mixture of TFA (trifluoroacetic acid) and dichloroethane (DCE, 50%) and was shaken at room temperature for 20 minutes. Solvent was removed from the mixture, and the residue (compound 30) was ready for the next step reaction.

HOBt resin (20 mg; 1.41 mmol/g, Argonaut) was swelled in a mixture of 100 μl dry THF and 100 μl of dry DMF. It was added to CLM 1 (2 eq, 0.056 mmol) dissolved in 200 μl of dry DMF and 6 eq (0.168 mmol) of DIC. The solution was shaken at room temperature overnight and washed with 3×dry DMF and 3×dry THF.

The resin was then added to the residue of the deBoc reaction (compound 30), which was dissolved in 200 μl dry THF. The mixture was shaken at room temperature overnight, and the resin was filtered and washed twice with 100 μl of dry DMF. The filtrate, compound 31, was collected and vacuum dried. Amines that have been used for the development of bi-ligand libraries of the invention using this reaction are provided in Table 4.

### Example 35

### Preparation of Bi-ligand Libraries of the Present Invention

This example provides a general procedure for preparing bi-ligand libraries from common ligand mimics of the invention according to the reaction scheme presented in FIG. 14. Compound numbers correspond to the numbers in the figure.

Et₃N resin (53 mg, 3.2 mmol/g, Fluka) was added to a mixture of 4-mercaptobenzoic acid (0.056 mmol, 8.6 mg) and alkyl bromide (0.067 mmol) in CH₃CN. The mixture was shaken at room temperature overnight, after which the resin was filtered and washed twice with 100 μl of CH₃CN. The filtrate was collected and vacuum dried.

HOBt resin (10 mg, 1.41 mmol/g, Argonaut) was swelled in 100 μl dry THF and was added to the residue of the last step reaction, which was dissolved in a mixture of 100 μl of dry DMF and 6 eq (0.084 mmol) of DIC. The solution was shaken at room temperature overnight and washed with 3×dry DMF and 2×dry THF.

The resin then was added to CLM 4 (0.5 eq, 0.007 mmol) dissolved in 200 μl dry DMF. The solution was shaken at room temperature overnight. The resin was filtered and washed twice with 100 μl of dry DMF. The filtrate was collected and vacuum dried. Alkylhalides that have been used for the development of bi-ligand libraries of the invention using this reaction are provided in Table 5.

### Example 36

### Screening of Selected Thiazolidinediones for Binding to Dehydrogenases and Oxidoreductases

This example describes the screening of two thiazolidinedione common ligand mimics for binding activity to a variety of dehydrogenases and oxidoreductases.

The thiazolidinedione compounds 4-[5-(2,4-dioxo-thiazolidin-5-ylidenemethyl)-furan-2-yl]-benzoic acid and 5-[5-(2,4-dioxo-thiazolidin-5-ylidenemethyl)-furan-2-yl]-2-hydroxy-benzoic acid were produced following the method of Examples 1 and 5. The compounds were screened for binding to the following enzymes: dihydrodipicolinate reductase (DHPR), lactate dehydrogenase (LDH), alcohol dehydrogenase (ADH), dihydrofolate reductase (DHFR), 1-deoxy-D-xylulose-5-phosphate reductase (DOXPR), glyceraldehyde-3-phosphate dehydrogenase (GAPDH), 3-isopropylmalate (IPMDH), inosine-5′-monophosphate dehydrogenase (IMPDH), aldose reductase (AR), and HMG CoA reductase (HMGCoAR).

DHPR

For DHPR analysis, the compounds were screened using a kinetic protocol that spectrophotometrically evaluates oxidation of NADPH.

Stock solutions of each of the reagents were prepared in the following concentrations. Dilutions of the stock solutions were prepared prior to running the assay in the concentrations indicated below. DHPR was diluted in 10 mM HEPES at a pH of 7.4. DHPS (dihydrodipicolinate synthase) was not diluted and was stored in eppindorf tubes.

| | Stock | Final | Volume needed |
|---|---|---|---|
| ddH₂O | | | 798 μl |
| HEPES (pH 7.8) | 1 M | 0.1 M | 100 μl |
| Pyruvate | 50 mM | 1 mM | 20 μl |
| NADPH | 1 mM | 6 μM | 6 μl |
| L-ASA | 28.8 mM | 40 μM | 13.9 μl |
| DHPS | 1 mg/ml | | 7 μl |
| DHPR | 1:1000 dilution of | | 5 μl |
| | 1 mg/ml stock | | |
| Inhibitor | 15 mM | 100 μM | 6.7 μl |
| | | | (0.67 DMSO) |
| DMSO | 100% | 5% | 43.3 μl |
| Total Assay volume = | | | 1000 μl |

The L-ASA (L-aspartate semialdehyde) solution was prepared in the following manner. 180 μM stock solution of ASA was prepared. 100 μl of the ASA stock solution was mixed with 150 μl of concentrated NaHCO₃ and 375 μl of H₂O. For use in the assay, 28.8 mM L-ASA was equal to 625 μl of the solution. The L-ASA stock solution was kept at a temperature of −20° C. After dilution, the pH of the 28.8 mM solution was checked and maintained between 1 and 2.

The DHPS reaction was monitored at 340 nm prior to and after addition of the inhibitor to detect background reaction with the inhibitor. The solution for background detection was a 945 μl solution containing 0.1 HEPES (pH 7.8), 1 mM pyruvate, 6 μM NADPH, 40 μM L-ASA, and 7 μl of 1 mg/ml DHPS at 25° C. in the volumes provided above. The sample solution was then mixed and incubated for 10 minutes. Next, 500 nM solutions of the inhibitors and enough DMSO to provide a final DMSO concentration of 5% of the total assay volume were added. The solution was mixed and incubated for an additional 6 minutes.

In DHPR samples, 5 μl of the diluted DHPR enzyme were added. The sample was mixed for 20 seconds and then the reaction was run for 10 minutes. After a 50 second lag, the samples were read in a Cary spectrophotometer at 340 nm. Reading of the samples was continued until 300 seconds. Cuvette #1 contained the control reaction (no inhibitor), and cuvette #2 contained the positive control reaction in which Cibacron Blue at 2.58 μM was substituted for inhibitor to yield 70 to 80% inhibition. The substrate was kept at a level at least 10 times the Km. The final concentration of L-ASA was about 1 mM.

LDH

For LDH analysis, the compounds were screened using a kinetic protocol that spectrophotometrically evaluates oxidation of NADH.

Stock solutions of each of the reagents were prepared in the following concentrations. Dilutions of the stock solutions were prepared prior to running the assay in the concentrations indicated below.

| | Stock | Final | Volume needed |
|---|---|---|---|
| ddH₂O | | | 780 μl |
| HEPES (pH 7.4) | 1 M | 0.1 M | 100 μl |
| Pyruvate | 50 mM | 2.5 mM | 50 μl |
| NADH | 1 mM | 10 μM | 10 μl |
| LDH | 1:2000 dilution of | | 10 μl |
| | 1 mg/ml stock | | |
| Inhibitor | 15 mM | 100 μM | 6.7 μl |
| | | | (0.67% DMSO) |
| DMSO | 100% | 5% | 43.3 μl |
| Total Assay volume = | | | 1000 μl |

The LDH reaction was monitored at 340 nm prior to and after addition of the inhibitor to detect background reaction with the inhibitor. Solutions of 100 μl of the inhibitors in DMSO were prepared to provide a final DMSO concentration of 5% of the total assay volume. These solutions were incubated for 6 minutes at 25° C. in a 990 μl of a solution containing 0.1 M HEPES, pH 7.4, 10 μM NADH, and 2.5 mM of pyruvate. The reaction was then initiated with 10 μl of LDH from Rabbit Muscle (0.5 μ/ml; 1:2000 dilution of 1.0 mg/ml). After the enzyme was added, the solution was mixed for 20 seconds, and the reaction was run for 10 minutes. After a 50 second lag, the samples were read in a Cary spectrophotometer at 340 nm. Reading of the samples was continued until 300 seconds. Cuvette #1 contained the control reaction (no inhibitor), and cuvette #2 contained the positive control reaction in which Cibacron Blue at 10.3 μM was substituted for inhibitor to yield 50 to 70% inhibition. The substrate was kept at a level at least 10 times the Km.

ADH

For ADH analysis, the compounds were screened using a kinetic protocol that spectrophotometrically evaluates reduction of NAD+.

Stock solutions of each of the reagents were prepared in the following concentrations. Dilutions of the stock solutions were prepared prior to running the assay in the concentrations indicated below.

| | Stock | Final | Volume needed |
|---|---|---|---|
| DdH₂O | | | 787 μl |
| HEPES (pH 8.0) | 1 M | 0.1 M | 100 μl |
| EtOH | 10 M | 130 mM | 13 μl |
| NAD+ | 2 mM | 80 μM | 40 μl |
| ADH | 1:400 dilution of | | 10 μl |
| | 1 mg/ml stock | | |
| Inhibitor | 15 mM | 100 μM | 6.7 μl |
| | | | (0.67% DMSO) |
| DMSO | 100% | 5% | 43.3 μl |
| Total Assay volume = | | | 1000 μl |

The ADH reaction was monitored at 340 nm prior to and after addition of the inhibitor to detect background reaction with the inhibitor. Solutions of 100 μl of the inhibitors in DMSO were prepared to provide a final DMSO concentration of 5% of the total assay volume. These solutions were incubated for 6 minutes at 25° C. in a 990 μl of a solution containing 0.1 M HEPES, pH 8.0, 80 μM NAD+, and 130 mM of ethanol. The reaction was then initiated with 10 μl of ADH from Bakers Yeast (3.3 μg/ml; 1:400 dilution of 1.0 mg/ml). After the enzyme was added, the solution was mixed for 20 seconds, and the reaction was run for 10 minutes. After a 50 second lag, the samples were read in a Cary spectrophotometer at 340 nm. Reading of the samples was continued until 300 seconds. Cuvette #1 contained the control reaction (no inhibitor), and cuvette #2 contained the positive control reaction in which Cibacron Blue at 15.5 μM was substituted for inhibitor to yield 50 to 60% inhibition. The substrate was kept at a level at least 10 times the Km. The final concentration of pyruvate was about 2.5 mM.

Where only a simple read was desired, as in the case of NAD+concentration determination, 13 μl (10 M stock) of ethanol was used to drive the reaction, and 10 μl of pure enzyme (1 mg/ml) was used. NAD+was soluble at 2 mM, which allowed the concentration determination step to be skipped. In this situation, the procedure was as follows. All of the ingredients except for the enzyme were mixed together. The solution was mixed well and the absorbance at 340 nm read. The enzyme was added and read again at OD 340 after the absorbance stopped changing, generally 10 to 15 minutes after the enzyme was added.

DHFR

For DHFR analysis, the compounds were screened using a kinetic protocol that spectrophotometrically evaluates oxidation of NADH.

Stock solutions of each of the reagents were prepared in the following concentrations. Dilutions of the stock solutions were prepared prior to running the assay in the concentrations indicated below. H₂ folate was dissolved in DMSO to about 10 mM and then diluted with water to a concentration of 0.1 mM.

| | Stock | Final | Volume needed |
|---|---|---|---|
| ddH₂O | | | 616 μl |
| Tris-HCl (pH 7.0) | 1 M | 0.1 M | 100 μl |
| KCl | 1 mM | 0.15 M | 150 μl |
| H₂ Folate | 0.1 mM | 5 μM | 50 μl |
| NADPH | 2 mM | 52 μM | 26 μl |
| DHFR | 1:85 dilution of 4 | | 8 μl |
| | mg/ml stock | | |
| Inhibitor | 15 mM | 100 μM | 6.7 μl |
| | | | (0.67% DMSO) |
| DMSO | 100% | 5% | 43.3 μl |
| Total Assay volume = | | | 1000 μl |

The DHFR reaction was monitored at 340 nm prior to and after addition of the inhibitor to detect background reaction with the inhibitor. Solutions of 100 μl of the inhibitors in DMSO were prepared to provide a final DMSO concentration of 5% of the total assay volume. These solutions were incubated for 6 minutes at 25° C. in a 992 μl of a solution containing 0.1 M Tris-HCl, pH 7.0, 150 mM KCl, 5 μM H₂ folate, and 52 μM NADH. The oxidation reaction was then initiated with 8 μl of DHFR (0.047 mg/ml). After the enzyme was added, the solution was mixed for 20 seconds, and the reaction was run for 10 minutes. After a 50 second lag, the samples were read in a Cary spectrophotometer at 340 nm. Reading of the samples was continued until 300 seconds. Cuvette #1 always contained the control reaction (no inhibitor), and cuvette #2 always contained the positive control reaction in which Cibacron Blue at 3 μM was substituted for inhibitor to yield 50 to 70° inhibition. The substrate was kept at a level at least 10 times the Km.

DOXPR

For DOXPR analysis, the compounds were screened using a kinetic protocol that spectrophotometrically evaluates oxidation of NADPH.

Stock solutions of each of the reagents were prepared in the following concentrations. Dilutions of the stock solutions were prepared prior to running the assay in the concentrations indicated below. DOXPR was diluted in 10 mM HEPES at a pH of 7.4.

| | Stock | Final | Volume needed |
|---|---|---|---|
| ddH₂O | | | 707 μl |
| HEPES (pH 7.4) | 1 M | 0.1 M | 100 μl |
| DOXP | 10 mM | 1.15 mM | 115 μl |
| NADPH | 1 mM | 8 μM | 8 μl |
| MnCl₂ | 100 mM | 1 mM | 10 μl |
| DOXPR | 1:200 dilution of | | 10 μl |
| | 2 mg/ml stock | | |
| Inhibitor | 15 mM | 100 μM | 6.7 μl |
| | | | (0.67% DMSO) |
| DMSO | 100% | 5% | 43.3 μl |
| Total Assay volume = | | | 1000 μl |

The DOXPR reaction was monitored at 340 nm prior to and after addition of the inhibitor to detect background reaction with the inhibitor. Solutions of the inhibitors in DMSO were prepared to provide a final DMSO concentration of 5% of the total assay volume. These solutions were incubated for 6 minutes at 25° C. in a 990 μl of a solution containing 0.1 M HEPES, pH 7.4, 1 mM MnCl₂ 1.15 mM DOXP, and 8 μM NADPH. The oxidation reaction was then initiated with 10 μl of DOXP reductoisomerase (10 μg/ml). After the enzyme was added, the solution was mixed for 20 seconds, and the reaction was run for 10 minutes. After a 50 second lag, the samples were read in a Cary spectrophotometer at 340 nm. Reading of the samples was continued until 300 seconds. Cuvette #1 contained the control reaction (no inhibitor), and cuvette #2 contained the positive control reaction in which Cibacron Blue at 10.32 μM was substituted for inhibitor to yield 70 to 80% inhibition. The substrate was kept at a level at least 10 times the Km.

GAPDH

For GAPDH analysis, the compounds were screened using a kinetic protocol that spectrophotometrically evaluates reduction of NAD+.

Stock solutions of each of the reagents were prepared in the following concentrations. Dilutions of the stock solutions were prepared prior to running the assay in the concentrations indicated below.

| | | |
|---|---|---|
| | | Volume |
| Stock | Final | needed |

| | | | | | |
|---|---|---|---|---|---|
| ddH₂O | | | | | 739 μl |
| Triethanolamine | 1 | M | 25 | mM | 125 μl |
| (pH 7.5) | | | | | |
| GAP | 50 | mM | 145 | μM | 3 μl |
| NAD+ | 5 | mM | 0.211 | mM | 42 μl |
| Sodium Arsenate | 200 | mM | 5 | mM | 25 μl |
| 2-BME | 500 | mM | 3 | mM | 6 μl |
| GAPDH | 1:200 | dilution | | | 10 μl |
| | | of 1 | | | |
| | | mg/ml | | | |
| | | stock | | | |
| Inhibitor | 12.5 | mM | 100 | μM | 8 μl |
| | | | | | (total 5% |
| | | | | | DMSO) |
| DMSO | 100% | | 5% | | 42 μl |

Total Assay volume = 1000 μl

The GAPDH reaction was monitored at 340 nm prior to and after addition of the inhibitor to detect background reaction with the inhibitor. Solutions of 100 μl of the inhibitors incubated for 6 minutes at 250C in a 990 μl of a solution containing 125 mM triethanolamine, pH 7.5, 145 μM glyceraldehyde 3-phosphate (GAP), 0.211 mM NAD, 5 mM sodium arsenate, and 3 mM β-metcaptoethanol (2-BME). The reaction was then initiated with 10 μl of E. coli GAPDH (1:200 dilution of 1.0 mg/ml). After the enzyme was added, the solution was mixed for 20 seconds, and the reaction was run for 10 minutes. After a 50 second lag, the samples were read in a Cary spectrophotometer at 340 nm. Reading of the samples was continued until 300 seconds. The final concentration of DMSO in a cuvette was about 5% of the total assay volume. Cuvette #1 contained the control reaction (no inhibitor).

GAP for use in this experiment was deprotected from the diethyl acetal in the following manner. Water was boiled in recrystallizing dish. Dowex (1.5 mg) and GAP (200 mg; SIGMA G-5376) were weighed and placed in a 15 ml conical tube. The Dowex and GAP were resuspended in 2 ml dH₂O, followed by shaking of the tube until the GAP dissolved. The tube was then immersed, while shaking, in the boiling water for 3 minutes. Next, the tube was placed in an ice bath to cool for 5 minutes. As the sample cooled, a resin settled to the bottom of the test tube, allowing removal of the supernatant with a pasteur pipette. The supernatant was filtered through a 0.45 or 0.2 μM cellulose acetate syringe filter.

The filtered supernatant was retained, and another 1 ml of dH₂o was added to the resin tube. The tube was then shaken and centrifuged for 5 minutes at 3,000 rpm. The supernatant was again removed with a pasteur pipette and passed through a 0.45 or 0.2 μM cellulose acetate syringe filter. The two supernatant aliquots were then pooled to provide a total GAP concentration of about 50 mM. The GAP was then divided into 100 μl aliquots and stored at −20° C. until use.

IMPDH

For IMPDH analysis, the compounds were screened using a kinetic protocol that spectrophotometrically evaluates reduction of NAD+.

Stock solutions of each of the reagents were prepared in the following concentrations. Dilutions of the stock solutions were prepared prior to running the assay in the concentrations indicated below.

| | Stock | | Final | | Volume needed |
|---|---|---|---|---|---|
| ddH₂O | | | | | 447 μl |
| Tris-HCl (pH 8.0) | 1 | M | 0.1 | M | 100 μl |
| KCl | 1 | M | 0.25 | M | 250 μl |
| NAD+ | 2 | mM | 30 | μM | 15 μl |
| IMP | 6 | mM | 600 | μM | 100 μl |
| Glycerol | 10% | | 0.3% | | 30 μl |
| IMPDH | 0.75 | mg/ml, | | | 8 μl |
| | | undiluted | | | |
| Inhibitor | 15 | mM | 100 | μM | 6.7 μl |
| | | | | | (0.67% DMSO) |
| DMSO | 100% | | 5% | | 43.3 μl |

Total Assay volume = 1000 μl

The IMPDH reaction was monitored at 340 nm prior to and after addition of the inhibitor to detect background reaction with the inhibitor. Solutions of 100 μl of the inhibitors in DMSO were prepared to provide a final DMSO concentration of 5% of the total assay volume. These solutions were incubated for 6 minutes at 37° C. in a 992 μl of a solution containing 0.1 M Tris-HCl, pH 8.0, 0.25 M KCl, 0.3% glycerol, 30 μM NAD+, and 600 μM IMP (inosine monophosphate). The reaction was then initiated with 8 μμl of IMPDH (0.75 μg/ml). After the enzyme was added, the solution was mixed for 20 seconds, and the reaction was run for 10 minutes. After a 50 second lag, the samples were read in a Cary spectrophotometer at 340 nm. Reading of the samples was continued until 300 seconds. Cuvette #1 contained the control reaction (no inhibitor), and cuvette #2 contained the positive control reaction in which Cibacron Blue was substituted for inhibitor. The substrate was kept at a level at least 10 times the Km.

HMGCoAR

For HMGCoAR analysis, the compounds were screened using a kinetic protocol that spectrophotometrically evaluates oxidation of NADPH.

Stock solutions of each of the reagents were prepared in the following concentrations. Dilutions of the stock solutions were prepared prior to running the assay in the concentrations indicated below. The enzyme was diluted in 1 M NaCl. To prepare the dilution buffer, 10 μl of HMGCoAR (1 mg/ml) was mixed with 133 μl of 3 M NaCl solution and 257 μl of 25 mM KH₂PO₄ buffer (pH 7.5; containing 50 mM NaCl, μl mM EDTA (ethylenediaminetetraacetic acid), and 5 mM DTT (dithiothreitol).

| | Stock | | Final | | Volume needed |
|---|---|---|---|---|---|
| ddH₂O | | | | | 841 μl |
| KH₂P0₄ (pH 7.5) | 1 | M | 25 | mM | 25 μl |
| HMGCoA | 10 | mM | 160 | mM | 16 μl |
| NADPH | 1 | mM | 13 | μM | 13 μl |
| NaCl | 1 | M | 50 | mM | 50 μl |
| EDTA | 50 | mM | 1 | mM | 20 μl |
| DTT | 500 | mM | 5 | mM | 10 μl |
| HMGCoAR | 1:40 | dilution of | | | 5 μl |
| | | 0.65 mg/ml | | | |
| | | stock | | | |
| Inhibitor | 10 | mM | 100 | μM | 10 μl |
| DMSO | 100% | | 2% | | 10 μl |

Total Assay volume = 1000 μl

The HMGCoAR reaction was monitored at 340 nm prior to and after addition of the inhibitor to detect background reaction with the inhibitor. Solutions of 500 nM of the inhibitors in DMSO were prepared to provide a final DMSO concentration of 2% of the total assay volume. These solutions were incubated for 6 minutes at 25° C. in a 994 μl of a solution containing 25 mM KH₂PO₄, pH 7.5, 160 μM HMGCoA, 13 μM NADPH, 50 mM NaCl, 1 mM EDTA, and 5 mM DTT. The reaction was then initiated with 5 μl of HMGCoAR enzyme (1:40 dilution of 0.65 mg/ml). After the enzyme was added, the solution was mixed for 20 seconds, and the reaction was run for 10 minutes. After a 50 second lag, the samples were read in a Cary spectrophotometer at 340 nm. Reading of the samples was continued until 300 seconds. Cuvette #1 contained the control reaction (no inhibitor), and cuvette #2 contained the positive control reaction in which Cibacron Blue at 2.05 μM was substituted for inhibitor to yield 50 to 70% inhibition. The substrate was kept at a level at least 10 times the Km.

IPMDH

For IPMDH analysis, the compounds were screened using a kinetic protocol that spectrophotometrically evaluates reduction of NAD.

Stock solutions of each of the reagents were prepared in the following concentrations. Dilutions of the stock solutions were prepared prior to running the assay in the concentrations indicated below.

| | Stock | | Final | | Volume needed |
|---|---|---|---|---|---|
| ddH₂O | | | | | 407 μl |
| KH₂P0₄ (pH 7.6) | 1 | M | 20 | mM | 20 μl |
| KCl | 1 | M | 0.3 | M | 300 μl |
| MNCl₂ | 20 | mM | 0.2 | mM | 10 μl |
| NAD | 3.3 | mM | 109 | μM | 33 μl |
| IPM | 2 | mM | 340 | μM | 170 μl |
| E. coli IPMDH | 1:300 | dilution of | | | 10 μl |
| | | 2.57 mg/ml | | | |
| | | stock | | | |
| Inhibitor | 16 | mM | 200 | μM | 12.5 μl |
| DMSO | 100% | | 5% | | 37.5 μl |

Total Assay volume = 1000 μl

The IPMDH reaction was monitored at 340 nm prior to and after addition of the inhibitor to detect background reaction with the inhibitor. Inhibitor was incubated for 5 minutes at 37° C. in a 990 μl of a solution containing 20 mM potassium phosphate, pH 7.6, 0.3 M potassium chloride, 0.2 mM manganese chloride, 109 μM NAD, and 340 μM DL-threo-3-isopropylmalic acid (IPM). The reaction was then initiated with 10 μl of E. coli isopropylmalate dehydrogenase (1:300 dilution of 2.57 mg/ml). After the enzyme was added, the solution was mixed for 20 seconds, and the reaction was run for 10 minutes. After a 50 second lag, the samples were read in a Cary spectrophotometer at 340 nm. Reading of the samples was continued until 300 seconds. The final concentration of DMSO in the cuvette was 5% of the total assay volume. Cuvette #1 contained the control reaction (no inhibitor), and cuvette #2 contained the positive control reaction in which Cibacron Blue was substituted for inhibitor to yield 30 to 70% inhibition. The substrate was kept at a level at least 10 times the Km.

AR

For AR analysis, the compounds were screened using a kinetic protocol that spectrophotometrically measures enzyme activity.

Stock solutions of each of the reagents were prepared in the following concentrations. Dilutions of the stock solutions were prepared prior to running the assay in the concentrations indicated below.

| | Stock | | Final | | Volume needed |
|---|---|---|---|---|---|
| ddH₂O | | | | | 565.5 μl |
| KH₂PO₄ (pH 7.5) | 1 | M | 100 | mM | 100 μl |
| Ammonium Sulfate | 1 | M | 0.3 | M | 300 μl |
| EDTA | 500 | mM | 1 | mM | 2 μl |
| NADPH | 1 | mM | 3.8 | μM | 3.8 μl |
| Glyceraldehyde | 100 | mM | 171 | μM | 1.7 μl |
| DTT | 100 | mM | 0.1 | mM | 1 μl |
| Human ALDR | 1:5 | dilution of | | | 10 μl |
| | | 0.55 mg/ml | | | |
| | | stock | | | |
| Inhibitor | 12.5 | mM | 200 | μM | 16 μl |

Total Assay volume = 1000 μl

The AR reaction was monitored at 340 nm prior to and after addition of the inhibitor to detect background reaction with the inhibitor. Solutions of 100 μl of the inhibitors in DMSO were prepared to provide a final DMSO concentration of 5% of the total assay volume. These solutions were incubated for 5 minutes at 25° C. in a 990 μl of a solution containing 100 mM potassium phosphate, pH 7.5, 0.3 M ammonium sulfate, 1.0 mM ethylenediaminetetraacetic acid (EDTA), 3.8 μM B-Nicotinamide adenine dinucleotide phosphate (NADPH), 171 μM DL-glyceraldehyde and 0.1 mM DL-dithiothreitol. The reaction was then initiated with 10 μl of Human Aldose Reductase (1:5 dilution of 0.55 mg/ml). After the enzyme was added, the solution was mixed for 20 seconds, and the reaction was run for 10 minutes. After a 50 second lag, the samples were read in a Cary spectrophotometer at 340 nm. Reading of the samples was continued until 300 seconds. The final DMSO concentration in the cuvette was 5%. Cuvette #1 contained the control reaction (no inhibitor), and cuvette #2 contained the positive control reaction in which Cibacron Blue was substituted for inhibitor to yield 30 to 70% inhibition. The substrate was kept at a level at least 10 times the Km.

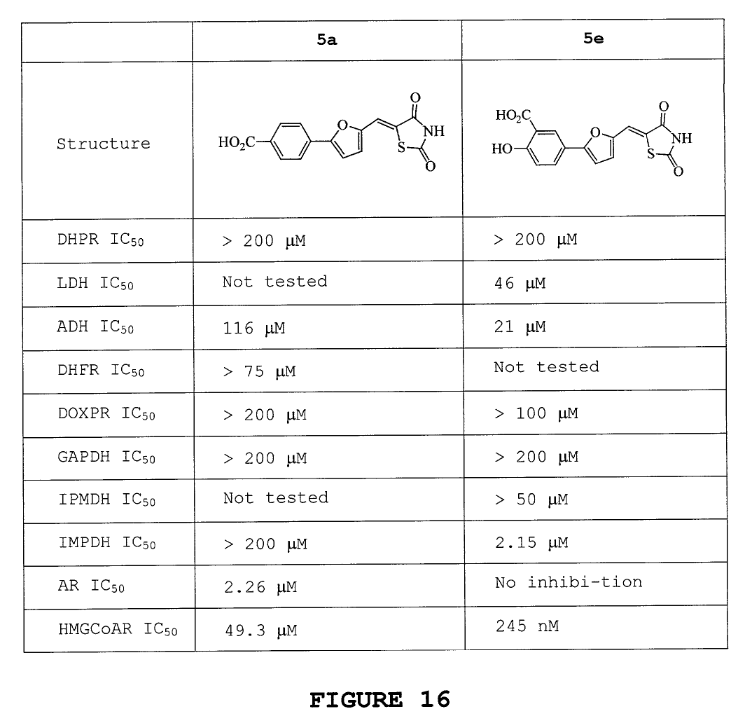

IC₅₀ data for these compounds are presented in FIG. 16. The compound 4-[5-(2,4-dioxo-thiazolidin-5-ylidenemethyl)-furan-2-yl]-benzoic acid (compound Sa) exhibited IC₅₀ values of 116 μM for ADH, 49.3 μM for HMGCoAR, and 2.26 μM for AR, respectively. The IC₅₀ values for DHPR, DOXPR, GAPDH, and IMPDH were greater than 200 μM, and the IC₅₀ value for DHFR was greater than 75 μM.

The compound 5-[5-(2,4-dioxo-thiazolidin-5-ylidenemethyl)-furan-2-yl]-2-hydroxy-benzoic acid (compound 5e) exhibited IC₅₀ values of 46 μM for LDH, 21 μM for ADH, 2.15 μM for IMPDH, and 245 nM for HMGCoAR, respectively. The IC₅₀ values for DHPR and GAPDH were greater than 200 μM. The IC₅₀value for DOXPR was greater than 100 μM, while the IC₅₀ value for IPMDH was greater than 50 μM. No inhibition of AR was seen.

### Example 37

### Screening of Selected Thiazolidinediones and Rhodanines for Binding to Dehydrogenases and Oxidoreductases

This example describes the screening of thiazolidinedione and rhodanine common ligand mimics for binding activity to a variety of dehydrogenases and oxidoreductases.

The following compounds were produced by the methods of Examples 1, 5, 2, and 12, respectively: 4-[5-(2,4-dioxo-thiazolidin-5-ylidenemethyl)-furan-2-yl]-benzoic acid; 5-[5-(2,4-dioxo-thiazolidin-5-ylidenemethyl)-furan-2-yl]-2-hydroxy-benzoic acid; 3-[5-(2,4-dioxo-thiazolidin-5-ylidenemethyl)-furan-2-yl]-benzoic acid; 2-hydroxy-5-[5-(4-oxo-2-thioxo-thiazolidin-5-ylidenemethyl)-furan-2-yl]-benzoic acid. The compounds were screened for binding to the following enzymes: HMG CoA reductase (HMGCoAR), inosine-5′-monophosphate dehydrogenase (IMPDH), 1-deoxy-D-xylulose-5-phosphate reductase (DOXPR), dihydrodipicolinate reductase (DHPR), dihydrofolate reductase (DHFR), 3-isopropylmalate (IPMDH), glyceraldehyde-3-phosphate dehydrogenase (GAPDH), aldose reductase (AR), alcohol dehydrogenase (ADH), and lactate dehydrogenase (LDH). The assay procedures employed were those described in Example 36.

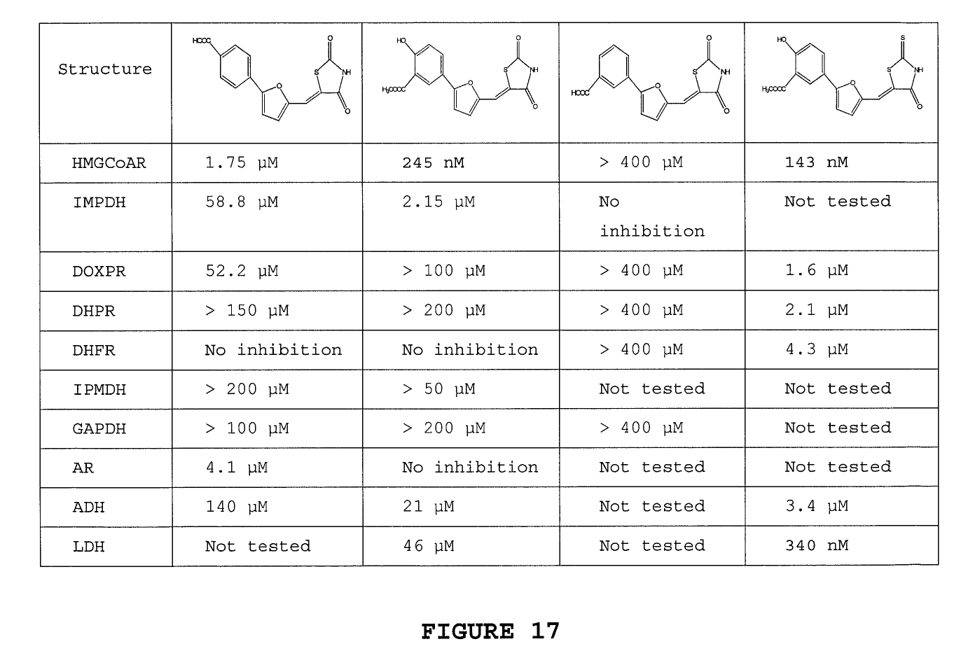

IC₅₀ data for these compounds are presented in FIG. 17. The compound 4-[5-(2,4-dioxo-thiazolidin-5-ylidenemethyl)-furan-2-yl]-benzoic acid exhibited IC₅₀ values of 1.75 μM for HMGCoAR, 4.1 μM for AR, 52.2 μM for DOXPR, 58.8 μM for IMPDH, and 140 μM for ADH, respectively. The IC₅₀ values for GAPDH, DHPR, and IPMDH were greater than 100 μM, greater than 150 μM, and greater than 200 μM, respectively. No inhibiiton of DHFR was seen.

No inhibition of DHFR or AR was seen with 5-[5-(2,4-dioxo-thiazolidin-5-ylidenemethyl)-furan-2-yl]-2-hydroxy-benzoic acid. However, the compound exhibited IC₅₀ values of 245 μM for HMGCoAR, 2.15 μM for IMPDH, 21 μM for ADH, and 46 μM for LDH, respectively. The IC₅₀ values for DHPR and GAPDH were greater than 200 μM, and the IC₅₀ value for IPMDH was greater than 50 μM.

No inhibition of IMPDH seen with 3-[5-(2,4-dioxo-thiazolidin-5-ylidenemethyl)-furan-2-yl]-benzoic acid. The IC₅₀ values for HMGCoAR, DOXPR, DHPR, DHFR, and GAPDH with this compound were greater than 400 μM.

The compound 2-hydroxy-5-[5-(4-oxo-2-thioxo-thiazolidin-5-ylidenemethyl)-furan-2-yl]-benzoic acid exhibited IC₅₀ values of 143 nM for HMGCoAR, 340 nM for LDH, 1.6 μM for DOXPR, 2.1 μM for DHPR, 3.4 μM for ADH, and 4.3 μM for DHFR, respectively.

### Example 38

### Screening of Biligands for Binding to Dihydrodipicolinate Reductase (DHPR)

This example describes the screening of bi-ligands having thiazolidinedione or rhodanine common ligand mimics for binding activity to dihydrodipicolinate reductase (DHPR).

Bi-ligands were produced by the methods of Examples 14 to 18. The bi-ligands were screened for binding to E. coli DHPR. The bi-ligands were screened using a kinetic protocol that spectrophotometrically evaluates oxidation of NADPH.

Stock solutions of each of the reagents were prepared in the following concentrations. Dilutions of the stock solutions were prepared prior to running the assay in the concentrations indicated below. Dilution of DHPR was prepared in 10 mM HEPES at a pH of 7.4. DHPS was not diluted and was stored in eppindorf tubes.

| | Stock | | Final | | Volume needed |
|---|---|---|---|---|---|
| ddH₂O | | | | | 798 μl |
| HEPES (pH 7.8) | 1 | M | 0.1 | M | 100 μl |
| Pyruvate | 50 | mM | 1 | mM | 20 μl |
| NADPH | 1 | mM | 6 | μM | 6 μl |
| L-ASA | 28.8 | mM | 40 | μM | 13.9 μl |
| DHPS | 1 | mg/ml | | | 7 μl |
| DHPR | 1:1000 | dilution of | | | 5 μl |
| | | 1 mg/ml | | | |
| | | stock | | | |
| Inhibitor | 10 | μM | 500 | nM | 50 μl |
| DMSO | 100% | | 5% | | 0 μl |

Total Assay volume = 1000 μl

The L-ASA solution was prepared in the following manner. 180 μM stock solution of ASA was prepared. 100 μl of the ASA stock was mixed with 150 μl of concentrated NaHCO3 and 375 μl of H₂O. For use in the assay, 28.8 mM L-ASA equal 625 μl of the solution. The L-ASA stock solution was kept at a temperature of −20° C. After dilution, the pH of the 28.8 mM solution was checked and maintained between 1 and 2.

First, the DHPS reaction was monitored at 340 nm prior to and after addition of the inhibitor to detect background reaction with the inhibitor. The solution for background detection was a 945 μl solution containing 0.1 HEPES (pH 7.8), 1 mM pyruvate, 6 μM NADPH, 40 μM L-ASA, and 7 μl of 1 mg/ml DHPS at 25° C. in the volumes provided above. The sample solution was then mixed and incubated for 10 minutes. Next, 500 nM solutions of the inhibitors and enough DMSO to provide a final DMSO concentration of 5% of the total assay volume were added. The solution was mixed and incubated for an additional 6 minutes.

In DHPR samples, 5 μl of the diluted DHPR enzyme were added. The sample was mixed for 20 seconds and then the reaction was run for 10 minutes. After a 50 second lag, the samples were read in Cary spectrophotometer at 340 nm. Reading of the samples was continued until 300 seconds. Cuvette #1 contained the control reaction (no inhibitor), and cuvette #2 contained the positive control reaction in which Cibacron Blue at 2.58 μM was substituted for inhibitor to yield 70 to 80% inhibition. The substrate and NADPH or NAHD were kept near their Km values.

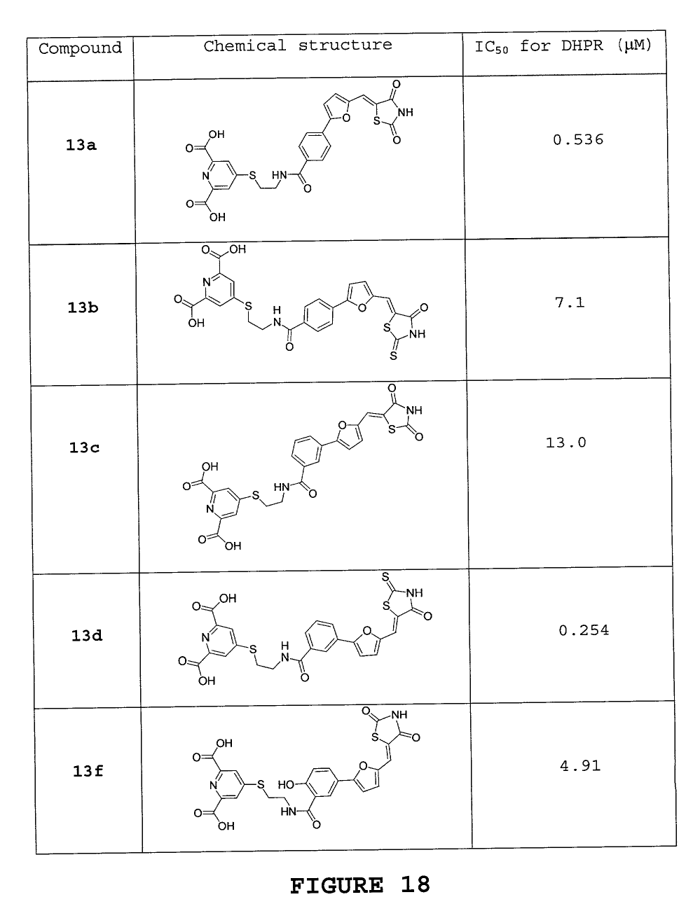

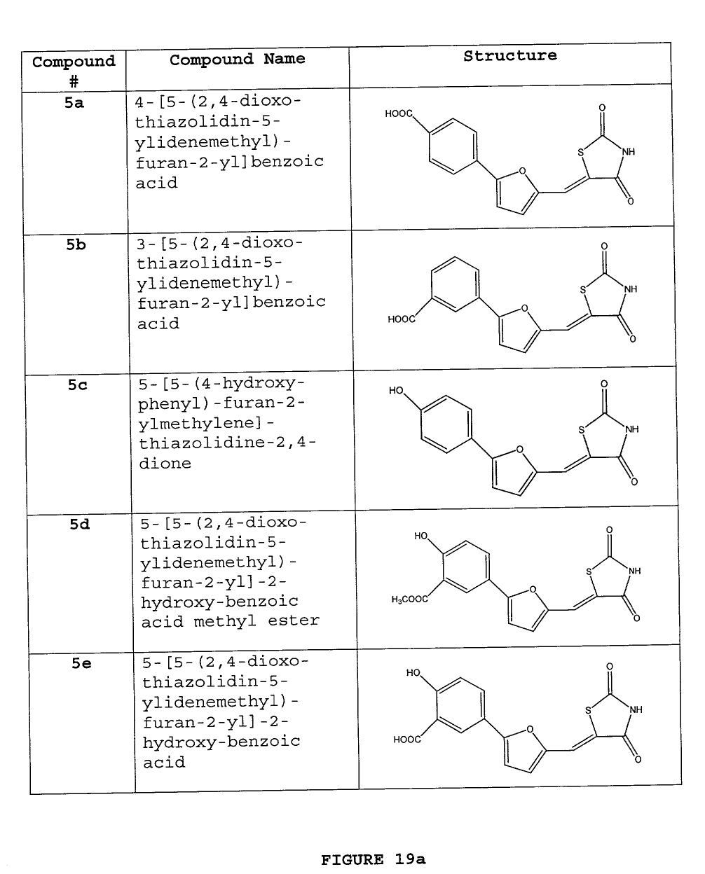

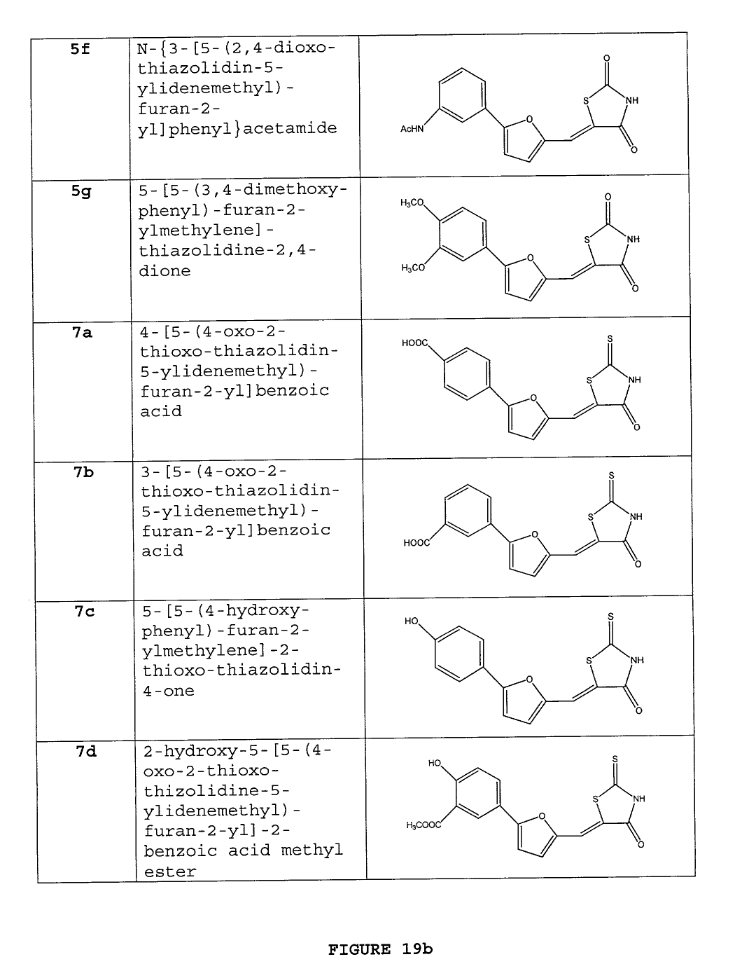

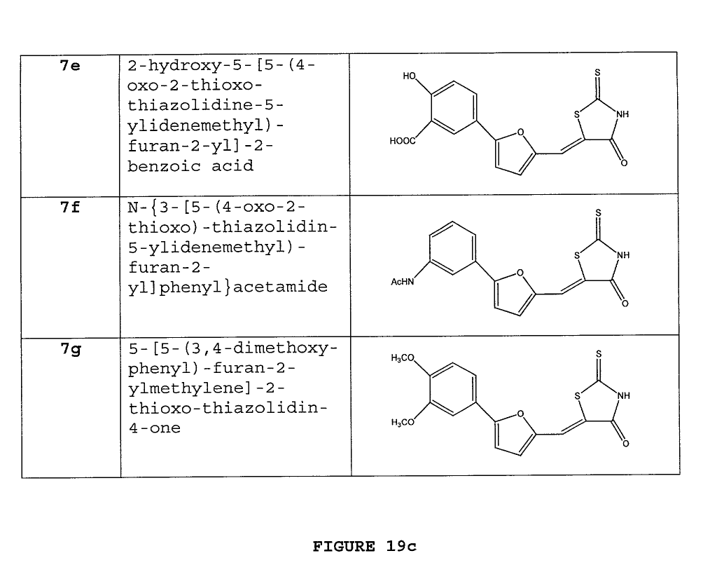

IC₅₀ data for these compounds are presented in FIG. 18. The rhodanine and thiazolidinedione derivative bi-ligands 13a, 13b, 13c, 13d and 13f exhibited IC₅₀ values for dihydrodipicolinate reductase (DHPR) of about 0.536 μM, 7.1 μM, 13 μM, 0.254 μM, and 4.91 μM respectively.

1. A compound comprising the formula: 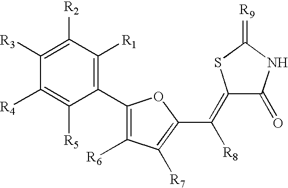
wherein
R₁ to R₈ each independently are selected from the group consisting of H, alkyl, alkenyl, alkynyl, aryl, heterocycle, COOH, COOAlkyl, CONR₁₀R₁₁, C(O)R₁₂, OH, OAlkyl, OAc, SH, SR₁₂, SO₃H, S(O)R₁₂, SO₂NR₁₀R₁₁, S(O)₂R₁₂, NH₂, NHR₁₂, NR₁₀R₁₁, NHCOR₁₂, N₃, NO₂, PH₃, PH₂R₁₂, H₂PO₄, H₂PO₃, H₂PO₂, HPO₄R₁₂, PO₂R₁₁R₁₂, CN, and X;
R₉ is O, S, or NR₁₂; and
R₁₀, R₁₁, and R₁₂ each independently are selected from the group consisting of hydrogen, alkyl, alkenyl, alkynyl, aryl, and heterocycle, or R₁₀ and R₁₁ together with the nitrogen to which they are attached can be joined to form a heterocyclic ring;
with the proviso that at least one of R₁ to R₈ is other than hydrogen.

2. The compound of claim 1, wherein at least one of R₁ to R₈ is COOH.

3. The compound of claim 1, wherein at least one of R₁ to R₈ is OH.

4. The compound of claim 1, wherein at least one of R₁ to R₈ is OAlkyl.

5. The compound of claim 1, wherein at least one of R₁ to R₈ is COOAlkyl.

6. The compound of claim 1, wherein at least one of R₁ to R₈ is NHCOR₇.

7. The compound of claim 1, wherein two or more of R₁ to R₈ are substituted.

8. The compound of claim 1, having the formula 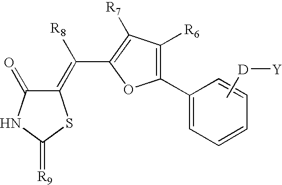
wherein
D is alkylene, alkenylene, alkynylene, aryl, or heterocycle; and
Y is OH, NHR₁₂, SH, COOH, SO₂OH, X, CN, N₃, CONH₂, CONHR₁₂, C≡CH, or CH═CH₂.

9. The compound of claim 1, having the formula 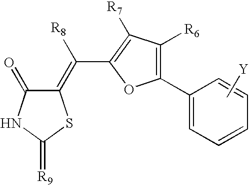
wherein Y is OH, NHR₁₂, SH, COOH, SO₂OH, X, CN, N₃, CONH₂, CONHR₁₂, C≡CH, or CH═CH₂.

10. The compound of claim 1, having the formula 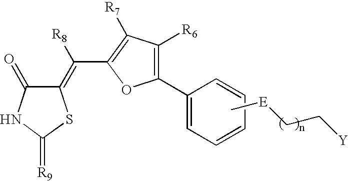
wherein
E is O, S, NR₁₂, CR₁₁C₁₂, CONR₁₂, SO₂NR₁₂, NR₁₁CONR₁₂, NR₁₁CNHNR₁₂, NR₁₂COO, C≡C, or CH═CH;
Y is OH, NHR₁₂, SH, COOH, SO₂OH, X, CN, N₃, CONH₂, CONHR₁₂, C≡CH, or CH═CH₂; and
n is an integer between 0 and 5, inclusive.

11. The compound of claim 1, having the formula 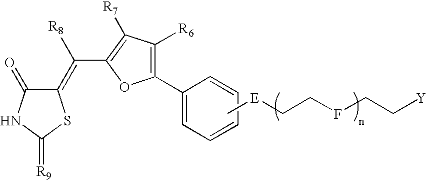
wherein
E and F each independently are selected from the group consisting of O, S, NR₁₂, CR₁₁C₁₂, CONR₁₂, SO₂NR₁₂, NR₁₁CONR₁₂, NR₁₁CNHNR₁₂, NR₁₂COO, C≡C, and CH═CH;
Y is OH, NHR₁₂, SH, COOH, SO₂OH, X, CN, N₃, CONH₂, CONHR₁₂, C≡CH, or CH═CH₂; and
n is an integer between 0 and 5, inclusive.

12. The compound of claim 1, having the formula 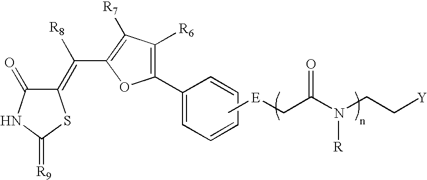
wherein
E is O, S, NH, NR₁₂, CR₁₁C₁₂, CONR₁₂, SO₂NR₁₂, NR₁₁CONR₁₂, NR₁₁CNHNR₁₂, NR₁₂COO, C≡C, or CH═CH;
Y is OH, NHR₁₂, SH, COOH, SO₂OH, X, CN, N₃, CONH₂, CONHR₁₂, C≡CH, or CH═CH₂;
R is hydrogen, alkyl, alkenyl, alkynyl, aryl, or heterocycle; and
n is an integer between 0 and 5, inclusive.

13. The compound of claim 1, having the formula 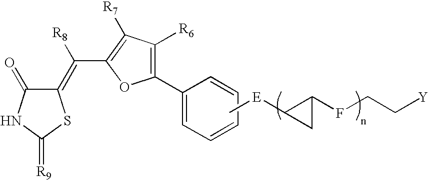
wherein
E and F each independently are selected from the group consisting of O, S, NR₁₂, CR₁₁C₁₂, CONR₁₂, SO₂NR₁₂, NR₁₁CONR₁₂, NR₁₁CNHNR₁₂, NR₁₂COO, C≡C, and CH═CH;
Y is OH, NHR₁₂, SH, COOH, SO₂OH, X, CN, N₃, CONH₂, CONHR₁₂, C≡CH, or CH═CH₂; and
n is an integer between 0 and 5, inclusive.

14. The compound of claim 1, having the formula 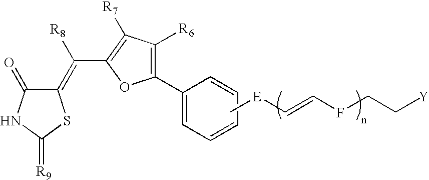
wherein
E is O, S, NR₁₂, CR₁₁C₁₂, CONR₁₂, SO₂NR₁₂, NR₁₁CONR₁₂, NR₁₁CNHNR₁₂, NR₁₂COO, C≡C, or CH═CH;
F independently is selected from the group consisting of O, S, NR₁₂, CR₁₁R₁₂, CONR₁₂, NR₁₁CONR₁₂, NR₁₁CNHNR₁₂, NR₁₂COO, C═C, and CH═CH;
Y is OH, NHR₁₂, SH, COOH, SO₂OH, X, CN, N₃, CONH₂, CONHR₁₂, C≡CH, or CH═CH₂; and
n is an integer between 0 and 5, inclusive.

15. The compound of claim 1, having the formula 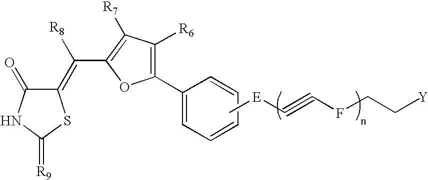
wherein
E is O, S, NR₁₂, CR₁₁C₁₂, CONR₁₂, SO₂NR₁₂, NR₁₁CONR₁₂, NR₁₁CNHNR₁₂, NR₁₂COO, C≡C, or CH═CH;
F independently is selected from the group consisting of O, S, NR₁₂, CR₁₁R₁₂, CONR₁₂, NR₁₁CONR₁₂, NR₁₁CNHNR₁₂, NR₁₂COO, C═C, and CH═CH;
Y is OH, NHR₁₂, SH, COOH, SO₂OH, X, CN, N₃, CONH₂, CONHR₁₂, C≡CH, or CH═CH₂; and
n is an integer between 0 and 5, inclusive.

16. The compound of claim 1, having the formula 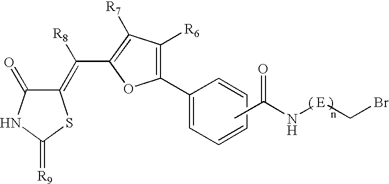
wherein
E is O, S, NR₁₂, CR₁₁C₁₂, CONR₁₂, SO₂NR₁₂, NR₁₁CONR₁₂, NR₁₁CNHNR₁₂, NR₁₂COO, C≡C, or CH═CH; and
n is an integer between 0 and 5, inclusive.

17. The compound of claim 1, having the formula 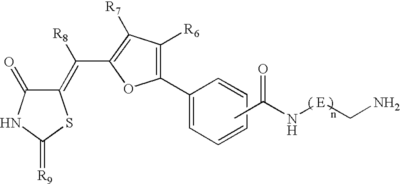
wherein
E is CH₂, CH₂CH₂OCH or CH₂CH₂SCH and n is an integer between 1 and 10, inclusive.

18. The compound of claim 17, wherein n is greater than 4 and E is CH₂CH₂OCH or CH₂CH₂SCH.

19. The compound of claim 1, having the formula

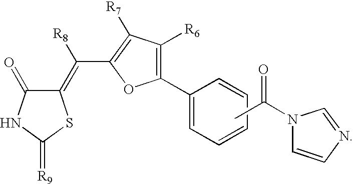

20. A compound comprising the formula: 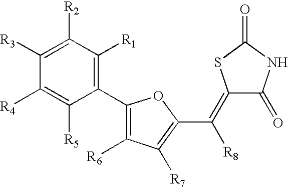
wherein
R₁ to R₈ each independently are selected from the group consisting of H, alkyl, alkenyl, alkynyl, aryl, heterocycle, COOH, COOAlkyl, CONR₁₀R₁₁, C(O)R₁₂, OH, OAlkyl, OAc, SH, SR₁₂, SO₃H, S(O)R₁₂, SO₂NR₁₀R₁₁, S(O)₂R₁₂, NH₂, NHR₁₂, NR₁₀R₁₁, NHCOR₁₂, N₃, NO₂, PH₃, PH₂R₁₂, H₂PO₄, H₂PO₃, H₂PO₂, HPO₄R₁₂, PO₂R₁₁R₁₂, CN, and X;
R₁₀, R₁₁, and R₁₂ each independently are selected from the group consisting of hydrogen, alkyl, alkenyl, alkynyl, aryl, and heterocycle, or R₁₀ and R₁₁ together with the nitrogen to which they are attached can be joined to form a heterocyclic ring;
with the proviso that at least one of R₁ to R₈ is other than hydrogen.

21. The compound of claim 20, wherein at least one of R₁ to R₈ is COOH.

22. The compound of claim 20, wherein at least one of R₁ to R₈ is OH.

23. The compound of claim 20, wherein at least one of R₁ to R₈ is COOAlkyl.

24. The compound of claim 20, wherein at least one of R₁ to R₈ is OAlkyl.

25. The compound of claim 20, wherein two or more of R₁ to R₈ are substituted.

26. The compound of claim 20, having the formula:

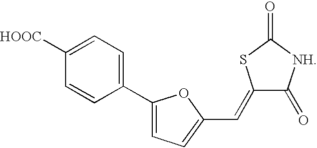

27. The compound of claim 20, having the formula:

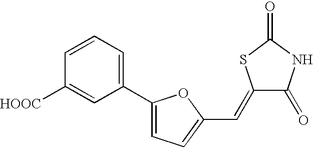

28. The compound of claim 20, having the formula:

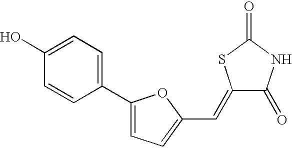

29. The compound of claim 20, having the formula:

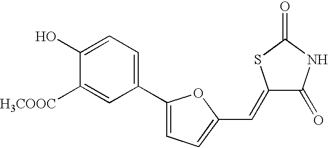

30. The compound of claim 20, having the formula:

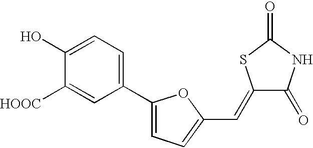

31. The compound of claim 20, having the formula:

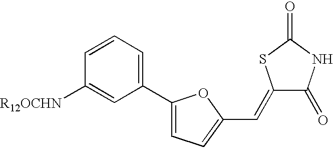

32. The compound of claim 20, having the formula:

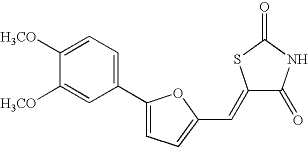

33. The compound of claim 20,having the formula 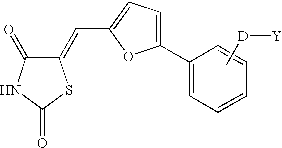
wherein
D is alkylene, alkenylene, alkynylene, aryl, or heterocycle; and
Y is OH, NHR₁₂, SH, COOH, SO₂OH, X, CN, N₃, CONH₂, CONHR₁₂, C≡CH, or CH═CH₂.

34. The compound of claim 20, having the formula 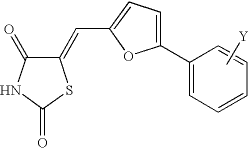
wherein Y is OH, NHR₁₂, SH, COOH, SO₂OH, X, CN, N₃, CONH₂, CONHR₁₂, C≡CH, or CH═CH₂.

35. The compound of claim 20, having the formula 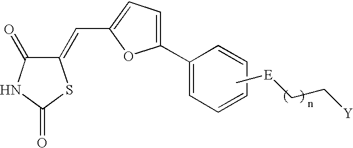
wherein
E is O, S, NR₁₂, CR₁₁C₁₂, CONR₁₂, SO₂NR₁₂, NR₁₁CONR₁₂, NR₁₁CNHNR₁₂, NR₁₂COO, C≡C, or CH═CH;
Y is OH, NHR₁₂, SH, COOH, SO₂OH, X, CN, N₃, CONH₂, CONHR₁₂, C≡CH, or CH═CH₂; and
n is an integer between 0 and 5, inclusive.

36. The compound of claim 20, having the formula 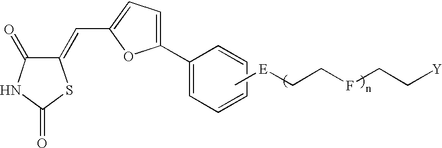
wherein
E and F each independently are selected from the group consisting of O, S, NR₁₂, CR₁₁C₁₂, CONR₁₂, SO₂NR₁₂, NR₁₁CONR₁₂, NR₁₁CNHNR₁₂, NR₁₂COO, C≡C, and CH═CH;
Y is OH, NHR₁₂, SH, COOH, SO₂OH, X, CN, N₃, CONH₂, CONHR₁₂, ═C≡CH, or CH═CH₂; and
n is an integer between 0 and 5, inclusive.

37. The compound of claim 20, having the formula 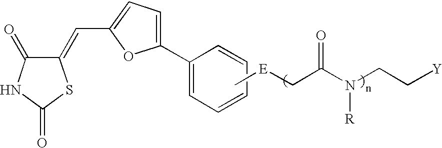
wherein
E is O, S, NR₁₂, CR₁₁C₁₂, CONR₁₂, SO₂NR₁₂, NR₁₁CONR₁₂, NR₁₁CNHNR₁₂, NR₁₂COO, C≡C, or CH═CH;
Y is OH, NHR₁₂, SH, COOH, SO₂OH, X, CN, N₃, CONH₂, CONHR₁₂, C≡CH, or CH═CH₂;
R is hydrogen, alkyl, alkenyl, alkynyl, aryl, or heterocycle; and
n is an integer between 0 and 5, inclusive.

38. The compound of claim 20, having the formula 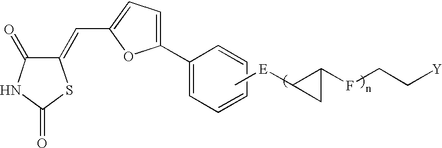
wherein
E and F each independently are selected from the group consisting of O, S, NR₁₂, CR₁₁C₁₂, CONR₁₂, SO₂NR₁₂, NR₁₁CONR₁₂, NR₁₁CNHNR₁₂, NR₁₂COO, C≡C, and CH═CH;
Y is OH, NHR₁₂, SH, COOH, SO₂OH, X, CN, N₃, CONH₂, CONHR₁₂, C≡CH, or CH═CH₂; and
n is an integer between 0 and 5, inclusive.

39. The compound of claim 20, having the formula 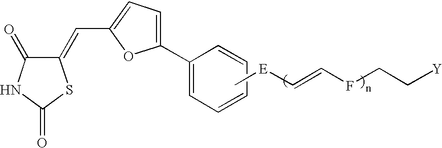
wherein
E is O, S, NR₁₂, CR₁₁C₁₂, CONR₁₂, SO₂NR₁₂, NR₁₁CONR₁₂, NR₁₁CNHNR₁₂, NR₁₂COO, C≡C, or CH═CH;
F independently is selected from the group consisting of O, S, NR₁₂, CR₁₁R₁₂, CONR₁₂, NR₁₁CONR₁₂, NR₁₁CNHNR₁₂, NR₁₂COO, C═C, and CH═CH;
Y is OH, NHR₁₂, SH, COOH, SO₂OH, X, CN, N₃, CONH₂, CONHR₁₂, C≡CH, or CH═CH₂; and
n is an integer between 0 and 5, inclusive.

40. The compound of claim 20, having the formula 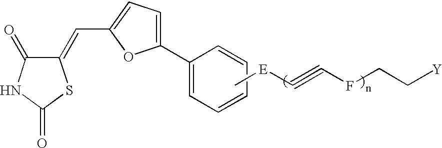
wherein
E is O, S, NR₁₂, CR₁₁C₁₂, CONR₁₂, SO₂NR₁₂, NR₁₁CONR₁₂, NR₁₁CNHNR₁₂, NR₁₂COO, C≡C, or CH═CH;
F independently is selected from the group consisting of O, S, NR₁₂, CR₁₁R₁₂, CONR₁₂, NR₁₁CONR₁₂, NR₁₁CNHNR₁₂, NR₁₂COO, C═C, and CH═CH;
Y is OH, NHR₁₂, SH, COOH, SO₂OH, X, CN, N₃, CONH₂, CONHR₁₂, C≡CH, or CH═CH₂; and
n is an integer between 0 and 5, inclusive.

41. The compound of claim 20,having the formula 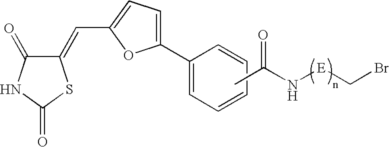
wherein
E is O, S, NR₁₂, CR₁₁C₁₂, CONR₁₂, SO₂NR₁₂, NR₁₁CONR₁₂, NR₁₁CNHNR₁₂, NR₁₂COO, C≡C, or CH═CH; and
n is an integer between 0 and 5, inclusive.

42. The compound of claim 20,having the formula 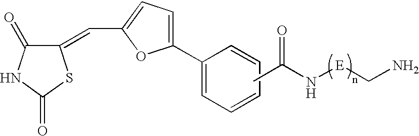
wherein
E is O, CH₂, S, NR₁₂, CR₁₁C₁₂, CONR₁₂, SO₂NR₁₂, NR₁₁CONR₁₂, NR₁₁CNHNR₁₂, NR₁₂COO, C≡C, or CH═CH; and
n is an integer between 0 and 5, inclusive.

43. The compound of claim 42, wherein n is greater than 4 and E is CH₂CH₂OCH or CH₂CH₂SCH.

44. The compound of claim 20,having the formula

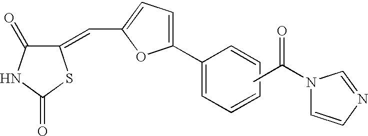

45. A compound comprising the formula: 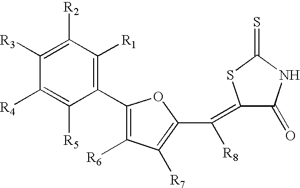
wherein
R₁ to R₈ each independently are selected from the group consisting of H, alkyl, alkenyl, alkynyl, aryl, heterocycle, COOH, COOAlkyl, CONR₁₀R₁₁, C(O)R₁₂, OH, OAlkyl, OAc, SH, SR₁₂, SO₃H, S(O)R₁₂ SO₂NR₁₀R₁₁, S(O)₂R₁₂, NH₂, NHR₁₂, NR₁₀R₁₁, NHCOR₁₂, N₃, NO₂, PH₃, PH₂R₁₂, H₂PO₄, H₂PO₃, H₂PO₂, HPO₄R₁₂, PO₂R₁₁R₁₂, CN, and X;
R₁₀, R₁₁, and R₁₂ each independently are selected from the group consisting of hydrogen, alkyl, alkenyl, alkynyl, aryl, and heterocycle, or R₁₀ and R₁₁ together with the nitrogen to which they are attached can be joined to form a heterocyclic ring;
with the proviso that at least one of R₁ to R₈ is other than hydrogen.

46. The compound of claim 45, wherein at least one of R₁ to R₈ is COOH.

47. The compound of claim 45, wherein at least one of R₁ to R₈ is OH.

48. The compound of claim 45, wherein at least one of R₁ to R₈ is OAlkyl.

49. The compound of claim 45, wherein at least one of R₁ to R₈ is COOAlkyl.

50. The compound of claim 45, wherein at least one of R₁ to R₈ is NHAc.

51. The compound of claim 45, having the formula:

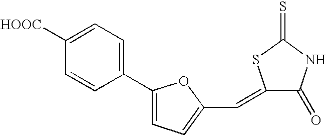

52. The compound of claim 45, having the formula:

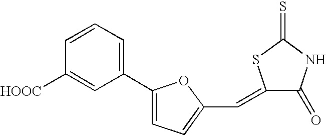

53. The compound of claim 45, having the formula:

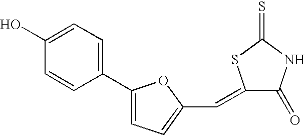

54. The compound of claim 45, having the formula:

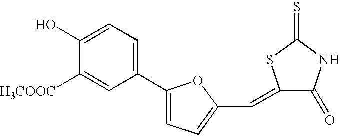

55. The compound of claim 45, having the formula:

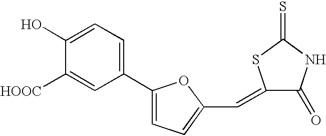

56. The compound of claim 45, having the formula:

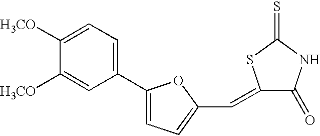

57. The compound of claim 45,having the formula 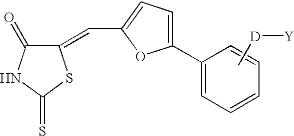
wherein
D is alkylene, alkenylene, alkynylene, aryl, or heterocycle; and
Y is OH, NHR₁₂, SH, COOH, SO₂OH, X, CN, N₃, CONH₂, CONHR₁₂, C≡CH, or CH═CH₂.

58. The compound of claim 45,having the formula 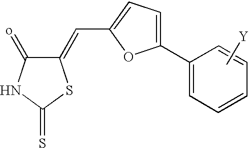
wherein
wherein Y is OH, NHR₁₂, SH, COOH, SO₂OH, X, CN, N₃, CONH₂, CONHR₁₂, C≡CH, or CH═CH₂.

59. The compound of claim 45,having the formula 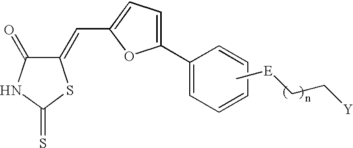
wherein
E is O, S, NR₁₂, CR₁₁C₁₂, CONR₁₂, SO₂NR₁₂, NR₁₁CONR₁₂, NR₁₁CNHNR₁₂, NR₁₂COO, C≡C, or CH═CH;
Y is OH, NHR₁₂, SH, COOH, SO₂OH, X, CN, N₃, CONH₂, CONHR₁₂, C≡CH, or CH═CH₂; and
n is an integer between 0 and 5, inclusive.

60. The compound of claim 45, having the formula 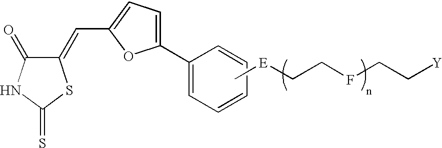
wherein
E and F each independently are selected from the group consisting of O, S, NR₁₂, CR₁₁C₁₂, CONR₁₂, SO₂NR₁₂, NR₁₁CONR₁₂, NR₁₁CNHNR₁₂, NR₁₂COO, C≡C, and CH═CH;
Y is OH, NHR₁₂, SH, COOH, SO₂OH, X, CN, N₃, CONH₂, CONHR₁₂, C≡CH, or CH═CH₂; and
n is an integer between 0 and 5, inclusive.

61. The compound of claim 45,having the formula 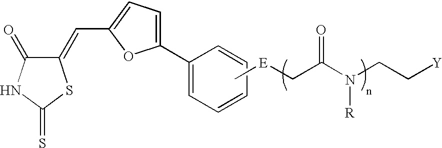
wherein
E is O, S, NR₁₂, CR₁₁C₁₂, CONR₁₂, SO₂NR₁₂, NR₁₁CONR₁₂, NR₁₁CNHNR₁₂, NR₁₂COO, C≡C, or CH═CH;
Y is OH, NHR₁₂, SH, COOH, SO₂OH, X, CN, N₃, CONH₂, CONHR₁₂, C≡CH, or CH═CH₂;
R is hydrogen, alkyl, alkenyl, alkynyl, aryl, or heterocycle; and
n is an integer between 0 and 5, inclusive.

62. The compound of claim 45,having the formula 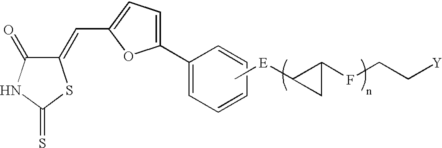
wherein
E and F each independently are selected from the group consisting of O, S, NR₁₂, CR₁₁R₁₂, CONR₁₂, SO₂NR₁₂, NR₁₁CONR₁₂, NR₁₁CNHNR₁₂, NR₂COO, C≡C, and CH═CH;
Y is OH, NHR₁₂, SH, COOH, SO₂OH, X, CN, N₃, CONH₂, CONHR₁₂, C≡CH, or CH═CH₂; and
n is an integer between 0 and 5, inclusive.

63. The compound of claim 45,having the formula 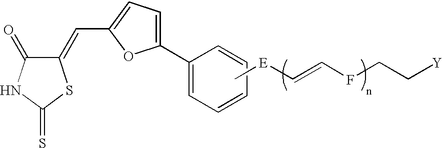
wherein
E is O, S, NR₁₂, CR₁₁C₁₂, CONR₁₂, SO₂NR₁₂, NR₁₁CONR₁₂, NR₁₁CNHNR₁₂, NR₁₂COO, C≡C, or CH═CH;
F independently is selected from the group consisting of O, S, NR₁₂, CR₁₁R₁₂, CONR₁₂, NR₁₁CONR₁₂, NR₁₁CNHNR₁₂, NR₁₂COO, C═C, and CH═CH;
Y is OH, NHR₁₂, SH, COOH, SO₂OH, X, CN, N₃, CONH₂, CONHR₁₂, C≡CH, or CH═CH₂; and
n is an integer between 0 and 5, inclusive.

64. The compound of claim 45,having the formula 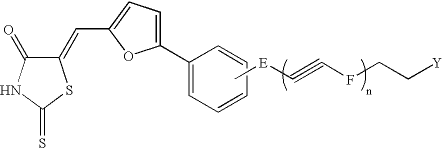
wherein
E is O, S, NR₁₂, CR₁₁C₁₂, CONR₁₂, SO₂NR₁₂, NR₁₁CONR₁₂, NR₁₁CNHNR₁₂, NR₁₂COO, C≡C, or CH═CH;
F independently is selected from the group consisting of O, S, NR₁₂, CR₁₁R₁₂, CONR₁₂, NR₁₁CONR₁₂, NR₁₁CNHNR₁₂, NR₁₂COO, C═C, and CH═CH;
Y is OH, NHR₁₂, SH, COOH, SO₂OH, X, CN, N₃, CONH₂, CONHR₁₂, C≡CH, or CH═CH₂; and
n is an integer between 0 and 5, inclusive.

65. The compound of claim 45,having the formula 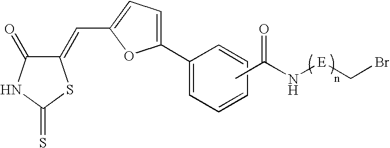
wherein
E is selected O, S, NR₁₂, CR₁₁C₁₂, CONR₁₂, SO₂NR₁₂, NR₁₁CONR₁₂, NR₁₁CNHNR₁₂, NR₁₂COO, C≡C, or CH═CH; and
n is an integer between 0 and 5, inclusive.

66. The compound of claim 45,having the formula 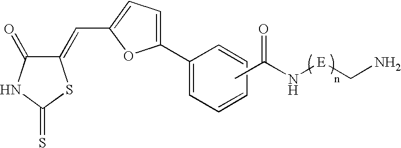
wherein
E is CH₂, CH₂CH₂OCH or CH₂CH₂SCH and n is an integer between 1 and 10, inclusive.

67. The compound of claim 66, wherein n is greater than 4 and E is CH₂CH₂OCH or CH₂CH₂SCH.

68. The compound of claim 45, having the formula

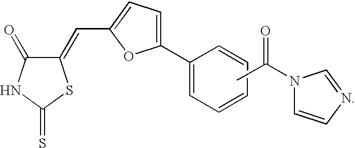

69. A combinatorial library of two or more compounds comprising a common ligand variant of a compound of the formula: 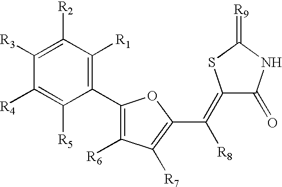
wherein
R₁ to R₈ each independently are selected from the group consisting of H, alkyl, alkenyl, alkynyl, aryl, heterocycle, COOH, COOAlkyl, CONR₁₀R₁₁, C(O)R₁₂, OH, OAlkyl, OAc, SH, SR₁₂, SO₃H, S(O)R₁₂, SO₂NR₁₀R₁₁, S(O)₂R₁₂, NH₂, NHR₁₂, NR₁₀R₁₁, NHCOR₁₂, N₃, NO₂, PH₃, PH₂R₁₂, H₂PO₄, H₂PO₃, H₂PO₂, HPO₄R₁₂, PO₂R₁₁R₁₂, CN, and X;
R₉ is O, S, or NR₁₂; and
R₁₀, R₁₁, and R₁₂ each independently are selected from the group consisting of hydrogen, alkyl, alkenyl, alkynyl, aryl, and heterocycle, or R₁₀ and R₁₁ together with the nitrogen to which they are attached can be joined to form a heterocyclic ring.

70. The combinatorial library of claim 69, wherein at least one of R₁ to R₈ is COOH.

71. The combinatorial library of claim 69, wherein at least one of R₁ to R₈ is OH.

72. The combinatorial library of claim 69, wherein at least one of R₁ to R₈ is OAlkyl.

73. The combinatorial library of claim 69, wherein at least one of R₁ to R₈ is COOAlkyl.

74. The combinatorial library of claim 69, wherein at least one of R₁ to R₈ is NHCOR₇.

75. The combinatorial library of claim 69, wherein two or more of R₁ to R₅ are substituted.

76. The combinatorial library of claim 69, having the formula 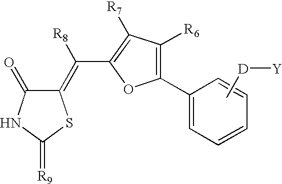
wherein
D is alkylene, alkenylene, alkynylene, aryl, or heterocycle; and
Y is OH, NHR₁₂, SH, COOH, SO₂OH, X, CN, N₃, CONH₂, CONHR₁₂, C≡CH, or CH═CH₂.

77. The combinatorial library of claim 69, having the formula 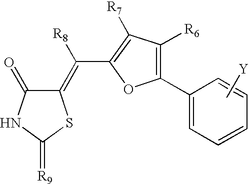
wherein Y is OH, NHR₁₂, SH, COOH, SO₂OH, X, CN, N₃, CONH₂, CONHR₁₂, C≡CH, or CH═CH₂.

78. The combinatorial library of claim 69, having the formula 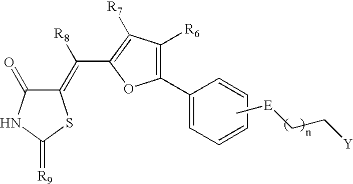
wherein
E is O, S, NR₁₂, CR₁₁C₁₂, CONR₁₂, SO₂NR₁₂, NR₁₁CONR₁₂, NR₁₁CNHNR₁₂, NR₁₂COO, C≡C, or CH═CH;
Y is OH, NHR₁₂, SH, COOH, SO₂OH, X, CN, N₃, CONH₂, CONHR₁₂, C≡CH, or CH═CH₂; and
n is an integer between 0 and 5, inclusive.

79. The combinatorial library of claim 69, having the formula 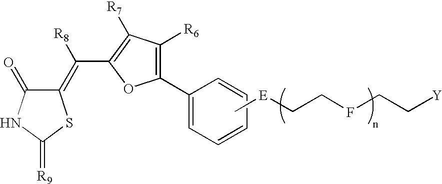
wherein
E and F each independently are selected from the group consisting of O, S, NR₁₂, CR₁₁C₁₂, CONR₁₂, SO₂NR₁₂, NR₁₁CONR₁₂, NR₁₁CNHNR₁₂, NR₁₂COO, C≡C, and CH═CH;
Y is OH, NHR₁₂, SH, COOH, SO₂OH, X, CN, N₃, CONH₂, CONHR₁₂, C≡CH, or CH═CH₂; and
n is an integer between 0 and 5, inclusive.

80. The combinatorial library of claim 69, having the formula 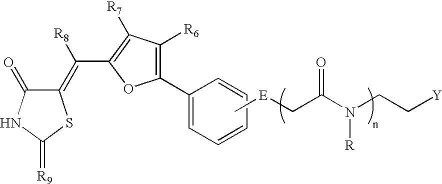
wherein
E is O, S, NR₁₂, CR₁₁C₂, CONR₁₂, SO₂NR₁₂, NR₁₁CONR₁₂, NR₁₁CNHNR₁₂, NR₁₂COO, C≡C, or CH═CH;
Y is OH, NHR₁₂, SH, COOH, SO₂OH, X, CN, N₃, CONH₂, CONHR₁₂, C≡CH, or CH═CH₂;
R is hydrogen, alkyl, alkenyl, alkynyl, aryl, or heterocycle; and
n is an integer between 0 and 5, inclusive.

81. The combinatorial library of claim 69, having the formula 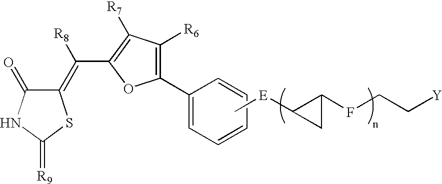
wherein
E and F each independently are selected from the group consisting of O, S, NR₁₂, CR₁₁C₁₂, CONR₁₂, SO₂NR₁₂, NR₁₁CONR₁₂, NR₁₁CNHNR₁₂, NR₁₂COO, C≡C, and CH═CH;
Y is OH, NHR₁₂, SH, COOH, SO₂OH, X, CN, N₃, CONH₂, CONHR₁₂, C≡CH, or CH═CH₂; and
n is an integer between 0 and 5, inclusive.

82. The combinatorial library of claim 69, having the formula 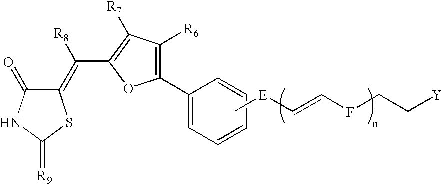
wherein
E is O, S, NR₁₂, CR₁₁C₁₂, CONR₁₂, SO₂NR₁₂, NR₁₁CONR₁₂, NR₁₁CNHNR₁₂, NR₁₂COO, C≡C, or CH═CH;
F independently is selected from the group consisting of O, S, NR₁₂, CR₁₁R₁₂, CONR₁₂, NR₁₁CONR₁₂, NR₁₁CNHNR₁₂, NR₁₂COO, C═C, and CH═CH;
Y is OH, NHR₁₂, SH, COOH, SO₂OH, X, CN, N₃, CONH₂, CONHR₁₂, C≡CH, or CH═CH₂; and
n is an integer between 0 and 5, inclusive.

83. The combinatorial library of claim 69, having the formula 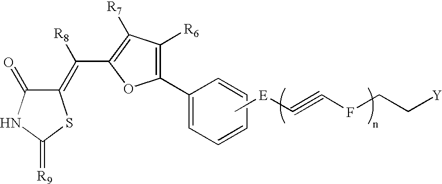
wherein
E is O, S, NR₁₂, CR₁₁C₁₂, CONR₁₂, SO₂NR₁₂, NR₁₁CONR₁₂, NR₁₁CNHNR₁₂, NR₁₂COO, C≡C, or CH═CH;
F independently is selected from the group consisting of O, S, NR₁₂, CR₁₁R₁₂, CONR₁₂, NR₁₁CONR₁₂, NR₁₁CNHNR₁₂, NR₁₂COO, C═C, and CH═CH;
Y is OH, NHR₁₂, SH, COOH, SO₂OH, X, CN, N₃, CONH₂, CONHR₁₂, C≡CH, or CH═CH₂; and
n is an integer between 0 and 5, inclusive.

84. The combinatorial library of claim 69, having the formula 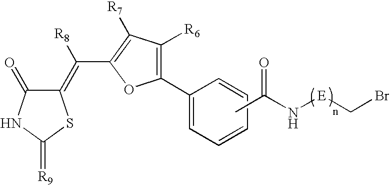
wherein
E is O, S, NR₁₂, CR₁₁C₁₂, CONR₁₂, SO₂NR₁₂, NR₁₁CONR₁₂, NR₁₁CNHNR₁₂, NR₁₂COO, C≡C, or CH═CH; and
n is an integer between 0 and 5, inclusive.

85. The combinatorial library of claim 69, having the formula 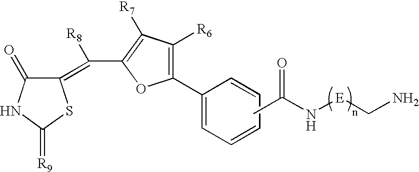
wherein
E is CH₂, CH₂CH₂OCH or CH₂CH₂SCH and n is an integer between 1 and 10, inclusive.

86. The combinatorial library of claim 85, wherein n is greater than 4 and E is CH₂CH₂OCH or CH₂CH₂SCH.

87. The combinatorial library of claim 69, having the formula

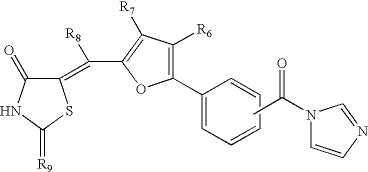

88. A combinatorial library of two or more compounds comprising a common ligand variant of a compound of the formula: 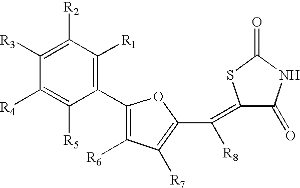
wherein
R₁ to R₈ each independently are selected from the group consisting of H, alkyl, alkenyl, alkynyl, aryl, heterocycle, COOH, COOAlkyl, CONR₁₀R₁₁, C(O)R₁₂, OH, OAlkyl, OAc, SH, SR₁₂, SO₃H, S(O)R₁₂, SO₂NR₁₀R₁₁, S(O)₂R₁₂, NH₂, NHR₁₂, NR₁₀R₁₁, NHCOR₁₂, N₃, NO₂, PH₃, PH₂R₁₂, H₂PO₄, H₂PO₃, H₂PO₂, HPO₄R₁₂, PO₂R₁₁R₁₂, CN, and X;
R₁₀, R₁₁, and R₁₂ each independently are selected from the group consisting of hydrogen, alkyl, alkenyl, alkynyl, aryl, and heterocycle, or R₁₀ and R₁₁ together with the nitrogen to which they are attached can be joined to form a heterocyclic ring.

89. The combinatorial library of claim 88, wherein at least one of R₁ to R₈ is COOH.

90. The combinatorial library of claim 88, wherein at least one of R₁ to R₈ is OH.

91. The combinatorial library of claim 88, wherein at least one of R₁ to R₈ is COOAlkyl.

92. The combinatorial library of claim 88, wherein at least one of R₁ to R₈ is OAlkyl.

93. The combinatorial library of claim 88, wherein two or more of R₁ to R₈ are substituted.

94. The combinatorial library of claim 88, wherein at least one of the compounds is a common ligand variant of a compound having the formula:

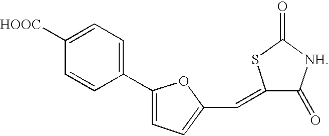

95. The combinatorial library of claim 88, wherein at least one of the compounds is a common ligand variant of a compound having the formula:

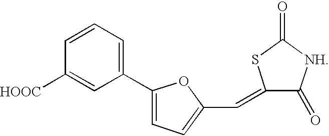

96. The combinatorial library of claim 88, wherein at least one of the compounds is a common ligand variant of a compound having the formula:

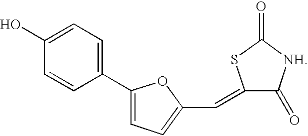

97. The combinatorial library of claim 88, wherein at least one of the compounds is a common ligand variant of a compound having the formula:

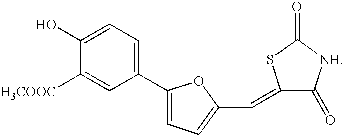

98. The combinatorial library of claim 88, wherein at least one of the compounds is a common ligand variant of a compound having the formula:

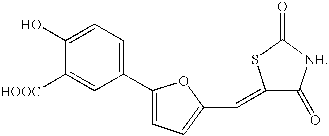

99. The combinatorial library of claim 88, wherein at least one of the compounds is a common ligand variant of a compound having the formula:

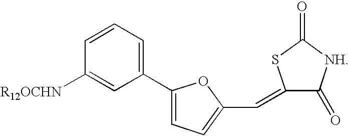

100. The combinatorial library of claim 88, wherein at least one of the compounds is a common ligand variant of a compound having the formula:

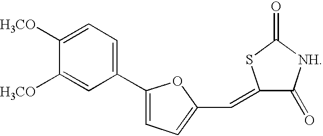

101. The combinatorial library of claim 88, wherein at least one of the compounds is a common ligand variant of a compound having the formula: 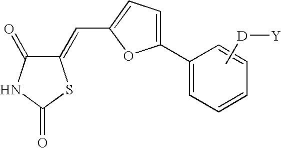
wherein
D is alkylene, alkenylene, alkynylene, aryl, or heterocycle; and
Y is OH, NHR₁₂, SH, COOH, SO₂OH, X, CN, N₃, CONH₂, CONHR₁₂, C≡CH, or CH═CH₂.

102. The combinatorial library of claim 88, wherein at least one of the compounds is a common ligand variant of a compound having the formula: 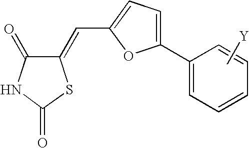
wherein Y is OH, NHR₁₂, SH, COOH, SO₂OH, X, CN, N₃, CONH₂, CONHR₁₂, C≡CH, or CH═CH₂.

103. The combinatorial library of claim 88, wherein at least one of the compounds is a common ligand variant of a compound having the formula: 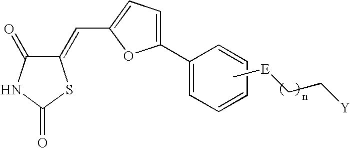
wherein
E is O, S, NR₁₂, CR₁₁C₁₂, CONR₁₂, SO₂NR₁₂, NR₁₁CONR₁₂, NR₁₁CNHNR₁₂, NR₁₂COO, C≡C, or CH═CH;
Y is OH, NHR₁₂, SH, COOH, SO₂OH, X, CN, N₃, CONH₂, CONHR₁₂, C≡CH, or CH═CH₂; and
n is an integer between 0 and 5, inclusive.

104. The combinatorial library of claim 88, wherein at least one of the compounds is a common ligand variant of a compound having the formula: 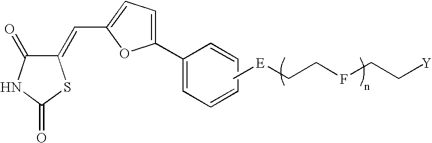
wherein
E and F each independently are selected from the group consisting of O, S, NR₁₂, CR₁₁C₁₂, CONR₁₂, SO₂NR₁₂, NR₁₁CONR₁₂, NR₁₁CNHNR₁₂, NR₁₂COO, C≡C, and CH═CH;
Y is OH, NHR₁₂, SH, COOH, SO₂OH, X, CN, N₃, CONH₂, CONHR₁₂, C≡CH, or CH═CH₂; and
n is an integer between 0 and 5, inclusive.

105. The combinatorial library of claim 88, wherein at least one of the compounds is a common ligand variant of a compound having the formula: 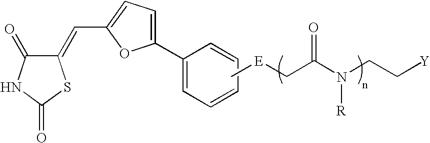
wherein
E is O, S, NR₁₂, CR₁₁C₁₂, CONR₁₂, SO₂NR₁₂, NR₁₁CONR₁₂, NR₁₁CNHNR₁₂, NR₁₂COO, C≡C, or CH═CH;
Y is OH, NHR₁₂, SH, COOH, SO₂OH, X, CN, N₃, CONH₂, CONHR₁₂, C≡CH, or CH═CH₂;
R is hydrogen, alkyl, alkenyl, alkynyl, aryl, or heterocycle; and
n is an integer between 0 and 5, inclusive.

106. The combinatorial library of claim 88, wherein at least one of the compounds is a common ligand variant of a compound having the formula: 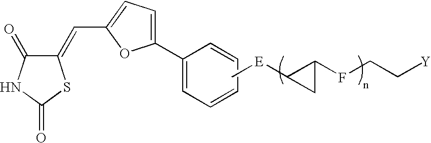
wherein
E and F each independently are selected from the group consisting of O, S, NR₁₂, CR₁₁C₁₂, CONR₁₂, SO₂NR₁₂, NR₁₁CONR₁₂, NR₁₁CNHNR₁₂, NR₁₂COO, C≡C, and CH═CH;
Y is OH, NHR₁₂, SH, COOH, SO₂OH, X, CN, N₃, CONH₂, CONHR₁₂, C≡CH, or CH═CH₂; and
n is an integer between 0 and 5, inclusive.

107. The combinatorial library of claim 88, wherein at least one of the compounds is a common ligand variant of a compound having the formula: 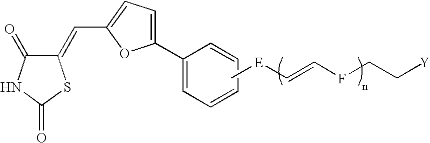
wherein
E is O, S, NR₁₂, CR₁₁C₂, CONR₁₂, SO₂NR₁₂, NR₁₁CONR₁₂, NR₁₁CNHNR₁₂, NR₁₂COO, C≡C, or CH═CH;
F independently is selected from the group consisting of O, S, NR₁₂, CR₁₁R₁₂, CONR₁₂, NR₁₁CONR₁₂, NR₁₁CNHNR₁₂, NR₁₂COO, C═C, and CH═CH;
Y is OH, NHR₁₂, SH, COOH, SO₂OH, X, CN, N₃, CONH₂, CONHR₁₂, C≡CH, or CH═CH₂; and
n is an integer between 0 and 5, inclusive.

108. The combinatorial library of claim 88, wherein at least one of the compounds is a common ligand variant of a compound having the formula: 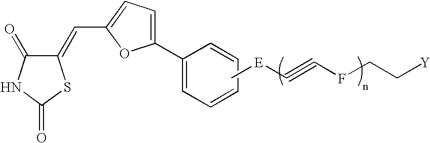
wherein
E is O, S, NR₁₂, CR₁₁C₁₂, CONR₁₂, SO₂NR₁₂, NR₁₁CONR₁₂, NR₁₁CNHNR₁₂, NR₁₂COO, C≡C, or CH═CH;
F independently is selected from the group consisting of O, S, NR₁₂, CR₁₁R₁₂, CONR₁₂, NR₁₁CONR₁₂, NR₁₁CNHNR₁₂, NR₁₂COO, C═C, and CH═CH;
Y is OH, NHR₁₂, SH, COOH, SO₂OH, X, CN, N₃, CONH₂, CONHR₁₂, C≡CH, or CH═CH₂; and
n is an integer between 0 and 5, inclusive.

109. The combinatorial library of claim 88, wherein at least one of the compounds is a common ligand variant of a compound having the formula: 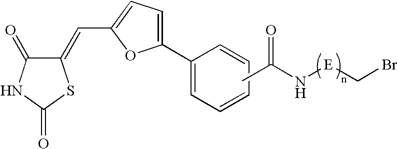
wherein
E is O, S, NR₁₂, CR₁₁C₁₂, CONR₁₂, SO₂NR₁₂, NR₁₁CONR₁₂, NR₁₁CNHNR₁₂, NR₁₂COO, C≡C, or CH═CH; and
n is an integer between 0 and 5, inclusive.

110. The combinatorial library of claim 88, wherein at least one of the compounds is a common ligand variant of a compound having the formula: 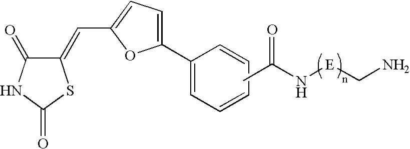
wherein
E is O, S, NR₁₂, CR₁₁C₁₂, CONR₁₂, SO₂NR₁₂, NR₁₁CONR₁₂, NR₁₁CNHNR₁₂, NR₁₂COO, C═C, or CH═CH; and
n is an integer between 0 and 5, inclusive.

111. The combinatorial library of claim 110, wherein n is greater than 4 and E is CH₂CH₂OCH or CH₂CH₂SCH.

112. The combinatorial library of claim 88, wherein at least one of the compounds is a common ligand variant of a compound having the formula:

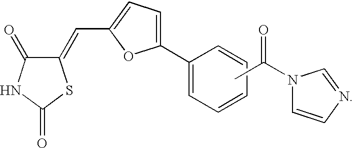

113. A combinatorial library of two or more compounds comprising a common ligand variant of a compound of formula: 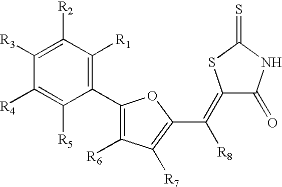
wherein
R₁ to R₈ each independently are selected from the group consisting of H, alkyl, alkenyl, alkynyl, aryl, heterocycle, COOH, COOAlkyl, CONR₁₀R₁₁, C(O)R₁₂, OH, OAlkyl, OAc, SH, SR₁₂, SO₃H, S(O)R₁₂, SO₂NR₁₀R₁₁, S(O)₂R₁₂, NH₂, NHR₁₂, NR₁₀R₁₁, NHCOR₁₂, N₃, NO₂, PH₃, PH₂R₁₂, H₂PO₄, H₂PO₃, H₂PO₂, HPO₄R₁₂, PO₂R₁₁R₁₂, CN, and X;
R₁₀, R₁₁, and R₁₂ each independently are selected from the group consisting of hydrogen, alkyl, alkenyl, alkynyl, aryl, and heterocycle, or R₁₀ and R₁₁ together with the nitrogen to which they are attached can be joined to form a heterocyclic ring.

114. The combinatorial library of claim 113, wherein at least one of R₁ to R₈ is COOH.

115. The combinatorial library of claim 113, wherein at least one of R₁ to R₈ is OH.

116. The combinatorial library of claim 113, wherein at least one of R₁ to R₈ is OAlkyl.

117. The combinatorial library of claim 113, wherein at least one of R₁ to R₈ is COOAlkyl.

118. The combinatorial library of claim 113, wherein at least one of R₁ to R₈ is NHCOR₇.

119. The combinatorial library of claim 113, wherein at least one of the compounds in the library is a common ligand variant of a compound having the formula:

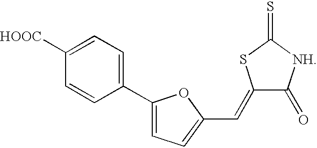

120. The combinatorial library of claim 113, wherein at least one of the compounds in the library is a common ligand variant of a compound having the formula:

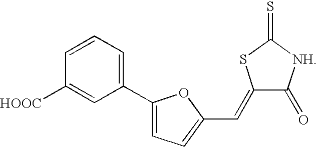

121. The combinatorial library of claim 113, wherein at least one of the compounds in the library is a common ligand variant of a compound having the formula:

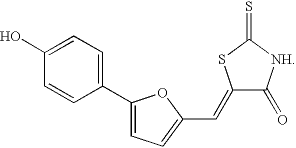

122. The combinatorial library of claim 113, wherein at least one of the compounds in the library is a common ligand variant of a compound having the formula:

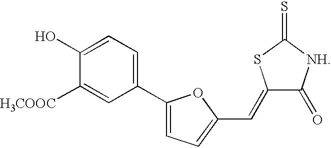

123. The combinatorial library of claim 113, wherein at least one of the compounds in the library is a common ligand variant of a compound having the formula:

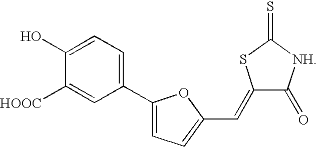

124. The combinatorial library of claim 113, wherein at least one of the compounds in the library is a common ligand variant of a compound having the formula:

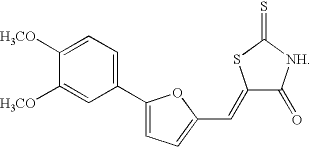

125. The combinatorial library of claim 113, wherein at least one of the compounds in the library is a common ligand variant of a compound having the formula: 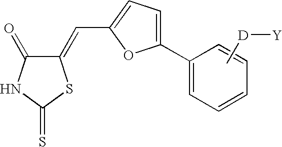
wherein
D is alkylene, alkenylene, alkynylene, aryl, or heterocycle; and
Y is OH, NHR₁₂, SH, COOH, SO₂OH, X, CN, N₃, CONH₂, CONHR₁₂, C≡CH, or CH═CH₂.

126. The combinatorial library of claim 113, wherein at least one of the compounds in the library is a common ligand variant of a compound having the formula: 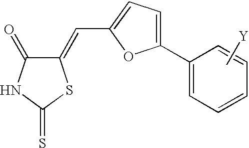
wherein
wherein Y is OH, NHR₁₂, SH, COOH, SO₂OH, X, CN, N₃, CONH₂, CONH₂, C≡CH, or CH═CH₂.

127. The combinatorial library of claim 113, wherein at least one of the compounds in the library is a common ligand variant of a compound having the formula: 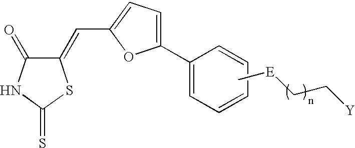
wherein
E is O, S, NR₁₂, CR₁₁C₁₂, CONR₁₂, SO₂NR₁₂, NR₁₁CONR₁₂, NR₁₁CNHNR₁₂, NR₁₂COO, C≡C, or CH═CH;
Y is OH, NHR₁₂, SH, COOH, SO₂OH, X, CN, N₃, CONH₂, CONH₂, C≡CH, or CH═CH₂; and
n is an integer between 0 and 5, inclusive.

128. The combinatorial library of claim 113, wherein at least one of the compounds in the library is a common ligand variant of a compound having the formula: 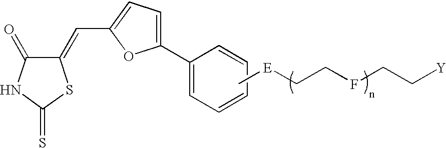
wherein
E and F each independently are selected from the group consisting of O, S, NR₁₂, CR₁₁C₁₂, CONR₁₂, SO₂NR₁₂, NR₁₁CONR₁₂, NR₁₁CNHNR₁₂, NR₁₂COO, C≡C, and CH═CH;
Y is OH, NHR₁₂, SH, COOH, SO₂OH, X, CN, N₃, CONH₂, CONH₂, C≡CH, or CH═CH₂; and
n is an integer between 0 and 5, inclusive.

129. The combinatorial library of claim 113, wherein at least one of the compounds in the library is a common ligand variant of a compound having the formula: 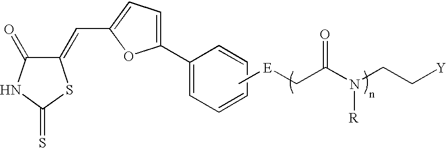
wherein
E is O, S, NR₁₂, CR₁₁C₁₂, CONR₁₂, SO₂NR₁₂, NR₁₁CONR₁₂, NR₁₁CNHNR₁₂, NR₁₂COO, C≡C, or CH═CH;
Y is OH, NHR₁₂, SH, COOH, SO₂OH, X, CN, N₃, CONH₂, CONH₂, C≡CH, or CH═CH₂;
R is hydrogen, alkyl, alkenyl, alkynyl, aryl, or heterocycle; and
n is an integer between 0 and 5, inclusive.

130. The combinatorial library of claim 113, wherein at least one of the compounds in the library is a common ligand variant of a compound having the formula: 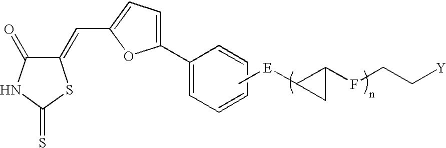
wherein
E and F each independently are selected from the group consisting of O, S, NR₁₂, CR₁₁C₁₂, CONR₁₂, SO₂NR₁₂, NR₁₁CONR₁₂, NR₁₁CNHNR₁₂, NR₁₂COO, C≡C, and CH═CH;
Y is OH, NHR₁₂, SH, COOH, SO₂OH, X, CN, N₃, CONH₂, CONH₂, C≡CH, or CH═CH₂; and
n is an integer between 0 and 5, inclusive.

131. The combinatorial library of claim 113, wherein at least one of the compounds in the library is a common ligand variant of a compound having the formula: 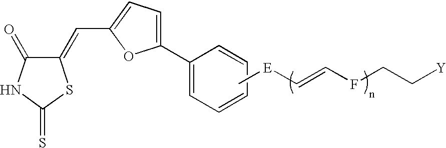
wherein
E is O, S, NR₁₂, CR₁₁C₁₂, CONR₁₂, SO₂NR₁₂, NR₁₁CONR₁₂, NR₁₁CNHNR₁₂, NR₁₂COO, C≡C, or CH═CH;
F independently is selected from the group consisting of O, S, NR₁₂, CR₁₁R₁₂, CONR₁₂, NR₁₁CONR₁₂ NR₁₁CNHNR₁₂, NR₁₂COO, C═C, and CH═CH;
Y is OH, NHR₁₂, SH, COOH, SO₂OH, X, CN, N₃, CONH₂, CONH₂, C≡CH, or CH═CH₂; and
n is an integer between 0 and 5, inclusive.

132. The combinatorial library of claim 113, wherein at least one of the compounds in the library is a common ligand variant of a compound having the formula: 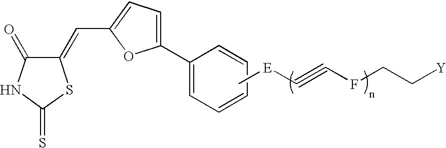
wherein
E is O, S, NR₁₂, CR₁₁C₁₂, CONR₁₂, SO₂NR₁₂, NR₁₁CONR₁₂, NR₁₁CNHNR₁₂, NR₁₂COO, C≡C, or CH═CH;
F independently is selected from the group consisting of O, S, NR₁₂, CR₁₁R₁₂, CONR₁₂, NR₁₁CONR₁₂, NR₁₁CNHNR₁₂, NR₁₂COO, C═C, and CH═CH;
Y is OH, NHR₁₂, SH, COOH, SO₂OH, X, CN, N₃, CONH₂, CONH₂, C≡CH, or CH═CH₂; and
n is an integer between 0 and 5, inclusive.

133. The combinatorial library of claim 113, wherein at least one of the compounds in the library is a common ligand variant of a compound having the formula: 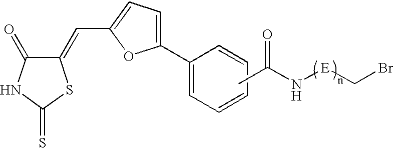
wherein
E is O, S, NR₁₂, CR₁₁C₁₂, CONR₁₂, SO₂NR₁₂, NR₁₁CONR₁₂, NR₁₁CNHNR₁₂, NR₁₂COO, C≡C, or CH═CH; and
n is an integer between 0 and 5, inclusive.

134. The combinatorial library of claim 113, wherein at least one of the compounds in the library is a common ligand variant of a compound having the formula: 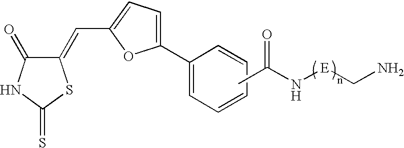
wherein
E is CH₂, CH₂CH₂OCH or CH₂CH₂SCH and n is an integer between 1 and 10, inclusive.

135. The combinatorial library of claim 134, wherein n is greater than 4 and E is CH₂CH₂OCH or CH₂CH₂SCH.

136. The combinatorial library of claim 113, wherein at least one of the compounds in the library is a common ligand variant of a compound having the formula:

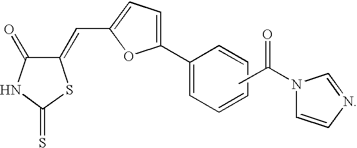

137. A combinatorial library of two or more bi-ligands comprising the reaction product of a specificity ligand and a common ligand mimic having the formula: 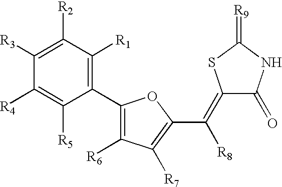
wherein
R₁ to R₈ each independently are selected from the group consisting of H, alkyl, alkenyl, alkynyl, aryl, heterocycle, COOH, COOAlkyl, CONR₁₀R₁₁, C(O)R₁₂, OH, OAlkyl, OAc, SH, SR₁₂, SO₃H, S(O)R₁₂, SO₂NR₁₀R₁₁, S(O)₂R₁₂, NH₂, NHR₁₂, NR₁₀R₁₁, NHCOR₁₂, N₃, NO₂, PH₃, PH₂R₁₂, H₂PO₄, H₂PO₃, H₂PO₂, HPO₄R₁₂, PO₂R₁₁R₁₂, CN, and X;
R₉ is O, S, or NR₁₂; and
R₁₀ R₁₁, and R₁₂ each independently are selected from the group consisting of hydrogen, alkyl, alkenyl, alkynyl, aryl, and heterocycle, or R₁₀ and R₁₁ together with the nitrogen to which they are attached can be joined to form a heterocyclic ring.

138. The combinatorial library of claim 137, wherein at least one of R₁ to R₈ is COOH.

139. The combinatorial library of claim 137, wherein at least one of R₁ to R₈ is OH.

140. The combinatorial library of claim 137, wherein at least one of R₁ to R₈ is OAlkyl.

141. The combinatorial library of claim 137, wherein at least one of R₁ to R₈ is COOAlkyl.

142. The combinatorial library of claim 137, wherein at least one of R₁ to R₈ is NHCOR₇.

143. The combinatorial library of claim 137, wherein two or more of R₁ to R₈ are substituted.

144. The combinatorial library of claim 137, wherein at least one of the compounds is a common ligand variant of a compound having the formula: 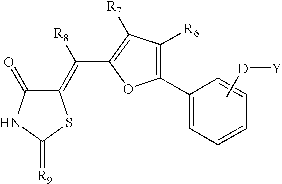
wherein
D is alkylene, alkenylene, alkynylene, aryl, or heterocycle; and
Y is OH, NHR₁₂, SH, COOH, SO₂OH, X, CN, N₃, CONH₂, CONHR₁₂, C═CH, or CH═CH₂.

145. The combinatorial library of claim 137, wherein at least one of the compounds is a common ligand variant of a compound having the formula: 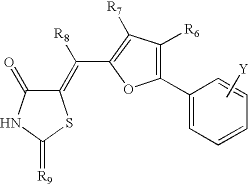
wherein Y is OH, NHR₁₂, SH, COOH, SO₂OH, X, CN, N₃, CONH₂,CONHR₁₂, C≡CH, or CH═CH₂.

146. The combinatorial library of claim 137, wherein at least one of the compounds is a common ligand variant of a compound having the formula: 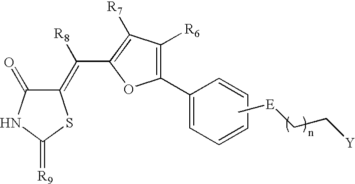
wherein
E is O, S, NR₁₂, CR₁₁C₁₂, CONR₁₂, SO₂NR₁₂, NR₁₁CONR₁₂, NR₁₁CNHNR₁₂, NR₁₂COO, C≡C, or CH═CH;
Y is OH, NHR₁₂, SH, COOH, SO₂OH, X, CN, N₃, CONH₂, CONHR₁₂, C≡CH, or CH═CH₂; and
n is an integer between 0 and 5, inclusive.

147. The combinatorial library of claim 137, wherein at least one of the compounds is a common ligand variant of a compound having the formula: 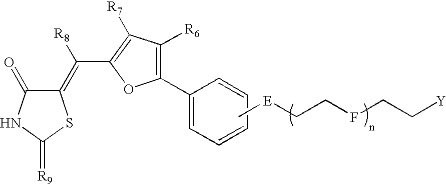
wherein
E and F each independently are selected from the group consisting of O, S, NR₁₂, CR₁₁C₁₂, CONR₁₂, SO₂NR₁₂, NR₁₁CONR₁₂, NR₁₁CNHNR₁₂, NR₁₂COO, C≡C, and CH═CH;
Y is OH, NHR₁₂, SH, COOH, SO₂OH, X, CN, N₃, CONH₂, CONHR₁₂, C≡CH, or CH═CH₂; and
n is an integer between 0 and 5, inclusive.

148. The combinatorial library of claim 137, wherein at least one of the compounds is a common ligand variant of a compound having the formula: 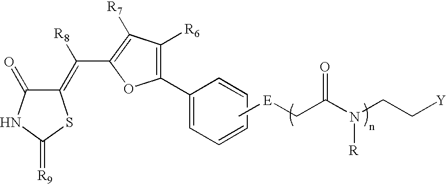
wherein
E is O, S, NR₁₂, CR₁₁C₁₂, CONR₁₂, SO₂NR₁₂, NR₁₁CONR₁₂, NR₁₁CNHNR₁₂, NR₁₂COO, C≡C, or CH═CH;
Y is OH, NHR₁₂, SH, COOH, SO₂OH, X, CN, N₃, CONH₂, CONHR₁₂, C≡CH, or CH═CH₂;
R is hydrogen, alkyl, alkenyl, alkynyl, aryl, or heterocycle; and
n is an integer between 0 and 5, inclusive.

149. The combinatorial library of claim 137, wherein at least one of the compounds is a common ligand variant of a compound having the formula: 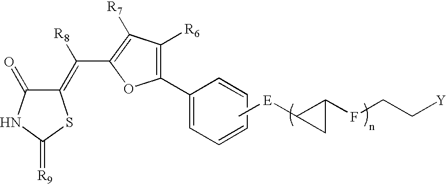
wherein
E and F each independently are selected from the group consisting of O, S, NR₁₂, CR₁₁C₁₂, CONR₁₂, SO₂NR₁₂, NR₁₁CONR₁₂, NR₁₁CNHNR₁₂, NR₁₂COO, C≡C, and CH═CH;
Y is OH, NHR₁₂, SH, COOH, SO₂OH, X, CN, N₃, CONH₂, CONHR₁₂, C≡CH, or CH═CH₂; and
n is an integer between 0 and 5, inclusive.

150. The combinatorial library of claim 137, wherein at least one of the compounds is a common ligand variant of a compound having the formula: 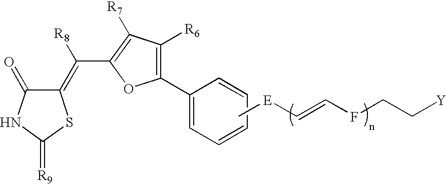
wherein
E is O, S, NR₁₂, CR₁₁C₁₂, CONR₁₂, SO₂NR₁₂, NR₁₁CONR₁₂, NR₁₁CNHNR₁₂, NR₁₂COO, C≡C, or CH═CH;
F independently is selected from the group consisting of O, S, NR₁₂, CR₁₁R₁₂, CONR₁₂, NR₁₁CONR₁₂, NR₁₁CNHNR₁₂, NR₁₂COO, C═C, and CH═CH;
Y is OH, NHR₁₂, SH, COOH, SO₂OH, X, CN, N₃, CONH₂, CONHR₁₂, C≡CH, or CH═CH₂; and
n is an integer between 0 and 5, inclusive.

151. The combinatorial library of claim 137, wherein at least one of the compounds is a common ligand variant of a compound having the formula: 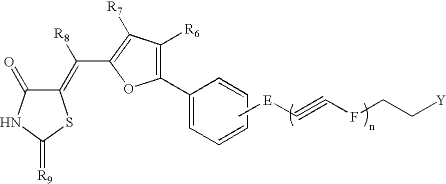
wherein
E is O, S, NR₁₂, CR₁₁C₁₂, CONR₁₂, SO₂NR₁₂, NR₁₁CONR₁₂, NR₁₁CNHNR₁₂, NR₁₂COO, C≡C, or CH═CH;
F independently is selected from the group consisting of O, S, NR₁₂, CR₁₁R₁₂, CONR₁₂, NR₁₁CONR₁₂, NR₁₁CNHNR₁₂, NR₁₂COO, C═C, and CH═CH;
Y is OH, NHR₁₂, SH, COOH, SO₂OH, X, CN, N₃, CONH₂, CONHR₁₂, C≡CH, or CH═CH₂; and
n is an integer between 0 and 5, inclusive.

152. The combinatorial library of claim 137, wherein at least one of the compounds is a common ligand variant of a compound having the formula: 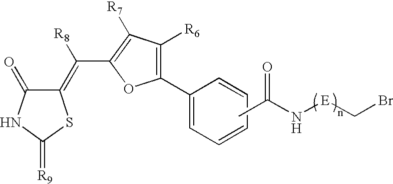
wherein
E is O, S, NR₁₂, CR₁₁C₁₂, CONR₁₂, SO₂NR₁₂, NR₁₁CONR₁₂, NR₁₁CNHNR₁₂, NR₁₂COO, C≡C, or CH═CH; and
n is an integer between 0 and 5, inclusive.

153. The combinatorial library of claim 137, wherein at least one of the compounds is a common ligand variant of a compound having the formula: 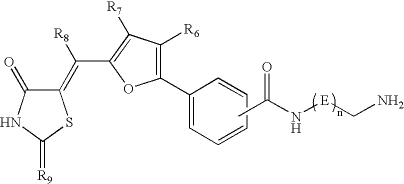
wherein
E is CH₂, CH₂CH₂OCH or CH₂CH₂SCH and n is an integer between 1 and 10, inclusive.

154. The combinatorial library of claim 153, wherein n is greater than 4 and E is CH₂CH₂OCH or CH₂CH₂SCH.

155. The combinatorial library of claim 137, wherein at least one of the compounds is a common ligand variant of a compound having the formula:

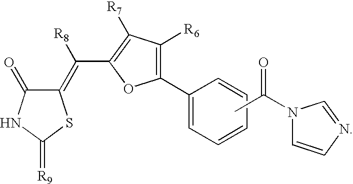

156. A combinatorial library of two or more bi-ligands comprising the reaction product of a specificity ligand and a common ligand mimic having the formula: 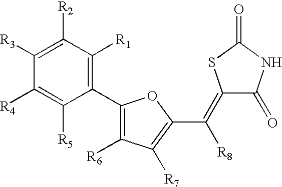
wherein
R₁ to R₈ each independently are selected from the group consisting of H, alkyl, alkenyl, alkynyl, aryl, heterocycle, COOH, COOAlkyl, CONR₁₀R₁₁, C(O)R₁₂, OH, OAlkyl, OAc, SH, SR₁₂, SO₃H, S(O)R₁₂, SO₂NR₁₀R₁₁, S(O)₂R₁₂, NH₂, NHR₁₂, NR₁₀R₁₁, NHCOR₁₂, N₃, NO₂, PH₃, PH₂R₁₂, H₂PO₄, H₂PO₃, H₂PO₂, HPO₄R₁₂, PO₂R₁₁R₁₂, CN, and X;
R₁₀, R₁₁, and R₁₂ each independently are selected from the group consisting of hydrogen, alkyl, alkenyl, alkynyl, aryl, and heterocycle, or R₁₀ and R₁₁ together with the nitrogen to which they are attached can be joined to form a heterocyclic ring.

157. The combinatorial library of claim 156, wherein at least one of R₁ to R₈ COOH.

158. The combinatorial library of claim 156, wherein at least one of R₁ to R₈ is OH.

159. The combinatorial library of claim 156, wherein at least one of R₁ to R₈ is COOAlkyl.

160. The combinatorial library of claim 156, wherein at least one of R₁ to R₈ is OAlkyl.

161. The combinatorial library of claim 156, wherein two or more of R₁ to R₈ are substituted.

162. The combinatorial library of claim 156, wherein the common ligand mimic comprises a compound of the formula:

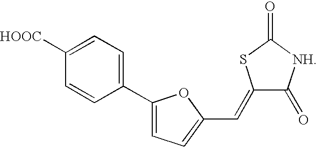

163. The combinatorial library of claim 156, wherein the common ligand mimic comprises a compound of the formula:

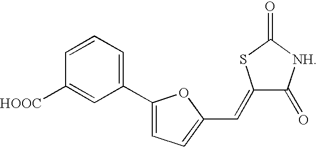

164. The combinatorial library of claim 156, wherein the common ligand mimic comprises a compound of the formula:

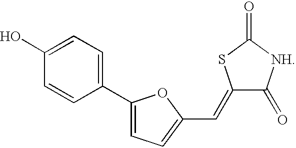

165. The combinatorial library of claim 156, wherein the common ligand mimic comprises a compound of the formula:

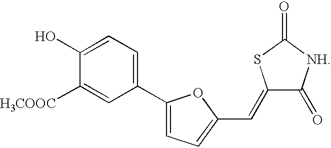

166. The combinatorial library of claim 156, wherein the common ligand mimic comprises a compound of the formula:

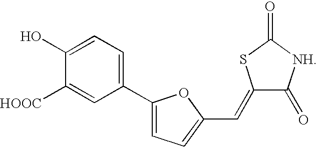

167. The combinatorial library of claim 156, wherein the common ligand mimic comprises a compound of the formula:

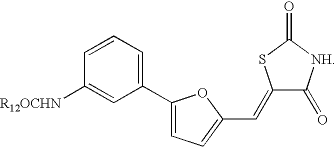

168. The combinatorial library of claim 156, wherein the common ligand mimic comprises a compound of the formula:

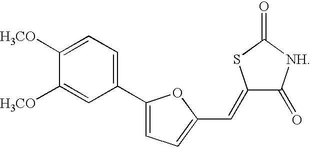

169. The combinatorial library of claim 156, wherein at least one of the compounds is a common ligand variant of a compound having the formula: 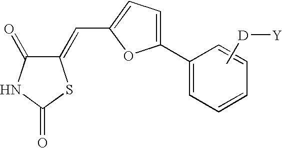
wherein
D is alkylene, alkenylene, alkynylene, aryl, or heterocycle; and
Y is OH, NHR₁₂, SH, COOH, SO₂OH, X, CN, N₃, CONH₂, CONHR₁₂, C≡CH, or CH═CH₂.

170. The combinatorial library of claim 156, wherein at least one of the compounds is a common ligand variant of a compound having the formula: 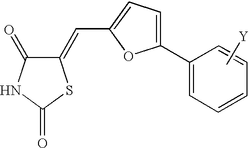
wherein Y is OH, NHR₁₂, SH, COOH, SO₂OH, X, CN, N₃, CONH₂, CONHR₁₂, C≡CH, or CH═CH₂.

171. The combinatorial library of claim 156, wherein at least one of the compounds is a common ligand variant of a compound having the formula: 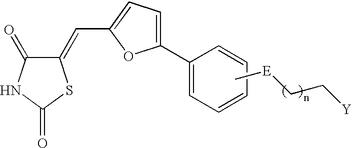
wherein
E is O, S, NR₁₂, CR₁₁C₁₂, CONR₁₂, SO₂NR₁₂, NR₁₁CONR₁₂, NR₁₁CNHNR₁₂, NR₁₂COO, C≡C, or CH═CH;
Y is OH, NHR₁₂, SH, COOH, SO₂OH, X, CN, N₃, CONH₂, CONHR₁₂, C≡CH, or CH═CH₂; and
n is an integer between 0 and 5, inclusive.

172. The combinatorial library of claim 156, wherein at least one of the compounds is a common ligand variant of a compound having the formula: 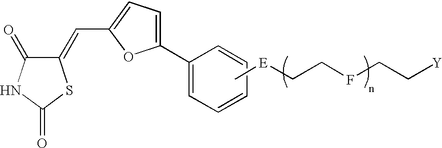
wherein
E and F each independently are selected from the group consisting of O, S, NR₁₂, CR₁₁C₁₂, CONR₁₂, SO₂NR₁₂, NR₁₁CONR₁₂, NR₁₁CNHNR₁₂, NR₁₂COO, C≡C, and CH═CH;
Y is OH, NHR₁₂, SH, COOH, SO₂OH, X, CN, N₃, CONH₂, CONHR₁₂, C≡CH, or CH═CH₂; and
n is an integer between 0 and 5, inclusive.

173. The combinatorial library of claim 156, wherein at least one of the compounds is a common ligand variant of a compound having the formula: 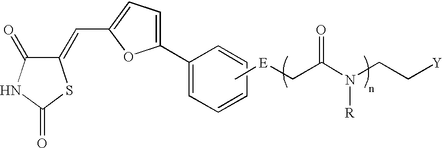
wherein
E and F each independently are selected from the group consisting of O, S, NR₁₂, CR₁₁C₁₂, CONR₁₂, SO₂NR₁₂, NR₁₁CONR₁₂, NR₁₁CNHNR₁₂, NR₁₂COO, C≡C, and CH═CH;
R is hydrogen, alkyl, alkenyl, alkynyl, aryl, or heterocycle; and
n is an integer between 0 and 5, inclusive.

174. The combinatorial library of claim 156, wherein at least one of the compounds is a common ligand variant of a compound having the formula: 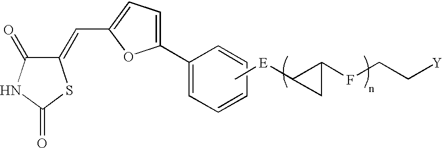
wherein
E and F each independently are selected from the group consisting of O, S, NR₁₂, CR₁₁C₁₂, CONR₁₂, SO₂NR₁₂, NR₁₁CONR₁₂, NR₁₁CNHNR₁₂, NR₁₂COO, C≡C, and CH═CH;
Y is OH, NHR₁₂, SH, COOH, SO₂OH, X, CN, N₃, CONH₂, CONHR₁₂, C≡CH, or CH═CH₂; and
n is an integer between 0 and 5, inclusive.

175. The combinatorial library of claim 156, wherein at least one of the compounds is a common ligand variant of a compound having the formula: 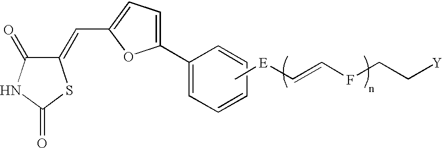
wherein
E is O, S, NR₁₂, CR₁₁C₁₂, CONR₁₂, SO₂NR₁₂, NR₁₁CONR₁₂, NR₁₁CNHNR₁₂, NR₁₂COO, C≡C, or CH═CH;
F independently is selected from the group consisting of O, S, NR₁₂, CR₁₁R₁₂, CONR₁₂, NR₁₁CONR₁₂, NR₁₁CNHNR₁₂, NR₁₂COO, C═C, and CH═CH;
Y is OH, NHR₁₂, SH, COOH, SO₂OH, X, CN, N₃, CONH₂, CONHR₁₂, C≡CH, or CH═CH₂; and
n is an integer between 0 and 5, inclusive.

176. The combinatorial library of claim 156, wherein at least one of the compounds is a common ligand variant of a compound having the formula: 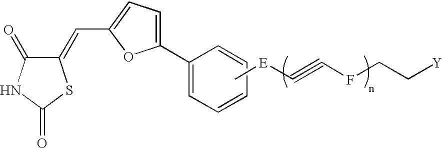
wherein
E is O, S, NR₁₂, CR₁₁C₁₂, CONR₁₂, SO₂NR₁₂, NR₁₁CONR₁₂, NR₁₁CNHNR₁₂, NR₁₂COO, C≡C, or CH═CH;
F independently is selected from the group consisting of O, S, NR₁₂, CR₁₁R₁₂, CONR₁₂, NR₁₁CONR₁₂, NR₁₁CNHNR₁₂, NR₁₂COO, C═C, and CH═CH;
Y is OH, NHR₁₂, SH, COOH, SO₂OH, X, CN, N₃, CONH₂, CONHR₁₂, C≡CH, or CH═CH₂; and
n is an integer between 0 and 5, inclusive.

177. The combinatorial library of claim 156, wherein at least one of the compounds is a common ligand variant of a compound having the formula: 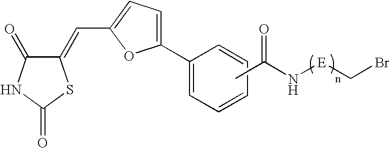
wherein
E is O, S, NR₁₂, CR₁₁C₁₂, CONR₁₂, SO₂NR₁₂, NR₁₁CONR₁₂, NR₁₁CNHNR₁₂, NR₁₂COO, C≡C, and CH═CH; and
n is an integer between 0 and 5, inclusive.

178. The combinatorial library of claim 156, wherein at least one of the compounds is a common ligand variant of a compound having the formula: 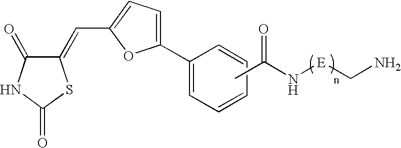
wherein
E is O, S, NR₁₂, CR₁₁C₁₂, CONR₁₂, SO₂NR₁₂, NR₁₁CONR₁₂, NR₁₁CNHNR₁₂, NR₁₂COO, C≡C, or CH═CH;
F independently is selected from the group consisting of O, S, NR₁₂, CR₁₁R₁₂, CONR₁₂, NR₁₁CONR₁₂, NR₁₂COO, C═C, and CH═CH;
Y is OH, NHR₁₂, SH, COOH, SO₂OH, X, CN, N₃, CONH₂, CONHR₁₂, C≡CH, or CH═CH₂; and
n is an integer between 0 and 5, inclusive.

179. The combinatorial library of claim 178, wherein n is greater than 4 and E is CH₂CH₂OCH or CH₂CH₂SCH.

180. The combinatorial library of claim 156, wherein at least one of the compounds is a common ligand variant of a compound having the formula:

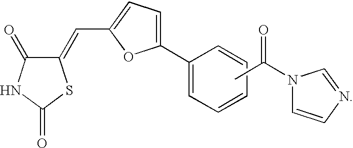

181. A combinatorial library of two or more bi-ligands comprising the reaction product of a specificity ligand and a common ligand mimic having the formula: 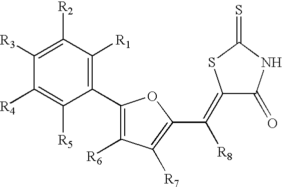
wherein
R₁ to R₈ each independently are selected from the group consisting of H, alkyl, alkenyl, alkynyl, aryl, heterocycle, COOH, COOAlkyl, CONR₁₀R₁₁, C(O)R₁₂, OH, OAlkyl, OAc, SH, SR₁₂, SO₃H, S(O)R₁₂, SO₂NR₁₀R₁₁, S(O)₂R₁₂, NH₂, NHR₁₂, NR₁₀R₁₁, NHCOR₁₂, N₃, NO₂, PH₃, PH₂R₁₂, H₂PO₄, H₂PO₃, H₂PO₂, HPO₄R₁₂, PO₂R₁₁R₁₂, CN, and X;
R₁₀, R₁₁, and R₁₂ each independently are selected from the group consisting of hydrogen, alkyl, alkenyl, alkynyl, aryl, and heterocycle, or R₁₀ and R₁₁, together with the nitrogen to which they are attached can be joined to form a heterocyclic ring.

182. The combinatorial library of claim 181, wherein at least one of R₁ to R₈ is COOH.

183. The combinatorial library of claim 181, wherein at least one of R₁ to R₈ is OH.

184. The combinatorial library of claim 181, wherein at least one of R₁ to R₈ is OAlkyl.

185. The combinatorial library of claim 181, wherein at least one of R₁ to R₈ is COOAlkyl.

186. The combinatorial library of claim 181, wherein at least one of R₁ to R₈ is NHCOR₇.

187. The combinatorial library of claim 181, wherein the common ligand mimic comprises a compound of the formula:

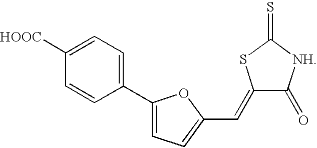

188. The combinatorial library of claim 181, wherein the common ligand mimic comprises a compound of the formula:

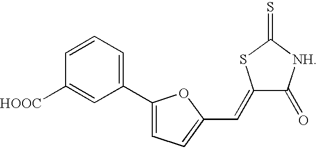

189. The combinatorial library of claim 181, wherein the common ligand mimic comprises a compound of the formula:

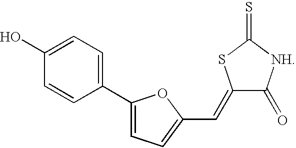

190. The combinatorial library of claim 181, wherein the common ligand mimic comprises a compound of the formula:

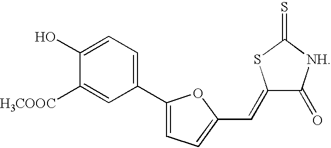

191. The combinatorial library of claim 181, wherein the common ligand mimic comprises a compound of the formula:

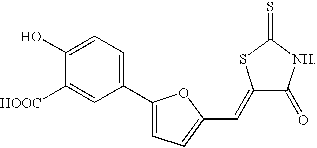

192. The combinatorial library of claim 181, wherein the common ligand mimic comprises a compound of the formula:

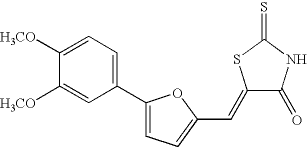

193. The combinatorial library of claim 181, wherein at least one of the compounds in the library is a common ligand variant of a compound having the formula: 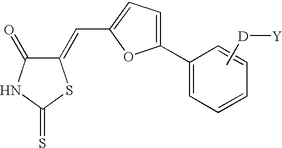
wherein
D is alkylene, alenylene, alkynylene, aryl, or heterocycle; and
Y is OH, NHR₁₂, SH, COOH, SO₂OH, X, CN, N₃, CONH₂, CONHR₁₂, C≡CH, or CH≡CH₂.

194. The combinatorial library of claim 181, wherein at least one of the compounds in the library is a common ligand variant of a compound having the formula: 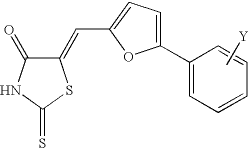
wherein
wherein Y is OH, NHR₁₂, SH, COOH, SO₂OH, X, CN, N₃, CONH₂, CONHR₁₂, C≡CH, or CH═CH₂.

195. The combinatorial library of claim 181, wherein at least one of the compounds in the library is a common ligand variant of a compound having the formula: 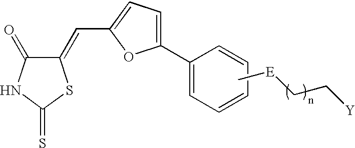
wherein
E is O, S, NR₁₂, CR₁₁C₁₂, CONR₁₂, SO₂NR₁₂, NR₁₁CONR₁₂, NR₁₁CNHNR₁₂, NR₁₂COO, C≡C, or CH═CH;
Y is OH, NHR₁₂, SH, COOH, SO₂OH, X, CN, N₃, CONH₂, CONHR₁₂, C≡CH, or CH═CH₂; and
n is an integer between 0 and 5, inclusive.

196. The combinatorial library of claim 181, wherein at least one of the compounds in the library is a common ligand variant of a compound having the formula: 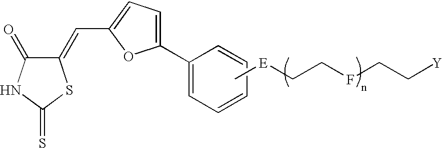
wherein
E and F each independently are selected from the group consisting of O, S, NR₁₂, CR₁₁C₁₂, CONR₁₂, SO₂NR₁₂, NR₁₁CONR₁₂, NR₁₁CNHNR₁₂, NR₁₂COO, C≡C, and CH═CH;
Y is OH, NHR₁₂, SH, COOH, SO₂OH, X, CN, N₃, CONH₂, CONHR₁₂, C≡CH, or CH═CH₂; and
n is an integer between 0 and 5, inclusive.

197. The combinatorial library of claim 181, wherein at least one of the compounds in the library is a common ligand variant of a compound having the formula: 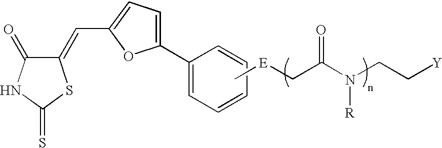
wherein
E is O, S, NR₁₂, CR₁₁C₁₂, CONR₁₂, SO₂NR₁₂, NR₁₁CONR₁₂, NR₁₁CNHNR₁₂, NR₁₂COO, C≡C, or CH═CH;
Y is OH, NHR₁₂, SH, COOH, SO₂OH, X, CN, N₃, CONH₂, CONHR₁₂, C≡CH, or CH═CH₂;
R is hydrogen, alkyl, alkenyl, alkynyl, aryl, or heterocycle; and
n is an integer between 0 and 5, inclusive.

198. The combinatorial library of claim 181, wherein at least one of the compounds in the library is a common ligand variant of a compound having the formula: 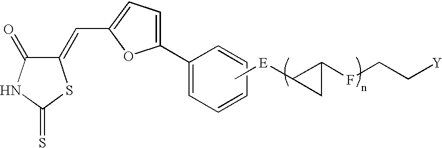
wherein
E and F each independently are selected from the group consisting of O, S, NR₁₂, CR₁₁C₁₂, CONR₁₂, SO₂NR₁₂, NR₁₁CONR₁₂, NR₁₁CNHNR₁₂, NR₁₂COO, C≡C, and CH═CH;
Y is OH, NHR₁₂, SH, COOH, SO₂OH, X, CN, N₃, CONH₂, CONHR₁₂, C≡CH, or CH═CH₂; and
n is an integer between 0 and 5, inclusive.

199. The combinatorial library of claim 181, wherein at least one of the compounds in the library is a common ligand variant of a compound having the formula: 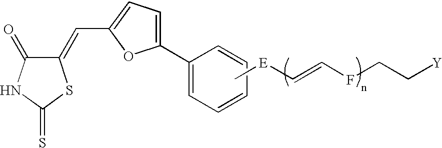
wherein
E is O, S, NR₁₂, CR₁₁C₁₂, CONR₁₂, SO₂NR₁₂, NR₁₁CONR₁₂, NR₁₁CNHNR₁₂, NR₁₂COO, C≡C, or CH═CH;
F independently is selected from the group consisting of O, S, NR₁₂, CR₁₁R₁₂, CONR₁₂, NR₁₁CONR₁₂, NR₁₁CNHNR₁₂, NR₁₂COO, C═C, and CH═CH;
Y is OH, NHR₁₂, SH, COOH, SO₂OH, X, CN, N₃, CONH₂, CONHR₁₂, C≡CH, or CH═CH₂; and
n is an integer between 0 and 5, inclusive.

200. The combinatorial library of claim 181, wherein at least one of the compounds in the library is a common ligand variant of a compound having the formula: 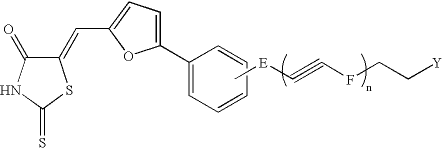
wherein
E is O, S, NR₁₂, CR₁₁C₁₂, CONR₁₂, SO₂NR₁₂, NR₁₁CONR₁₂, NR₁₁CNHNR₁₂, NR₁₂COO, C≡C, or CH═CH;
F independently is selected from the group consisting of O, S, NR₁₂, CR₁₁R₁₂, CONR₁₂, NR₁₁CONR₁₂, NR₁₁CNHNR₁₂, NR₁₂COO, C═C, and CH═CH;
Y is OH, NHR₁₂, SH, COOH, SO₂OH, X, CN, N₃, CONH₂, CONHR₁₂, C≡CH, or CH═CH₂; and
n is an integer between 0 and 5, inclusive.

201. The combinatorial library of claim 181, wherein at least one of the compounds in the library is a common ligand variant of a compound having the formula: 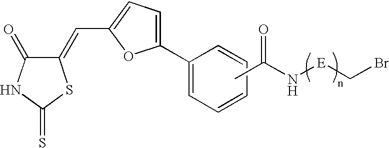
wherein
E is O, S, NR₁₂, CR₁₁C₁₂, CONR₁₂, SO₂NR₁₂, NR₁₁CONR₁₂, NR₁₁CNHNR₁₂, NR₁₂COO, C≡C, or CH═CH; and
n is an integer between 0 and 5, inclusive.

202. The combinatorial library of claim 181, wherein at least one of the compounds in the library is a common ligand variant of a compound having the formula: 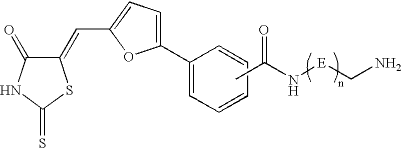
wherein
E is CH₂, CH₂CH₂OCH or CH₂CH₂SCH and n is an integer between 1 and 10, inclusive.

203. The combinatorial library of claim 202, wherein n is greater than 4 and E is CH₂CH₂OCH or CH₂CH₂SCH.

204. The combinatorial library of claim 181, wherein at least one of the compounds in the library is a common ligand variant of a compound having the formula: [chemical structure]
